SEQUENCE LISTING (38 sequences)

SEQ ID NO: 1 - DNA, 189415 bp, synthetic construct
  source 1..189415: mol_type = other DNA
gagaaagagataaaacttttttacgactccatcagaaagaggtttaatatttttgtgagaccatcgaagagagaaagagaaagagatagttagtctagatatttttcttagtacaaaagtcaatgttttaaaatatatggacaagaatttgtctgtataaaaacttgtgtgaaattttgtaccaaagaaaaaatgtgagcagtatcccctacatggattttactagatcatttatataccaaaaaatattatacgatctacgttttattatatgattttaacgtgtaaattataaacattattttatgatatacaattgtctggtaacctagatgggcataggggatgttgataagctcgacgagtatatgttgttggacgttattgtttaagaaatagttgatgcatcagaaagagaataaaaaatattttagtgagaccatcgaagagagaaagagataaaacttttttacgactccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatatttttgtgagaccatcgaagagagaaagagaataaaaatattttatgactccattgaagagagaaagagaaaatgagaatgagaataaaaatattttagtgacaccatcagaaagaggtttaatattttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaatatttttgtaaaactttttttatgagaccatcaaagagagaaagagaataaaaaaatatttttgtaaaactttttttatgagaccatcagaaagaggtttaatatttttgtgataccctgaaaggaaataggaataggaatagtgtcataatcgtatcacactattgagacagaaaaagaagaagtcgcgagaggtaactttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttccatcagcggattctgagatggatttaatctgaggacatttggtgaatccaaagttcattctcagacctccaccgatgatggagtaataagtggtaggaggatctacatcctcgactgattccacctcgggatctggatctgactcggactctgtaatttccgttacggattggcaaatcttatcatcggtcggtgtttggtcttgctttgtgactttgataataacatcgattcccatatgatgtttgttttcttcttccgtacacgatgaggatgattgctgaagactggcaggcacatgcatgccagtacgatatattgtttcatgattgctattgattgagtactgttctttatgattctacttccttaccgtgcaataaattagaatatattttctacttttacgagaaattaattattgtatttatgggtgaaaaacttactataaaaagcgggtgggtttggaattagtgatcagtttatgtatatcgcaactaccgggcatatggctacattacccacatgataagagattgtatcagtttcgtagtcttgagtattggtattactatatagtatatagatgtcgacgctagagttactgtctccgaatgcggcatgatagtatcattctttgctttcgttaactgtttggaggaagaatctttgttattgcatttaatctcgaaattcagagtgcacacctttctcctgtaaagaatcctgaagttgctaccttattaagaacggagaagtatccatcacgaaagacgggattacagtctttatgattcatagtaatagttagttccgacgttgagatggattcgctgagaccggtagtggtcgtccgagtacacgatgtgtcgttgacgggatacagattaatttccacatcgatatagttaaaggtatttctgggtacgggtttgagatcgtcgtacatgggaaatgaaatgtgactgtctgaatgtatggctttaagatagctgtgataccgtatacaggtcggtgtcggagattcgaatctctttaaggcgacttatgtcacgatgatggaatctatcttatcgaatgatatatttttcataaatacacttttatagtcctcgtttaaacagaatttactatgtagttccgcgaatgactcgtcccttaataggcagtaggctagtatcttttttacgtagtaatcgtcgtagggagagacatcttgtagaacaacgatttaatcataggtagagatactttcagtctgtggtggatgatgtcattcacaacatccgccttgtatatgatgtttctgttttcaaacaccaagtcgaataccgtctttagtcggaaggttgatgtcgtatccgatgtatgaggcaacattgttgttacaattttgaaaggcggtattatagtattcgtctttctgaatgtcgaacctatctagtagataccgtagtatattgagagtgtatccttgattatgttttatgaatagataaagtagatgttgtccttcttccttttgttcgtgccaattgagtaacattatgagaatatgacctgttgcacaatcgttccatgatgggtgtacaatcaagattattacgtatcctcgtatcggctcctcgagataaaagagcatacaccacacgaggactatgtttggtatactgttgaaggtaagtgtgtaaccgcgttaatgtttgctccataatctattatcgcgtagatgaatcgcttctcggctcgcatcttagtgtgacttaacttgtaataattgcttttgtagaacgtggatatgtgtttacagtagtaatgaagagaagtgagtccatcctcgtcgacgcaattagggtcggatcctttgtacagaacgtaatagtttaagctcccattgaatttatatctaagataacacagcaatagatcggatgatttactaaagtcatcaatggtgtccgttagtatatcaaagatcttgttatcgattgatagtgaatgaatcagatagtggtgtagaggaatatgtcctttttcatccttgctatcaaagttacgcatgccgtggtgtaacaatatctttaatacagatggattaaatcgtgtattcatcgtatagcaatgtaatggagagttacctcgtttattcagatcgcagtgtttaataactagcttaaacagatgagacgatgtatccacatcaaagaacgtaaaatacatatgacaaacattgttgacagaaacgtgaccttcattcttaccgtcgtccataaatacgttaggtatgtaccacatactgtcgcgaacgatgcgtacaatctcgtccatctcataatgatttactttttcataattaaagatgtgaaagaaaaacagaacaatatatttttttagtaatgtttatgcgagacatataaaataaactccgtgtttatgatcatttttaacagcaacacattcaatattgtattgttatttttatattatttacacaattaacaatatattattagtttatattactgaattaataatataaaattcccaatcttgtcataaacacacactgagaaacagcataaacacaaaatccatcaaaaatgtcgatgaaatatctgatgttgttgttcgctgctatgataatcagatcattcgccgatagtggtaacgctatcgaaacgacatcgccagaaattacaaacgctacaacagatattccagctatcagattatgcggtccagagggagatggatattgtttacacggtgactgtatccacgctagagatattgacggtatgtattgtagatgctctcatggttatacaggcattagatgtcagcatgtagtattagtagactatcaacgttcagaaaacccaaacactacaacgtcatatatcccatctcccggtattatgcttgtattagtaggcattattattattacgtgttgtctattatctgtttataggttcactcgacgaactaaactacctatacaagatatggttgtgccataatttttataaatttttttatgagtatttttacaaaaaaaatgtataaagtgtatgtcttatgtatatttataaaaatgctaagtatgcgatgtatctatgttatttgtatttatctaaacaatacctctacctctagatattatacaaaaattttttatttcggcatattaaagtaaaatctagttaccttgaaaatgaatacagtgggtggttccgtatcaccagtaagaacataatagtcgaatacagtatccgattgagattttgcatacaatactagtctagaaagaaatttgtaatcatcttctgtgacgggagtccatatatctgtatcatcgtctagtttatcagtgtcccatgctatattcctgttatcatcattagttaatgaaaataactctcgtgcttcagaaaagtcaaatattgtatccatacatacatctccaaaactatcgcttatacgtttatctttaacgatacctatacctagatggttatttactaacagacattttccagatctattgactataactcctatagtttccacatcaaccaagtaatgatcatctattgttatataacaataacataactcttttccatttttatcagtatgtatatctatatcaacgtcgtcgttgtagtgaatagtagtcattgatctattatatgaaacggatatgtctagaacggcaattgttttacgtccagttaacactttctttgatttaaagtctagagtctttgcaaacataatatccttatccgactttatatttcctgtagggtggtataattttattttgcctccacatatcggtgtttccaaatatattactagacaatattccatatagttattagttaagggtacccaattagaacacgtacgcttattatcatcatttggatcgtatttcataaaagttattgtactatcgatgtcaacacattctacattttttaatcgtctatatagtatttttctgatattttctataatatcagaattgtcttccatcggaagttgtatactatcggaatcagttacatgtttaaataattctctgatgtcattccttatacaatcaaattcattattaaacagtttaatagtctgtagacctttatcgtcgtaaatatccattgtcttattagttacgcttatttttatgtgttttacgttgctttattatattttataagaatgattgtttgacgaatcacgagaactattaagacacattattaggtatatattataaaaaagtttttgattacgatgttataagaggaaagaggacacattaacatcatacatcaattaactacattcttataacatcgtaatcaaaagaattgcaattttgatgtataacaactgtcaatgggttatggaattgtatattacatattatacggtatgttggtaacgacaaataccgatcggtaattgtctgccggtgtaatagaattatatatatctatctattacaccggctgagtatgcataataataagttgtggtagtatgatctccatatttataatttaggactttgtattcagtatttttggaatcataaaaaataaaaaaaagttttactaatttaaaatttaaaaagtatttacatttttttcactgtttagtcgcggatatggaattcgatcctgccaaaatcaatacatcatctatagatcatgtaacaatattacaatacatagatgaaccaaatgatataagactaacagtatgcattatccgaaatattaataacattacatattatatcaatatcacaaaaataaatacacatttggctaatcaatttcgggcttggaaaaaacgtatcgccggaagggactatatgactaacttatctagagatacaggaatacaacaatcaaaacttactgaaactatacgtaactgtcaaaaaaatagaaacatatatggtctatatatacactacaatttagttattaatgtggttattgattggataaccgatgtgattgttcaatcaatattaagagggttggtaaattggtacatagctaataatacctatactccaaatacacccaataatacaacaaccatttctgagttggatatcatcaaaatactggataaatacgaggacgtgtatagagtaagtaaagaaaaagaatgtggaatttgctatgaagttgtttactcaaaacgatagatactttggtttattggattcgtgtactcatatattttgcataacatgcatcaatatatggcataaaacacgaagagaaaccggtgcgtcggataattgtcctatatgtcgtacccgttttagaaacataacaatgagcaagttctataagctagttaactaataaataaaaagtttaatttgttgacgacgtatgtcgttatttttctcgtatgaaagattaaattcaattcaattcgttgtttctaatataatctgccgtattggatggattctcaagacaattgcatttagattatattatcatgaataaaaatagtagcacgcactacttcagccaaatattcttttttgaaacgccatctatcgtagtgaggacacaagtgaacctataattatcaaatttattagtatcagtcacatgaaggactttctgtagagtgacgattctaccatctatggtactaacggtttcatcctccttgataccctcacccaaatgttctataaatttagcatcctcgtccgatctcatatcctttgccaaccaatacatgtagctaaaattaggcataaatttcacacatccagtgcaacgaaattctccagaagatgttacgatgtttaggttaggacatttgatttcgtcggcattaacatatgggtgaacacacccatacatgaaagcgatgagaaataggattctcatcttgccaaaatatcactagaaaaaatttatttatcaattttaaaggtataaaaaatacttattgttgctcgaatattttgtatttgatggtatacggaagattagaaatgtaggtattatcatcaactgattctatggttttatgtattctatcatgtttcactattgcgtcggaaataatatcatatgcttccacatatattttattttgttttaactcataatactcacgtaattctggattattggcatatctatgaataattttagctccatgatcagtaaatattaatgagaacatagtattaccacctaccattatttttttcatttcgttcaattcttgattgcaaagatctatataatcattatagcgttgacttatggactctggaatcttagacgatgtacagtcatctataatcatggcatatttaatacattgttttatagcatagtagttatctacgatgttagatatttctctcaatgaatcaatcacacaatctaatgtaggtttatgacataatagcattttcagcagttcaatgtttctagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacatgtcacaaagaccctttttgtatgtataagtgtaaaaattataacattcatagttggatttacataggtgtccaatcgggatctctccatcatcgagataattgatggcatctcccttccttttttagtagatatttcatcgtgtaagaatcaatattaatatttctaaagtattcgtgtatagcctctttatttaccacagttccatattccactagagggatatcgccgaatgtcatatactcaattagtatatgttggaggacatccgagttcattgttttcaatatcaaaaagatggtttccttatcatttctccatagtggtacaatactacacattattccgtgcggctttccattttccaaaaacaatttgaccaaatctaaatctacatctttattgtatctataatcactatttagataatcagccataattactcgagtgcaacatgttagatcgtctatatatgaataagcagtgttatctattcctttcattaacaatttaacgatgtctatatctatatgagatgacttaatataatattgaagagctgtacaatagtttttatctatagaagacggcttgattccgtgattaattagacatttaacaacttccggacgcacatatgctctcgtatccgactttgaatacagatgagagatgatatacagatgcaatacggtaccgcaatttcgtagttgataatcatcatacgcgtatcagtactcgtcctcataaagaacactgcagccattttctatgaacaaatcaataattttaggaacaggatcattgtcattacataattttctataactgaacgatggttttcacatttaacactcaagtcaaatccatgttctaccaacacctttatcaagtcaacgtctacatttttggatttcatatagctgaatatattaaagtcatttatgttgctaaatccagtggcttctagtagagccatcgctatatcctttaactttaacatgtctactatttgtgtattcttctaatggggtagctgtctccaatttttgcgtaatggattagtgccactgtctagtagtagtttgacgacctcgacattattacaatgctcattaaaaaggtatgcgtgtaaagcattattcttgaattggttcctggtatcattaggatctctgtctctcaacatctgtttaagttcatcgagagccacctcctcattttccagatagtcaaacattttgactgaatgagctactgtgaactctatacacccacacaactaatgtcattaaatattatttttttgaatgtatttataccatgtcaaaaacttgtacaattattaataaaaataatttagtgtttaaattttaccagttccagattttacacctccgttaaccccactttttacaccactggacgatcctcctccccacattccaccgccaccagatgtataagttttagatcctttattactaccatcatgtccatggataaagacactccacatgccgccactactaccccctttagaagacatattaataagacttaaggacaagtttaacaataaaattaatcacgagtaccctactaccaacctacactattatatgattatagtttctatttttacagtaccttaactaaagtctctagtcacaagagcaatactaccaacctacactattatatgattatagtttctatttttataggaacgcgtacgagaaaatcaaatgtctaatttctaacggtagtgttgataaacgattatcgtcaatggatacctcctctatcatgtcgtctattttcttactttgttctattaacttattagcattatatattatttgattataaaacttatattgcttattagcccaatctgtaaatatcggattattaacatatcgtttctttgtaggtttatttaacatgtacatcactgtaagcatgtccgtaccatttattttaatttgacgcatatccgcaatttctttttcgcagtcggttataaattctatatatgatggatacatgctacatgtgtacttataatcgactaatatgaagtacttgatacatattttcagtaacgatttattattaccacctatgaataagtacctgtgatcgtctaggtaatcaactgttttcttaatacattcgatggttggtaatttactcagaataatttccaatatcttaatatataattctgctatttctgggatatatttatctgccagtataacacaaatagtaatacatgtaaacccatattttgttattatattaatgtctgcgccattatctattaaccattctactaggctgacactatgcgacttaatacaatgataaagtatactacatccatgtttatctattttgtttatatcatcaatatacggcttacaaagttttagtatcgataacacatccaactcacgcatagagaaggtagggaataatggcataatatttattaggttatcatcattgtcattatctacaactaagtttccattttttaaaatatactcgacaactttaggatctctattgccaaatttttgaaaatatttatttatatgcttaaatctatataatgtagctccttcatcaatcatacatttaataacattgatgtatactgtatgataagatacatattctaacaatagatcttgtatagaatctgtatatcttttaagaattgtggatattaggatattattacgtaaactattacacaattctaaaatataaaacgtatcacggtcgaataatagttgatcaactatataattatcgattttgtgatttttcttcctaaactgtttacgtaaatagttagatagaatattcattagttcatgaccactatagttactatcgaataacgcgtcaaatatttcccgtttaatatcgcatttgtcaagataataatagagtgtggtatgttcacgataagtataataacgcatctcttttttgtgtgaaattaaatagtttatcacgtccaaagatgtagcataaccatcttgtgacctagtaataatataataatagagaactgttttacccattctatcatcataatcagtggtgtagtcgtaatcgtaatcgtctaattcatcatcccaattataatattcaccagcacgtctaatctgttctattttgatcttgtatccatactgtatgttgctacatgtaggtattcctttatccaataatagtttaaacacatctacattgggatttgatgttgtagcgtatttctctacaatattaataccatttttgatactatttatttctatacctttcgaaattagtaatttcaataagtctatatcgatgttatcagaacatagatattcgaatatatcaaaatcattgatatttttatagtcgactgacgacaataacaaaatcacaacatcgtttttgatattattatttttcttggtaacgtatgcctttaatggagtttcaccatcatactcatataatggatttgcaccactttctatcaatgattgtgcactgctggcatcgatgttaaatgttttacaactatcatagagtatcttatcgttaaccatgattggttgttgatgctatcgcattttttggtttctttcatttcagttatgtatggatttagcacgtttgggaagcatgagctcatatgatttcagtactgtagtgtcagtactattagtttcgatcagatcaatgtctagatctatagaatcaaaacacgataggtcagaagataatgaatatctgtacgcttctttttgtactgtaacttctggttttgttagatggttgcatcgtgctttaacatcaatggtacaaattttatcctcgctttgtgtatcatattcgtctctagtataaaattctatattcagattatcatgcgatgtgtatacgctaacggtatcaataaacggagcacaccatttagtcataacagtaatccaaaattttttaaagtatatcttaacgaaagaagttgtgtcattgtctacggtgtatggtactagatcctcataagtgtatatatctagagtaatgtttaatttattaaatggttgataatatggatcctcatgacaatttccgaagatggaaatgagatatagacatgcaataaatctaatcgaagacatggttactccttaaaaaaatacgaataatcaccttggctatttagtaagtgtcatttaacactatactcatattaatccatggactcataatctctatacgggattaacggatgttctatatacggggatgagtagttttcttctttaactttatactttttactaatcatatttagactgatgtatgggtaatagtgtttaaagagttcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccatactgaatggatgaactaacgaatatcaacggcgttaatagtaatttactttttcatctttacatattgggtactagttttactatcataagtttataaattccacaagctactatggaataagccaaccatcttagtataccacacatgtcttaaagtttattaattaattacatgttgttttatatatatcgctacgaatttaaagagaaatcagtttaggaagaaaaaaattatctatctacatcatcacgtctctgtattctacgatagagtgctactttaagatgagacatatccgtgtcatcaaaaatatactccattaaaatgattattccggcagcgaacttgatattggatatatcacaacctttgttaatatctacgacaatagacagcagtcccatggttccataaacagtgagtttatctttctttgaagcgatagtttgtagagatcttataaaaccgtcaaacgacatcgcatttatatctttagctaattcatatatgttaccatcgtaatatctaaccgcgtctatcttaaacgtttccatcgctttaaagacgtttccgatagatggtctcatttcatcagtcatactgagccaacaaatataatcgtgtataacatctttgatagaatcagactctaaagaaaacgaatcggctttattatacgcattcatgataaacttaatgaaaaatgtttttcgttgtttaagttggatgaatagtatgtcttaataattgttattatttcattaattaatatttagtaacgagtacactctataaaaacgagaatgacataactagttatcaaagtgtctaggacgcgtaattttcatatggtatagatcctgtaagcattgtctgtattctggagctattttctttatcgcattagtaagttcagaatatgttataaatttaaatcgaataacgaacatgactttagtaaagtcgtctatattaactcttttattttctagccatcgtaataccatgtttaagatagtatattctctagttactacgatctcatcgttgtctagaatatcacatactgaatctacatccaattttagaaattggtctgtgttacatatctcttctatattattgttgatgtattgtcgtagaaaactattacgtagaccattttctttataaaacgaatatatagtactccaattatctttaccgatatatttgcacacataatccattctctcaatcactacatctttaagattttcgttgttaagatatttggctaaactatataattctattagatcatcaacagaatcagtatatatttttctagatccaaagacgaactctttggcgtcctctataatattcccagaaaagatattttcgtgttttagtttatcgagatctgatctgttcatatacgccatgattgtacggtacgttatgataaccgcataaaataaaaatccattttcatttttaaccaatactattcataattgagattgatgtaatactttgttactttgaacgtaaagacagtacacggatccgtatctccaacaagcacgtagtaatcaaatttggtgttgttaaacttcgcaatattcatcaatttagatagaaacttatactcatcatctgttttaggaatccatgtattattaccactttccaacttatcattatcccaggctatgtttcgtccatcatcgttgcgcagagtgaataattcttttgtattcggtagttcaaatatatgatccatgcatagatcggcaaagctattgtagatgtgatttttcctaaatctaatataaaactcgtttactagcaaacactttcctgatttatcgaccaagacacatatggtttctaaatctatcaagtggtggggatccatagttatgacgcagtaacatagattattacattcttgactgtcgctaatatctaaatatttattgttatcgtattggattctgcatatagatggcttgtatgtcaaagatatagaacacataaccaatttatagtcgcgctttacattctcgaatctaaagttaagagatttagaaaacattatatcctcggatgatgttatcactgtttctggagtaggatatattaaagtctttacagatttcgtccgattcaaataaatcactaaataatatcccacattatcatctgttagagtagtatcattaaatctattatattttatgaaagatatatcactgctcacctctatatttcgtacatttttaaactgtttgtataatatctctctgatacaatcagatatatctattgtgtcggtagacgataccgttacatttgaattaatggtgttccattttacaacttttaacaagttgaccaattcatttctaatagtatcaaactctccatgattaaatattttaatagtatccattttatatcactacggacacaaagtagctgacataaaccattgtataatttttatgttttatgtttattagcgtacacattttggaagttccggcttccatgtatttcctggagagcaagtagatgatgaggaaccagatagtttatatccgtacttgcacttaaagtctacattgtcgttgtatgagtatgatcttttaaacccgctagacaagtatccgtttgatattgtaggatgtggacatttaacaatctgacacgtgggtggatcggaccattctcctcctgaacacaggacaccagagttaccaatcaacgaatatccactattgcaactataagttacaacgctcccatcggtataaaaatcctcgtatccgttatgtcttccgttggatatagatggaggggattggcatttaacagattcacaaataggtgcctcgggattccataccatagatccagtagatcctaattcacaatacgatttagattcaccgatcaactgatatccgctattacaagagtacgttatactagagccaaagtctactccgccaatatcaagttggccattatcgatatctcgaggcgatgggcatctccgtttaatacattgattaaagagtgtccatccagtacctgtacatttagcatatataggtcccattttttgctttctgtatccaggtagacatagatattctatagtgtctcctatgttgtaattagcattagtttccacactattcttaaattttatattaatgggacgtgaaggaataggacagtatgatagaacgcatcctattcccaacaatgtcaggaacgtcacgctctccaccttcatatttatttatccgtaaaaatgttatcctggacatcgtacaaataataaaaaagcccatatatgtttgctattgtagaaattgtttttcacagttgctcaaaaacgatggcagtgacttatgagtttcatctttagtaaacatatcataatattcgatattacgagttgacatatcgaacaaattccaagtatttgattttggataatattcgtattttgcatctgctataattaagatataatcaccgcaagaacacacgaacatctttcctacatggttaaagtacatgtataattctatccatttgtcttccttaactatatatttgtatagataattacgagtctcataagtaattccagtaattgcatagatgtcaccatcgtactctacagcataaactatactatgatgtctaggcatgggagacttttttatccaacgatttttagtgaaacattctacatcgtttaatactacatatttctcatacgtggtataaactccacccattacatatatatcatcgtttacgaataccgacgcgcctgaatatctaggagtaattaagtttggaagtcttatccatttcgaagtgccgtgtttcaaatattctgccacacccgttgaaatagaaaattctaatcctcctattacatataactttccatcgttaacacaagtactaacttctgattttaacgacgacatattagtaaccgttttccattttttcgttttaagatctacccgcgatacggaataaacatgtctattgttaatcatgccgccaataatgtatagacaattatgtaaaacatttgcattatagaattgtctatctgtattaccgactatcgtccaatattctgttctaggagagtaatgggttattgtggatatataatcagagtttttaatgactactatattatgttttataccatttcgtgtcactggctttgtagatttggatatagttaatcccaacaatgatatagcattgcgcatagtattagtcataaacttgggatgtaaaatgttgatgatatctacatcgtttggatttttatgtatccactttaataatatcatagctgtaacatcctcatgatttacgttaacgtcttcgtgggataagatagttgtcagttcatcctttgataattttccaaattctggatcggatgtcaccgcagtaatattgttgattatttctaacatcgacgcattatatagttttttaattccatattgtttagaaaagttaaacatccttatacaatttgtggaattaatattatgaatcatagtttttacacatagatctactacaggcgtaacatcaattattacggcagcaactagtatcatttctacattgtttatggtgatgtttatcttcttccagcgcatatagtctaatagcgattcaaacgcgtgatagtttataccattcaatataatcacttcatcatttatatggtgctcctgaatgcgtttaaaaaaattatacggagacgccgtaataatttccttattcacttgtataatttccccattgatagaaaatatcacgctttccattcttgaagtactataagtaattatagtataatgtaaacgtttatatattcaatatttttataaaaatcattttgacattaattcctttttaaatttccgtctatcatctatagaaacgtattctatgaatttataaaatgcttttacgtgtcctatcgtaggcgatagaaccgctaaaaagcctatcgaatttctacaaaagaatctattatatggtatagggagagtataaaacattaaatgcccgtacttattaaagtattcagtagccaatcctaactctttcgaatacttattaatggctcttgttctgtacgaatctatttttttgaacaacggacctagtggtatatcttgttctatgtatctaaaataatgtctgactagatccgttagtttaatatcctcagtcatcttgtctagaatggcaaatctaactgcgggtttaggctttagtttagtttttatatctacatctatgtctttatctaacaccaaaaatataatagctaatattttattacaatcatccggatattcttctacgatctcactaactaatgtttctttggttatactagtatagtcacgatcagacaaataaagaaaatcagatgatcgatgaataatacatttaaattcatcatctgtaagatttttgagatgtctcattaaaatattattagggttagtactcattatcattcggcagctattacttattttattatttttcaccatatagatcaatcattagatcatcaaaatatgtttcaatcatcctaaagagtatggtgaatgactcttcccatctaatttctgaacgttcaccaatgtctctagccactttggcactaatagcgatcattcgcttagcgtcttctatattattaactggttgattcaatctatctagcaatggaccgtcggacagcgtcattctcatgttcttaatcaatgtacatacatcgccgtcatctaccaattcatccaacaacataagctttttaaaatcatcattataataggtttgatcgttgtcatttctccaaagaatatatctaataagtagagtcctcatgattagttaacaactattttttatgttaaatcaattagtacaccgctatgtttaatacttattcatattttagtttttaggattgagaatcaatacaaaaaattaatgcatcattaattttagaaatacttagtttccacgtagtcaatgaaacatttgaactcatcgtacaggacgttctcgtacaggacgtaactataaaccggtttatatttgttcaagatagatacaaatccgataactttttttacgaattctacgggatccactttaaaagtgtcataccgggttctttttattcttttaaacagatcaatggtgtgatgttgattaggtcttttacgaatttgatatagaatagcgttcacatatcctccataatggtcaatcgccatttgttcgtatgtcataaattctttaattatatgacactgtgtattatttagttcatccttgttcatcattaggaatctatccaaaatggcaattatactagaactataggtgcgttgtatacacatattgatgtgtctgtttatacaatccatgatatttggatccatgctactaccttcgggtaaaattgtagcatcatataccatttctagtactttaggttcattattatccattgcagaggacgtcatgatcgaatcataaaaaaatatattatttttatgttattttgttaaaaataatcatcgaatacttcgtaagatactccttcatgaacataatcagttacaaaacgtttatatgaagtaaagtatctacgatttttacaaaagtccggatgcataagtacaaagtacgcgataaacggaataataatagatttatctagtctatctttttctatagctttcatagttagatacatggtctcagaagtaggattatgtaacatcagcttcgataaaatgactgggttatttagtcttacacattcgctcatacatgtatgaccgttaactacagagtctacactaaaatgattgaacaatagatagtctaccattgtttcgtattcagatagtacagcgtagtacatggcatcttcacaaattatatcattgtctaatagatatttgacgcatcttatggatcccacttcaacagccatcttaaaatcggtaaaatcatattgctttcctttatcattaataatttctaaaacatcatctctatcataaaagatacaaatattaactgtttgatccgtaataacattgctagtcgatagcaatttgttaataagatgcgctgggctcaatgtcttaataagaagtgtaagaggactatctccgaatttgttttgtttattaacatccgttgatggaagtaaaagatctataatgtctacattcttgactgttttagagcatacaatatggagaggtgtatttccatcatgatctggttttgagggactaattcctagtttcatcatccatgagattgtagaagcttttggattgtctgacataagatgtctatgaatatgatttttgccaaatttatccactatcctggcttcgaatccgatggacattatttttttaaacactctttctgaaggatctgtacacgccaacaacggaccacatccttcttcatcaaccgagttgttaatcttggctccatactgtaccaataaatttattctctctatgacttcatcatctgttcccgagagataatatagaggtgttttattatgtttatcacacgcgtttggatctgcgccgtgcgtcagcagcatcgcgactattctattattattaattttagaagctatatgcaatggataatttccatcatcatccgtctcatttggagagtatcctctatgaagaagttcttcgacaaatcgttcatctagtcctttaattccacaatacgcatgtagaatgtgataattatttccagaaggttcgatagcttgtagcatattcctaaatacatctaaatttttactattatatttggcataaagagatagataatactcggccgacataatgttgtccattgtagtataaaaattaatatttctatttctatttctgtatatttgcaacaatttactctctataacaaatatcataacttagttcttttatgtcaagaaggcactggtttagttcatctataaatgtcacgccataactaccacgcatgccatactcagaattatgataaagatatttatccttggggtgtaggtaatggggattaatctttgttggatcagtctctaagttaacacatgtcacacatgatccatttatagttatatcacacgatgatgatttatgaattgattccggaagatcgctatcgtattttgtggttccacaattcatttccatacatgttattgtcacactaatattatgatgaactttatctagccgctgagtggtaaacaacagaacagatagtttattatctttaccaacaccctcagccgctgccacaaatctctgatccgtatccatgatggtcatgtttatttctagtccgtatccagtcaacactatgttagcatttctgtcgatatagctttcactcatatgacactcaccaataatagtagaattaatgtcgtaatttacaccaatagtgagttcggcggcaaagtaccaataccggtaatcttgtcgaggaggacatatagtattcttgtattctactgaatacccgagagatgcgatacaaaagagtaagactaatttgtaaaccatcttactcaaaatatgtaacaatagtacgatgcaatgagtaagacaataggaaatctatcttatatacacataattattctatcaattttaccaattagttagtgtaatgttaacaaaaatgtgggagaatctaattagtttttctttacacaattgacgtacatgagtctgagttccttgtttttgctaattatttcatccaatttattattcttgactatatcgagatcttttgtataggagtcagacttgtattcaacatgcttttctataatcattttagctatttcggcatcatccaatagtacattttccagattagcagaatagatattaatgtcgtatttgaacagagcctgtaacatctcaatgtctttattatctatagccaatttaatgtccggaatgaagagaagggaattattggtgtttgtcgacgtcatatagtcgagcaagagaatcatcatatccacgtgtccattttttatagtggtgtgaatacaactaaggagaatagccagatcaaaagtagatggtatctctgaaagaaagtaggaaacaatacttacatcattaagcatgacggcatgataaaatgaagttttccatccagttttcccatagaacatcagtctccaatttttcttaacaaacagttttaccgtttgcatgttaccactatcaaccgcataatacaatgcggtgtttcccttgtcatcaaattgtgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagagaaagcttttcagctgcttagacttccaagtattaattcgtgacagatccatgtctgaaacgagacgctaattagtgtatattttttcattttttataattttgtcatattgcaccagaattaataatatctctaatagatctgattagtagatacatggctatcgcaaaacaacatatacacatttaataaaaataatatttattaagaaaattcagatttcacgtacccatcaatataaataaaataatgattccttacaccgtacccatattaaggagattccaccttacccataaacaatataaatccagtaatatcatgtctgatgatgaacacaaatggtgtattaaattccagtttttcaggagatgatctcgccgtagctaccatgatagtagatgcctctgctacagttccttgttcgtcgacatctatctttgcattctgaaacattttataaatatataatgggtccctagtcatatgtttaaacaacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatatcatattcttcgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatgcacacagtattatctacgaaactttgataagttagatcagtgtacgtatatttagatgttttcagcttagctaatcctgatattaattctgtaaatgctggacccagatctctttttctcaaatccatagtcttcaataattctattctagtattacctgatgcaggcaatagcgacataaacatagaaaacgaataaccaaacggtgagaagacaatattatcatcttgaatatttttatacgctactataccggcattggtaaatccttgcagacgataggtagacactgaacacgttaacgatagtatcaataacgcaatcatgattttatggtattaataattaaccttatttttatgttcggtataaaaattattgatgtctacacatccttttgtaattgacatctatatatccttttgtataatcaactctaatcactttaacttttacagttttccctaccagtttatccctatattcaacatatctatccatatgcatcttaacactctctgccaagatagcttcaaagtgaggatagtcaaaaagataaatatatagagcataatcattctcgtatactctgccctttattacatcacccgcattgggcaacgaataacaaaatgcaagcatcttgttaacgggctcgtaaattgggataaaaattatgtttttatatctattttattcaagagaatattcaggaatttctttttccggttgtatctcatcgcagtatatatcatttgtacattgtttcatattttttaatagtctacaccttttagtaggactagtatcgtacaattcatagctgtattttgaattccaatcacgcataaaaatatcttccaattgttgacgaagacctaatccatcatccggtgtaatattaatagatgctccacatgtatccgtaaagtaatttcctgtccaatttgaggtacctatatacgccgttttatcggttaccatatatttggcatggtttaccctagaatacggaatgggaggatcagcatctggtacaataaatagctttacttctatatttatgtttttagattttagcatagcgatagatcttaaaaagtttctcatgataaacgaagatcgttgccagcaactaatcaatagcttaacggatacttgtctgtctatagcggctcttcttaattcatcttctatataaggccaaaacaaaatattgcctgccttcgaataaataatagggataaagttcataacagatacataaacgaatttactcgcatttctaatacatgacaataaagcggttaaatcattggttctttccatagtacatagttgttgcggtgcagaagcaataaatacagagtgtggaacaccacttacgttaatactaagaggatgatctgtattataatacgacggataaaagtttttccaattatatggtagattgttaactccaagataccagtatacctcaaaaatttgagtgagatccgctgccaagttcctattattgaagatcgcaatacccaattctttgacctgagttagtgatctccaatccatgttagcgcttcctaaataaatatgtgtattatcagatatccaaaattttgtatgaagaactcctcctaggatatttgtaatatctatgtatcgtacttcaactccggccatttgtagtctttcaacatcctttaatggtttgttagatttattgacggctactctaactcttactcctcttttgggtaattgtacaatctcgtttaatattatcgtgccgaaattcgtacccacttcatccgataaactccaataaaaagatgatatatctagtgtttttgtggtattggatagaatttccctccacatgttaaatgtagacaaatatactttatcaaattgcatacctataggaatagtctctgtaatcactgcgattgtattatccggattcattttatttgttaaaaaataatcctatatcacttcactctattaaaaatccaagtttctatttctttcatgactgattttttaacttcatccgtttccttatgaagatgatgtttggcaccttcataaatttttatttctctattacaatttgcatgttgcatgaaataatatgcacctaaaacatcgctaatctcattgtttgttccctggagtatgagagtcggggggtgttaatcttgggaattatttttctaaccttgttggtagccttcaagacctgactagcaaatccagccttaattttttcatgattgattaatgggtcgtattggtatttataaactttatccatatctctagatactgattctggacatagctttccgactggcgcatttggtgtgatggttcccataagtttggcagctagcagattcagttttgaaacagcatctgcattaactagaggagacattagaatcattgctgtaaacaagtttggattatcgtaagaggctagtatagaaattgttgctcccatggaatgcccaataagaagactggaactcctaaataagtagatttaatagttaccacgtgctgtaccacatctctaacatacgtaccaaagtcatcaatcatcattttttcaccattacttcttccatgtccaatatgatcatgtgagaatactaaaattcctaacgatgatatgttttcagctagttcgtcataacgtccagaatgtttaccagctccatgacttatgaatactaatgccttaggatatgtaataggtttccaatatatgtaatcattgtccagattgaacatacagtttgcactcatgattcacgttatataactatcaatattaacagttcgtttgatgatcatattatttttatgttttattgataattgtaaaaacatacaattaaatcaatatagaggaaggagacggctactgtcttttgtgagatagtcatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtctattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattgatgatataggggtcttcataacgcataattattacgttagcattctatatccgtgttaaaaaaaattatcctatcatgtatttgagagttttatatgtagcaaacatgatagctgtgatgccaataagctttagatattcacgcgtgctagtgttagggatggtattatctggtggtgaaatgtccgttatataatctacaaaacaatcatcgcatatagtatgcgatagtagagtaaacatttttatagtttttactggattcatacatcgtctacccaatttggttataaatgaaattgtcgccaatcttacacccaaccccttgttatccattagtatagtattaacttcgttatttatgtcataaactgtaaatgattttgtagatgccatatcatacatgatattcatgtccctattataatcattactaactttatcacaatatatgttgataatatctatatatgatctagtctttgtgggcaactgtctatacaagtcgtctaaacgttgtttactcatatagtatcgaacagccatcattacatggtcccgttccgttgatagataatcgagtatgttagtggacttgtcaaatctatataccatattttctggaagtggatatacatagtcgtgatcaacattattgctagcctcatcttctatatcatgtactataccattatctatatcatctacataatctacgatattattacacataaacatcgacaacatactattgtttattatctaagtcctgttgatccaaacccttgatctcctctatttgtactatctagagattgtacttcttccagttctggataatatatacgttgatagattagctgagctattctatctccagtatttacattaaacgtacattttccattattaataagaatgactcctatgtttcccctataatcttcgtctattacaccacctcctatatcaatgccttttagtgacagaccagacctaggagctattctaccatagcaaatcttaggcatggacatactaatatctgtcttaattaactgtctttctcctggagggatagtataatcgtaagcgctatacaaatcatatccggcagcacccggcgattgcctagtaggagatttagctctgttagtttccttaacaaatctaactggtgagttaatattcatgttgaacataaaactaatattttatttcaaaattatttaccatcccatatattccatgaataagtgtgatgattgtacacttctatagtatctatatacgattcacgataaaatcctcctatcaatagcagtttattatccactatgatcaattctggattatccctcggataaataggatcatctatcagagtccatgtattgctggattcacaataaaattccgcatttctaccaaccaagaataaccttctaccgaacactaacgcgcatgatttataatgaggataataagtggatggtccaaactgccactgatcatgattgggtagcaaatattctgtagttgtatcagtttcagaatgtcctcccattacgtatataacattgtttatagatgccactgctggattacatctaggtttcagaagactcggcatattaacccaagcagcatccccgtggaaccaacgctcaacagatgtgggatttggtagacctcctactacgtataatttattgttagcgggtatcccgctagcatacagtctggggctattcatcggaggaattggaatccaattgtttgatatataatttacagctatagcattgttatgtatttcattgttcatccatccaccgatgagatatactacttctccaacatgagtacttgtacacatatggaatatatctataatttgatccatgttcataggatactctatgaatggatacttgtatgatttgcgtggttgtttatcacaatgaaatattttggtacagtctagtatccattttacattatttatacctctgggagaaagataatttgacctgattacatttttgataaggagtagcagatttcctaatttatttcttcgctttatataccacttaatgacaaaatcaactacataatcctcatctggaacatttagttcatcgctttctagaataagtttcatagatagataatcaaaattgtctatgatgtcatcttccagttccaaaaagtgtttggcaataaagtttttagtatgacataagagattggatagtccgtattctatacccatcatgtaacactcgacacaatattcctttctaaaatctcgtaagataaagtttatacaagtgtagatgataaattctacagaggttaatatagaagcacgtaataaattgacgacgttatgactatctatatatacctttccagtatacgagtaaataactatagaagttaaactgtgaatgtcaaggtctagacaaaccctcgtaactggatctttatttttcgtgtatttttgacgtaaatgtgtgcgaaagtaaggagataactttttcaatatcgtagaattgactattatattgccacctatagcatcaataattgttttgaatttcttagtcatagacaatgctaatatattcttacagtacacagtattaacaaatatcggcatttatgtttctttaaaagtcaacatctagagaaaaatgattatctttttgagacataactcccattttttggtattcacccacacgtttttcgaaaaaattagtttttccttccaatgatatattttccatgaaatcaaacggattggtaacattataaatttttttaaatcccaattcagaaatcaatctatccgcgacgaattctatatatgttttcatcatttcacaattcattcctataagtttaactggaagagccgcagtaagaaattcttgttcaatggatactgcatctgttataatagatctaacggtttcttcactcggtggatacaataaatgtttaaacatcaaacatgcgaagtcgcagtgtagaccctcgtctctactaattagttcgttggaaaacgtgagtccgggcattaggccacgctttttaagccaaaatatggaagcgaatgatccggaaaagaagattccttctactgcagcaaaggcaataagtctctctccataaccggcgctgtcatgtatccacttttgagcccaatcggccttcttttttacacaaggcatcgtttctatggcattaaagagatagtttttttcattactatctttaacataagtatcgatcaaaagactatacatttccgaatgaatgttttcaatggccatctgaaatccgtagaaacatctagcctcggtaatctgtacttctgtacaaaatcgttccgccaaattttcattcactattccgtcactggctgcaaaaaacgccaatacatgttttataaaatatttttcgtctggtgttagtttattccaatcattgatatctttagatatatctacttcttccactgtccaaaatgatgcctctgcctttttatacatgttccagatgtcataatattggattgggaaaataacaaatctatttggatttggtgcaaggatgggttccataactaaattaacaatatcaataaattttttttcagttatctatatgcctgtacttggattttttgtacatcgatatcgccgcaatcactacaataattacaagtattattgatagcattgttattagtactatcataattaaattatcgacattcatgggtgctgaataatcgttattatcatcattatcattttgtaattgtgacatcatactaaataaatcgtttgcgagattgttgtgggaagcgggcatggaggatgcattatcattattatttaacgccttccatttggattcacaaatgttacgcacattcaacattttatggaaactataattttgtgaaaacagataacaagaaaactcgttatcgttcaaatttttaacgatagtaaaccgattaaacgtcgagctaatttctaacgctagcgactctgttggatatgggtttccagatatatatcttttcagttcccctacgtatctataatcatctgtaggaaatggaagatatttccatttatctactgttcctaatatcatatgtggtggtgtagtagaaccattaagcgcgaaagatgttatttcgcatcgtattttaacttcgcaataatttctggttagataacgcactctaccagtcaagtcaatgatattagcctttacagatatattcatagtagtcgtaacgatgactccatcttttagatgcgatactcctttgtatgtaccagaatcttcgtacctcaaactcgatatatttaaacaagttaatgagatattaacgcgttttatgaatgatgatatataaccagaagttttatcctcggtggctagcgctataaccttatcattataataccaactagtgtgattaatatgtgacacgttagtgtgggtacaaatatgtacattatcgtctacgtcgtattcgatacatccgcatacagccaacaaatataaaatgacaaatactctaacgccgttcgtacccatcttgatgcggtttaataaatgttttgatttcaatttattgtaaaaaaagattcggttttatactgttcgatattctcattgcttatattttcatctatcatctccacacagtcaaatccgtggttagcatgcacctcatcaaccggtaaaagactatcggactcttctatcattataactctagaatatttaatttggtcattattaatcaagtcaattatcttatttttaacaaacgtgagtattttactcattttttataaaaacttttagaaatatacagactctatcgtgtgtctatatcttctttttatatccaatgtatttatgtctgatttttcttcatttatcatatataatggtccaaattctacacgtgcttcggattcatccagatcattaaggttcttataattgtaacatccttctcttccctcttctacatcttccttcttattcttattcttagcgtcacagaatctaccacagcaggatcccatgacgagcgtcatattaaactaattcattttcaattataatatactggtaatgaccattaaaataaaaatattcttcataaccggtaagaaagtgaaaagttcacattgaaactatgtcagtagtatacatcatgaaatgagatgaaatgatgatatatatactctattttggtggaggattatatgatataattcgtggataatcatttttaagacacatttctttattcgtaaatcttttcacgttaaatgagtgtccatattttgcaatttcttcatatgatggcggtgtacgtggacgaggctgctcctgttcttgttgtagtcgccgactgtcgtgtttgcgtttagatccctccattatcgcgattgcgtagatggagtactattatataccttgtaattaaatttttttattaattaaacgtataaaaacgttccgtatctgtatttaagagccagatttcgtctaatagaacaaatagctacagtaaaaataactagaataattgctacacccactagaaaccacggatcgtaatacggcaatcggttttcgataataggtggaacgtatattttatttaaggacttaacaattgtctgtaaaccacaatttgcttccgcggatcctgtattaactatctgtaaaagcatatgttgaccgggcggagccgaacattctccgatatctaatttctgtatatctataatattattaacctccgcatacgcattacagttcttttctagcttggataccgcactaggtacatcgtctagatctattcctatttcttcagcgatagctcttctatccttttccggaagcaatgaaatcacttcaataaatgattcaaccatgagtgtgaaactaagtcgagaattactcatgcatttgttagttattcggagcgcgcaatttttaaactgtcctataacctctcctatatgaatagcacaagtgacattagtagggatagaatgttgagctaatttttgtaaataactatctataaaaagattatacaaagttttaaactctttagtttccgccatttatccagtctgagaaaatgtctctcataataaatttttccaagaaactaattgggtgaagaatggaaacctttaatctatatttatcacagtctgtcttggtacacatgatgaattcttctaatgctgtactaaattcgatatctttttcgatttctggatatgtttttaataaagtatgaacaaagaaatggaaatcgtaataccagttatgtttaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcagcatggtagttcgtaaattttaatgtataacagagccatctgtaaaaagataaactttatgtattgtaccaaagatttaaatcctaatttgatagctaactcggtatctactttatctgcagaatacagtgctaggggaaaaattataatatttcctctttcgtattcgtagttagttctcttttcatgttcgaaaaagtgaaacatgcggttaaaatagtttataacattaatattactgttaataactgccgggtaaaagtgggatagtaatttcacgaatttgatactgtcctttctctcgttaaacgcctttaaaaaaactttagaagaatatctcaatgagagttcctgaccatccatagtttgtatcaataatagcaacatatgaagaacccgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccctattaaaattcttaaaataatttaacaatctaactgacggagcccaattttggtgtaaatctaattgggacattatattgttaaaatacaaacagtctcctaatataacagtatctgataatctatggggagacatccattgatattcaggggatgaatcattggcaacacccatttattgtacaaaaagccccaatttacaaacgaaagtccaggtttgatagagacaaactattaactattttgtctctgtttttaatttctttagtaatgaaattattcacaatatcagtatcttctttatctaccagagattttactaacttgataaccttggctgtctcattcaatagggtagtaatatttgtatgtgtgatattgatatctttttgaattgtttcttttagaagtgattctttgatggtgccagcatacgaattacaataatgcagaaactcggttaacatgcaggaattatagtaagccaattccaattgttgcctgtattgtattagagtattaatatgcgcaatggtgtccttgcgtttctctgatagaatgcgagcagcgattttggcgttatcatttgacgatatttctggaatgacgaatcctgtttctactaactttttggtaggacaaagtgaaacaatcaagaagatagcttctcctcctatttgtggaagaaattgaactcctctagatgatctactgacgatagtatctccttgacagatattggaccgaattacagaagtacctggaatgtaaagccctgaaaccccctcattttttaagcagattgttgccgtaaatcctgcactgtgaccaagatagagagctcctttggtgaatccatctctatgtttcagtttaaccaagaaacagtcagctggtctaaaatttccatctctatctaatacagcatctaacttgatgtcaggaactatgaccggtttaatgttatatgtaacattgagtaaatccttaagttcataatcatcactgtcatcagttatgtacgatccaaacaatgtttctaccggcatagtggatacgaagatgctatccatcagaatgtttccctgattagtattttctatatagctattcttctttaaacgattttccaaatcagtaactatgttcatttttttaggagtaggacgcctagccagtatggaagaggattttctagatcctctcttcaacatctttgatctcaatggaatgcaaaaccccatagtgaaacaaccaacgataaaaataatattgtttttcactttttataattttaccatctgactcatggattcattaatatctttataagagctactaacgtataattctttataactgaactgagatatatacaccggatctatggtttccataattgagtaaatgaatgctcggcaataactaatggcaaatgtatagaacaacgaaattatactagagttgttaaagttaatattttctatgagctgttccaataaattatttgttgtgactgcgttcaagtcataaatcatcttgatactatccagtaaacagtctttaagttctggaatattatcatcccattgtaaagcccctaattcgactatcgaatatcctgctctgatagcagtttcaatatcgacggacgtcaatactgtaataaaggtggtagtattgtcatcatcgtgataaactactggaatatggtcgttagtaggtacggtaactttacacaacgcgatatataactttccttttgtaccatttttaacgtagttgggacgtcctgcagggtattgttttgaagaaatgatatcgagaacagatttgatacgatatttgttggattcctgattatttactataatataatctagacagatagatgattcgataaatagaaaaggtatatcgttggtaggataatacatccccattccagtattctcggatactctattaatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacagttattgggccagtatactgttagcaacggtatatcgaatagattactcatgtaacctactagaatgatagttcgtgtactagtcataatatctttaatccaatctaagaaatataaaattagatcttttacactgttaaagttaacaaaggtattacccggatacgtggatatcatatatggcattggtccattatcagtaatagctccataaactgatacggcgatggtttttatatgtgtttgatctaacgaggaagaaattcgcgcccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctgtaatcgttaaattttacaaatctataacatgctaaaccttttgacgacaaccattcattaatttctgatatggaatctgtattctcgataccgtattgttctaaagccagtgctatatctccctgttcgtgggaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatcttgtaatattcatcatcgacataatactatctatgttatcatcgtatattagtataccatgaccttcttcatttcgtgccaaaatgatatacagtcttaaatagttacgcaatatctcaatagtttcataattgttagctgttttcatcaagatttgtaccctgtttaacatgatggcgttctatacgtttctattttctattttttaaatttttaacgatttactgtggctagatacccaatctctctcaaatatttttttagcctcgcttacaagctgtttatctatactattaaaactgacgaatccgtgattttggtaatgggttccgtcgaaatttgccgaagtgatatgaacatattcgtcgtcgactatcaacaattttgtattattctgaatagtgaaaaccttcacagatagatcattttgaacacacaacgcgtctagacttctggcggttgccatagaatatacgtcgttcttatcccaattaccaactagaagtctgatcttaactcctctattaatggctgcttctataatggagttgtaaatgtcgggccaatagtagctattaccgtcgacacgtgtagtgggaactatggccaaatgttcaatatctatactagtcttagctgacctgagtttatcaataactacatcggtatctagatctctagaatatcccaataggtgttccggagaatcagtaaagaacactccacctataggattcttaatatgatacgcagtgctaactggcagacaacaagccgcagagcataaattcaaccatgaattttttgcgctattaaaggctttaaaagtatcaaatcttctacgaagatctgtggccagcgggggataatcagaatatacacctaacgttttaatcgtatgtatagatcctccagtaaatgacgcgtttcctacataacatctttcatcatctgacacccaaaaacaaccgagtagtagtcccacattattttttttatctatattaacggttataaaatttatatccgggcagtgactttgtagctctcccagatttcttttccctcgttcatctagcaaaactattattttaatccctttttcagatgcctcttttagtttatcaaaaataagcgctcccctagtcgtactcagaggattacaacaaaaagatgctatgtatatatatttcttagctagagtgataatttcgttaaaacattcaaatgttgttaaatgatcggatctaaaatccatattttctggtagtgtttctaccagcctacattttgctcccgcaggtaccgatgcaaatggccacatttagttaacataaaaacttatacatcctgttctatcaacgattctagaatatcatcggctatatcgctaaaattttcatcaaagtcgacatcacaacctaactcagtcaatatattaagaagttccatgatgtcatcttcgtttatttctatatccgtatccattgtagattgttgaccgattatcgagtttaaatcattactaatactcaatccttcagaatacaatctgtgtttcattgtaaatttataggcggtgtatttaagttggtagattttcaattatgtatcaatatagcaacagtagttcttgctcctccttgattctagcatcctcttcattattttcttctacgtacataaacatgtccaatacgttagacaacacaccgacgatggcggccgccacagacacgaatatgactaaaccgatgaccatttaaaaacccctctctagctttcacttaaactgtatcgattattcttttagaacatgtataatataaaaacattattctatttcgaatttaggcttccaaaaatttttcatccgtaaaccgataataatatatatagacttgttaatagtcggaataaatagattaatgcttaaactatcatcatctccacgattagagatacaatatttacattctttttgctgtttcgaaactttatcaatacacgttaatacaaacccaggaaggagatattgaaactgaggctgttgaaaatgaaacggtgaatacaataattcagataatgtaaaatcatgattccgtattctgatgatattagaactgctaatggatgtcgatggtatgtatctaggagtatctattttaacaaagcatcgatttgctaatatacaattatccttttgattaattgttattttattcatattcttaaaaggtttcatatttatcaattcttctacattaaaaatttccatttttaatttatgtagccccgcaatactcctcattacgtttcattttttgtctataatatccattttgttcatctcggtacatagattatccaattgagaagcgcatttagtagttttgtacattttaagtttattgacaaatcgtcgaaaactagttatagttaacattttattatttgataccctgatattaatacccctgccgttactattatttataactgatgtaatccacgtaacattagaattaattatcgatagtaatgcatcgacgcttccaaaattgtctattataaactcaccgataatttttttattgcatgttttcatattcattaggattatcaaatctttaatcttattacgattgtatgcgttgatattgcaagacgtcattctaaaagacggaggatctccatcaaatgccaaacaatcacgtacaaagtacatggaaataggttttgttctattgcgcatcatagatttatatagaacacccgtagaaatactaatttgttttactctataaaatactaatgcatctatttcatcgttttgtataacgtctttccaagtgtcaaattccaaatttttttcattgatagtaccaaattcttctatctctttaactacttgcatagataggtaattacagtgatgcctacatgccgttttttgaaactgaatagatgcgtctagaagcgatgctacgctagtcacaatcaccactttcatatttagaatatatgtatgtaaaaatatagtagaatttcattttgtttttttctatgctataaatgaattctcattttgcatctgctcatactccgttttatatcaataccaaagaaggaagatatttggttctaaaagccgttaaagtatgcgatgttagaactgtagaatgtgaaggaagtaaagcttcctgcgtactcaaagtagataaaccctcatcacccgcgtgtgagagaagaccttcgtccccttccagatgcgagagaatgaataacccaggaaaacaagttccgtttatgaggacggacatgctacaaaatatgttcgcggctaatcgcgataatgtagcttctagacttttgtcctaaaatactattatatccttttcgatattaataaatccgtgtcgtccaggttttttatctctttcagtatgtgaatagataggtattttatctctattcatcatcgaatttaagagatccgataaacattgtttgtattctccagatgtcagcatctgatacaacaatatatgtgcacataaacctctggcacttatttcatgtaccttccccttatcactaaggagaatagtatttgagaaatatgtatacatgatattatcatgaattagatatacagaatttgtaacactctcgaaatcacacgatgtgtcggcgttaagatctaatatatcactcgataacacattttcatctagatacactagacattttttaaagctaaaatagtctttagtagtgacagtaactatgcgattattttcatcgatgatacatttcatcggcatattattacgcttaccatcaaagactataccatgtgtatatctaacgtattctagcatggttgccatacgcgcattaaacttttcaggatctttggatagatcttccaatctatctatttgagaaaacatttttatcatgttcaatagttgaaacgtcggatccactatatagatattatctataaagattttaggaactacgttcatggtatcctggcgaatattaaaactatcaatgatatgattatcgttttcatcttttatcaccatatagtttctaagatatgggattttacttaatataatattatttcccgtgataaattttattagaaaggccaaatctataagaaaagtcctagaattagtctgaagaatatctatatcgccgtatagtatatttggattaattagatatagagaatatgatccgtaacatatacaacttttattatggcgtctaagatattcttccatcaacttattaacatttttgactagggaagatacattatgacgtcccattacttttgccttgtctattactgcgacgttcatagaatttagcatatctcttgccaattcttccattgatgttacattataagaaattttagatgaaattacatttggagctttaatagtaagaactcctaatatgtccgtgtatgtggtcactaatacagattgtagttctataatcgtaaataatttacctatattatatgtttgagtctgtttagaaaagtagctaagtatacgatcttttatttctgatgcagatgtattaacatcggaaaaaaatctttttttattcttttttactaaagatacaaatatgtctttgttaaaaacagttattttttgaatatttctagcttgtaattttaacatatgatattcgttcacactaggtactctgcctaaataggtttctataatctttaatgtaatattaggaaaagtattctgatcaggattcctattcattttgaggatttaaaactctgattattgtctaatatggtctctacgcaaactttttcacagagcgatagagtttttgataactcgtttttcttaagaaatataaaactactgtctccagagctcgctctatcttttattttatctaattcgatacaaactcctgatactggttcagaaagtaattcattaattttcagtcctttatagaagatatttaatatagataatacaaaatcttcagtttttgatatcgatctgattgatcctagaactagatatattaataacgtgctcattaggcagtttatggcagcttgataattagatatagtatattccagttcatatttattagataccgcattgcccagattttgatattctatgaattcctctgaaaataaatccaaaataactagacattctattttttgtggattagtgtactctcttccctctatcatgttcactactggtgtccacgatgataaatatctagagggaatataatatagtccataggatgccaatctagcaatgtcgaataactgtaattttattcttcgctcttcattatgaattgattcttgaggtataaacctaacacaaattatattattagacttttcgtatgtaatgtctttcatgttataagtttttaatcctggaatagaatctattttaatgaggcttttaaacgcagagttctccaacgagtcaaagcataatactctgttgtttttcttatatacgatgttacgattttcttctttgaatggaataggtttttgaattagtttataattacaacataatagataaggaagtgtgcaaatagtacgcggaaaaaacataatagctcccctgttttcatccatggttttaagtaaatgatcactggcttctttagtcaatggatattcgaacattaaccgtttcatcatcattggacagaatccatattttttaatgtaaagagtgatcaaatcattgtgtttattgtaccatcttgttgtaaatgtgtattcggttatcggatctgctcctttttctattaaagtatcgatgtcgatctcgtctaagaattcaactatatcgacatatttcatttgtatacacataaccattactaacgtagaatgtataggaagagatgtaacgggaacagggtttgttgattcgcaaactattctaatacataattcttctgttaatacgtcttgcacgtaatctattatagatgccaagatatctatataattattttgtaagatgatgttaactatgtgatctatataagtagtgtaataattcatgtattttgatatatgttccaactctgtctttgtgatgtctagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacttgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgataactagaatcagaatctaatgatgacgtaaccaagaagtttatctactgccaatttagctgcattatttttagcatctcgtttagattttccatctgccttatcgaatactcttccgtcgatatctacacaggcataaaatgtaggagagttactaggccccactgattcaatacgaaaagaccaatctctcttagttatttggcagtactcattaataatggtgacagggttagcatctttccaatcaataatttttttagccggaataacatcatcaaaagacttatgatcctctctcattgatttttcgcgggatacatcatctattatggcgtcagccataacatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatgtttttaatagccgcacacacaatctctgcgtcagaacgctcgtcaatatagatcttagacatttttagagagaactaacacaaccagcaataaaactaatttattttatcatttttttattcatcatcctctggtggttcgtcgtttctatcgaatgtggatctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcatgtctaactagtggaggttcgtcagcggctctagtttgaatcatcatcggcgtagtattcctacttttacagttaggacacggtgtattgtatttctcgtcgagaacgttaaaataatcgttgtaactcacatcctttattttatctatattgtattctactcctttcttaatgcattttataccgaataagagatagcgaaggaattctttttcggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggctaccgccattgatgtttgttcattggatgagtaactactaatgtatacattttccatttataacacttatgtattaactttgttcatttatattttttcattattatgttgatattaacaaaagtgaatatatatatatgttaataattgtattgtggttatacggctacaattttataatgagtgaaagtcagtgtccgatgatcaatgacgatagctttactctgaaaagaaagtatcaaatcgatagtgcggagtcaacaataaaaatggataagaagaggataaagtttcagaatagagccaaaatggtaaaagaaataaatcagacaataagagcagcacaaactcattacgagacattgaaactaggatacataaaatttaagagaatgattaggactactactctagaagatatagcaccatctattccaaataatcagaaaacttataaactattctcggacatttcagccatcggcaaagcatcacagaatccgagtaagatggtatatgctctgctgctttacatgtttcccaatttgtttggagatgatcatagattcattcgttatagaatgcatccaatgagtaaaatcaaacacaagatcttctctcctttcaaacttaatcttattagaatattagtggaagaaagattctataataatgaatgcagatctaataaatggaaaataattggaacacaagttgataaaatgttgatagctgaatctgataaatatacaatagatgcaaggtataacctaaaacccatgtatagaatcaagggagaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaaaaatcttcaagagtgcattgactttgttagtgaataggcatttcatctttctccaatactaattcaaattgttaaattaataatggatagtataaatagttattagtgataaaatagtaaaaataattattagaataagagtgtagtatcatagataactctcttctataaaaatggattttattcgtagaaagtatcttatatacacagtagaaaataatatagattttttaaaggatgatacattaagtaaagtaaacaattttaccctcaatcatgtactagctctcaagtatctagttagcaattttcctcaacacgttattactaaggatgtattagctaataccaatttttttgttttcatacatatggtacgatgttgtaaagtgtacgaagcggttttacgacacgcatttgatgcacccacgttgtacgttaaagcattgactaagaattatttatcgtttagtaacgcaatacaatcgtacaaggaaaccgtgcataaactaacacaagatgaaaaatttttagaggttgccgaatacatggacgaattaggagaacttataggcgtaaattatgacttagttcttaatccattatttcacggaggggaacccatcaaagatatggaaatcatttttttaaaactgtttaagaaaacagacttcaaagttgttaaaaaattaagtgttataagattacttatttgggcatacctaagcaagaaagatacaggcatagagtttgcggataatgatagacaagatatatatactctatttcaacaaactggtagaatcgtccatagcaatctaacagaaacgtttagagattatatctttcccggagataagactagctattgggtgtggttaaacgaaagtatagctaatgatgcggatatcgttcttaatagacacgccattaccatgtatgataaaattcttagttatatatactctgagataaaacagggacgcgttaataaaaacatgcttaagttagtttatatctttgagcctgaaaaagatatcagagaacttctgctagaaatcatatatgatattcctggagatatcctatctattattgatgcaaaaaacgacgattggaaaaaatattttattagtttttataaagctaattttattaacggtaatacatttattagtgatagaacgtttaacgaggacttattcagagttgttgttcaaatagatcccgaatatttcgataatgaacgaattatgtctttattctctacgagtgctgcggacattaaacgatttgatgagttagatattaataacagttatatatctaatataatttatgaggtgaacgatatcacattagatacaatggatgatatgaagaagtgtcaaatctttaacgaggatacgtcgtattatgttaaggaatacaatacatacctgtttttgcacgagtcggatcccatggtcatagagaacggaatactaaagaaactgtcatctataaaatccaagagtagacggctgaacttgtttagcaaaaacattttaaaatattatttagacggacaattggctcgtctaggtcttgtgttagatgattataaaggagacttgttagttaaaatgataaaccatcttaagtctgtggaggatgtatccgcattcgttcgattttctacagataaaaaccctagtattcttccatcgctaatcaaaactattttagctagttataatatttccatcatcgtcttatttcaaaggtttttaagagataatctatatcatgtagaagaattcttggataaaagcatccatctaaccaagacggataagaaatatatacttcaattgataagacacggtagatcatagaacagaccaaatatattattaataatttgtatatacatagatataattatcacatattaaaaattcacacatttttgataaatgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcagagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataagtataatcccattctaatactttaacctgatgtattagcatcttattagaatattaacctaactaaaagacataacataaaaactcattacatagttgataaaaagcggtaggatataaatattatggctgccaccgttccgcgttttgacgacgtgtacaaaaatgcacaaagaagaattctagatcaagaaacattttttagtagaggtctaagtagaccgttaatgaaaaacacatatctatttgataattacgcgtatggatggataccagaaactgcaatttggagtagtagatacgcaaacttagatgcaagtgactattatcccatttcgttgggattacttaaaaagttcgagtttctcatgtctctatataaaggtcctattccagtatacgaagaaaaagtaaatactgaattcattgctaatggatcgttctctggtagatacgtatcatatcttcgaaagttttctgctcttccaacaaacgagtttattagttttttgttactgacttccattccaatctataatatcttgttctggtttaaaaatactcagtttgatattactaaacacacattattcagatacgtctatacagataatgccaaacacctggcgttggctaggtatatgcatcaaacaggagactataagcctttgtttagtcgtctcaaagagaattatatatttaccggtcccgttccaataggtatcaaagatataaatcaccctaatcttagtagagcaagaagtccatccgattatgagacattagctaatattagtactatattgtactttaccaagtatgatccggtattaatgtttttattgttttacgtacctgggtattcaattactacaaaaattactccagccgtagaatatctaatggataaactgaatctaacaaagagcgacgtacaactgttgtaaattattttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtttcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgccgagtatttcatcgttgtatatttcttcttcgattgcataatcagattcttatataccgcctcaaactctattttaaaattattaaacaatactctattattaatcagtcgttctaactcctttgctatttctatggacttatctacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttaacatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacataatagtacgactttaaatccattttcccaaatatctttacgaattactttacatataatatcctcatcaacagtcacataattacctgtggttaaaacctttgcaaatgcagcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacatagtcgaacgttagttccaacaattgtttagctattcgtaacaaaactatttcagaacataaaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtcactacgtaataaaagtaatgaggaaatcgtataaatacggtctcaccatttcgacatctggatttcagatataaaaatctgttttcaccgtgactttcaaaccaattaatgcaccgaacatccatttatagaatttagaaatatattttcatttaaatgaatcccaaacattggggaagagccgtatggaccattatttttatagtactttcgcaagcgggtttagacggcaacatagaagcgtgtaaacgaaaactatatactatagttagcactcttccatgtcctgcatgtagacggcacgcgactatcgctatagaggacaataatgtcatgtctagcgatgatctgaattatatttattattttttcatcagattatttaacaatttggcatctgatcccaaatacgcgatcgatgtgacaaaggttaaccctttataaacttaacccattataaaacttatgattagtcacgactgaaataaccgcgtgattattttttggtataattctacacggcatggtttctgtgactatgaattcaacccccgttacattagtgaaatctttaacaaacagcaagggttcgtcaaagacataaaactcattgtttacaatcgaaatagaccccctatcacacttaaaataaaaaatatccttatcctttaccaccaaataaaattctgattggtcaatgtgaatgtattcacttaacagttccacaaatttatttattaactccgaggcacatacatcgtcggtattttttatggcaaactttactcttccagcatccgtttctaaaaaaatattaacgagttccatttatatcatccaatattattgaaatgacgttgatggacaaatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgttcaagactgtcaatctataatagatagtagctataatatattctatgatggtattgaagaagatgacaaccttggcatattgatcatttaacacagacatggtatcaacagatagcttgaatgaaagagaatcagtaattggaataagcgtcttctcgatggagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcgtctgtgcgtagatcacatcgtacactttttaattctctatagaagagcgacagacatctggagcaattacagacagcaatttctttattctctacagatgtaagatacttgaagacattcctatgatgatgcagaattttggataacacggtattgatggtatctgttaccataattcctttgatggctgatagtgtcagagcacaagatttccaatctttgacaatttttagcaccattatctttgttttgatatctatatcagacagcatggtgcgtctgacaacacaaggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatttacatcagttttgtattccaaattaaacatggcaacggatttaattttatattcctctattaagtcctcgtcgataataacagaatgtagataatcatttaatccatcgtacatggttggaagatgctcgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagtgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagattttaggtactcatctactctacaagaattttggagagccttaacgatatcagtgacgtttattatttcaggaggaaaaaacctaacattgagaatatcggaattaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacatgtcggcgacatgattaagtattgttttttcattatttttatattttctcaacaagttctcaataccccaatagatgatagaatatcacccaatgcgtccatgttgtctatttccaacaggtcgctatatccaccaatagaagttttcccaaaaaagattctaggaacagttctaccaccagtaatttgttcaaaatagtcacgcaattcattttcgggtttaaattctttaatatcgacaatttcatacgctcctcttttgaaactaaacttatttagaatatccagtgcatttctacaaaaaggacatgtatacttgacaaaaattgtcactttgttattggccaacctttgttgtacaaattcctcggccattttaatatttaagtgatataaaactatctcgacttatttaactctttagtcgagatatatggacgcagatagctatatgatagccaactacagaaggcaaacgctataaaaaacataattacgacgagcatatttataaatatttttattcagcattacttgatatagtaatattaggcacagtcaaacattcaaccactctcgatacattaactctctcattttctttaacaaattctgcaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatctgtcaaaaccaattcctttttgcctgtatcatccagttttccatcctttgtaaagaaattattttctactagactattaataagactgataaggattcctccataattgcacaatccaaactttttcacaaaactagactttacaagatctacaggaatgcgtacttcaggtttcttagcttgtgattttttcttttgtggacattttcttgtgaccaactcatctaccatttcattgattttagcagtgaaataagctttcaatgcacgggcactgatactattgaaaacgagttgatcttcaaattccgccatttaagttcaccaaacaacttttaaatacaaatatatcaatagtagtagaataagaactataaaaaaaataataattaaccaataccaaccccaacaaccggtattattagttgatgtgactgttttctcatcacttagaacagatttaacaatttctataaagtctgtcaaatcatcttccggagaccccataaatacaccaaatatagcggcgtacaacttatccatttatacattgaatattggcttttctttatcgctatcttcatcatattcatcatcaatatcaacaagtcccagattacgagccagatcttcttctacattttcagtcattgatacacgttcactatctccagagagtccgataacgttagccaccacttctctatcaatgattagtttcttgagtgcgaatgtaatttttgtttccgttccggatctatagaaaactacaggtgtgataattgccttggccaattgtctttctcttttactgagtgattctagttcaccttctatagatctgagaatggatgattctccagtcgaaacatattctaccatggctccgtttaatttgttgatgaagatggattcatccttaaatgttttctctgtaatagtttccaccgaaagactatgcaaagaatttggaatgcgttccttgtgcttaatgtttccatagacggcttctagaagttgatacaacataggactagccgcggtaacttttatttttagaaagtatccatcgcttctatcttgtttagatttatttttataaagtttagtctctccttccaacataataaaagtggaagtcatttgactagataaactatcagtaagttttatagagatagacgaacaattagcgtattgagaagcatttagtgtaacgtattcgatacattttgcattagatttactaatcgattttgcatactctataacacccgcacaagtctgtagagaatcgctagatgcagtaggtcttggtgaagtttcaactctcttcttgattaccttactcatgattaaacctaaataattgtactttgtaatataatgatatatattttcactttatctcatttgagaataaaaatgtttttgtttaaccactgcatgatgtacagatttcggaatcacaaaccaccggtggttttattttatccttgtccaatgtgaattgaatgggagcggatgcgggtttcgtacgtagatagtacattcccgtttttagaccgagactccatccgtaaaaatgcatactcgttagtttggaataactcggatctgctatatggatattcatagattgactttgatcgatgaaggctcccctgtctgcagccatttttatgatcgtcttttgtggaatttcccaaatagttttataaactcgcttaatatcttctggaaggtttgtattctgaatggatccaccatctgccataatcctattcttgatctcatcattccataattttctctcggttaaaactctaaggagatgcggattaactacttgaaattctccagacaatactctccgagtgtaaatattactggtatacggttccaccgactcattatttcccaaaatttgagcagttgatgcagtcggcataggtgccaccaataaactatttctaagaccgtatgttctgattttatcttttagaggttcccaattccaaagatccgacggtacaacattccaaagatcatattgtagaataccgttactggcgtacgatcctacatatgtatcgtatggtccttccttctcagctagttcacaactcgcctctaatgcaccgtaataaatggtttcgaagatcttcttatttagatcttgtgcttccaggctatcaaatggataatttaagagaataaacgcgtccgctaatccttgaacaccaataccgataggtctatgtctcttattagagatttcagcttctggaataggataataattaatatctataattttattgagatttctgacaattactttgaccacatccttcagtttgagaaaatcaaatcgcccatctattacaaacatgttcaaggcaacagatgccagattacaaacggctacctcattagcatccgcatattgtattatctcagtgcaaagattactacacttgatagttcctaaattttgttgattactctttttgttacacgcatccttataaagaatgaatggagtaccagtttcaatctgagattctataatcgctttccagacgactcgagcctttattatagatttgtatctcctttctctttcgtatagtgtatacaatcgttcgaactcgtctccccaaacattgtccaatccaggacattcatccggacacatcaacgaccactctccgtcatccttcactcgtttcataaagagatcaggaatccaaagagctataaatagatctctggttctatgttcctcgtttcctgtattctttttaagatcgaggaacgccataatatcagaatgccacggttccaagtatatggccataactccaggccgtttgtttcctccctgatctatgtatctagcggtgttattataaactctcaacattggaataataccgtttgatataccattggtaccggagatatagcttccactggcacgaatattactaattgatagacctattccccctgccattttagagattaatgcgcatcgttttaacgtgtcatagataccctctatgctatcatcgatcatgttaagtagaaaacagctagacatttggtgacgactagttcccgcattaaataaggtaggagaagcgtgcgtaaaccatttttcagaaagtagattgtacgtctcaatagctgagtctatatcccattgatgaattcctactgcgacacgcattaacatgtgctgaggtctttcaacgatcttgttgtttattttcaacaagtaggatttttccaaagttttaaaaccaaaatagttgtatgaaaagtctcgttcgtaaataataaccgagttgagtttatccttatatttgttaactatatccatggtgatacttgaaataatcggagaatgtttcccatttttaggattaacatagttgaataaatcctccatcacttcactaaatagtttttttgtttccttgtgtagatttgatacggctattctggcggctagaatggcataatccggatgttgtgtagtacaagtggctgctatttcggctgccagagtgtccaattctaccgttgttactccattatatattccttgaataaccttcatagctattttaataggatctatatgatccgtgtttaagccataacataattttctaatacgagacgtgattttatcaaacatgacattttccttgtatccatttcgtttaatgacaaacatttttgttggtgtaataaaaaaattatttaacttttcattaatagggatttgacgtacgtagcgtacaaaatgattgttcctggtatatagataaagagtcctatatatttgaaaatcgttacggctcgattaaactttaatgattgcatagtgaatatatcattaggatttaactccttgactatcagggcggcaccagaaattaccatcaaaagcattaatacagttatgcctatcgcagttagaacggttatagcatccaccatttatatctaaaaattagatcaaagaatatgtgacaaagtcctagttgtatattgagaattgacaaaacaatgtttcttacatattttttttttattagtaaccgacttaatagtaggaactggaaaactagacttgattattctataagtatagatacccttccaaataatattctctttgataaaagttccagaaaatgtagaattttttaaaaagttatcttttgctattaccaagattgtgtttagacgcttattattaatatgagtgatgaaatccacaccgcctctagatatcgcctttatttccacattagatggtaaatccaatagtgaaactatctttttaggaatgtatggactcgcgtttagaggagtgaacgtcttgggcgtcggaaaggatgattcgtcaaacgaataaacaatttcacaaatggatgttaatgtattagtaggaaattttttgacgctagtggaattgaagattctaatggatgatgttctacctatttcatccgataacatgttaatttccgacaccaacggttttaatatttcgatgatatacggtagtctctctttcggacttatatagcttattccacaatacgagtcattatatactccaaaaaacaaaataactagtataaaatctgtatcgaatgggaaaaacgaaattatcgacataggtatagaatccggaacattgaacgtattaatacttaattctttttctgtggtaagtaccgataggttattgacattgtatggttttaaatattctataacttgagacttgatagatattagtgatgaattgaaaattatttttatcaccacgtgtgtttcaggatcatcgtcgacgcccgtcaaccaaccgaatggagtaaaataaatatcattaatatatgctctaaatattagtatttttattaatcctttgattatcatcttctcgtacgcgaatgattccatgatcaagagtgatttgagaacatcctccggagtattaatgggcttagtaaacagtacatcgttgcaataataaaagttatccaagttaaaggatattatgcattcgtttaaagatatcacctcatctgacggagacaattttttggtaggttttagagactttgaagctacttgtttaacaaagttattcatcgtcgtttactattctatttaattttgtagttaatttatcacatatcacattaattgactttttggtccacttttccatacgtttatattcttttaatcctgcgttatccgtttccgttatatccagtgatagatcgtgcaggttaaatagaatgctcttaaataatgtcattttcttatccgctaaaaatttaaagaatgtataaacctttttcagagatttgaaactcttaggtggtgtcctagtacacaatatcataaacaaactaataaacattccacattcagattccaacagctgattaacttctacattaatacagcctattttcgctccaaatgtacattcgaaaaatctgaataaaacatcgatgtcacaatttgtattatccaatacagaatgtctgtgattcgtgttaaaaccatcggagaaggaatagaaataaaaattattatagtggtggaattcagttggaatattgcctccggagtcataaaaggatactaaacattgttttttatcataaattacacatttccaatgagacaaataacaaaatccaaacattacaaatctagaggtagaacttttaattttgtctttaagtatatacgataagatatgtttattcataaacgcgtcaaatttttcatgaatcgctaaggagtttaagaatctcatgtcaaattgtcctatataatccacttcggatccataagcaaactgagagactaagttcttaatacttcgattgctcatccaggctcctctctcaggctctattttcatcttgacgacctttggattttcaccagtatgtattcctttacgtgataaatcatcgattttcaaatccatttgtgagaagtctatcgccttagatactttttcccgtagtcgaggtttaaagaaatacgctaacggtatactagtaggtaactcaaagacatcatatatagaatggtaacgcgtctttaactcgtcggttaactctttcttttgatcgagttcgtcgctactattgggtctgctcaggtgccccaactctactagttccaacatcataccgataggaatacaagacactttgccagcggttgtagatttatcatatttctccactacatatccgttacaatttgttaaaaatttagatacatctatattgctacataatccagctagtgaatatatatgacataataaattggtaaatcctagttctggtattttactaattactaaatctgtatatctttccatttatcatggaaaagaatttaccagatatcttcttttttccaaactgcgttaatgtattctcttacaaatattcacaagatgaattcagtaatatgagtaaaacggaacgtgatagtttctcattggccgtgtttccagttataaaacatagatggcataacgcacacgttgtaaaacataaaggaatatacaaagttagtacagaagcacgtggaaaaaaagtatctcctccatcactaggaaaacccgcacacataaacctaaccgcgaagcaatatatatacagtgaacacacaataagctttgaatgttatagttttctaaaatgtataacaaatacagaaatcaattcgttcgatgagtatatattaagaggactattagaagctggtaatagtttacagatattttccaattccgtaggtaaacgaacagatactataggtgtactagggaataagtatccatttagcaaaattccattggcctcattaactcctaaagcacaacgagagatattttcagcgtggatttctcatagacctgtagttttaactggaggaactggagtgggtaagacgtcacaggtacccaagttattgctttggtttaattatttatttggtggattctctactctagataaaatcactaactttcacgaaagaccagtcattctatctcttcctaggatagctttagttagattgcatagcaataccattttaaaatcattgggatttaaggtactagatggatctcctatttctttacggtacggatctataccggaagaattaataaacaaacaaccaaaaaaatatggaattgtattttctacccataagttatctctaacaaaactatttagttatggcactcttattatagacgaagttcatgagcatgatcaaataggagatattattatagcagtagcgagaaagcatcatacgaaaatagattctatgtttttaatgactgccacgttagaggatgaccgagaacggctaaaagtatttttacctaatcccgcatttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtaaagtttttgtccccgctccttttggaggatcgcaagaatttatttctaaatctatgagagatcaacgaaaaggaagagtaggaagagttaatcctggtacatacgtctatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataactttgagaggacgggagaattaactagtattgtacgagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaatcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtacctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaattagtccatcatttgttgcgtcaagaactactatcggatgttgttgggtatctctagtgttacacatggccttactaaagtttgggtaaataactatgatatctctattaattatagatgcatatatttcatttgtcaaggatattagtatcgacttgctatcgtcattaatacgtgtaatgtaatcatataaatcatgcgatagccaaggaaaattcaaatagatgttcatcatataatcgtcgctataattcatattaatactttgacattgactaatttgtaatatagcctcgccacgaagaaagctctcgtattcagtttcatcgataaaggataccgttaaatataactggttgccgatagtctcatagtctattaagtggtaagtttcgtacaaatacagaatccctaaaatattatctaatgttggattaatctttaccataactgtataaaatggagacggagtcataactattttaccgtttgtacttactggaatagacgaaggaataatctccggacatgctggtaaagacccaaatgtctgtttgaagaaatccaatgttccaggtcctaatctcttaacaaaaattacgatattcgatcccgatatcctttgcattctatttaccagcatatcacgaactatattaagattatctatcatgtctattctcccaccgttatataaatcgcctccgctaagaaacgttagtatatccatacaatggaatacttcatttctaaaatagtattcgttttctaattctttaatgtgaaatcgtatactagaaagggaaaaattatctttgagttttccgttagaaaagaaccacgaaactaatgttctgattgcgtccgattccgttgctgaattaatggatttacaccaaaaactcatataacttctagatgtagaagcattcgctaaaaaattagtagaatcaaaggatataagtagatgttccaacaagtgagcaattcccaagatttcatctatatcattctcgaatccgaaattagaaattcccaagtagatatcctttttcatccgatcgttgatgaaaatacgaactttattcggtaagacaatcatttactaaggagtaaaataggaagtaatgttcgtatgtcgttatcatcgtataaattaaaggtgtgttttttaccattaagtgacattataattttaccaatattggaattataatataggtgtatttgcgcactcgcgacggttgatgcatcggtaaatatagctgtatctaatgttctagtcggtatttcatcatttcgctgtctaataatagcgttttctctatctgtttccattacagctgcctgaagtttattggtcggataatatgtaaaataataagaaatacatacgaataacaaaaataaaataagatataataaagatgccatttagagatctaattttgtttaacttgtccaaattcctacttacagaagatgaggaatcgttggagatagtgtcttccttatgtagaggatttgaaatatcttatgatgacttgataacttactttccagataggaaataccataaatatatttctaaagtatttgaacatgtagatttatcggaggaattaagtatggaattccatgatacaactctgagagatttagtctatcttagattgtacaagtattccaagtgtatacggccgtgttataaattaggagataatctaaaaggcatagttgttataaaggacaggaatatttatattagagaagcaaatgatgacttgatagaatatctcctcaaggaatacactcctcagatttatacatattctaatgagcgcgtccccataactggttcaaaattaattctttgtggattttctcaagttacatttatggcgtatacaacgtcgcatataacaacaaataaaaaggtagatgttctcgtttccaaaaaatgtatagatgaactagtcgatccaataaattatcaaatacttcaaaatttatttgataaaggaagcggaacaataaacaaaatactcaggaagatattttattcggtaaccggtggccaaactccataatttgctttttctatttcggattttagaatttccaaattcaccagcgatttatcggttttggtgaaatccaaggatttattaatgtccacaaatgccatttgttttgtctgtggattgtatttgaaaatggaaacgatgtagttagatagatgcgctgcaaagtttcctattagggttccgcgctttacgtcacccagcatacttgaatcaccatcctttaaaaaaaatgataagatatcaacatggagtatatcatactcggattttaattcttctactgcatcactgacattttcacaaatactacaatacggtttaccgaaaataatcagtacgttcttcatttatgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctactgataatcatcctaagatgataaaaaacttaactcaactgtttaaatttgttccctcggcagaggataactatttagcaaaattaacggcgttagtgaatggatgtgatttctttcctggactctatggggcatctataacacccaccaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatgttcctccttgtcaatgcactgttcaagaatttatattttccgcattagatgaaaaatggaacaattttaaatcatcttatttagagaccgttccgttaccctgccaattaatgtatgcattagaaccacgcaaggagattgatgtttcagaagttaaaactttatcatcttatatagatttcgaaaatactaaatcagatatcgatgttataaaatctatatcttcgatcttcggatattctaacgaaaactgtaacactatagtgttcggcatctataaggataatttactactgagtataaatagttcattttactttaacgatagtctgttaataaccaatactaaaagtgataatataataaatataggttactagattaaaaatggtgttccaactcgtgtgctctacatgcggcaaagatatttctcacgaacgatataaattgattatacgaaaaaaatcattaaaggatgtactcgtcagtgtaaagaacgaatgttgtaggttaaaattatctacacaaatagaacctcaacgtaacttaacagtgcaacctctattggatataaactaatatggatccggttaattttatcaagacatatgcgcctagaggttctattatttttattaattataccatgtcattaacaagtcatttgaatccatcgatagaaaaacatgtgggtatttattatggtacgttattatcggaacacttggtagttgaatctacctatagaaaaggagttcgaatagtcccattggatagtttttttgaaggatatcttagtgcaaaagtatacatgttagagaatattcaagttatgaaaatagcagctgatacgtcattaactttattgggtattccgtatggatttggtcatgatagaatgtattgttttaaattggtagctgaatgttataaaaatgccggtattgatacatcgtctaaacgaatattaggtaaagatatttttctgagccaaaacttcacagatgataatagatggataaagatatatgattctaataatttaacattttggcaaattgattaccttaaagggtgagttaatatgcataactactcctccgttgttttttccctcgttctttttcttaacgttgtttgccatcactctcataatgtaaagatattctaaaatggtaaacttttgcatatcggacgcagaaattggtataaatgttgtaattgtattatttcccgtcaatggactagtcacagctccatcagttttatatcctttagagtatttctcactcgtgtctaacattctagagcattccatgatctgtttatcgttgatattggccggaaagatagattttttattttttattatattactattggcaattgtagatataacttctggtaaatatttttctaccttttcaatctcttctattttcaagccggctatatattctgctatattgttgctagtatcaataccttttctggctaagaagtcatatgtggtattcactatatcagttttaactggtagttccattagcctttccacttctgcagaataatcagaaattggttctttaccagaaaatccagctactataataggctcaccgatgatcattggcaaaatcctatattgtaccagattaatgagagcatatttcatttccaataattctgctagttcttgagacattgatttatttgatgaatctagttggttctctagatactctaccatttctgccgcatacaataacttgttagataaaatcagggttatcaaagtgtttagcgtggctagaatagtgggcttgcatgtattaaagaatgcggtagtatgagtaaaccgttttaacgaattatatagtctccagaaatctgtggcgttacatacatgagccgaatgacatcgaagattgtccaatatttttaatagctgctctttgtccattatttctatatttgactcgcaacaattgtagataccattaatcaccgattcctttttcgatgccggacaatagcacaattgtttagctttggactctatgtattcagaattaatagatatatctcttaatacagattgcactatacattttgaaactatgtcaaaaattgtagaacgacgctgttctgcagccatttaactttaaataatttacaaaaatttaaaatgagcatccgtataaaaatcgataaactgcgccaaattgtggcatatttttcagagttcagtgaagaagtatctataaatgtagactcgacggatgagttaatgtatatttttgccgccttgggcggatctgtaaacatttgggccattatacctctcagtgcatcagtgttctaccgcggagccgaaaacattgtgtttaatcttcctgtgtccaaggtaaaatcgtgtttgtgtagttttcacaatgatgccatcatagatatagaacctgatctggaaaataatctagtaaaactttctagttatcatgtagtaagtgtcgattgtaacaaggaactgatgcctattaggacagatactactatttgtctaagtatagatcaaaagaaatcttacgtgtttaattttcacaagtatgaagaaaaatgttgtggtagaaccgtcattcatttagaatggttgttgggctttatcaagtgtattagtcagcatcagcatttggctattatgtttaaagatgacaatattattatgaagactcctggtaatactgatgcgttttccagggaatattctatgactgaatgttctcaagaactacaaaagttttctttcaaaatagctatctcgtctctcaacaaactacgaggattcaaaaagagagtcaatgtttttgaaactagaatcgtaatggataatgacgataacattctaggaatgttgttttcggatagagttcaatcctttaagatcaacatctttatgacgtttttagattaatactttcaatgagataaatatgggtggcggagtaagtgttgagctccctaaacgggatccgcctccgggagtacccactgatgagatgttattaaacgtggataaaatgcatgacgtgatagctcccgctaagcttttagaatatgtgcatataggaccactagcaaaagataaagaggataaagtaaagaaaagatatccagagtttagattagtcaacacaggacccggtggtctttcggcattgttaagacaatcgtataatggaaccgcacccaattgctgtcgcacttttaatcgtactcattattggaagaaggatggaaagatatcagataagtatgaagagggtgcagtattagaatcgtgttggccagacgttcacgacaccggaaaatgcgatgttgatttattcgactggtgtcagggggatacgttcgatagaaacatatgccatcagtggatcggttcagcctttaataggagtaatagaactgtagagggtcaacaatcgttaataaatctgtataataagatgcaaacattatgtagtaaagatgctagtgtaccaatatgtgaatcatttttgcatcatttacgcgcacacaatacagaagatagcaaagagatgatcgattatattctaagacaacagtctgcggactttaaacagaaatatatgagatgtagttatcccactagagataagttagaagagtcattaaaatatgcggaacctcgagaatgttgggatccagagtgttcgaatgccaatgttaatttcttgctaacacgtaattataataatttaggactttgcaatattgtacgatgtaatactagcgtgaacaacttacagatggataaaacttcctcattaagattgtcatgtggattaagcaatagtgatagattttctactgttcccgtcaatagagcaaaagtagttcaacataatattaaacactcgttcgacctaaaattgcatttgatcagtttattatctctcttggtaatatggatactaattgtagctatttaaatgggtgccgcggcaagcatacagacgacggtgaatacactcagcgaacgtatctcgtctaaattagaacaagaagcgaatgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtgccagctatgtttactgctgcgttaaacattcagacgagtgtaaacactgttgttagagattttgaaaattatgtgaaacagacttgtaattctagcgcggtcgtcgataacaaattaaagatacaaaacgtaatcatagatgaatgttacggagccccaggatctccaacaaatttggaatttattaatacaggatctagcaaaggaaattgtgccattaaggcgttgatgcaattgacgactaaggccactactcaaatagcacctaaacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgaaaatatattgataatattttaatagattaacatggaagttatcactgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaattatcaaatgttgttggcgttggtggcgctagtcatcacattaactattttttattactttatactataatagtactagactgacttctaacaaacatctcacctgccataaataaatgcttgatattaaagtcttctatttctaacactattccatctgtggaaaataatactctgacattatcgctaattgacacatcggtgagtgatatgcctataaagtaataatcttctttgggcacatataccagtgtaccaggttctaacaacctatttactggtgctcctatagcatactttttctttaccttgagaatatccatcgtttgcttggtcaatagcgatatgtgattttttatcaaccactcgaaaaagtaattggagtgttcatatcctctacgggctattgtctcatggccgtgtatgaaatttaagtaacacgactgtggtagatttgttctatagagccggttgccgcaaatagatagaactaccaatatgtctgtacaaatgttaaacattaattgattaacagaaaaaacaatgttcgttctgggaatagaaaccagatcaaaacaaaattcgttagaatatatgccacgtttatacattgaatataaaataactacagtttgaaaaataacagtatcatttaaacatttaacttgcggggttaatctcacaactttactgtttttgaactgttcaaaatatagcatagatccgtgagaaatacgtttagccgcctttaatagaggaaatcccaccgcctttctggatctcaccaacgacgatagttctgaccagcaactcatttcttcatcatccacctgttttaacatataataggcaggagatagatatccgtcattgcaatattccttctcgtaggcacacaatctaatattgataaaatctccattctcttctctgcatttattatcttgtttcggtggctgattaggctgtagtcttggtttaggctttggtatatcgttgttgaatctattttggtcattaaatctttcatttcttcctggtatatttttatcacctcgtttggttggatttttgtctatattatcgtttgtaacatcggtacgggtattcatttatcacaaaaaaaacttctctaaatgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgaaaatatcacaggagcaatgtttaccacaatgtataatataagaaacaatttgggtctaggagatgataaactaactattgaagccattgaaaactatttcttggatcctaacaatgaagttatgcctcttattattaataatacggatatgactgccgtcattcctaaaaaaagtggtaggagaaagaataagaacatggttatcttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatgtcctgaatgtgtattctccttccacatccatgagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttctttatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgtgatatcgcattttctaacgtgatggatatattaaagtcgaataaagtgaacaataattaattctttattgtcatcatgaacggcggacatattcagttgataatcggccccatgttttcaggtaaaagtacagaattaattagacgagttaaacgttatcaaatagctcaatataaatgcgtgactataaaatattctaacgataatagatacggaacgggactatggacgcatgataagaataattttgaagcattggaagcaactaaactatgcgatgttttggaattaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagataaaaataataggaggtaatgatatgtatcaatcggtgtgtagaaagtgttacatcgactcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaattagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacgtaaaatatctagcaaaaattctatgtctaaaaacagaaatagcaagagatccgtacgcggtaataaatagaaacgtgctactgagatatactaccgatatagagtataatgatttagttactttaataaccgttagacataaaattgattctatgaaaactgtgtttcaggtatttaacgaatcatccataaattatactccggttgatgatgattatggagaaccaatcattataacatcgtatcttcaaaaaggtcataacaagtttcctgtaaattttctatacatagatgtggtaatatctgacttatttcctagctttgttagactagatactacagaaactaatatagttaatagtgtactacaaacaggtgatggtaaaaagactcttcgtcttcccaaaatgttagagacggaaatagttgtcaagattctctatcgccctaatataccattaaaaattgttagatttttccgcaataacatggtaactggagtagagatagccgatagatctgttatttcagtcgctgattaatcaattagtagagatgagataagaacattataataatcaataatatatcttatatcttatatcttatatcttatatcttgtttagaaaaatgctaatattaaaatagctaacgctagtaatccaatcggaagccatttgatatctataatagggtatctaatttcctgatttaaatagcggacagctatattctcggtagctactcgtttggaatcacaaacattatttacatctaatttactatctgtaatggaaacgtttcccaatgaaatggtacaatccgatacattgcattttgttatatttttttttaaagaggctggtaacaacgcatcgcttcgtttacatggctcgtaccaacaataatagggtaatcttgtatctattcctatccgtactatgcttttatcaggataaatacatttacatcgtatatcgtctttgttagcatcacagaatgcataaatttgttcgtccgtcatgataaaaatttaaagtgtaaatataactattatttttatagttgtaataaaaagggaaatttgattgtatactttcggttctttaaaagaaactgacttgataaaaatggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaaaacttgtgggaaaacggaattggaatgtttcggtcactggggtaaagtaagtatttataaaactcatatagttaagcctgaatttatttcagaaattattcgtttactgaatcatatatgtattcactgcggattattgcgttcacgagaaccgtattccgacgatattaacctaaaagagttatcgggacacgctcttaggagattaaaggataaaatattatccaagaaaaagtcatgttggaacagcgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgctgattattagaccggctattagtttttggatagatagtatacccaaagaaaccaatgaattaacttacttattaggtatgatcgttaagaattgtaacttgaatgctgatgaacaggttatccagaaggcggtaatagaatacgatgatattaaaattatttctaataacactaccagtatcaatttatcatatatcacatccggcaaaaataatatgattagaagttatatcgtcgcccggcgaaaagatcagaccgctagatctgtaattggtcccagtacatctatcaccgttaatgaggtaggaatgcccgcatatattagaaatacacttacagaaaagatatttgttaatgcctttacagtggataaagttaaacaactattagcgtcaaaccaagttaaattttactttaataaacgattaaaccaattaacaagaatacgccaaggaaagtttatcaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgccaactctcaaaatgctgatttcgacggggatgaggaatggatgatattagaacaaaatcctaaagctgtaattgaacaaagtattcttatgtatccgacgacgttactcaaacacgatattcatggagcccccgtttatggatctattcaagatgaaatcgtagcagcgtattcattgtttaggatacaagatctttgtttagatgaagtattgaacatcttggggaaatatggaagagagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcctacagaactaatgtatattctaggtacgtatggacaacaaaggattgatggtgaaccagcagagactcgagtattgggtagagtcttaccttactatcttccagactctaaggatccagaaggaagaggttacattcttaattctttaacaaaaggattaacgggttctcaatattacttttcgatgctggttgcaagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaagaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacttgactaatttgagtaagaataagaccgaaattatcactctggtatccgatgatatctctaaacttcaatctgttaagattaatttcgaatttgtatgtttgggagaattaaatccagacatcactcttcgaaaagaaacagataggtatgtagtagatataatagtcaatagattatacatcaagagagcagaaattaccgaattagtcgtcgaatatatgattgaacgatttatctcctttagcgtcattgtaaaggaatggggtatggaaacattcattgaggatgaggataatattagatttactgtctacctaaatttcgttgaaccggaagaattgaatcttagtaagtttatgatggttcttccgggtgccgccaacaagggcaagattagtaaattcaagattcctatctctgactatacgggatatgacgacttcaatcaaacaaaaaagctcaataagatgactgtagaactcatgaatctaaaagaattgggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaattcttatcaataacatatttttctatgatctgtcttttaaacgatggattttccacaaatgcgcctctcaagtccctcatagaatgatacacgtataaaaaatatagcataggcaatgactccttatttttagacattagatatgccaaaatcatagccccgcttctatttactcccgcagcacaatgaaccaacacgggctcgtttcgttgatcacatttagataaaaaggcggttacgtcgtcaaaatatttactaatatcggtagttgtatcatctaccaacggtatatgaataatattaatattagagttaggtaatgtatatttatccatcgtcaaatttaaaacatatttgaacttaacttcagatgatggtgcatccatagcatttttataatttcccaaatacacattattggttacccttgtcattatagtgggagatttggctctgtgcatatctccagttgaacgtagtagtaagtatttatacaaacttttcttatccatttataacgtacaaatggataaaactactttatcggtaaacgcgtgtaatttagaatacgttagagaaaaggctatagtaggcgtacaagcagccaaaacatcaacacttatattctttgttattatattggcaattagtgcgctattactctggtttcagacgtctgataatccagtctttaatgaattaacgagatatatgcgaattaaaaatacggttaacgattggaaatcattaacggatagcaaaacaaaattagaaagtgatagaggtagacttctagccgctggtaaggatgatatattcgaattcaaatgtgtggatttcggcgcctattttatagctatgcgattggataagaaaacatatctgccgcaagctattaggcgaggtactggagacgcgtggatggttaaaaaggcggcaaaggtcgatccatctgctcaacaattttgtcagtatttgataaaacacaagtctaataatgttattacttgtggtaatgagatgttaaatgaattaggttatagcggttattttatgtcaccgcattggtgttccgattttagtaatatggaatagtgttagataaatgcggtaacgaatgttcctgtaaggaaccataacagtttagatttaacgttaaagatgagcataaacataataaacaaaattacaatcaaacctataacattaatatcaaacaatccaaaaaatgaaatcagtggagtagtaaacgcgtacataactcctggataacgtttagtagctgccgttcctattctagaccaaaaattcggtttcatgttttcgaaacggtgttctgcaacaagtcggggatcgtgttctacatatttggcggcattatccagtatctgcctattgatcttcatttcgttttcaattctggctatttcaaaataaaatcccgatgatagacctccagactttataatttcatctacgatgttcagcgccgtagtaactctaataatataggctgataagctaacatcataccctcctgtatatgtgaatatggcatgatttttgtccattacaagctcggttttaactttattgcctgtaataatttctctcatctgtaggatatctatttttttgtcatgcattgccttcaagacgggacgaagaaacgtaatatcctcaataacgttatcgttttctacaataactacatattctacctttttattttctaactcggtaaaaaaattagaatcccatagggctaaatgtctagcgatatttcttttcgtttcctctgtacacatagtgttacaaaaccctgaaaagaagtgagtatacttgtcatcatttctaatgtttcctccagtccactgtataaacgcataatccttgtaatgatctggatcatccttgactaccacaacatttcttttttctggcataacttcgttgtcctttacatcatcgaacttctgatcattaatatgctcatgaacattaggaaatgtttctgatggaagtctatcaataactggcacaacaataacaggagttttcgccgccgccatttagttattgaaattaatcatatacaactctttaatacgagttatattttcgtctatccattgtttcacatttacatatttcgacaaaaagatataaaatgcgtattccaatgcttctctgtttaatgaattactaaaatatacaaacacgtcactgtctggcaataaatgatatcttagaatattgtaacaatttattttgtattgcacatgttcgtgatctatgagttcttcttcgaatggcataggatctccgaatctgaaaacgtataaataggagttagaataataatatttgagagtattggtaatatataaactctttagcggtataattagtttttttctctcaatttctatttttagatgtgatggaaaaatgactaattttgtagcattagtatcatgaactctaatcaaaatcttaatatcttcgtcacacgttagctctttgaagtttttaagagatgcatcagttggttcgaccgatggagtaggtgcaacaattttttgttcgatgtatgtatgtactggagccattgttttaactataatggtgcttgtatcgaaaaactttaatgcagatagcggaagctcttcgccgcgactttctacatcgtaattgggttctaacgccgatctctgaatggatactagttttctaagttctaatgtgattctctgaaaatgtaaatccaattcctccggcattatagatgtgtatacatcggtaaataaaactatagtatccaacgatcccttctcgcaaattctagtcttaaccaaaaaatcgtatataaccacggagatggcgtatttaagagtggattcttctaccgttttgttcttggatgtcatataggaaactataaagtccgcactactgttaagaatgattactaacgcaactatatagttcaaattaagcattttggaaacataaaataactctgtagacgatacttgactttcgaataagtttgcagacaaacgaagaaagaacagacctctcttaatttcagaagaaaactttttttcgtattcctgacgtctagagtttatatcaataagaaagttaagaattagtcggttaatgttgtatttcattacccaagtttgagatttcataatattatcaaaagacatgataatattaaagataaagcgctgactatgaacgaaatagctatatggttcgctcaaaaatatagtcttgttaaacgtggaaacgataactgtatttttaatcacgtcagcggcatctaaattaaatataggtatatttattccacacactctacaatatgccacaccatcttcataataaataaattcgttagcaaaattattaattttagtgaaatagttagcgtcaactttcatagcttccttcaatctaatttgatgctcacacggtgcgaattccactctaacatcccttttccatgcctcaggttcatcgatctctataatatctagttttttgcgtttcacaaacacaggctcgtctctcgcgatgagatctgtatagtaactatgtaaatgataactagatagaaagatgtagctatatagatgacgatcctttaagagaggtataataactttaccccaatcagatagactgttgttatggtcttcggaaaaagaatttttataaatttttccagtattttccaaatatacgtacttaacatctaaaaaatccttaatgataataggaatggataatccgtctattttataaagaaatacatatcgcacattatacttttttttggaaatgggaataccgatgtgtctacataaatatgcaaagtctaaatattttttagagaatcttaattggtccaaattcttttccaagtacggtaatagatttttcatattgaacggtatcttcttaatctctggttctagttccgcattaaatgatgaaactaagtcactatttttataactaacgattacatcacctctaacatcatcatttaccagaatactgatcttcttttgtcgtaaatacatgtctaatgtgttaaaaaaaagatcatacaagttatacgtcatttcatctgtggtattcttgtcattgaaggataaactcgtactaatctcttctttaacagcctgttcaaatttatatcctatatacgaaaaaatagcaaccagtgtttgatcatccgcgtcaatattctgttctatcgtagtgtataacaatcgtatatcttcttctgtgatagtcgatacgttataaaggttgataacgaaaatatttttatttcgtgaaataaagtcatcgtaggattttggacttatattcgcgtctagtagatatgcttttatttttggaatgatctcaattagaatagtctctttagagtccatttaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatggcgtggtcaattacgaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggccgctgtagagctcgatgatagcgacggggatgatgaacctatggtacaagttgaagctggtaaagtaaatcatagtgctagaagcgatctttctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaaatgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagcgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataaaccgctattgaaactgacggatgattctagtcccgaagaatttctgttcaacaaactaagtgaacgaaaggtatacgaatgtatcaaacagtttggtattagaatcaaggatctccgaacgtatggagtcaattatacgtttttatataatttttggacaaatgtaaagtccatatctcctcttccatcaccaaaaaagttaatagcgttaactatcaaacaaactgctgaagtggtaggtcatactccatcaatttcaaaaagagcttacatggcaacgactattttagaaatggtaaaggataaaaattttttagatgtagtatctaaaactacgttcgatgaattcctatctatagtcgtagatcacgttaaatcatctacggatggatgatatagatctttacacaaataattacaagaccgataaatggaaatggataagcgtatgaaatctctcgcaatgaccgctttctttggggagctaagcacattagatattatggcattgataatgtctatatttaaacgccatccaaacaataccattttttcagtggataaggatggtcagtttatgattgatttcgaatacgataattataaggcttctcaatatttggatctgaccctcactccgatatttggagatgaatgcaagactcacgcatcgagtatagccgaacaattggcgtgtgcggatattattaaagaggatattagcgaatacatcaaaactactccccgtcttaaacgatttataaaaaaataccgcaatagatcagatactcgcatcagtcgagatacagaaaagcttaaaatagctctagctaaaggcatagattacgaatatataaaagacgcttgttaataagtaaatgaaaaaaaactagtcgtttataataaaacacaatatggatgccaacatagtatcatcttctactattgcaacgtatatagacgctttagcgaagaatgcttcagaattagaacagaggtctaccgcatacgaaataaataatgaattggaactagtatttattaagccgccattaattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccaatctaaaagttctttgttgcacgctattaatcatccaaagtcaaggcctaatacatctctggaaatagaattcacacctagagacaatgaaaaagttccatatgatgaactaataaaggaattgacgactctatcacgtcatatatttatggcttctccagagaatgtaattctttctccgcctattaacgcacctataaagacttttatgttgcctaaacaagatatagtaggtctggatctggaaaatctatatgccgtaactaagactgacggcattcctataactatcagagttacatcaaacgggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatatgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagaatcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagttgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgctgacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccgtctacaaaaaactttttcgaactaaatagaggagccattaaatgtgaaggtttagatgtcgaagacttacttagttactatgttgtttatgtcttttctaagcggtaaataataatatggtatgggttctgatatccccgttctaaatgcattaaataattccaatagagcgatttttgttcctataggaccttccaactgtggatactctgtattgttaatagatatattaatacttttgtcgggtaacagaggttctacgtcttctaaaaataaaagtttgataacatctggcctgttcataaataaaaacttggcgattctatatatactcttattatcaaatctagccattgtcttatagatgtgagctactgtaggtgtaccatttgattttctttctaatactatatatttctctcgaagaagttcttgcacatcatctgggaataaaatactactgttgagtaaatcagttattttttttatatcgatattgatggacatttttatagttaaggataataagtatcccaaagtagataacgacgataacgaagtatttatacttttaggaaatcacaatgactttatcagatcaaaattaacaaaattaaaggagcatgtatttttttctgaatatattgtgactccagataaatatggatctttatgcgtcgaattaaatgggtctagttttcagcacggcggtagatatatagaggtggaggaatttatagatgctggaagacaagttagatggtgttctacatccaatcatatatctgaagatatacccgaagatatacacactgataaatttgtcatttatgatatatacacttttgacgctttcaagaataaacgattggtattcgtacaggtacctccgtcgttaggagatgatagctatttgactaatccgttattgtctccgtattatcgtaattcagtagccagacaaatggtcaatgatatgatttttaatcaagattcatttttaaaatatttattagaacatctgattagaagccactatagagtttctaaacatataacaatagttagatacaaggataccgaagaattaaatctaacgagaatatgttataatagagataagtttaaggcgtttgtattcgcttggtttaacggcgtttcggaaaatgaaaaggtactagatacgtataaaaaggtatctaatttgatataatgaattcagtgactgtatcacacgcgccatatactattacttatcacgatgattgggaaccagtaatgagtcaattggtagagttttataacgaagtagccagttggctgctacgagacgagacgtcgcctattcctgataagttctttatacagttgaaacaaccgcttagaaataaacgagtatgtgtgtgcggtatagatccgtatccgaaagatggaactggtgtaccgttcgaatcaccaaattttacaaaaaaatcaattaaggagatagcttcatctatatctagattaaccggagtaattgattataaaggttataaccttaatataatagacggggttataccctggaattattacttaagttgtaaattaggagaaacaaaaagtcacgcgatctactgggataagatttccaagttactgctgcagcatataactaaacacgttagtgttctttattgtttgggtaaaacagatttctcgaatatacgggccaagttagaatccccggtaactaccatagtcggatatcatccagcggctagagaccgccaattcgagaaagatagatcatttgaaattatcaacgttttactggaattagacaacaaggcacctataaattgggctcaagggtttatttattaatgctttagtgaaattttaacttgtgttctaaatggatgcggctattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggcgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaacaagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacaacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttagatggtaataaactgtttaatattgcacaaagaattttagacactaactctgttttattaaccgaacgaggagaccatatagtttggattaataattcatggaaatttaacagcgaagaacccttgataacaaaactaattttgtcaataagacatcaactacctaaggaatattcaagcgaattactctgtccaagaaaacgaaagactgtagaagctaacatacgagacatgttagtagattcagtagagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagctatatgaaaaaacattatctagttgtttatgtggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtttttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacagaaaatatcatgttcctattatgaaactatatcctacaccggaagagattccggactttgcattctatctcaaaataggtactctgttagtatctagctctgtaaagcatattccattaatgacggacctctccaaaaagggatatatattgtacgataatgtggtcactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaatatatgctcatatatttatagaagatatcacatatctaaatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatatcaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttctactattgtctggatctcccattactaacacacctaatactctgggtcatattatagatttaatgtccgaagagacgatagattttggtgagattattagtcgtggtaagaaagtaattcagacacttcttaacgaacgcggtgtgaatgtacttaaggatttgcttaaaggaagaatatcatattacgaaatgcctgataaagatctaccaacgataagatatcacggacgtaagtttctagatactagagtagtatattgtcacatgtctaaacttcaagagagagattatatgattactagacgacagctatgttatcatgaaatgtttgataaaaatatgtataacgtgtcaatggcagtattgggacaacttaatctgatgaataatttagatactttatttcaggaacaggataaggaattgtacccaaatctgaaaataaataatggcgtgttatacggagaagaattggtaacgttaaacattagttccaaatttaaatactttattaatcggatacagacactcaacggaaaacattttatatacttttctaattctacatatggtggattggtaattaaatatatcatgctcagtaatggatattctgaatataatggttctcagggaactaatccacatatgataaacggcaaaccaaaaacatttgctatcgttactagtaaaatgaaatcgtctttagaggatctattagatgtgtataattctcctgaaaacgatgatggcagtcaattgatgtttttgttttcatcaaacattatgtccgaatcctatactctaaaagaggtaaggcatatttggtttatgactatcccagatactttttctcaatacaaccaaattcttggacgatctattagaaaattctcttacgccgatatttctgaaccagttaatgtatatcttttagccgccgtatattccgatttcaatgacgaagtaacgtcattaaacgattacacacaggatgaattgattaatgttttaccatttgacatcaaaaagctgttgtatctaaaatttaagacgaaagaaacgaatagaatatactctattcttcaagagatgtctgaaacgtattctcttccaccacatccatcaattgtaaaagttttattgggagaattggtcagacaatttttttataataattctcgtattaagtataacgactccaagttacttaaaatggttacatcagttataaaaaataaagaagacgctaggaattacatagatgatattgtaaacggtcacttctttgtatcgaataaagtatttgataaatctcttttatacaaatacgaaaacgatattattacagtaccgtttagactttcctacgaaccatttgtttggggagttaactttcgtaaagaatataacgtggtatcttctccataaaactgatgaaatatataaagaaataaatgtcgagctttgttaccaatggataccttccagttacattggaaccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgtcgactcataaaaaagagaatagcggtaagtataaacacgaatactatggcaataattgcgaatgttttattctcttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatgcggttttccatcatgattagacgacgacaatagtgttctgaatttagatagttgatcagaatgaatgtttattggcgttggaaaaattatccatacagcgtctgcagagtggttgatagttgttcctagatatgtaaaataatccaacttactaggcagcaaattgtctagataaaatactgaatcaaacggtgcagacgtattggcggatctaatggaatccaattgattaactatcttttgaaaatatacatttttatgatccaatacttgtaagaatatagaaataatgataagtccatcatcgtgtttttttgcctcttcataagaactatatttttttttattccaatgaacaagattaatctctccagagtatttgtacacatctatcaagtgattggatccataatcgtcttcctttccccaatatatatgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcatagtaaaaatgaaatgataactgtttaaaaatagctcttagtatgggaattacaatggatgaggaagtgatatttgaaactcctagagaattaatatctattaaacgaataaaagatattccaagatcaaaagacacgcatgtgtttgctgcgtgtataacaagtgacggatatccgttaataggagctagaagaacttcattcgcgttccaggcgatattatctcaacaaaattcagattctatctttagagtatccactaaactattacggtttatgtactacaatgaactaagagaaatctttagacggttgagaaaaggttctatcaacaatatcgatcctcactttgaagagttaatattattgggtggtaaactagataaaaaggaatctattaaagattgtttaagaagagaattaaaagaggaaagtgatgaacgtataacagtaaaagaatttggaaatgtaattctaaaacttacaacacgggataaattatttaataaagtatatataagttattgcatggcgtgttttattaatcaatcgttggaggatttatcgcatactagtatttacaatgtagaaattagaaagattaaatcattaaatgattgtattaacgacgataaatacgaatatctgtcttatatttataatatgctagttaatagtaaatgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtctagaaagaaacgattatttctgaattattccaattatttgtccaaacaggaaagaagtatactatcgtcatttttttctctagatccagctactactgataatgatagaatagatgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctttaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattgattagaaaatataaattaatttaccatcgtgtatttttataacgggattgtccggcatatcatgtagatagttaccgtctacatcgtatactcgaccatctacgcctttaaatcctctatttattgacattaatctattagaattggaataccaaatattagtaccctcaattagtttattggtaatattttttttagacgatagatcgatggctcttgaaaccaaggttttccaaccggactcattgtcgatcggtgagaagtctttttcattagcatgaatccattctaatgatgtatgtttaaacactctaaacaattggacaaattcttttgatttgctttgaatgatttcaaataggtcttcgtctacagtaggcataccattagataatctagccattataaagtgcacgtttacatatctacgttctggaggagtaagaacgtgactattgagacgaatggctcttcctactatctgacgaagagacgcctcgttccatgtcatatctaaaatgaagatatcattaattgagaaaaaactaataccctcgcctccactagaagagaatacgcatgttttaatgcattctccgttagtgtttgattcttggttaaactcagccaccgccttgattctagtatcttttgttctagatgagaactctatattagagataccaaagactttgaaatatagtaataagatttctattcctgactgattaacaaatggttcaaagactagacatttaccatgggatgctaatattcccaaacatacatctataaatttgacgcttttctcttttaattcagtaaatagagagatatcagccgcactagcatcccctttcaatagttctccctttttaaaggtatctaatgcggatttagaaaactctctatctcttaatgaatttttaaaatcattatatagtgttgctatctcttgcgcgtattcgcccggatcacgattttgtctttcaggaaagctatcgaacgtaaacgtagtagccatacgtctcagaattctaaatgatgatatacctgtttttatttcagcgagtttagccttttgataaatttcttcttgctttttcgacatattaacgtatcgcattaatactgttttcttagcgaatgatgcagacccttctacgtcatcaaaaatagaaaactcgttattaactatgtacgaacataggcctcctagtttggagactaattctttctcatcaactagacgtttattctcaaatagcgattggtgttgtaaggatcctggtcgtagtaagttaaccaacatggtgaattcttgcacactattaacgataggtgtagccgataaacaaatcatcttatggttttttaatgcgatggtcttagataaaaaattatatactgaacgagtaggacggatcttaccatcttctttgattaatgatttagaaatgaagttatgacattcatcaataatgacgcatattctactcttggaattaatagttttgatattagtaaaaaatttatttctaaaattttgatcatcgtaattaataaaaatacaatccttcgttatctctggagcgtatctgagtatagtgttcatccaaggatcttctatcaaagcctttttcaccaataagataatagcccaattcgtataaatatccttaagatgtttgagaatatatacagtagtcattgttttaccgacacccgtttcatggaacaataaaagagaatgcatactgtctaatcctaagaaaactcttgctacaaaatgttgataatccttgaggcgtactacgtccgaccccatcatttcaacgggcatattagtagttctgcgcaatgcataatcgatataggccgcgtgtgatttactcatttatgagtgataagtaataactatgttttaaaaatcacagcagtagtttaactagtcttctctgatgtttgttttcgatactttttgaatcagaagtcatactagaataaagcaacgagtgaacgtaatagagagcttcgtatactctattcgaaaactctaagaacttattaatgaattccgtatccactggattgtttaaaatactaaattgaacactgttcacatccttccaagaagaagacttagtgacggacttaacatgagacataaataaatccaaattttttttacaaacatcactagccaccataatggcgctatctttcaaccagctatcgcttacgcattttagcagtctaacatttttaaagagactacaatatattctcatagtatcgattacacctctaccgaataaagttggaagtttaataatacaatatttttcgtttacaaaatcaaataatggtcgaaacacgtcgaaggttaacatcttataatcgctaatgtatagattgttttcagtgagatgattattagatttaatagcatctcgttcacgtttgaacagtttattgcgtgcgctgaggtcggcaactacggcgtccgctttagtactcctcccataatactttacgctattaatctttaaaatttcatagactttatctagatcgctttctggtaacatgatatcatgtgtaaaaagttttaacatgtcggtcggcattctatttagatcattaactctagaaatctgaagaaagtaattagctccgtattccagactaggtaatgggcttttacctagagacagattaagttctggcaatgtttcataaaatggaagaaggacatgcgttccctcccggatattttttacaatttcatccatttacaactctatagtttgttttcattattattagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattagccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctcaacaatctcggcagactccggataaccagtcggtgggccatcactaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaacgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatataaagttggaatttgactatcgactgcgaagacattagaccgtttaatagagtcatccccaccgatcaaagaattaatgatagtattattcattttctatttaaaatggaaaaagcttacaataaactccgtagagaaatatctataatttgtgagttttccttaaagtaacagcttccgtaaacgccgtctttatctcttagtaagtttattgtatttataaccttttccttatcttcatagaatactaaaggcaacaaagaaatttttggttcttctctaagagctacgtgagacttaaccatagacgccaacgaatccctacatattttagaacagaaatacccaacttcaccacccttgaatgtctcaatactaataggtttaaaaaccaaatcttgattacaaaaccaacacttatcaattacactatttgtcttaatagacacatctgccatagatttataatactttggtagtatacaagcgagtgcttcttctttagcgggcttaaagactgctttaggtgctgaaataaccacatctggaaggcttactcgcttagccatttaattacggaactatttttttatacttctaatgagcaagtagaaaacctctcatctacaaaaacatactcgtgtccataatcctctaccatagttacacgttttttagatctcatatgtgctaaaaagttttcccatactaattggttactattatttttcgtataatttttaacagtttgaggttttagatttttagttacagaagtgatatcgaatattttatccaaaaagaatgaataattaattgtcttagaaggagtgttttcttggcaaaagaataccaagtgcttaaatatttctactacttcattaatcttttctgtactcagattcagtttctcatcttttacttgattgattatttcaaagactaacttataatcctttttatttattctctcgttagccttaagaaaactagatacaaaatttgcatctacatcatccgtggatatttgatttttttccatgatatccaagagttccgagataatttctccagaacattgatgagacaataatctccgcaatacatttctcaaatgaataagtttattagacacatggaagtttgactttttttgtacctttgtacatttttgaaataccgactcgcaaaaaatacaatattcatatccttgttcagatactataccgttgtgtctacaaccgctacataatcgtagattcatgttaacactctacgtatctcgtcgtccaatattttatataaaaacattttatttctagacgttgccagaaaatcctgtaatatttttagttttttgggctgtgaataaagtatcgccctaatattgttaccgtcttccgccaatatagtagttaaattatccgcacatgcaaaagaacaccgcttaggcggattcagtacaatgttatatttttcgtaccaactcatttaaatatcataatctaaaatagttctgtaatatgtctagcgctaatatattgatcataatcctgtgcataaattaagatacaacaatgtctcgaaatcatcgacatggcttcttccatagttagaagatcgtcgtcaaagttagcaacgtgattcatcaacatttgctgttttgaggcagcaaatactgaaccgtcgccattcaaccattcataaaaaccatcgtctgaatccattgataatttcttgtactggtttttgagagctcgcatcaatctagcatttctagctcccggattgaaaacagaaagaggatcgtacatccagggtccattttctgtaaatagaatcgtataatgtcccttcaagaagatatcagacgatccacaatcaaagaattggtctccgagtttgtaacaaactgcggactttaacctatacatgataccgtttagcatgatttctggtgatacgtcaatcggagtatcatctattagagatctaaagccggtgtaacattctccaccaaacatattcttattctgacgtcgttctacataaaacatcattgctccattaacgataacaggggaatgaacagcactacccatcacattagttcccaatggatcaatgtgtgtaactccagaacatcttccatatcctatgttaggaggagcgaacaccactcttccactattgccatcgaatgccatagaataaatatccttggaattgatagaaatcggactgtcggatgttgtgatcatcttcataggattaacaactatgtatggtgccgcctgaagtttcatatcgtaactgatgccgtttataggtctagccacagaaaccaacgtaggtctaaatccaactatagacaaaatagaagccaatatctgttcctcatctgtcataacttgagagcatccagtatgaataatcttcattagatggggatctaccgcatcatcatcgttacaataaaaaattcccattctaatgttcataattgcttttctaatcatggtatgcatgtttgctctctgaatctctgtggaaattagatctgatacacctgtaatcactatcggattatcctccgtaagacgattaaccaacaacatataattataagactttacttttctaaattcataaagttgctggattaggctataggtgtctccatgtacatacgcgttctcgagcgcaggaagtttaataccgaatagtgccatcagaataggatgaatatagtaattagtttctggttttctataaataaaagacaaatcttgtgaactagacatatcggtaaaatgcatggattggaatcgtgtagtcgacagaagaatatgatgattagatggagagtatattttatctaactctttgagttggtcaccgattctaggactagctcgagaatgaataagtactaaaggatgagtacatttcacagaaacactagcattgttcaatgtgctctttacatgggtaaggagttgaaatagctcgtttctatttgttctgacaatatttagtttattcataatgttaagcatatcctgaatagtaaagttagatgtgtcatacttgttagtagttagatatttagcaattgcattcccatcatttctcaatctcgtactccaatcatgtgtagatgctacttcgtcgatggaaaccatacaatcctttttgataggctgttgagattgattatttcctgcacgtttaggtttggtacgttgatttctagcccctgcagatataaagtcatcgtctacaattttggataatgaattgcatacactacaagacaaagatttatcagaagtgtgaatatgatcttcatctaccaaagaaagagtttgattagtataactagattttagtcctgcgttagatgttaaaaaaacatcgctattgaccacggcttccattatttatattcgtagtttttactcgaaagcgtgattttaatatccaatcttattacttttggaatcgttcaaaacctttgactagttgtagaatttgatctattgccctacgcgtatactcccttgcatcatatacgttcgtcaccagatcgtttgtttcggcctgaagttggtgcatatctttttcaacactcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatacttccatccgattgttgtgtacatgtagttgttggtaacgtctgagaaggttgggtagatggcggtgtcgtcgtcttttgatctttattaaatttagagataatatcctgaacagcattgctcggcgtcaacgctggaaggagtgaactcgccggcgcatcagtatctgcagacagccaatcaaaaagattagacatatcagatgatgtattagtttgttgtcgtggttttggtgtaggagcagtactactaggtagaagaataggagccgatgtaggtgtcggaaccggaaccggctgtggagttatatgaatagttggttgtagcggttggataggctgtctgctggcggccatcatattatctctagctagttgttctcgcaactgtctttgataatacgactcttgagactttagtcctatttcaatcgcttcatcctttttcgtatccggatcctttttttcagaataatagattgacgactttggtgtagaggattctgccagcccctgtgagaacttgttaaagaagtccatttaaggctttaaaattgaattgcgattataagattaaatggcagacacagacgatattatcgactatgaatccgatgatctcactgaatacgaggatgatgaagaagaggaagaagatggagagtcactagaaactagtgatatagatcccaaatcttcttataagattgtagaatcagcatccactcatatagaagatgcgcattccaatcttaaacatatagggaatcatatatctgctcttaaacgacgctatactagacgtataagtctatttgaaatagcgggtataatagcagaaagctataacttgcttcaacgaggaagattacctctagtttcagaattttctgacgaaacgatgaagcaaaatatgctacatgtaattatacaagagatagaggagggttcttgtcctatagtcatcgaaaagaacggagaattgttgtcggtaaacgattttgacaaagatggtctaaaattccatctagactatattatcaaaatttggaaacttcaaaaacgatattagaatttatacgaatatcgttctctaaatgtcacaatcaagtctcgcatgttcagcaatttattgtcgtactttatatcgtgttcattaacgatatcttgcaaaatagtaatgattctatcttccttcgatagatattcttcagagattattgtcttatattctttcttgttatccgatatgaatttgataagactttgaacattattaatacccgtctgtttaattttttctacagatattttagttttggcagattctatcgtatctgtcaatagacatccaacatcgacattcgacgtcaattgtctataaatcaacgtataaattttagaaataacattagcgaattgttgtgcattgatgtcgttattctgaaacagtatgattttaggtagcattttcttaacaaagagaacgtatttattgttactcagttgaacagatgatatatccagattactaacgcatctgattccgtataccaaactttcagaagaaatggtgtacaattgtttgtattcattcaatgtctccttttcagaaattagtttagagtcgaatactgcaataattttcaagagatagttttcatcagataagattttatttagtgtagatatgataaaactattgttttgttggagaacttgatacgccgcgttctctgtagtcgacgctctcaaatgggaaacgatctccattatttttttggaatcggatactatatcttcggtatcttgacgcagtctagtatacatagagttaagagagattagagtttgtacattaagcaacatgtctctaaatgtggctacaaacttttcctttttcacatcatctagtttattatataccgatttcacaacggcaccagatttaaggaaccagaatgaaaaactctgataactacaatatttcatcatagttacgattttatcatcttctatagttggtgtaatagcgcatacctttttctccaagactggaaccaacgtcataaaaatgtttaaatcaaaatccatatcaacatctgatgcgctaagaccagtctcgcgttcaagattatctttactaatggtgacgaactcatcgtatagaactctaagtttgtccattatttatttacagatttagttgtttaatttatttgtgctcttccagagttgggatagtatttttctaacgtcggtattatattattaggatctacgttcatatgtatcataatattaatcatccacgttttgataaatctatctttagcttctgaaataacgtatttaaacaaaggagaaaaatatttagctacggcatcagacgcaataacattttttgtaaatgtaacgtatttagacgacagatcttcgttaaaaagttttccatctatgtagaatccatcggttgttaacaccattcccgcgtcagattgaataggagtttgaatagtttgttttggaaatagatccttcaataacttatagttgggtgggaaaaaatcgattttatcactagactctttcttttttactatcattacctcatgaactatttcttgaatgagtatatgtattttctttcctatatcggacgcgttcattggaaaatataccatgtcgttaactataagaatatttttatcctcgtttacaaactgaataatatcagatgtagttcgtaaacgaactatatcatcaccagcacaacatctaactatatgatatccactagtttcctttagccgtttattatcttgttccatattagcagtcattccatcatttaagaaggcgtcaaaaataatagggagaaatgacattttggattctgttacgactttaccaaaattaaggatatacggacttactatctttttctcaacgtcaatttgatgaacacacgatgaaaatgtgcttctatgagattgatcatgtagaaaacaacaagggatacaatatttccgcatatcatgaaatatattaagaaatcccaccttattatatttccccaaaggatccatgcacgtaaacattatgccgttatcattaataaagacttctttctcatcggatctgtaaaagttgttactgatttttttcattccaggatctagataattaataatgatgggttttctattcttattctttgtattttggcatatcctagaccagtaaacagtttccactttggtaaaatcagcagacttttgaacgctattaaacatggcattaatggcaataactaaaaatgtaaaatatttttctatgttaggaatatggtttttcactttaatagatatatggtttttggccaaaatgatagatatttttttatccgaggatagtaaaatattattagtcgccgtctctataaaaatgaagctagtctcgatatccaattttattctagaattgataggagtcgccaaatgtaccttatacgttatatctcccttgatgcgttccatttgtgtatctatatcggacacaagatctgtaaatagttttacgttattaatcatcacggtatcgccgtcgctagataacgctaatgtaccatccaagtcccaaatggagagatttaactgttcatcgtttagaataaaatgattaccggtcatattaataaagtgttcatcgtatctagataacaacgacttataattaatgtccaagtcttgaactcgctgaatgatcttttttaacccagttagttttagattggtacgaaatatattgttaaactttgattctatagtaatgtccaaatctagttgtggaaatacttccatcaacattgtttcaaacttgataatattattatctacatcttcgtacgatccaaattccggaatagatgtatcgcacgctctggccacccagataaccaaaaagtcacacgctccaggatatacattgtataaaaagctatcgttttttagtagggtttttttctgcgtgtatacgaagggattaaaaatagtattatcaacgtaactatattccaaattattcttatgagaatagataataatatcgtccttaatatctaacaaatttcctaaatatccctttaattgagtcattcgaagcgtcaatagaatatgtctcttaactatttccggctgttgtatatttaaatgacttcgtaaaaaataatatatgggcgacttctcatctatgtaatcatatggagtgagatatagggctcgttctacctcctgccccttacccacctgtaataccaattgcggacttactatatatcgcatatttatatcgtggggtaaagtgaaaatctactaccgatgatgtaagtcttacaatgttcgaaccagtaccagatcttaatttggaggcctccgtagaactaggggaggtaaatatagatcaaacaacacctatgataaaggagaatagcggttttatatcccgcagtagacgtctattcgcccatagatctaaggatgatgagagaaaactagcactacgattctttttacaaagactttattttttagatcatagagagattcattatttgttcagatgcgttgacgctgtaaaagacgtcactattaccaaaaaaaataacattatcgtggcgccttatatagcacttttaactatcgcatcaaaaggatgcaaacttacagaaacaatgattgaagcattctttccagaactatataatgaacatagtaagaaatttaaattcaactctcaagtatccatcatccaagaaaaactcggataccagtttggaaactatcacgtttatgattttgaaccgtattactctacagtagctctggctattcgagatgaacattcatctggcatttttaatatccgtcaagagagttatctggtaagttcattatctgaaataacatatagattttatctaattaatctaaaatctgatcttgttcaatggagtgctagtacgggcgctgtaattaatcaaatggtaaatactgtattgattacagtgtatgaaaagttacaactggtcatagaaaatgattcacaatttacatgttcattggctgtggaatcaaaacttccaataaaattacttaaagatagaaatgaattatttacaaaattcattaacgagttaaaaaagaccagttcattcaagataagcaaacgcgataaggatacgctactaaaatattttacttaggactggagttagaatttatagacgactcatttcgtttatcattattactaccatcattattagtattcttcttgttatcttgttcagaaatatacagcaatgctatgcctaatactaaatacattatcatgcttgcaatggctctaacaacgacgaaccaaaatgaatttggtcgtagcttttgttcacaaaaatacataaagaaatgtctacataaatctatggcgccattggctacttgaaatagcgccagtcctcctacagattttaatatagctgtataacatgacatttattcatcatcaaaagagacagagtcaccatctgtcatatttagattttttttcatgtgttcaaagtatcctctactcatttcattataatagtttatcatacttagaattttaggacggatcaatgagtaagacttgactagatcgtcagtagtaatttgtgcatcgtctattctgcatccgcttcgtcgaataatgtatagcatcgctttgagattctccatagctatcaagtctttatacaatgacatggaaatatctgtgaatactttatacttctccaacatcgatgccttaacatcatcgcctactttagcattgaaaatacgttctattgtgtagatggatgtaacaagatttttaaacaacaatgccatcttacacgatgattgcctcaagtctccaatcgtttgtttagaacgattagctacagagtccaatgcttggctgactagcatattattatctttagaaattgtattcttcaatgaggcgtttatcatatctgtgatttcgttagtcatattacagtctgactgggttgtaatgttatccaacatatcacctatggatacggtacacgtaccagcatttgtaataatcctatctaagatgttgtatggcattgcgcagaaaatatcttctcctgtaatatctccactctcgataaatctactcagattattcttaaatgccttattctctggagaaaagatatcagtgtccatcatttcattaatagtatacgcagaaaagataccacgagtatcaattctatccaagatacttatcggttccgagtcacagataatggtttcctctccttcgggagatcctgcatagaaatatctaggacaatagtttctatactgtctgtaactctgataatctctaaagtcactaactgataccatgaaattgagaagatcaaacgctgaagtaattaatttttctgcctcgtttttactacaactagttttcatcaatgtagtgacgatgtattgtttagttacttttggtctaatactgatgatagagatattattgcttcccataatggatcttctagtagtcaccttaaagcccattgatgcgaatagcagatagataaagtcttggtatgactcctttctaatatagtacggactacctttgtcacccaactttatacccacataagccataacaacctctttaatagccgtttcatgaggtttatcagccatgagcctgagtagttggaagaatctcatgaatcccgtctcagaaagtcctatatgcatgatagatttatctttcctgggaaactctcgtatagtcatagatgaaatactcttcaaagtttctgaaataagattagtaacagtcttacctccgactactctgggtaacaaacaaactctaataggtgttttctctgcggagataatatcagaaaggatagagcaataagtagtattattgtgattataaagaccgaatacataacaggtagaatttataaacatcatgtcctgaaggtttttagacttgtattcctcgtaatccataccgtcccaaaacatggatttggtaactttgatagccgtagatctttgttccttcgccaacaggttaaagaaattaataaagaatttgttgtttctatttatgtccacaaattgcacgtttggaagcgccacggttacattcactgcagcattttgaggatcgcgagtatgaagtacgatgttattgtttactggtatatctggaaagaaatctaccagtctaggaataagagattgatatcgcatagaaatagtaaagtttataatctcatcatcgaagagcattttgttaccattgtaataaatatccactctgtcatatgtataaatgaagtactgttcaaacatgatgagatgtttatatgttggcatagtagtgagatcgacgtttggtaatggcaatgtattaagattaactccataatgtctagcagcatctgcgatgttataagcgtcgtcaaagcggggtcgatcttgtattgttatatattgtctaacacctataagattatcaaaatcttgtctgcttaatacaccgttaacaatttttgccttgaattcttttattggtgcattaataacatccttatagaggatgttaaacaaataagtgttatcaaagttaagatctggatatttcttttctgctagaacatccattgagtcggagccatctggtttaatataaccaccgataaatctagctctgtattctgtatccgtcaatctaatattaagaaggtgttgagtgaaaggtggaagatcgtaaaagctgtgagtattaatgataggattagtttccgaactaatgttaattggggtattaataatatctatatttccagcgttaagtgtaacattaaacagttttaattcacgtgaagtagtatcaattaaataattaatgcccaatttggatatagcagcctgaagctcatcttgtttagttacggatcctaatgagttattaagcaatatatcgaacggatgaacgaaggttgttttgagtttgtcgcatactttgtaatctagacatagatgcggaagaacggtagaaactatacgaaataaatattcagagtcctctaattgatcaagagtaactattgacttaataggcatcatttatttagtattaaatgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattcaccggccactattatcgaacgagtacaacctcatactactattattgacgatactccacctcctacgtttcgtagagagttattaatatcggaacaacgtcaacaacgagaaaaaagatttaatattacagtatcgaaaaatgctgaagcaataatggaatctagatctatgataacttctatgccaacacaaacaccatccttgggagtagtttatgataaagataaaagaattcagatgttagaggatgaagtggttaatcttagaaatcaacgatctaatacaaaatcatctgataatttagataattttaccaaaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacgaaaaattatcgtcactaacgtgattattattgtcataaatattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaatttaatacattcccatatccagacaacaatcgtctggattaatctgttcctgtcgtctcataccggacgacatattaatctttttattagtgggcatctttttagatggtttctttttcccagcattaactgagtcgatacctagaagatcgtgattgatctctccgaccattccacgaacttctaattggccgtctctgacggtaccataaactattttaccagcattagtaacagcttggacaatctgaccatccatcgcattgtacgatgtagtagtaactgttgttctacgtttaggagcaccagaagtatttttggagcccttggaggctgatgtagaagaagacgaggattttgattttggtttacatgtaatacattttgaactctttgattttgtatcacatgcgccggcagtcacatctgtttgagaattaagattattgttgcctcctttgacggctgcatctccaccgatttgcgctagtagatttttaagctgtggtgtaatcttattaactgtttcgatataatcatcgtaactgcttctaacggctaaattttttttatccgccatttagaagctaaaaatatttttatttatgcagaagatttaactagattatacaatgaactaatatgatccttttccagattatttacaaacttggtattttttggttctggaggaggcgaatttaaattcggacttggattcagattttgtaagttcttgatcttattatacatcgagtataggatggcgacagtaactgctacacaaataccgatcaaaagaagaataccaatcatttattgacaataacttcactattgatcaagtatgcaatatatcatcttttcactaaataagtagtaataatgattcaacaatgtcgagatatatggacgataataatttagttcatggaaatatcgctatgattggtgtgaatgactccgctaactctgtggggtgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatgtccatttaactaataaaaattttaaatcgccgaatgaacaaagtggaatataaaccatataaaaacaatagtttgtactgcaaaaataatatctatttttgttttcgaagatatggtaaaattaaatagtagtacacagcatgttataactaacagcagcaacggctcgtaattacttatcatttactagacgaaaaggtggtgggatattttcttgctcaaataatacgaatatatcacccatccattttatgcgatgtttatatactctaatctttaatagatctatagacgacgggtttaccaacaatatagattttatcgattcatctaatttaaacccttccttaaacgtgaatgatctattatctggcataacgatgactctacctgatgaatcggacaatgtactgggccatgtagaataaattatcaacgaattatcgtctacgaacatttatatcatttgttttaattttaggacgcgaataaatggatataaaatagaaaataacagatattacaaccagtgttatggccgcgcccaaccaggtaggcagttttattttatcttttactacaggttctcctggatgtacgtcaccaacggcggacgtagttctagtacaattagacgtaagttccgcttgggaattttttaacgctaaagagttaacgttaatcgtgcacccaacgtatttacatctagttctttgaacatcttgattataatataaccattttctatctctagattcgtcggtgcactcatgtaaccaacataccctaggtcctaaatatttatctccggaattagattttggataattcgcgcaccaacaatttctatttcctttatgatcgttacaaaagacgtataatgccgtatccccaaaagtaaaataatcaggacgaataattctaataaactcagaacaatatctcgcatccatatgtttggagcaaatatcggaataagtagacatagccggtttccgttttgcacgtaaccattctaaacaattggggtttccaggatcgtttctacaaaatccagtcatgaaatcgtcacaatgttctgtcttgtaattattattaaatatttttggacagtgtttggtatttgtcttagaacaacattttgctacgctatcactatcgcccaggagataatccttttttataaaatgacatcgttgcccggatgctatataatcagtagcgtgttttaaatccttaatatattcaggagttacctcgttctgataatagattaatgatccaggacgaaatttgaaagaactacatggttctccatgaattaatacatattgtttagcaaattcaggaactataaaactactacaatgatctatcgacataccatctatcaaacaaaacttgggtttaatttctcccggagatgtttcataatagtacgtataactttcttctgcaaacttaacagctctattatattcaggataattaaaacctaattccatatatttgtctcgtatatctgctattcctggtgctattttgattctattaagagtaacagctgcccccattcttaataatcgtcagtatttaaactgttaaatgttggtatatcaacatctaccttatttcccgcagtataaggtttgttgcaggtatactgttcaggaatggttacatttatacttcttctatagtcctgtctttcgatgttcatcacatatgcaaagaacagaataaacaaaataatgtaagaaataatattaaatatctgtgaattcgtaaatacattgattgccataataattacagcagctacaatacacacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcatttatatattaaaaaatgtcactattaaagatggagtataatctttatgccgaactaaaaaaaatgacttgtggtcaacccctaagtctttttaacgaagacggggatttcgtagaagttgaaccgggatcatcctttaagtttctgatacctaagggattttacgcctctccttccgtaaagacgagtctagtattcgagacattaacaacgaccgataataaaatcactagtatcaatccaacaaatgcgccaaagttatatcctcttcaacgcaaagtcgtatctgaagtagtttctaatatgaggaaaatgatcgaatcaaaacgtcctctatacattactcttcacttggcgtgtggatttggtaagactattaccacgtgttatcttatggctacacacggtagaaaaaccgtcatttgcgtacccaataaaatgttaatacatcaatggaagacacaggtagaggcagtcggattggaacataagatatccatagatggagtaagtagtctattaaaggaactaaagactcaaagtccggatgtattaatagtagtcagtagacatctgacaaacgatgccttttgtaaatatatcaataagcattatgatttgttcatcttggatgaatcacatacgtataatctgatgaacaatacagcagttacaagatttttagcgtattatcctccgatgatgtgttattttttaactgctacacctagaccatctaacagaatttattgtaacagtattattaatattgccaagttatccgatctaaaaaaaactatctatgcggtagatagtttttttgagccatattccacagacaatattagacatatgataaaacgattagatggaccatctaataaatatcatatatatactgagaagttattatctgtagacgagcctagaaatcaacttattcttgataccctggtagaagaattcaagtcaggaactattaatcgcattttagttattactaaactacgtgaacatatggtattcttctacaaacgattattagatcttttcggaccagaggttgtatttataggagacgcccaaaatagacgtactccagatatggtcaaatcaatcaaggaactaaatagatttatattcgtatccaccttattttattccggtactggtttagatattcctagtttggattcgttgttcatttgctcggcagtaatcaacaatatgcaaatagagcaattactagggagggtatgtcgagaaacagaactattagataggacggtatatgtatttcctaacacatccatcaaagaaataaagtacatgataggaaatttcatgcaacgaattattagtctgtctgtagataaactaggatttaaacaaaaaagttatcggaaacatcaagaatccgatcccacttctgtatgtacaacatcctccagagaagaacgtgtattaaatagaatatttaactcgcaaaatcgttaagaagtttaagcgacgatccgcatgctgcgcaggccagtgtattacccctcatagtattaatataatccaatgatacttttgtgatgtcggaaatcttaaccaatttagactgacaggcagaacacgtcatgcaatcatcatcgtcatcgataactgtagtcttgggcttctttttgcggctcttcattccggaacgcacattggtgctatccatttaggtagtaaaaaataagtcagaatatgccctatagcacgatcgtgcaaaacctggtatatcgtctctatctttatcacaatatagtgtatcgacatctttattattattgacctcgtttatcttggaacatggaatgggaacatttttgttatcaacggccatctttgccttaattccagatgttgtaaaattataactaaacagtctatcatcgacacaaatgaaattcttgtttagacgtttgtagtttacgtatgcggctcgttcgcgtctcattttttcagatattgcaggtactataatattaaaaataagaatgaaataacataggattaaaaataaagttatcatgacttctagcgctgatttaactaacttaaaagaattacttagtctgtacaaaagtttgagattttcagattctgcggctatagaaaagtataattctttggtagaatggggaacatctacttactggaaaataggcgtgcaaaaggtagctaatgtcgagacgtcaatatctgattattatgatgaggtaaaaaataaaccgtttaatattgatccgggctattacattttcttaccggtatattttgggagcgtctttatttattcgaagggtaaaaatatggtagaacttggatctggaaactcttttcaaataccagatgatatgcgaagtgcgtgtaacaaagtattagacagcgataacggaatagactttctgagatttgttttgttaaacaatagatggataatggaagatgctatatcaaaatatcagtctccagttaatatatttaaactagctagtgagtacggattaaacatacccaaatatttagaaattgaaatagaggaagacacattatttgacgacgagttatactctattatagaacgctctttcgatgataaatttccaaaaatatccatatcgtatattaagttgggagaacttaggcggcaagttgtagactttttcaaattctcgttcatgtatattgagtccatcaaggtagatcgtataggagataatatttttattcctagcgttataacaaaatcaggaaaaaagatattagtaaaagatgtagaccatttaatacgatccaaggttagagaacatacatttgtaaaagtaaaaaagaaaaacacattttccattttatacgactatgatggaaacggaacagaaactagaggagaagtaataaaacgaattatagacactataggacgagactattatgttaacggaaagtatttctctaaggttggtagtgcaggcttaaagcaattgactaataaattagatattaatgagtgcgcaactgtcgatgagttagttgatgagattaataaatccggaactgtaaaacgaaaaataaaaaaccaatcagcatttgatttaagcagagaatgtttgggatatccagaagcggattttataacgttagttaataacatgcggttcaaaatagaaaattgtaaggttgtaaatttcaatattgaaaatactaattgtttaaataacccgagtattgaaactatatatggaaactttaaccagttcgtctcaatctttaatatcgtcaccgatgtcaaaaaaagattattcgagtgaaataatatgcgcctttgatataggtgcaaaaaatcctgccagaactgttttagaagtcaaggataactccgttagggtattggatatatcaaaattagactggagttctgattgggaaaggcgcatagctaaagatttgtcacaatatgaatacactacagttcttctagaacgtcagcctagaaggtcgccgtatgttaaatttatctattttattaaaggctttttatatcatacatcggctgccaaagttatttgcgtctcgcctgtcatgtctggtaattcatatagagatcgaaaaaagagatcggtcgaagcatttcttgattggatggacacattcggattgcgagactccgttccggatagacgcaaattagacgatgtagcggatagtttcaatttggctatgagatacgtattagataaatggaatactaattatacaccttataataggtgtaaatctagaaattacataaaaaaaatgtaataacgttagtaacgccattatggataatctatttacctttctacatgaaatagaagatagatatgccagaactatttttaactttcatctaataagttgcgatgaaataggagatatatatggtcttatgaaagaacgcatttcctcagaggatatgtttgataatatagtgtataataaagatatacatcctgccattaagaaactagtgtattgcgacatccaacttactaaacacattattaatcagaatacgtatccggtatttaacgattcttcacaagtgaaatgttgtcattatttcgacataaactcagataatagcaatattagctctcgtacagtagagatatttgagagggaaaagtcatctcttgtatcatatattaaaactaccaataagaagagaaaggtcaattacggcgaaataaagaaaactgttcatggaggcactaatgcaaattacttttccggtaaaaagtctgacgagtatctgagtactacagttagatccaacattaatcaaccttggatcaaaaccatctctaagaggatgagagttgatatcattaatcactctatagtaacgcgtggaaaaagctctatattacaaactatagaaattatttttactaatagaacatgtgtgaaaatattcaaggattctactatgcacattattctatccaaggacaaggatgaaaaggggtgtatacacatgattgacaaattattctatgtctattataatttatttctgttgttcgaggatatcatccaaaacgagtactttaaagaagtagctaatgttgtaaaccacgtactcacggctacggcattagatgagaaattattcctaattaagaaaatggctgaacacgatgtttatggagttagcaatttcaaaatagggatgtttaacctgacatttattaagtcgttggatcataccgttttcccctctctgttagatgaggatagcaaaataaagttttttaaggggaaaaagctcaatattgtagcattacgatctctggaggattgtataaattacgtgactaaatccgagaatatgatagaaatgatgaaggaaagatcgactattttaaatagcatagatatagaaacggaatcggtagatcgtctaaaagaattgcttctaaaatgaaaaaaaacactgattcagaaatggatcaacgactcggatataagtttttggtgcctgatcctaaagccggagttttttatagaccgttacatttccaatatgtatcgtattctaattttatattgcatcgattgcatgaaatcttgaccgtcaagcggccactcttatcgtttaagaataatacagaacgaattatgatagaaattagcaatgttaaagtgactcctccagattactcacctataatcgcgagtattaaaggtaagagttatgacgcattagccacgttcactgtaaatatctttaaagaggtaatgaccaaagagggtatatccatcactaaaataagtagttatgagggaaaagattctcatttgataaaaattccgctactaataggatacgggaataaaaatccacttgatacagccaagtatcttgttcctaatgtcataggtggagtctttatcaataaacaatctgtcgaaaaagtaggaattaatctagtagaaaagattacaacatggccaaaatttagggttgttaagccaaactcattcactttctcgttttcctccgtatcccctcctaatgtattaccgacaagatatcgccattacaagatatctctggatatatcacaattggaagcgttgaatatatcatcgacaaagacatttataacggtcaatattgttttgctgtctcaatatttatctagagtgagtctagaattcattagacgtagtttatcatacgatatgcctccagaagttgtctatctagtaaacgcgataatagatagtgctaaacgaattactgaatctattactgactttaatattgatacatacattaatgacctggtggaagctgaacacattaaacaaaaatctcagttaacgattaacgagttcaaatatgaaatgctgcataactttttacctcatatgaactatacacccgatcaactaaagggattttatatgatatctttactaagaaagtttctctactgtatcttccacacttctagatatccagatagagattcgatggtttgtcatcgcatcctaacgtacggcaaatattttgagacgttggcacatgatgaattagagaattacataggcaacatccgaaacgatatcatgaacaatcacaagaacagaggcacttacgcggtaaacattcatgtactaacaactcccggacttaatcacgcgttttctagcttattgagtggaaagttcaaaaagtcagacggtagttatcgaacacatcctcactattcatggatgcagaatatttctattcctaggagtgttggattttatccggatcaagtaaagatttcaaagatgttttctgtcagaaaataccatccaagtcaatatctttacttttgttcatcagacgttccggaaagaggtcctcaggtaggtttagtatctcaattgtctgtcttgagttccattacaaatatactaacgtctgagtatttggatttggaaaagaaaatttgtgagtatatcagatcatattataaagatgatataagttactttgaaacaggatttccaatcactatagaaaatgctctagtcgcatctcttaatccaaatatgatatgtgattttgtaactgactttagacgtagaaaacggatgggattcttcggtaacttggaggtaggtattactttagttagggatcacatgaatgaaattcgcattaatattggagcgggaagattagtcagaccattcttggttgtggataacggagagctcatgatggatgtgtgtccggagttagaaagcagattagacgacatgacattctctgacattcagaaagagtttccgcatgtcatcgaaatggtagatatagaacaatttacttttagtaacgtatgtgaatcggttcaaaaatttagaatgatgtcaaaggatgaaagaaagcaatacgatttatgtgactttcctgccgaatttagagatggatatgtggcatcttcattagtgggaatcaatcacaattctggacccagagctattcttggatgtgctcaagctaaacaagctatctcttgtctgagttcggatatacgaaataaaatagacaatggaattcatttgatgtatccagagaggccaatcgtgattagtaaggctttagaaacttcaaagattgcggctaattgcttcggccaacatgttactatagcattaatgtcgtacaaaggtatcaatcaagaggatggaattatcatcaaaaaacaatttattcagagaggcggtctcgatatagttaccgcaaagaaacatcaagtagaaattccattggaaaactttaataacaaagaaagagataggtctaacgcctattcaaaattagaaagtaatggattagttagactgaatgctttcttggaatccggagacgctatggcacgaaatatctcatcaagaactcttgaagatgattttgctagagataatcagattagcttcgatgtttccgagaaatataccgatatgtacaaatctcgcgttgaacgagtacaagtagaacttactgacaaagttaaggtacgagtattaaccatgaaagaaagaagacccattctaggagacaaatttaccactagaacgagtcaaaagggaacagtcgcgtatgtcgcggatgaaacggaacttccatacgacgaaaatggtatcacgccagatgtcattattaattctacatccatcttctctagaaaaactatatctatgttgatagaagttattttaacagccgcatattctgctaagccgtacaacaataagggagaaaaccgacctgtctgttttcctagtagtaacgaaacatccatcgatacatatatgcaattcgctaaacaatgttatgagcattcaaatccgaaattgtctgatgaagaattatcggataaaatcttttgtgaaaagattctctatgatcctgaaacggataagccttatgcatccaaagtattttttggaccaatttattacttgcgtctgagacatttaactcaggacaaggcaaccgttagatgtagaggtaaaaagacgaagctcattagacaggcgaatgagggacgaaaacgtggaggaggtatcaagttcggagaaatggagagagactgtttaatagcgcatggcgcagccaatactattacagaagttttgaaagattcggaagaagattatcaagatgtgtatgtttgtgaaaattgtggagacatagcagcacaaatcaagggtattaatacatgtcttagatgttcaaaacttaatctctctcctctcttaacaaaaattgataccacgcacgtatctaaagtatttcttactcaaatgaacgccagaggcgtaaaagtcaaattagatttcgaacgaagacctccttcgttttataaaccattagataaagttgatctcaagccgtcttttctggtgtaatattctagtttggtagtagatacatatcaatatcatcaaattcgagatccgaattataaaatgggcgtggattgttaactatagaatcggacgtctgatattcgaaaatctgtggagtttcaggttttggtggaggtgtaactgctacttgggatactgaagtctgatattcagaaagctgtggatgttctggttcggcatccaccgatggtgtcacatcactaatcggttcggtaacgtctgtggatggaggtgctacttctacagaacctgtagcctcagttgtcaacggagatacatttttaatgcgagaaaatgtataatttggtaatggtttctcatgtggatctgaagaagaggtaagatatctactagaaagataccgatcacgttctagttctcttttgtagaacttaactttttctttctccgcatctagttgatattccaacctcttcacgttactacgttcagattccaattcacgttcgcatgggttacctccgcagtttttacgagcgatttcacgttcagccttcatgcgtctctccctctctctatcgagtttatcagagcagtctttctgaaggcgatcgaactccataaatttctccaacgctttgattgtttccatagatttccgaacttcagcttctaggacggcgattctttttctttcgaattcacagctggatgtacaaccgtttccattaccgccatctctaagtttcttttctagatcggcaacatttcatccccatgccttttacattcctcgagtctactgtcgtcgaaatatcgttccagctccttttcgacatcaataactttagcacgttgtctctcaagctctcttttgtagttatctgattccctggcacgtttaagatcttcatgcaattgagtcagctcttaacttcctctcttgcttcttcgtcatagtacgcgcaatcactgtgagatccattgttaccacgtctacactcggcgagctcgcgtttaagagattcaatttcccgtttgtattggtccatgtttccattgctaccaccattagatttacaggctgctagttgtcgttcgagatcagaaatacgggttttcttggaattgatttcgtcgatgtacttggcatcgaaacacttattaagttctttttccaattctacgattttatttctttcgcgagtcaattccctcctgtagtaactatctgttttgtcagattcacgctctctacgtagactttcttgcaagttactaatttgttccctagcacgtccgagtttagttttatatgctgaatagagttctgattcatcctttgagcagatctctagcgatcgtttaagattcctgattctagtctttagcctatttacctcctcagaagatgttccgttaccgttgcgtttacactcgttaagctgtctatcaagatccatgattctatctctaagacgttgcatctctctttccatatcagcattgctttcattattacgtctgcagtcactcaactgtctttcaatatctgagattctatctctaagacgtcgcatctctctctgtttcagcattggtttcattattacgtctacagtcgttcaactgtctttcaagatctgatattctagattggagtctgctaatctctgtagcattttcacggcattcactcagttgtctttcaagatctgaaattttagattggagtctgctaatctctgtaagatttcctcctccgctctcgatgcagttggtcaacttattctctagttctctaatacgcgaacgcagtgcatcaacttcttgcgtgtcttcctggttgcgtgtacattcatcgagtctagattcgagatctctaacgcgtcgtcgttcttcctcaagttctctgcgtactacagaaagcgtgtccctatcttgttgatatttagcaatttctgattctagagtactgattttgcttacgtagttactaatagttgtcttggccttatcaagatcctccttgtatttgtcgcattccttgatatccctacgaagtctggacagttcccattcgacattacgacgtttatcgatttcagctcggagatcgtcatcgcgttgttttagccacatacgactgagttcaagttctcgttgacaagatccatctacttttccattcctaatagtatccagttccttttctagttctgaacgcatttctcgttccctatcaagcgattctctcaattctcggatagtcttcttatcaatttctaataaatctgaaccatcatctgtcccattttgaatatccctgtgttctttgatctcttttgtaagtcggtcgattctttcggttttataaacagaatccctttccaaagtcctaatcttactgagtttatcactaagttctgcattcaattcggtgagttttctcttggcttcttccaactctgttttaaactctccactatttccgcattcttcctcgcatttatctaaccattcaattagtttattaataactagttggtaatcagcgattcctatagccgttcttgtaattgtgggaacataattaggatcttctaatggattgtatggcttgatagcatcatctttatcattattagggggatggacaaccttaattggttggtcctcatctcctccagtagcgtgtggttcttcaataccagtgttagtaataggcttaggcaaatgcttgtcgtacgcgggcacttcctcatccatcaagtatttataatcgggttctacttcagaatattcttttctaagagacgcgacttcgggagttagtagaagaactctgtttctgtatctatcaacgctggaatcaatactcaagttaaggatagcgaatacctcatcgtcatcatccgtatcttctgaaacaccatcatatgacatttcatgaagtctaacgtattgataaatagaatcagatttagtattaaacagatccttaacctttttagtaaacgcatatgtatattttagatctccagatttcataatatgatcacatgccttaaatgtcagtgcttccatgatataatctggaacactaatgggtgacgaaaaagatacagcaccatatgctacgttgataaataaatctgaaccactaagtagataatgattaatgttaaggaaaagaaaatattcagtgtataggtatgtcttggcgtcatatcttgtactaaacacgctaaacagtttgttaatgtgatcaatttccaatagattaattagagcagcgggaataccaacaaacatattaccacatccgtattttctatgaatatcacatatcatgttaaaaaatcttaatagaagagcgaatatctcgtctgacttaatgagacgtagttcagcagcaacataagtcataactgtaaatagaacatactttcctgtagtgttgattctagactccacatcaacaccattattaaaaatagttttatatacatctttaatctgctctccgttaatcgtcgaacgttctagtatacggaaacactttgatttcttatctgtagttaatgacttagtgatatcacgaagaatattacgaattacatttcttgtttttcttgagagacctgattcagaactcaactcatcgttccatagtttttctacctcagtggcgaaatctttggagtgcttggtacatttttcaataaggttcgtgacctccatttattataaaaaatttattcaaaacttaactacaatcgggtaattataagatcgtaaatctcccatgtggcggaatactaccatctatcgcatgtggatggacagtaggtaatggccatgggaacagtaatgattgcatatttatctttcttgctagtattactgcatattgtcccaatgtttcgatgtgatgttctaacctatcaactgccgctgtatcacaacaatagtgtccgatgaaattaagattatgatccaatgtgtttaatatatgattatcaagtcttatacgatccgcgtcttttttgacaggatcaggttcttctacaggaagaagtttcggcctcttatgatattcatgtctgggaaacggtggtctagggtgaggctccggtatcggagtgggttttggattataatcatcatcgtctatgacatcatcatcatcttcgacttcgatatttattttgctatcttgatgatgtcctgtatcagttgcattttcagcactcgactgaatattagcgcattcattgtctattattaccatatttctaaacccaaaatgtatgtgttgaacatcagtactatcgttgatgagtcttatagcatgaattcgcttatcgttatcgggtttatcttctgtcaccttagcaattccttttttattaaactctacataatcatatccatttctattgtttgttctaatataaacgagtatagcatcattgctaaatttttcaatagtatcgaaaacagaatatcctaaaccatataatatatattcagggacactcaaactaaatgtccaggattctcctaaatacgtaaactttaatagtgcgaaatcattcaaaaatctaccacttatagatagatagtacataaatgcgtatagtagtctacctatctctttattatgaaaaccggcattacgatcatatatgtcgtgatatacctgtgatccgtttacgttaaaccataaatacatgggtgatcctataaacatgaatttatttctaattctcagagctatagttaattgaccgtgtaatatttgcttacatgcatacttgatacgctcattaataaaatttttatcattgctcgttatctcagaatcgtatatataaggagtaccatcgtgattcttaccagatattatacaaaatactatatataaaatatattgaccaacgttagtaatcatataaatgtttaacgttttaaattttgtattcaatgatccattatcatacgctagcatggtcttatgatattcattctttaaaatataatattgtgttagccattgcattggggctcctaatggagattttttattctcatccattttaggataggctttcataaagtccctaataacttcgtgaataatgtttctatgttttctactgatgcatgtatttgcttcgatttttttatcccatgtttcatctatcatagatttaaacgcagtaatgctcgcaacattaacatcttgaaccgttggtacaattccgttccataaatttataatgttcgccatttatataactcattttttgaatatacttttaattaacaaaagagttaagttactcatatgggcgccgtccagtctgaacatcaatctttttagccagagatatcatagccgctcttagagtttcagcgtgattttccaacctaaatagaacttcatcgttgcgtttacaacacttttctatttgttcaaactttgttgttacattagtaatctttttttccaaattagttagccgttgtttgagagtttcctcattgtcgtcttcatcggctttaacaattgcttcgcgtttagcctctggctttttagcagcctttgtagaaaaaaattcagttgctggaattgcaagatcgtcatctccggggaaaagagttccgtccatttaaagtacagattttagaaactgacactctgcgttatttatatttggtacaacacatggattataaatatcgatgttaataacatcagaaaatgtaaagtctatacattgttgcatcgtgttaaattttctaatggatctagtattattgggtccaacttctgcctgaaatccaaatatggaagcggatacaaaaccgtttcctggataaaccacacatctccacttttgctttacatcagaaattgtgtcgttgacatcttgaactctcctatctaatgccggtgttccacctatagattttgaatattcgaatgctgcatgagtagcattaaattccttaatattgccataattttcatatattgagtaaccctggataaaaagtaaacacaccgcagccgtcgctaccacaataaaaaaaattgatagagagttcatttataatctattagaagctgacaaaatttttttacacgcatcagacaatgctttaataaatagttcaacatctacttttgtcatatcgaaccgatggtatgattctaacctagaattacatccgaaaaagttgactatgttcatagtcattaagtcattaacaaacaacattccagactctggattataagacgatactgtttcgtcacaattacctaccttaatcatgtgattatgaatattggctattagagcaccttctaagaaatctataatatctttgaaacacgatttaaaatcaaaccacgaatatacttctacgaagaaagttagtttacccataggagaaataactataaatggagatctaaatacaaaatccggatctatgatagttttaacattattatattctctattaaatacctccacatctaaaaatgttaattttgaaactatgtcttcgtttattaccgtacctgaactaaacgctataagctctattgtttgagaactctttaaacgatattcttgaaatacatgtaacaaagtttcctttaactcggtcggtttatctaccatagttacagaatttgtatccttatctataatataataatcaaaatcgtataaagttatataattatcgcgttcagattgggatcttttcaaatagactaaaaaccccatttctctagtaagtatcttatgtatatgtttgtaaaatatcttcatggtgggaatatgctctaccgcagttagccattcctcattgacagcggtagatgtattagacaaaactattccaatgtttaacaagggccattttacgagattattaaatccttgtttgataaatgtagccaatgagggttcgagttcaacgacgattgaattctcttcccgcggatgctgcatgatgaacgacgggatgttgttcgattgatttggaattctttttcgactttttgtttatattaaatattttaaaatttatagcggatagcaattcatgtaccacggataatgtagacgcgtattgcgcatcgatatctttattattagataaatttatcaataaatgtgagaagtttgcctcgttaaggtcttccatttaaatattatataaacatttgtgtttgtatcttattcgtcttttatggaatagttttttactagtaaagctgcaattacacactttgtccgtaaaacataaatataaacaccagcttttatcaatcgttccaaaaagtcgacggcggacatttttaacatggcatctattttaaatacacttaggtttttggaaaaaacatcattttataattgtaacgattcaataactaaagaaaagattaagattaaacataagggaatgtcatttgtattttataagccaaagcattctaccgttgttaaatacttgtctggaggaggtatatatcatgatgatttggttgtattggggaaggtaacaattaatgatctaaagatgatgctattttacatggatttatcatatcatggagtgacaagtagtggagcaatttacaaattgggatcgtctatcgatagactttctctaaataggactattgttacaaaagttaataattatgatgatacattttttgacgacgatgattgatcgctattgcacaattttgtttttttactttctaatatagcgtttagattctttttcatgtgcgaatattgatttactaaaatatcgatgtttaacttttgttctatgacgtccttatcagcggtatcggtacatatacgtaattcaccttcacaaaatacggagtcttcgataataatagccaatcgattattggatctagcggtctgtatcatattcaacatgtttaatatatcctttcgtttcccctttacaggcatcgatcgtagcatattttccgcgtctgagatggaaatgttaaaactacaaaaatgcgtaatgttagcccgtcctaatattggtacgtgtctataagtttggcatagtagaataatagacgtgtttaaatgccttccaaagtttaagaattctattagagtattgcattttgatagtttatcgcctacatcatcaaaaataagtaaaaagtgtgctgattttttatgattttgtgcgacagcaatacatttttctatgttacttttagttcgtatcagattatattctagagattcctgactactaacgaaattaatatgatttggccaaatgtatccatcataatctggattataaacgggtgtaaacaagaatatatgtttatattttttaactagtgtagaaaacagagatagtaaatagatagtttttccagatccagatcctcccgttaaaaccattctaaacggcatttttaataaattttctcttgaaaattgtttttcttggaaacaattcataattatatttacagttactaaattaatttgataataaatcaaaatatggaaaactaaggtcgttagtagggaggagaacaaagaaggcacatcgtgacataaataacatttattatcatgatgacaccagaaaacgacgaagagcagacatctgtgttctccgctactgtttacagagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgactacctggctcattgattatgttaaggatacatggggatctgatggtaatccaattacaaaaactacatccgattatcaagattctgatgtatcacaagaagttagaaagtatttttgtgttaaaacaatgaactaatatttatttttgtacattaataaatgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtgacaacaaaaaatgaattaataataagtcgttaacgtacgccgccatggacgccgcgtttgttattactccaatgggtgtgttgactataacagatacattgtatgatgatctcgatatctcaatcatggactttataggaccatacattataggtaacataaaaactgtccaaatagatgtacgggatataaaatattccgacatgcaaaaatgctactttagctataagggtaaaatagttcctcaggattctaatgatttggctagattcaacatttatagcatttgtgccgcatacagatcaaaaaataccatcatcatagcatgcgactatgatatcatgttagatatagaagataaacatcagccattttatctattcccatctattgatgtttttaacgctacaatcatagaagcgtataacctgtatacagctggagattatcatctaatcatcaatccttcagataatctgaaaatgaaattgtcgtttaattcttcattctgcatatcagacggcaatggatggatcataattgatgggaaatgcaatagtaattttttatcataaaagttgtaaagtaaataataaaacaataaatattgaactagtagtacgtatattgagcaatcagaaatgatgctggtacctcttatcacggtgaccgtagttgcgggaacaatattagtatgttatatattatatatttgtaggaaaaagatacgtactgtctataatgacaataaaattatcatgacaaaattaaaaaagataaagagttctaattccagcaaatctagtaaatcaactgatagcgaatcagactgggaggatcactgtagtgctatggaacaaaacaatgacgtagataatatttctaggaatgagatattggacgatgatagcttcgctggtagtttaatatgggataacgaatccaatgttatggcgcctagcacagaacacatttacgatagtgttgctggaagcacgctgctaataaataatgatcgtaatgaacagactatttatcagaacactacagtagtacttaatgaagataccaaacagaatcctaactattcatccaatcctttcgtaaattataataaaaccagtatttgtagcaagtcaaatccgttcattacagaactcaacaataaatttagtgagaataatccgtttagacgagcacatagcgatgattatcttaataagcaagaacaagatcatgaacacgatgatatagaatcattggtgtgattagtttcctttttataaaattgaagtaatatttagtattattgctgccgtcacgttgtacaaatggagatattccctgtattcggcatttctaaaattagcaattttattgctaataatgactgtagatattatatagatacagaacatcaaaaaattatatctgatgagatcaatagacagatggatgaaacggtacttcttaccaacatcttaagcgtagaagttgtaaatgacaatgagatgtaccatcttattcctcatagattatcgacgattatactctgtattagttctgtcggaggatgtgttatctctatagataatgacgtcaatggcaaaaatattctaacctttcccattgatcatgctgtaatcatatccccactgagtaaatgtgtcgtagttagcaagggtcctacaaccatattggttgttaaagcggatatacctagcaaacgattggtaacatcgtttacaaacgacatactgtatgtaaacaatctatcactgattaattatttgccgttgtctgtattcattattagacgagttaccgactatttggatagacacatatgcgatcagatatttgcgaataataagtggtattccattataaccatcgacaataagcagtttcctattccatcaaactgtataggtatgtcctctgccaagtacataaattctagcatcgagcaagatactttaatacatgtttgtaacctcgagcatccattcgacttagtatacaaaaaaatgcagtcgtacaattctgtacctatcaaggaacaaatattgtacggtagaattgataatataaatatgagcattagtatttctgtggattaatagatttctagtatggggatcattaatcatctctaatctctaaatacctcataaaacgaaaaaaaagctattatcaaatactgtacggaatggattcattctcttctctttttatgaaactctgttgtatatctactgataaaactggaagcaaaaaatctgataaaaagaataagaataagatcaaggattattataaaataacaatagttcctggttcctcttccacgtctactagctcgtggtattatacacatgcctagtaatagtctctttgcgttgacggaaagcagactagaaataacaggctaaaatgttcagacaccataatagttcccaacccagataataacagagtaccatcaacacattcctttaaactcaatcccaaacccaaaaccgttaaaatgtatccggccaattgatagtagataatgaggtgtacagcgcatgataatttacacagtaaccaaaatgaaaatactttagtaattataagaaatatagatggtaacgtcatcatcaacaatccaataatatgccggagagtaaacattgacggataaaacaaaaatgctccgcataactctatcatggcaataacacaaccaaatacttgtaagattcctaaattagtagaaaatacaacggatatcgatgtataagtgatctcgagaaataataagaataaagtaatgcccgtaaagataaacatcaacattgtttggtaatcattaaaccaattagtatgaagttgaactaatttcacagtagattttattccagtattatccccgcatgtataagtacctggtaagatatctttatattccataatcaatgagacatcactatctgataacgaatgaagtctagcactagtatgccatttacttaatattgtcgtcttggaagttttattataagttaaaatatcatggttatccaatttccatctaatatactttgtcggattatctatagtacacggaataatgatggtatcattacatgctgtatactctatggtctttgtagttgttataacaaccaacgtatagaggtatatcaacgatattctaactcttgacattttttatttatttaaaatgatacctttgttatttattttattctattttgctaacggtattgaatggcataagtttgaaacgagtgaagaaataatttctacttacttattagacgacgtattatacacgggtgttaatggggcggtatacacattttcaaataataaactaaacaaaactggtttaactaataataattatataacaacatctataaaagtagaggatgcggaaccaataacggaaatcccaaatgttggaaaatagacggttcagacgacccaaaacatagaggtagaggatacgctccttatcaaaatagcaaagtaacgataatcagtcacaacggatgtgtactatctgacataaacatatcaaaagaaggaattaaacgatggagaagatttgacggaccatgtggttatgatttatacacggcggataacgtaattccaaaagatggtttacgaggagcattcgtcgataaagatggtacttatgacaaagtttacattcttttcactgatactatcggctcaaagagaattgtcaaaattccgtatatagcacaaatgtgcctaaacgacgaaggtggtccatcatcattgtctagtcatagatggtcgacgtttctcaaagtcgaattagaatgtgatatcgacggaagaagttatagacaaattattcattctagaactataaaaacagataatgatacgatactatatgtattcttcgatagtccttattccaagtccgcattatgtacctattctatgaataccattaaacaatctttttctacgtcaaaattggaaggatatacaaagcaattgccgtctccagctcctggtatatgtttaccagctggaaaagttgttccacataccacgtttgaagtcatagaaaaatataatgtactagatgatattataaagcctttatctaaccaacctatcttcgaaggaccgtctggtgttaaatggttcgatataaaggagaaggaaaatgaacatcgggaatatagaatatacttcataaaagaaaattctatatattcgttcgatacaaaatctaaacaaactcgtagctcgcaagtcgatgcgcgactattttcagtaatggtaacttcgaaaccgttatttatagcagatatagggataggagtaggaatgccacaaatgaaaaaaatacttaaaatgtaatcttaatcgagtacaccgcacgacaatgaacaaacataagacagattatgctggttatgcttgctgcgtaatatgcggtctaattgttggaattatttttacagcgacactattaaaagttgtagaacgtaaattagttcatacaccatcaatagataaaacgataaaagatgcatatattagagaagattgtcctactgactggataagctataataataaatgtatccatttatctactgatcgaaaaaacctgggaggaaggacgtaatgcatgcaaagctctaaatccaaattcggatctaattaagatagagactccaaacgagttaagttttttaagaagccttagacgaggctattgggtaggagaatccgaaatattaaaccagacaaccccatataattttatagctaaaaatgccacgaagaatggaactaaaaaacggaaatatatttgtagtacaacgaatactcccaaactgcattcgtgttacactatataacaattacactacatttttatcataacactacttcggttagatgttttagaaaaaaataaatatcgccgtaccgttcttgtttttataaaaataacaattaacaattatcaaattttttctttaatattttacgtggttgaccattcttggtggtaaaataatctcttagtgttggaatggaatgctgtttaatgtttccgcactcatcgtatattttgacgtatgcagtcacatcgtttacgcaatagtcagactgtagttctatcatgcttcctacatcagaaggaggaacagttttaaagtctcttggttttaatctattgccattagttttcatgaaatcctttgttttatccacttcacattttaaataaatgtccactatacattcttctgttaattttactagatcgtcatgggtcatagaatttataggttccgtagtccatggatccaaactagcaaacttcgcgtatacggtatcgcgattagtgtatacaccaactgtatgaaaattaagaaaacagtttaataaatcaacagaaatatttaatcctccgtttgatacagatgcgccatatttatggatttcggattcacacgttgtttgtctgaggtgttcgtctagtgttgcttctacgtaaacttcgattcccatatattctttattgtcagaatcgcataccgatttatcatcatacactgtttgaaaactaaatggtatacacatcaaaataataaataataacgagtacattctgcaatattgttatcgtaattggaaaattagtgttcgagtgagtcggattatgtgagtactggattgtatattttattttatattttgtaataagaataaaatgctaatgtcaagtttattccaatagatgtcttattaaaaaacatatataataaataacaatggctgaatggcataaaattatcgaggatatctcaaaaaataataagttcgaggatgccgccatcgttgattacaagactacaaagaatgttctagctgctattcctaacagaacatttgccaagattaatccgggtgaaattattcctctcatcactaatcgtaatattctaaaacctcttattggtcagaaatattgtattgtatatactaactctctaatggatgagaacacgtatgctatggagttgcttactgggtacgcccctgtatctccgatcgttatagcgagaactcataccgcacttatatttttgatgggtaagccaacaacatccagacgtgatgtgtatagaacgtgtagagatcacgctacccgtgtacgcgcaactggtaattaaaataaaaagtaatattcatatgtagtgtcaattttaaatgatgatgatgaaatggataatatccatattgacgatgtcaataatgccggtattggcatacagctcatcgatttttagatttcattcagaggatgtggaattatgttatgggcatttgtattttgataggatctataatgtagtaaatataaaatataatccgcatattccatatagatataattttattaatcgcacgttaaccgtagatgaactagacgataatgtcttttttacacatggttattttttaaaacacaaatatggttcacttaatcctagtttgattgtctcattatcaggaaacttaaaatataatgatatacaatgctcagtaaatgtatcgtgtctcattaaaaatttggcaacgagtacatctactatattaacatctaaacataagacttattctctacatcggtccacgtgtattactataataggatacgattctattatatggtataaagatataaatgacaagtataatgacatctatgattttactgcaatatgtatgctaatagcgtctacattgatagtgaccatatacgtgtttaaaaaaataaaaatgaactcttaattatgctatgctattagaaatggataaaatcaaaattacggttgattcaaaaattggtaatgttgttaccatatcgtataacttggaaaagataactattgatgttacacctaaaaagaaaaaagaaaaggatgtattattagcgcaatcagttgctgtcgaagaggcaaaagatgtcaaggtagaagaaaaaaatattatcgatattgaagatgacgatgatatggatgtagaaagcgcataatacgatctataaaaataagtatataataaatactttttatttacggtactcttgtagtggtgataccctactcaattatttttttaaaaaaatacttattctgattcttctagccatttccgtgttcgttcgaatgccacatcgacgttaaagataggggagtagttgaaatctagttctgcattgttggtacgcacctcaaatgtagtgttggatatcttcaacgtatagttgttgagtagtgatggttttctaaatagaattctcttcatatcattcttgcacgcgtacatttttagcatccatcttggaattctagatccttgttctattcccaatggtttcatcaatagaagattaaacatatcgtacgaacacgatggagagtaatcgtagcaaaagtaagcatttcctttaatcttagatcccggatactggatatattttgcagccaacacgtgcatccatgcagcatttcctacatatacccggctatgcaccgcgtcatcatcgactgtacgatacataatgttaccgtgttgcttacattgctcgtaaaagactttcgtcaatttgtctccttctccgtaaattccagtgggtcttaggcaacaagtatacaattttgctccattcatgattacggaattattggctttcataaccagttgctcggccatacgtttactttttgcgtatacatgtcctggtgatatatcataaagggtatgctcatggccgatgaatggatcaccgtgtttattgggtcctattgcttccatgctactagtatagatcaaatacttgattcctaggtccacacaagctgccaatatagtctgtgttccataatagtttactttcatgatttcattatcggtgtattttccaaatacatccactagagcagccgtatgaataatcagatttaccccatctagcgcttctctcaccttatcaaagtcgtttatatcacattgtatatagtttataaccttaactttcgaggttattggttgtggatcttctacaatatctatgactctgatttcttgaacatcatctgcactaattaacagttttactatatacctgcctagaaatccggcaccaccagtaaccgcgtacacggccattgctgccactcataatatcagactacttattctattttactaaataatggctgtttgtataatagaccacgataatatcagaggagttatttactttgaaccagtccatggaaaagataaagttttaggatcagttattggattaaaatccggaacgtatagtttgataattcatcgttacggagatattagtcaaggatgtgattccataggcagtccagaaatatttatcggtaacatctttgtaaacagatatggtgtagcatatgtttatttagatacagatgtaaatatatttacaattattggaaaggcgttatctatttcaaaaaatgatcagagattagcgtgtggagttattggtatttcttacataaatgaaaagataatacattttcttacaattaacgagaatggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgacaccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaagtataggtgtatggcattagccgaacatatatctgatagtattccaatatctgagctatctagactacgatacaatctatgtaagtatctacgcggacacactgagagcatagaggatgaatttgattattttgaagacgatgattcgtctacatgttctgccgtaaccgacagggaaacggatgtataattttttttatagcgtgaaggatatgataaaaaatataattgttgtatttatcccattccaatcaccttatatgattctgtaaaaaaattatactgtaacacaataaaggagtcttatagatgtatagaggtcagatactggtttgataaactgtttattccacataagtatgtttgactttatggttagacccgcatactttaacaaatcactgaaaattggagttaggtattgacctctcagaatcagttgccgttctggaacattaaatgtattttttatgatatactccaacgcatttatgtgggcatacaacaagtcattactaatggagtattccaagagttttagttgtctagtatttaacaagagaagagatttcaacagactgtttatgaactcgaacgccgcctcattgtcgcttatattgatgatgtcgaattctcccaatatcatcactgatgagtagctcatcttgttatcgggatccaagttttctaaagatgtcattaaaccctcgatcatgaatggatttatcatcatcgtttttatgttggacatgagcttagtccgtttgtccacatctatagacgacgatttctgaattatttcatatatccctctctttaactccaggaacttgtcaggatggtctactttaatatgttctcgtctaagagatgaaaatctttggatggttgcacgcgacttttctttaaaggatgacgttgcccaagatcctctcttaaatgaatccatcttatccttggacaagatggacagtctattttccttagatggtttaatatttttgttacccatgatctataaaggtagacctaatcgtctcggatgaccatatatttattttcagttttattatacgcataaattgtaaaaaatatgttaggtttacaaaaatgtctcgtggggcattaatcgtttttgaaggattggacaaatctggaaaaacaacacaatgtatgaacatcatggaatctataccggcaaacacgataaaatatcttaactttcctcagagatccactgtcactggaaagatgatagatgactatctaactcgtaaaaaaacctataatgatcatatagttaatctattattttgtgcaaatagatgggagtttgcatcttttatacaagaacaactagaacagggaattactttaatagttgatagatacgcattttctggagtagcgtatgccgccgctaaaggcgcgtcaatgactctcagtaagagttatgaatctggattgcctaaacccgacttagttatattcttggaatctggtagcaaagaaattaatagaaacgtcggcgaggaaatttatgaagatgttacattccaacaaaaggtattacaagaatataaaaaaatgattgaagaaggagatattcattggcaaattatttcttctgaattcgaggaagatgtaaagaaggagttgattaagaatatagttatagaggctatacacacggttactggaccagtggggcaactgtggatgtaatagtgaaattacattttttataaatagatgttagtacagtgttataaatggatgaagcatattactctggcaacttggaatcagtactcggatacgtgtccgatatgcataccgaactcgcatcaatatctcaattagttattgccaagatagaaactatagataatgatatattaaacaaggacattgtaaattttatcatgtgtagatcaaacttggataatccatttatctctttcctagatactgtatatactattatagatcaagagatctatcagaccgaattgattaattcattagacgacaatgaaattatcgattgtatagttaacaagtttatgagcttttataaggataacctagaaaatatagtagatgctatcattactctaaaatatataatgaataatccagattttaaaactacgtatgccgaagtactcggttccagaatagccgatatagatattaaacaagtgatacgtaagaatatactacaattgtctaatgatatccgcgaacgatatttgtgaaaaatattaaaaaaaaatactttttttattaaatgacgtcgcttcgcgaatttagaaaattatgctgtgatatatatcacgcatcaggatataaagaaaaatctaaattaattagagactttataacagatagggatgataaatatttgatcattaagctattgcttcccggattagacgatagaatttataacatgaacgataaacaaattataaaattatatagtataatatttaaacaatctcaggaagatatgctacaagatttaggatacggatatataggagacactattaggactttcttcaaagagaacacagaaatccgtccacgagataaaagcattttaactttagaagaagtggatagttttttaactacgttatcatccgtaactaaagaatcgcatcaaataaaattattgactgatgtagcatctgtttgtacatgtaatgatttaaaatgtgtagtcatgcttattgataaagatctaaaaattaaagcgggccctcggtacgtacttaacgctattagtcctcatgcctatgatgtgtttagaaaatctaataacttgaaagagataatagaaaatgcatctaaacaaaatctagactctatatctatttctgttatgactccaattaatcccatgttagcggaatcgtgtgattctgtcaataaggcgtttaaaaaatttccatcaggaatgtttgcggaagtcaaatacgatggtgaaagagtacaagttcataaaaataataacgagtttgccttctttagtagaaacatgaaaccagtactctctcataaagtggattatctcaaagaatacataccgaaagcatttaaaaaagctacgtctatcgtattggattctgaaattgttcttgtagacgaacataatgtaccgctaccgtttggaagtttaggaatacacaaaaagaaagaatataaaaactctaacatgtgtttgttcgtgtttgactgtttgtactttgatggattcgatatgacggacattccattgtacgaacgaagatcttttctcaaagatgttatggttgaaatacccaatagaatagtattctcagagttgacgaatattagtaacgagtctcagttaactgacgtattggatgatgcactaacgagaaaattagaaggattggtcttaaaagatattaatggagtatacgaaccgggaaagagaagatggttaaaaataaagcgagactatttgaacgagggttccatggcagattctgccgatttagtagtactaggtgcctactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccgaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatagttacatacaaactgaaaattaaaataacaccatttagttggtggtcgccatggatggtgttattgtatactgtctaaacgcgttagtaaaacatggcgaggaaataaatcatataaaaaatgatttcatgattaaaccatgttgtgaaagagtttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgttattcctgttatcatatctataggaaaggataaagatgtctgtgaactattaatctcatcagacatatcgtgtgcatgcgtggagttaaattcatatcacgtagccattcttcccatgaatgtttccttttttaccaaaggaaatgcctcgttgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggtaatttaataagaaatcgagactacattcccgggagacgaggctatagctactacgtttacggtatagcctctagataatttttttaagcacgaaataaaaaacataattttaaaccaatctatttcatactattttgtgtgatcaccatggacataaagatagatattagtatttctggtgataaatttacggtgactactaggagggaaaatgaagaaagaaaaaaatatctacctctccaaaaagaaaaaactactgatgttatcaaacctgattatcttgagtacgatgacttgttagatagagatgagatgtttactattctagaggaatattttatgtacagaggtctattaggcctcagaataaaatatggacgactctttaacgaaattaaaaaattcgacaatgatgcggaagaacaattcggtactatagaagaactcaagcagaaacttagattaaattctgaagagggagcagataactttatagattatataaaggtacaaaaacaggatatcgtcaaacttactgtatacgattgcatatctatgataggattgtgtgcatgcgtggtagatgtttggagaaatgagaaactgttttctagatggaaatattgtttacgagcgattaaactgtttattaatgatcacatgcttgataagataaaatctatactgcagaatagactagtatatgtggaaatgtcatagaaagttaatgagagcaaaaatatataaggttgtattccatatttgttattttttctgtaatagttaaaaaaatacattcgatggtctatctatcagattattatgtgttataaggtactttttctcataataaactagagtatgagtaagatagtgtttttcaaaacatataaatctaaaattgatggatgagatatacagctattaatttcgaaaatatataaatctaaaattgatggataagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatccccaattattctaatggatgtcatcaatgtagaaaatgcccaaccggatcatttgataaggtaaagtgtaccggaacacagaacagtaaatgttcgtgtcttcctggttggtattgcgctactgattcttcacagactgaagattgttgaaattgtgtaccaaaaaggagatgtccatgcggatactttggtggaatagatgaacaaggaaatcctatttgtaaatcgtgctgtattggtgaatattgcaactacctacgtaattatagacttgatccattttctccatgcaaactatctaaatgtaattaattatgattttgatgataatgttaccatacattatatcgctacttggttagtgtattattcagtatgaagacctattaataattacttatcttttgacgatcttgttataattataatataaaaacttatggcatagtaactcataattgctgacgcgataaattcgtaataatctgttttgttcaaatttttataaggaatctacaggcataaaaataaaaatataatttataatatactcttacagcgcgccatcatgaatagcagcagtaaattaattgctgttattaatggatttagaaatagtggacgattttgtgatattaatatagttattaatgatgaaaggataaacgctcacagactcatcctatctggagcctccgaatatttttccattctgttttccaataattttatcgattctaatgaatacgaagttaatctaagtcatttagattatcaaagtgttaacgatttgatcgattacatttatgggatacctttgagcctaactaacgataacgtgaaatatattctttcaaccgctgattttttacaaattggatctgccattactgagtgcgaaaaatacatacttaaaaatctttgttctagaaactgtatcgatttctacatatacgctgataaatataataacaagaaaatagaatcagcgtcgtttaacacaatattacgaaatattttgagactcatcaacgatgaaaactttaaatacttaacagaggaatcaatgataaaaattttaagcgatgatatgttaaatataaaaaatgaggattttgcaccactaattctcattaaatggttagagagtactcaacaatcatgcaccgtcgagttacttagatgcctcagaatatcattgctttccccacaagttataaaatcactttatagtcatcaactggttagttcaatctacgaatgtataacattcttaaacaatatagcattcttggatgaatcatttcctagataccatagcatcgagttgatatctatcggtataagtaattcgcatgataagatttccataaactgctacaatcataaaaaaaatacatgggaaatgatatcttcacgtagatataggtgtagtttcgcagtggccgtcctggataatattatctatatgatgggtggatatgatcagtccccgtatagaagttcaaaggttatagcgtacaatacatgtacaaattcttggatatatgatataccagagctaaaatatcctcgttctaattgtgggggactggctgatgacgaatacatttattgtataggcggcatacgcgatcaggattcatcgttgacatctagtattgatagatggaagccatcaaaaccatattggcagaagtatgctaaaatgcgcgaaccaaaatgtgatatgggggttgcgatgttaaacggattaatatatgtcatgggtggaatcgttaaaggtgacacgtgtaccgacgcactagagagtttatcagaagatggatggatgaagcatcaacgtcttccaataaaaatgtccaatatgtcgacgattgttcatgatggcaagatttatatatctggaggttacaacaatagtagtgtagttaatgtaatatcgaatctagtccttagctataatccgatatatgatgaatggaccaaattatcatcattaaacattcctagaattaatcccgctctatggtcagcgcataataaattatatgtaggaggaggaatatctgatgatgttcgaactaatacatctgaaacatacgataaagaaaaagattgttggacattggataatggtcacgtgttaccacgcaattatataatgtataaatgcgaaccgattaaacataaatatccattggaaaaaacacagtacacgaatgattttctaaagtatttggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccagaaactagttctgagaaaccagaggatatagataattttaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagtcacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaatattctgtattacatattatatatataataaacgttcacgtaaatacaaaacagagaacaaagtctagatttttgacttacataaatgtctgggatagtaaaatctatcatattgagcggaccatctggttcaggaaagacagccatagccaaaagactatgggaatatatttggatttgtggtgtcccataccactagatttcctcgtcctatggaacgagaaggtgtcgattaccattacgttaacagagaggccatctggaagggaatagccgccggaaactttctagaacatactgagtttttaggaaatatttacggaacttctaaaactgctgtgaatacagcggctattaataatcgtatttgtgtgatggatctaaacatcgatggcgttagaagtcttaaaaatacgtacctaatgccttactcggtgtatataagacctacctctcttaaaatggttgagaccaagcttcgttgtagaaacactgaagcggatgatgagattcatcgtcgtgtgatgttggcaaaaactgacatggatgaggcaggtgaagccggtctattcgacactattatcattgaagatgatgtgaatttagcatatagtaagttaattcagatactacaggaccgtattagaatgtattttaacactaattagagacttaagacttaaaacttgataattaataatataactcgtttttatatgtggctatttcaacgtctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggattcggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtctaatggcgaacatgttccatttataagaaatccaaataaaatggataacggtactctagaatttacacctatagattcgcataaaggatacgttgtatctagacgtggagatctagaaacacttggatattgtatgattagatggttgggaggtatcttgccatggactaagatatctgaaacaaagaattgtgcattagtaagtgccacaaaacagaaatatgttaacaatactgcgactttgttaatgaccagtttgcaatatgcacctagagaattgctgcaatatattaccatggtaaactctttgacatattttgaggaacccaattacgacgagtttcggcacatattaatgcagggtgtatattattaagtgtggtgtttggtcgatgtaaaatttttgtcgataaaaattaaaaaataacttaatttattattgatctcgtgtgtacaaccgaaatcatggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaaacaggaaacgctgtatatggaaaagttcaacatgattattctactatcaaagagaaggccaaagaaatgaatgcactcagttcaggacctatcatcgattaccacgtctggataggagattgtatctgtcaagttactgctgtggacgtacatggaaaggaaattatgagaatgagattcaaaaagggtgcggtgctacagatcccaaatctggtaaaagttaaacttggggagaatgatacagaaaatctttcttctactatatcggcggcaccatcgaggtaaccacctctcaagaagaccgcgtgaataatgtactcatgaaacgtttggaaactatacgccatatgtggtctgttgtatatgatcattttgatattgtgaatggtaaagaatgctgttatgtgcatacgcattcatctaatcaaaatcctataccgagtactgtaaaaacaaatttgtacatgaagactatgggatcatgcattcaaatggattccatggaagctctagagtatcttagcgaactgaaggaatcaggtggatggagtcccagaccagaaatgcaggaatttgaatatccagatggagtggaagacactgaatcaattgagagattggtagaggagttcttcaatagatcagaacttcaggctggtaaattagtcaaatttggtaattctattaattgttaaacatacatctgtttcagctaagcaactaagaacacgtatacggcagcagcttccttttatactctcatcttttaccaacacaaagggtggatatttgttcattggagttgataataatacacacaaagtatttggattcacggtgggttacgactacctcagactgatagagaatgatatagaaaagcatatcaaaagactttgtgttgtgcatttctgtgagaagaaagaggacatcaagtacacgtgtcgattcatcaaggtatataaacctggggatgaggctacctcgacatacgtgtgcgctatcaaagtggaaagatgctgttgtgctgtgtttgcagattggccagaatcatggtatatggatactaatggtatcaagaagtattctccagatgaatgggtgtcacatataaaattttaattaatgtaactatagagaacaaataataggttgtaatatcatatagacaataactaacaattaattagtaactgttatctcttttttaactaaccaactaactatatacctattaatacatcgtaattatagttcttaacatctattaatcattgattcgcttctttaattttttataaactaacattgttaattgaaaagggataacatgttacagaatataaattatatatggatttttttaaaaaggaaatacttgactggagtgtatatttatctcttcattatatagcacgcgtgtgttccaattcttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcaggttatcatcatccattaatataaaagaatatgtatatcaattagttaataatgatcatccagataataggataagactaatgcttgaaaatggacgtagaacaagacattttttgtcctatatatcagatacagttaatatctatatatgtattttaataaatcatggattttatatagatgcagaagacagttacggttgtacattattacatagatgtatatatcactataagaaatcagaatcagaatcatacaatgaattaattaagatattgttaaataatggatccgatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaataaattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatatacaacgaaacatctatttacgacgctgtcagttataatgcgtataatacgttggtctatctattaaacagaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaaaataaacaacataatgcagatttattgaaaatgtgtataaaatatactgcgtgtatgaccgattatgatactcttatagatgtacagtcgctacagcaatataaatggtatattttaaaatgtttcgatgaaatagatatcatgaagagatgttatataaaaaataaaactgtattccaattagttttttgtatcaaagacattaatactttaatgagatatggtaaacatccttctttcgtgaagtgcactagtctcgacgtatacggaagtcgtgtacgtaatatcatagcatctattagatatcgtcagagattaattagtctattatccaagaagctggatgcgggagataaatggtcgtgttttcctaacgaaataaaatataaaatattggaaaactttaacgataacgaactatccacatatctaaaaatcttataaacactattaaaatataaaatctaagtaggataaaatcacactacatcattgtttccttttagtgctcgacagtgtatactatttttaacactcataaataaaaatgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgcgaaacagataaatggaaatacgaaaatccatgcaagaaaatgtgcacagtttctgattatgtctctgaattatatgataagccattatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaaatataaatccgttaaaataattaataatttaataacaaacaagtatcaaaagattaaagacttatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcaacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccatgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggactatgcgtcgaagtaaaaattggaccaccgactgtaacattgactgaatacgacgaccatatcaatttgtacatcgagcatccgtatgctactagaggtagcaaaaagattcctatttacaaacgcggtgacatgtgtgatatctacttgttgtatacggctaacttcacattcggagattctaaagaaccagtaccatatgatatcgatgactacgattgcacgtctacaggttgcagcatagactttgtcacaacagaaaaagtgtgcgtgacagcacagggagccacagaagggtttctcgaaaaaattactccatggagttcgaaagtatgtctgacacctaaaaagagtgtatatacatgcgcaattagatccaaagaagatgttcccaatttcaaggacaaaatggccagagttatcaagagaaaatttaataaacagtctcaatcttatttaactaaatttctcggtagcacatcaaatgatgttaccacttttcttagcatgcttaacttgactaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacgatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatgttacaaatgtagaagatatcataaatgaaatagatagagagaaagaagaaatactaaaaaatgtagaaattgaaaataataaaaacattaacaagaatcatccaagtggatatattagagaagcactcgttattaatacaagtagtaatagtgattccattgataaagaagttatagaatgtatcagtcacgatgtaggaatatagatcatatctactaatttttataatcgatacaaaacataaaaaacaactcgttattacatagcaggcatggaatccttcaagtattgttttgataacgatggcaagaaatggattatcggaaatactttatattctggtaattcaatactatataaggtcagaaaaaatttcactagttcgttctacaattacgtaatgaaaatagatcacaaatcacacaagccattgttgtctgaaatacgattctatatatctgtattggatcctttgactatcgacaactggacacgggaacgtggtataaagtatttggctattccagatctgtatggaattggagaaaccgatgattatatgttcttcgttataaagaattcgggaagagtattcgccccaaaggatactgaatcagtcttcgaagcatgcgtcactatgataaacacgttagagtttatacactctcgaggatttacccatggaaaaatagaaccgaggaatatactgattagaaataaacgtctttcactaattgactattctagaactaacaaactatacaagagtggaaactcacatatagattacaacgaggacatgataacttcaggaaatatcaattatatgtgtgtagacaatcatcttggagcaacagtttcaaaacgaggagatttagaaatgttgggatattgcatgatagaatggttcggtggcaaacttccatggaaaaacgaaagtagtataaaagtaataaaacaaaaaaaagaatataaaaaatttatagctactttctttgaggactgttttcctgaaggaaatgaacctctggaattagttagatatatagaattagtatacacgttagattattctcaaactcctaattatgacagactacgtaaactgtttatacaagattgaaattatattctttttttatagagtgtggtagtgttacggatatctaatattaatattagactatctctatcgcgctacacgaccaatatcgattactatggatatcttcagggaaatcgcatcttctatgaaaggagagaatgtattcatttctccagcgtcaatctcgtcagtattgacaatactgtattatggagctaatggatccactgctgaacagctatcaaaatatgtagaaacggaggagaacacggataaggttagcgctcagaatatctcattcaaatccatgaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaaccattgtctcctgatacctgtctcctagcaattagtgccgtatactttaaagcaaaatggttgatgccattcgaaaaggaatttaccagtgattatcccttttacgtatctccgacggaaatggtagatgtaagtatgatgtctatgtacggcaaggcatttaatcacgcatctgtaaaggaatcattcggcaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggttcgtataatcttgtggatactctagtaaagtcaggactgacagaggtgttcggttcaactggagattatagcaatatgtgtaattcagatgtgagtgtcgacgctatgattcacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaattcttttcgttggtagatattgctctccgacaactaattgttaaccattttttttaaaaaaatagaaaaaacatgtggtattagtgcaggtcgttgttcttccaattgcaattggtaagatgacggccaactttagtacccacgtcttttcaccacagcactgtggatgtgacagactgaccagtattgatgacgtcagacaatgtttgactgaatatatttattggtcgtcctatgcataccgcaacaggcaatgcgctggacagttgtattccacactcctctcttttagagatgatgcggaatcagtgttcatcgacattcgcgagctggtaaaaaatatgccgtgggatgatgtcaaagattgtacagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctcaattacgtggattataacatcatattccggcgtatgaattgatgagttgtacatcttgacattttctttcttctcttctccctttcttctcttctcccttcctccctcttctccctttcccagaaacaaacttttttacccactataaaataaaatgagtatactacctattatatttcttcctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcggacatcgacgccttagaaataagagacttaaacaacggacacctggaattattaccatagaagatgttagaaaaaatgatgctggttattatacatgtgttttagaatatatatacagaggtaaaacatataacgtaaccagaattgtaaaattagaggtacgggataaaataataccttctactatgcaattaccagatggcattgtaacttcaataggtagtaatttgactattgcgtgtagagtatcgttgagacctcccacaacggatgcagacgtcttttggataagtaatggtatgtattacgaagaagatgatggggacggagacggtagaataagtgtagcaaataaaatctatatgaccgataagagacgtgttattacatcccggttaaacattaatcctgtcaaggaagaagatgctacaacgtttacgtgtatggcgtttactattcctagcatcagcaaaacagttactgttagtataacgtgaatgtatgttgttacatttccatgtcaattgagtttataagaatttttatacattatcttccaacaaacaattgacgaacgtattgctatgattaactcccacgatactatgcatattattaatcattaacttgcagactatacctagtgctattttgacatactcatgttcttgtgtaattgcggtatctatattattaaagtacgtaaatctagctatagttttattatttaattttagataatataccgtctccttatttttaaaaattgccacatcctttattaaatcatgaatgggaatttctatgtcatcgttagtatattgtgaacaacaagagcagatatctataggaaagggtggaatgcgatacattgatctatgtagttttaaaacacacgcgaactttgaagaatttatataaatcattccatcgatacatccttctatgttgacatgtatatatccaggaattcttttattaatgtcaggaaatgtataaactaaaacattgcccgaaagcggtgcctctatctgcgttatatccgttcttaacttacaaaatgtaaccaatacctttgcatgacttgttttgttcggcaacgttagtttaaacttgacgaatggattaattacaatagcatgatccgcgcatctattaagtttttttactttaacgcccttgtatgtttttacagagactttatctaaatttctagtacttgtatgtgttataaatataacgggatatagaactgaatcacctaccttagatacccaattacattttatcagatccagataataaacaaattttgtcgccctaactaattctatattgttatatattttacaattggttatgatatcatgtaataacttggagtctaacgcgcatcgtcgtacgtttatacaattgtgatttagtgtagtatatctacacatgtatttttccgcactatagtattctggactagtgataaaactatcgttatatctgtcttcaatgaactcatcgagatattgctctctgtcatattcatacacctgcataaactttctagacatcttacaatccgtgttattttaggatcatatttacatatttacgggtatatcaaagatgttagattagttaatgggaatcgtctataataatgaatattaaacaattatatgaggacttttaccacaaagcatcataaaaatgagtcgtcgtctgatttatgttttaaatatcaaccgcaaatcaactcataaaatacaagagaatgaaatatatacatattttagtcattgcaatatagaccatacttctacagaacttgattttgtagttaaaaactatgatctaaacagacgacaacatgtaactgggtatactgcactacactgctatttgtataataattactttacaaacgatgtactgaagatattattaaatcatgacgtaaatgtaacgatgaaaaccagtagcggacgtatgcctgtttatatattgcttactagatgttgcaatatttcacatgatgtagtgatagatatgatagacaaagataaaaaccacttattacatagagactattccaacctattactagagtatataaaatctcgttacatgttattaaaggaagaggatatcgatgagaacatagtatccactttattagataagggaatcgatcctaactttaaacaagacggatatacagcgttacattattattatttgtgtctcgcacacgtttataaaccaggtgagtgtagaaaaccgataacgataaaaaaggccaagcgaattatttctttgtttatacaacatggagctaatctaaacgcgttagataattgtggtaatacaccattccatttgtatcttagtattgaaatgtgtaataatattcatatgactaaaatgctgttgacttttaatccgaatttcaaaatatgtaataatcatggattaacgcctatactatgttatataacttccgactacatacaacacgatattcttgttatgttaatacatcactatgaaacaaatgttggagaaatgccgatagatgagcgtcgtatgatcgtattcgagtttatcaaaacatattctacacgtccggcagattcgataacttatttgatgaataggtttaaaaatataaatatttatacccgctatgaaggaaagacattattacacgtagcatgtgaatataataatacacacgtaatagattatcttatacgtatcaacggagatataaatgcgttaaccgacaataacaaacacgctacacaactcattatagataacaaagaaaattccccgtataccatcgattgtttactgtatatacttagatatattgtagataagaatgtgataagatcgttggtggatcaacttccatctctacctatcttcgatataaaatcatttgagaaattcatatcctactgtatacttttagatgacacattttacgataggcacgttaagaatcgcgattctaaaacgtatcgatacgcattttcaaaatacatgtcgtttgataaatacgatggtataataactaaatgtcacgacgaaacaatgttactcaaactgtccactgttctagacactacactatatgcagttttaagatgccataattcgaaaaagttaagaagatacctcaacgagttaaaaaaatataataacgataagtcctttaaaatatattctaatattatgaatgagagataccttaatgtatattataaagatatgtacgtgtcaaaggtatatgataaactatttcctgttttcacagataaaaattgtctactaacattactaccttcagaaattatatacgaaatattatacatgctgacaattaacgatctttataatatatcgtatccacctaccaaagtatagttgtatttttctcatgcgatgtgtgtaaaaaaactgatattatataaatattttagtgccgtataataaagatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggagaggctagaaaagaatagacggcgacaggtttctaataaacgtgttaaacatggtgatttatggatagccaactatacatctaaattcagtaaccgtaggtatttgtgtaccgtaactacaaagaatggtgactgtgttcagggtatagttagatctcatattaaaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaagaaattaattattcataatccagagttagaagatagtggaagatacaactgttacgttcattacgacgacgttagaatcaagaatgatatcgtagtatcaagatgtaaaatacttacggttataccgtcgcaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaaatacacaatgcatttttatatacattactgaataattattattattatttatatcgtatttgtgctataacgcgactatctaggtatttgtatctcactgatagagaacatataaatatagactctattaaacagttgtgtaaaatatcaaatcctaatagatgtggatgtacggctttacatgagtacttttataattatagatcagtcaacggaaaatacaagtatagatacaacggttactatcaatattatttatctagcgattatgaaaattataatgaatattattatgatgattatgatagaactggtatgaacagtgagagtgataatatatcaatcaaaacagaatatgaattctatgatgaaacacaagatcaaagtacacaactagtaggttacgacattaaactcaaaaccaatgaggatgattttatggctatgatagatcagtgggtgtccatgattatatagatgaatcaattaataaagtagtatatggaagagagtctcacgtaagatggcgggatatatggcaagaacataatgatggcgtatacagtataggaaaggagtgcatagataatatatacgaagacaaccataccgtagacgaattctacaagatagacagcgtatcagatgtagatgacgcggaacacatatctccgataactaatgatgtatctacacaaacatgggaaaagaaatcagagttagatagatacatggaaatgtatcctcgtcatagatatagtaagcattctgtctttaagggattttctgacaaagttagaaaaaatgatttagacatgaatgtggtaaaagaattactttctaacggtgcatctctaacaattaaggatagcagtaataaggatccaataaccgtttattttcgaagaacgataatgaatttagaaatgattgatattattaacaaacatacaactattgatgaacgaaagtatatagtacactcctatctaaaaaattataaaaatttcgattatccatttttcaggaagttagttttgactaataaacattgtctcaacaattattataatataagcgacagcaaatatggaacaccgctacatatattggcgtctaataaaaaattaataactcctaattacatgaagttattagtgtataacggaaatgatataaacgcacgaggtgaagatacacaaatgcgaactccattacacaaatatttgtgtaaatttgtatatcataatattgaatatggtatccgatactataatgaaaagattatagacgcatttatagagttaggagccgatctaactattccaaataacgatggaatgataccagtagtttactgtatacactcaaatgcagaatatggttataacaatattactaacataaagataatacgtaaactacttaatcttagtagacgtgcgtcacataatctatttagagatcgagtcatgcacgattatataagtaatacatatattgatcttgagtgtttagatattattagatcgttggatggattcgatatcaatggttactttgaaggacgtacaccacttcattgcgctatacaacataacttcactcagattgctaagtacttattagatcgaggagctgatatagtcgtacccaacacattgattatacatcagtacatacagtaaatagcatagatatggaggaggatacaaatatttcaaataaagttataaggtacaacactgtcaataatatatggaagacattacctaacttctggactggaactataaatccaggcgtggtctcgcataaagatgatatatatgttgtatgcgacatcaaagatgaaaaaaatgttaagacttgtatatttagatataacacgaatacgtataacggatgggaattggttacgacgacagaaagcagattatcagctctgcatactattcttcatgacaataccataatgatgttacattgttatgaatcgtatatgttacaagatacatttaatgtgtacactcgcgaatggaatcatatgtgtcatcaacattcgaatagttatatcatgtacaatatactacccatctactaaatataatagaataaaataaatgagtatgatcattttagataacgattgattttatcattaccgcttcattcttatattctttgcttacggaacctatatttagaaacatctactaacgattttttatgcttgcattattaatggtatgtaatatgattgattgtgtacgcaataccaatttgttaagtatgaatacggggtacaaacataaactgaaatttaacattatttatttatgatatatatcgttatcgttattgtttggtctataccatggatatctttaaagaactaatcttaaaacaccctgatgaaaatgttttgatttctccagtttctattttatctactttatctattctaaatcatggagcagctggttctacagctgaacaactatcaaaatatatagagaatatgaatgagaatacacccgatgataagaaggatgacaataatgacatggacgtagatattccgtattgtgcgacactagctaccgcaaataaaatatacggtagcgatagtatcgagttccacgcctccttcctacaaaaaataaaagacgattttcaaactgtaaactttaataatgctaaccaaacaaaggaactaatcaacgaatgggttaagacgatgacaaatggtaaaattaattccttattgactagtccgctatccattaatactcgtatgacagttgttagcgccgtccattttaaagcaatgtggaaatatccattttctaaacatcttacatatacagacaagttttatatttctaagaatatagttaccagtgttgatatgatggtgggtaccgagaataacttgcaatatgtacatattaatgaattattcggaggattctctattatcgatattccatacgagggaaactctagtatggtaattatactaccggacgacatagaaggtatatataacatagaaaaaaatataacagatgaaaaatttaaaaaatggtgtggtatgttatctactaaaagtatagacttgtatatgccaaagtttaaagtggaaatgacagaaccgtataatctggtaccgattttagaaaatttaggacttactaatatattcggatattatgcagattttagcaagatgtgtaatgaaactatcactgtagaaaaatttctacatacgacgtttatagatgttaatgaggagtatacagaagcatcggccgttacaggagtatttatgactaacttttcgatggtatatcgtacgaaggtctacataaaccatccattcatgtacatgattaaagacaacacaggacgtatactttttatagggaaatactgctatccgcaataaatataaacaaatagacttttatcacgtttatctatgtctaaatattacaaatagtaatagtataaactaaagctgataatacttaaaaaaataataatatcatttacaattaatagtataaactaaaaattaaacaaatcgttattataagtaatatcaaaatgatgatatacggattaatagcgtgtcttatattcgtgacttcatccatcgctagtccactttatattcccgttattccacccattacggaagataaatcgttcaatagtgtagaggtattagtttccttgtttagagatgaccaaaaagactatacggtaacttctcagttcaataactacactatcgataccaaagactggactatcggcgtactatccacacctgatggtttggatataccattgactaatataacttattggtcacggtttactataggtcgtgcattgttcaaatcagagtctgaggatattttccaaaagaaaatgagtattctaggtgtttctatagaatgtaagaagtcgtcgacattacttacttttttgaccgtgcgtaaaatgactcgagtatttaataaatttccagatatggcttattatcgaggagactgtttaaaagccgtttatgtaacaatgacttataaaaatactaaaactggagagactgattacacgtacctctctaatggggggttgcctgcatactatcgtaatggggtcgatggttgattattgattagtatattccttattcacacaaaaagaacatttttataaacatgaaaccactgtctaaatgtaattatgatcttgatttatagatgaagatcagcctttagaggattttaaccagtatgtttaatatgaaaaaaataaacataacatattttgagattaagcgctattgtgcttaattattttgctctataaactgaatatatagccacaattattgacgggcttgtttatgaccggcaatcatgaatttacagaaattatctctggctatatatcttactgcgacatgttcgtggtgttatgaaacatgcataagaaaaactgcgttgtatcatgacattcaattggagcatgtagaagacaataaagatagtgtagcgtcgctaccgtacaagtagtcaatcaaagagaacgtagtagattgttggctacatttaattggacagatatagctgagggtgttagaaatgagttcattaaaatatgtgatatcaacggaacatatttatataattatactattgctgttagtataattattgattccacggaagaactaccaacagttactccaattacaacaacatatgaaccttctacatataattatactatcgatgatagcactgttattactactgaagaactacaagtgactcctcatatggatctccatcgatgatacatgtattaaaatactttccgaataagtcttttaaatattgtattaattatgaaaaactatgctatgcgagtatgatgcaaagatgtttaatgatacgatactagattttatctctagcgagagatgtcgttagaatcatttatcataactacgtttaataataattcatcaacgaatatcgataacatgtgtcatttatacgttaaagtctgtccgtcttctctattgtttagactgtttgtagaatgctgtgatataaacaaactagtagaaggtacgactccgttacactgttatctaatgaatgaaggatttgaatcatctgttttaaaaaacctattaaaggagtatgtcatgaatacgtttaatgttcatgacatccattacacaaatatttaactcatgatgaagttgagaatgatatgctttctgatagtatagatagctttagctaatataaaaatatattaatccactatatattctagacttgatttaaaaccgataaactactactacgtactgtataagttgttaaaaaaaggagcagaccctaattatgtagatgatagaggtaatacttttcttcattacttctgcatctatatgtccacttatgagaaaacgtcatttaataagatgcatcgtgaaaagaaatttattaaagagttggtaaaatatgaaaccgaaagtaaataatataggaaatacacctctacataactacgtatctcaatatgatatcactctcattcctcatccacaacccattaaaaaaatggaaattaaagccctctattagcataaacggctacaggtctacctttacaatggcctctccttgtgcccagttcagaccctgtcattgccacgctactaaggactccctgaataccgtggccgacgtcagacattgtctgactgaatacatcctgtgggtttctcatagatggacccatagagaaagcgcagggtctctctacaggcttctcatctctttcagaactgatgcaacggagctctttggtggtgagttgaaggattcacttccgtgggacaatatcgacaattgcgtggagatcattaaatgtttcatcagaaatgactccatgaaaaccgccgaagaacttcgtgcaatcattggactttgtactcaatcagctatcgtctctggaagagtcttcaacgataagtatatcgacatactacttatgctgcgaaagattctgaacgagaacgactatctcaccctcttggatcatatccgcactgctaaatactaaatctccttcatgctctctcactacactttttatcatcttatgaggaatgattgccttcatcatttttcgtgaaataggaataattagcaccagaatagctatggattgcacatgtattctatgtcgtctactggatgaagatgtgacgtacaaaaaaataaaactagaaattgaaacgtgtcacaacttatcaaaacatatagatagacgaggaaacaatgcgctacattgttacgtcttcaataaatgcgatacagacattaagattgttcgactgttactctctcgcggagtcgagagactttgtagaaacaacgaaggattaactccgctaggagcatacagtaagcatagatacgtaaaatctcaaattgtgcatctactgatatccagctattcgaattcctctaacgaactcaagtcgaatataaatgatttcgatctgtattcgtatatgtcttcggataatatcgacttacgtctgctaaaatacctaattgtggataaacggatacgtccgtccaagaatacgaattatgcaatcaatggtctcggattggtggatatatacgtaacgacgcctaatccgagaccagaagtattgctatggcttcttaaatcagaatgttacagcaccggttacgtatttcgtacctgtatgtacgacagtgatatgtgtaagaactctcttcattactatatatcgtctcatagagaatctcaatctctatccaaggatgtaattaaatgtttgatcaataacaatgtttccatccatggcagagacgaaggaggatctttacccatccaatactactggtcttgctcaaccatagatatagagattgttaaattattaataaaggatgtggacacgtgtagagtatacgacgtcagccctatattagaggcggattatctaaacaagcgatttagagtaaccccatataatgtagacatggaaatcgttaatcttcttattgagagacgtcatactcttgtcgacgtaatgcgtagtattacttcttacgattccagagactataaccactacatcatcgataacattctaaagagatttagacaacaggatgaatccatcgtacaagccatgttgataaactacttacattacggcgatatggtcgttcgatgcatgttagataacggacaacaactatcctctgcacgactactttgttaataataatctcgtcgatgtaaacgtcgtaaggtttatcgtggaaaataatggacacatggctgtaaatcacgtatcgaacaatggccgtctatgtatgtacggtctgatattatcgagatttaataattgcgggtatcactgttatgaaaccatactaatagatgtatttgatatactaagcaagtacatggataatatagatatgatcgataatgagaataaaactctactatattacgcggtcgatgtcaataatatacaatttgcaaagcggttattggaatatggagcgagtgttacaacatcacgctcgataatcaatacggccatccagaaaagtagttacagaagagaaaacaaaacgaggatagttgatttattacttagctaccatcccactctagagactatgattgacgcatttaatagagatatacgctatctatatcctgaaccattattcgcctgtatcagatacgccttaatcctagatgatgattttccttctaaagtaagtatgatatctccggtcgtcataaggaactaaagcgctatagagtagacattaatagaatgaagaatgcctacatatcaggcgtctccatgtttgatatattatttaaacgaagcaaacgccacagattgagatacgcaaagaatccgacatcaaatggtacaaaaaagaactaacgtccatcattacagaaactgtaaagaacaatgagaggatcgactccatagtggacaacattaatacagacgataacttgatttcgaaattacccatggagatactttattactccattaaataatttatcatggagcgataatgtcctgtttcatttgtttccatgacatattacaaaatcgattccgtccaagatgataaaaacatttaccggcatcataaacacggagtttattttatatgtctcgcataaacattactaaaaaaatatattgttctgtttttctttcacatctttaattatgaaaaagtaaatcattatgagatggacgagattgtacgcatcgttcgcgacagtatgtggtacatacctaacgtatttatggacgacggtaagaatgaaggtcacgtttctgtcaacaatgtttgtcatatgtattttacgttctttgatgtggatacatcgtctcatctgtttaagctagttattaaacactgcgatctgaataaacgaggtaactctccattacattgctatacgatgaatacacgatttaatccatctgtattaaagatattgttacaccacggcatgcgtaactttgatagcaaggatgaaaaaggacatattcctctacaccactatctgattcattcactatcaatcgataacaagatctttgatatactaacggacaccattgatgactttagtaaatcatccgatctattgctgtgttatcttagatataaattcaatgggagcttaaactattacgttctgtacaaaggatccgaccctaattgcgtcgacgaggatggactcacttctcttcattactactgtaaacacatatccacgttctacaaaagcaattattacaagttaagtcacactaagatgcgagccgagaagcgattcatctacgcgataatagattatggagcaaacattaacgcggttacacacttaccttcaacagtataccaaacatagtcctcgtgtggtgtatgctcttttatctcgaggagccgatacgaggatacgtaataatcttgattgtacacccatcatggaacgattgtgcaacaggtcatattctcataatgttactcaattggcacgaacaaaaggaagaaggacaacatctactttatctattcataaaacataatcaaggatacactctcaatatactacggtatctactagataggttcgacattcagaaagacgaatactataataccgcctttcaaaattgtaacaacaatgttgcctcatacatcggatacgacatcaaccttccgactaaagacggtattcgacttggtgtttgaaaacagaaacatcatatacaaggcggatgttgtgaatgacatcatccaccacagactgaaagtatctctacctatgattaaatcgttgttctacaagatgtctctccctacgacgattactacgtaaaaaagatactagcctactgcctattaagggacgagtcattcgcggaactacatagtaaattctgtttaaacgaggactataaaagtgtatttatgaaaaatatatcattcgataagatagattccatcatcgtgacataagtcgccttaaagagattcgaatctccgacaccgacctgtatacggtatcacagctatcttaaagccatacattcagacagtcacatttcatttcccatgtacgacgatctcaaacccgtacccagaaatacctttaactatatcgatgtggaaattaatctgtatcccgtcaacgacacatcgtgtactcggacgaccactaccggtctcagcgaatccatctcaacgtcggaactaactattactatgaatcataaagactgtaatcccgtctttcgtgatggatacttctccgttcttaataaggtagcaacttcaggattctttacaggagaaaggtgtgcactctgaatttcgagattaaatgcaataacaaagattcttcctccaaacagttaacgaaagcaaagaatgatactatcatgccgcattcggagacagtaactctagcgtcgacatctatatactatatagtaataccaatactcaagactacgaaactgatacaatctcttatcatgtgggtaatgtagccatatgcccggtagttgcgatatacataaactgatcactaattccaaacccacccgctttttatagtaagtttttcacccataaatacaataattaatttctcgtaaaagtagaaaatatattctaatttattgcacggtaaggaagtagaatcataaagaacagtactcaatcaatagcaatcatgaaacaatatatcgtactggcatgcatgtgcctgccagtcttcagcaatcatcctcatcgtgtacggaagaagaaaacaaacatcatatgggaatcgatgttattatcaaagtcacaaagcaagaccaaacaccgaccgatgataagatttgccaatccgtaacggaaattacagagtccgagtcagatccagatcccgaggtggaatcagtcgaggatgtagatcctcctaccacttattactccatcatcggtggaggtctgagaatgaactttggattcaccaaatgtcctcagattaaatccatctcagaatccgctgatggaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaagttacctctcgcgacttcttctttttctgtctcaatagtgtgatacgattatgacactattcctattcctatttcctttcagggtatcacaaaaatattaaacctctttctgatggtctcataaaaaaagttttacaaaaatatttttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaaagttttacaaaaatatttttattctctttctctctttgatggtctcataaaaaatattaaacctctttctgatggtgtcactaaaatatttttattctcattctcattttctctttctctcttcaatggagtcataaaatatttttattctctttctctcttcgatggtctcacaaaaatattaaacctctttctgatggtgtcactaaaatatttttattctcattctcattttctctttctctcttcaatggagtcataaaatatttttattctctttctctcttcgatggtctcacaaaaatattaaacctctttctgatggagtcgtaaaaaagttttatctctttctctcttcgatggtctcactaaaatattttttattctctttctgatgcatcaactatttcttaaacaataacgtccaacaacatatactcgtcgagcttatcaacatcccctatgcccatctaggttaccagacaattgtatatcataaaataatgtttataatttacacgttaaaatcatataataaaacgtagatcgtataatattttttggtatataaatgatctagtaaaatccatgtaggggatactgctcacattttttctttggtacaaaatttcacacaagtttttatacagacaaattcttgtccatatattttaaaacattgacttttgtactaagaaaaatatctagactaactatctctttctctttctctcttcgatggtctcacaaaaatattaaacctctttctgatggagtcgtaaaaaagttttatctctttctc

SEQ ID NO: 2 - DNA, 138203 bp, synthetic construct
  source 1..138203: mol_type = other DNA
ccggcgaagccgcggatccgctgttccggcgaaggcaggacagacattttttccagcccataaattaaaaagaatcaacagaaaattatcatgatgataaaaacttttaggattgtatttagttgaacgtttcgtcaataaatcaacctcaaattttttcacatgtaatttcttgttctttttaatttatgggctggaaaaaatgtctgtcctgccttcgccggaacagcggatccgcggcttcgccggggcggcggccggctcccgcggcggctgagccgcgctgccgcgagaacgcggaccaggagttcctgcgggaggagctacggcagaggctggaactgctgaatgctttcgaggacgggcgtccgcgggaacgcgactccgcggaggcggcaccccgcagccgcgagacctcgctctggagtcagggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggagtcgggagtcagggaaacagaagtcaagtagtcgggtagacaggagtcaggtggtcgggtgaccgcgccgtccgagtcccgcaaaaagtttttagactctgagagaggccgacctgcctccaacggttccgcggaaaagtttttataaaaagttttcgggagaggccgactgccttccaacggttcctcctctcgcgtgccgcgggcggccgctctcccgcgacggtcccgccaacgcgtttacaaagttttaaaagttttcgagagaggccgacctgtcttccaacggttgcgcgaaaaggttctgcggaggtttggaagagccgcccgccctccgacatcctccgaaaagttttcgcaaaaagtttttaaaggtttcgcgaggagttttcgagggaggcgacctgcctccgaggttccgcgtaaacgtttttacaaagcgtcggaggtgggggcgacctgcccttcctctcctccgaaaagctcttgagagtgtgggagaggcggccggccttcgcggtcgcgcgaaaaggttctgcgggagttcgcgggagctgacccgccctccgcgcccctccgaaaagtttttgcacgaagtcttttggcgttctcgagaggacgcctaccccgacggtaacgcggaacgtctcgggaggtcggcctctccgctcccgcggtcgcgggcggcggcccgtctccggaggttccgcgaaaagttttataaaaagttttgagggaggtaccgacctcctaaagttttacagagagttctcgcaaaagttttgggaggtcgacctgacctcctaaagttatcgaagagttctcgcgaagagttctcgcaaagagtttgagaggccgacctgcctccaacggttccgcggaaaagttttataagagttttgagagaggtcgacctaccttccaacggttccgcggaaaagttttataagagttttgagagaggtcgacctactctccgaggttccgcggaaagttttataaaaagtttttacaaagtttttgagggaggtcgacaccctccggatcctccgcctcgcgtgccgcgggcggcctccaagagttttacggaacgttctggagaggagaggccgacctcccaaagattttgcggaacgttttggagaggaggactggcctcgaaaagattttacaaagagtttggagggaggactggcctccaacggttccgcgaaaaagttttgcaagagtttggagagaggtcgaccaccctccgaggttccgcgaaagtgtttgcggagagtccgagagaggcagacactgcctgcgaaaagttttcgcggacggttgagcgggtcaccgacctccgacagtttacaaacgttttacggagagttagagaggcagcagaccgacctccgaacagtttagttttacaaagttttggagggtgaactgtcatcgggaaagttttacgaagagttttgggagaggccgacggttagccgagcgagcgcgcgagcgagtttacgttctctcgctcgtgtgcgcgttaactcgcactggttccctctctaactcggagtgggcgagcgagtggttgactcgtcctccgctctcactctgagtggtgataactgttaaccattaactcgtcctccactctctcactctctcactctgagtgaggattgacttgttaactcgtcctccactctctcgctctctcgctctgagtgagtgaggattaactgttcaactcgtcaactcgtcctctgcctctcatccaagactgagtgggtagttgactgctcttgttctcttactccgaggggtgattgagttagcaacagctactcgttctcttgttctctcacaccgagtgagcgagtgagcaacgttacttgttctctcacaccgagtgagcgagtgagcaacggttaactcgttctctcgttctctcgttctattattccgaggagtggcgagtgaggtaacggttactgttacttgttctctgttcccttcctcggtgagcggtgcgtcaacttgttctcgtgagtgagtacggtcacttgttctctcgttctctacctcgaggagtgagtcaacttgttctcgtgagtgagttgaccgtaaccgttcccttacctcaagtgagtggcggtcgcttgttctcgtgagtgggttaaccgcgttcccttaccggagtgagcgggcgggcgataaaaataatcaattgactgattcgctcgtgagcgagcgaagggcggcggacaagggcgcgggatgctggtctaatctactaaggccgattacaaaaacggatgggagaccgggagggagagggtcacagctccgagcggtgcatccgcgccagctggcggcgccactcggccgcgggccgcgcggccgcccaggccgcgttgtagccgcccgccaccgcgacgcaggtcagctcgaactccggcccgagcgcgcgcacgtcgtagatgtgcacggtcgcgacgttcagcagcagcgcgcccttgcgcagcgaggcgaaggtcgcgtgcatgccggcggcgagcgggtacaccggcgagagcgtcccgccgccgtgcgcgaccaccgccatgtgccgcccgtcgccgccgacggtcaggcaggtcaccgaggcggagccgttcgcgcggacggggcccgcctccacgagggcggccggcagcggcggcgccgcggggcggaagagcccgccgaggaggaagcccagcgccaccagcgcgagcgcgccgagcagcctgcgcggcgaggggtgcatgcttgttggctgcggtgttggcgtctggcgggtggaaggcgggtgtggggtgtccgcggctgcggcgggagggtctcgactgctaggcggtcctttttcactttgctccgtggcgcctggtccggggcaagggctgcggcgggcgcccgggcgggcgggccgctaccccgccgcgcggcccgcggccgccagcgccgcggccagccgccgccaccgcgggcccgccgcgcgcagcagccccgccgccgagcgccccgcgccgccgcgggcgcgcgccgaggccgcggccccgcgccgcgccagcagcagcggcagccgcgcgtccagcgggccgccgcggcgcagcgccgcgcgcagcagcgccgccagcggcagccgccccgccgcgtccgggcccgccgcgcgcgcgcccgccgccagcagcgcgcgcaccagcgccggcgaggggcgccgcgcgcggaccaggtgctccacgagcagggtggtgagcaaggattctcgagaagtaggagtcatgtgtgacgacaaggagagacgttatattaggcgcgtcctacttcactttgaagatggtgtaaagtgttaaaacttgaacaccgttcactccaccactgccgttaccgtgtcctgccccaaaagcaaccacagtgctttttccaccacctgttccaaatccgttccaaaagctcccatccattgttgttagaactttcagatgtttctctaggttgtttagttccactgcaagtttcgaccattatcgttactggacatgctgttggtaatgagtttaataaccaatcataaaaatagttataatttgttataaagctaataaagtagcaaacactttaatgttatattttgcctaaccctccgttaacaccaccattaacaccaccacttaagcttttactaccaccactaccacctccaacacacattcttttctctaaaggtccccaaattccacctcctgaacttggacgttttacagcacctccgggtgtacttgcgtaccctttagaagttccactgtgactgtagatatgatactgtccttctccaggcatgattaaagtgtgttgtaattagtgttatctacgcaactgtgcgagactctcgaataaaaagaagctacattttacaattttgattagctgatgtaccacgctgtatcgcggccaccacaagcacccgatccagtagaaccaaatccagagtcgccgcggtcagtgttgtccaagcagttaacctcttgaactgctgggcacgatatgcgttcgcatattagctgagctatcctgtctcccttcttaacctcaaagtcactgtttccaaagttaaacagcaccactccgacgttgcctcggtagtcttcgtcgatcacgccagcgcccacgtcgataaagtgtttgactgcaaggccagaacgtggtgctatgcgtccgtagcaaccagaagggggctttatcagaaggtcagtaaatactacgcgactgcaatgcgaagggatgacacagtcgtatgcactacataggtctaatcctgcggcaccaggagatcctctggctggtatagtggcgttttggctgaggcgaacaacctgaagagtttccgtgtggcagaactccatggctagggtggcgagcggccgatcgactacggggtgtacaatttacactttctccagaaaaatcaggggcgggtcagcatggcgcggcgcaagtccagcagcgagtcgtacgacaggaagcacaggatggaggtcacgatctccggcggcagggcgcacgggcacatgaggccggcgatctgctcggccagcgagacgcgcagccgcatcatgcagatcttgccgaagagcgccgtcccgtagatggggaactcggccgcgcgctccaaaaaggcgttctgcacgaagagcgccttcgcgtcgtccgacgcgcgcagcacgtccaacagcgtcgcgtccgtgtggcagcgcaccgcgcgcatgcttgcgatctcctgctcgcacgcgcggatcacggtcgcgtagtccgccagcgcgcgctccgccagcatgcgcgcgccctcgccgcgcagcgccagctcctgcacgcacagcagcgcggcctccgagcgtcggaacacgtggccccattgctcggatgtgatcagcgcgcgcgcgagcagctccgtcggcggccggcgcgcgagcacggccgccgtcgcgcgcacgttgttgcggcgcagcatctccgaaaccgcgcataggcccgaggccgacatgtgctcgagctccgcgcccatgcgcaccagccggcagcaggcgccgtggctgaacaccgccgcgcggtgcagcgcggtctgcaggttgttgttgcgcaggttcaggtccagcccgcgctcgagcacgaagtccacgacgccgcgctcgcagctcccgtaggtcgccatgtagtgcagcatggtgttcccacacgcgtctacggcggccgggtccacgcctaggcccgtgagcgtgcgcaccatgccctcggagatcttggccgtgcgcgcgaggtggtgcagcgttgtgcgcccgtacgcgtccacgacgcacgcgtccgcgcccgcgcgcagcatcatgtccacgagcgcggcggagacgccgccggagcacagcagcgccgccagcggcgtcaagccgttgcagtcgcaggcgtttgggttcgcgccgcgctcgagcagcagccgcagcacgtcctcgcggatccactggttcttggcgtacacgtgcagcggcgttacgccgtaggtgttgccctcgttcacgcgcgcgcccgcgtccagcagcagccgcgcgacctcgagctcggcgccgtcggggccgcagaaagccaggaaggaggagagcacgctgtcgcagacaacgacgctggcgtcgcagaccacgtccgcgcccgcctccagcatgagcgcgaccacctccggccgcacgccgtcgtactgcacgtaggcgtgcagcggcgtgcggccgcaggagtccttggctttcacgtccgcaccggcctccagcagcacgcgcacgatctccgcgcactgctcgtgccgcgcgaagtgcacgcagaggtgcagcggcgtgcgcccgtgctcgccgcggaagttcacgtctgcgtcggtggctacgagcgcgcggaccgtttcgaggtccacctgcccggactccaggtagcggaagagcaggtccgcgtgcgggaccacgacggactcccgcgagagcatggcggcgtttacaaatattgaaatcttttttcactcatctttatgggcgctgaacgcgcaataagggtgagagtaaaaaacttctacaaaaagcgtacaaaaggtacaaaaggtaaaaaaggcggggcggggacgggctggggtgctgcgagctgaattggcctctacacagggacgccctcgccggagccggtgagccggtagccggcgccggcgatcatggtcaagcgctgcacgagctcgttgcgcttgacgccggcctctgaaacgcacaccatgtggtggatgtaccgctcgatgcactcgcagcgcgggagagtggagtcaagatcggatgcgagttgcagaatgtcatcccagagctcggagaacttgctgtacagttctcggaggtctctctccattcgagccgtaagagagtcaggatgcggtgttccttcgggagtctgagcgaacaccgcgaacaggctggttatgccgtgttctagaatagagtggttccgcgttaatgccgcagacaagggtcgtcgtccgcgcaacgactggcggcagagcgctgtttgtgccgcaccgcccattcctctggcgatcgcatccaccgacgcagtgatcatctgcgcgccgacgtcattgtagcgcgcgttaaactcagtaatcatgattacgagattgcagatttcatagtagcacttttccaagtcgacgcgcagtttcacgatctggttgacaatcttgcacgcctttcgccgcgtctccgccacgttggcgactcggacttgcgcttcctggtcgatggacggcggaaacacttcaaacccaaggtcgcacagttcagcggtggggactagcgtcacgatgatgtactccgcatcgccacccacttgcggcaggaagaacaccgaccgcgcggcgggaacgaccagaacgtcgccttcctgcatgttggtttttagaaacttagtgttgttcacggagatgccggccatgccctcgtttttgacacatattatggtgacgtacgcggcgaccgtgggggccatgtggtggcgcatgtaccactcgtcgtgcttgagtttcagaccgtgagattcgccaacctcgaagtgcatgttggcgtctctgacgtagcgcgagaactcgctgcgacagattcgcgcgggcgcccggtggaacgtcgactcgaagagactgatggctgtccattcgcccacatgagtgaccaccgaagaagtgttttcgatccgagtctcgaacaccgagtccacgagcaccggacagttggttccgggcaccgtcagcaccaagggccgcgcctccacgggggcgacggacgaagccacggagtcggtgtccccgtacccgtagtcgtcgtcggagtcgccgcctccgtcggccccgtcgcgcggcctccgcagcggcatgcagccggcggtgggaacgcactggtttcggccacggccgaagcggccaaacagtctcgccagggctgacatccttggacggccacaccaaaaccaaaaaaacatattttatcagttatttgtcgattttcaccggctcaccgagggcaggacctcctggatcccggacacccccgccaggcagcgggccgcgcgctcgcgcacccaaaagcggtcgtagccgtgccggagcacgaaggccgccgtggcgtggcagtccacgcgctcgatgaagccgtggacggcgcggcgcgcgtagctcgccgcgaaggcgcggaccaccgccgagcagcgccccgagggcgagtcgtccgtctccagcgccagcggcatgctcgcgatgcgcgacatcaggttggaggtctgcgggatgttgagctcgcgcgtggcggtcatctgcgcctcgagcccggccttgagcacctcgtcgcagcggccccactccagcgcgcagaccacgcggatctcgtaccccttgagccgcagcgcggtctcgatgtccacggaggtgagcaccgcgctgaagcgcaggcgctcttcgtccgcggggtcgaagagcacggggatcttaacctccgcgctgcgcgtgacctcgcagagcgcgatcgcgagcagcccgcgcgtgagcttgctcaccatgcgcggcttgcccacggggtacagctggctcgcgacctcgcgcagcgggtacgccagtcggaagcagcgcgcgtccgcgggcaccgggctcgcgcccgtctcctcgaggaagagcgcggcctcaaccatgttcagcgcggagaagtgcaccgggcaggcggcgcagccgcgcgcggcgttcgcgagcaccatctcgcgcagcccgcggaaggccgccatgtcgcaggaggggaagatgcgcgcgagcgcggcctggtgcgcgagcgccgcgtccgagagcgcctgcgcggccgcggcggcggcctcctcggcggcggccgcgctctcgtccgcggagaccacgtcttcgggcacgtccacgcagacgccgccccagaactcgcagtactcggagaagagcgtcgcgggcgcaaagcgcgcgaggtccacgaaggcgacgcggttgccgagcctggagagcagcgtgttctccgagatgcgcgtccagcccttgccggcgagctccatgacctgccgcgtgtcgaagagggagctgtagaagccgtacacggtgatgttttccttgcacgtcgtcagccacatgaggaagtcgcgcaccaccagcttcgcgcagtctccggagaacacggggccggcgttcgtcgcgatggagttcaggcgcacggtgccgtcactgccgaagcggtacacgaaccaggcggccacgctgttgccagagggcgtgtgaacgtgtggctgcgcccaggagtcggcgctcgcggcggtgcgcacgtcgtgcgagagcacctcggtgtcggggcgcgagtaggtgctggggtctttgatccagatggcgtagctgcccacgcagcacacgttcatgaggtcgagcagcgtctgccggcgcagcggcgtgccgagccggcgcacggcgtcgtgcgagaccatgcgcaggtcgtagaggcccacgtccgagagccactggttgagctcgtccatggacagggcgtcgcgggggggcgggctgtcttcgaaggcggcgcggagctcgggctccgtctccgcgcgctgccgcaggatgtccaggaaggggctggaggagtcggggatgtagcagtcggggtcgtgcctggacactatagcgaaccgctgcgtcgcgggcggcggggctagcgcgtcggcgcgtgcgtcgatgaaggtgcacgatatacgcacggacttgagcgaggggaggacgactgcggcggcgcgcgcgccctccgcgtcgaagatcatcgtctttccgtccctcgcctttgcgagcgcgtattctccaggcacgaggtccgtcggcggcggctcgtcccaggcctgccggtcagggacgccgccgcacacctttccccagaaccccagcatcctccaaaatacctataaggacggccaatagcggggcttgcgggcgttcggaccttccgcgctttaattttaatttattggcttgcagaactccgagcgccagtcccgctcgaagaccgcggacaggtccttgacgatgtcgcccttctcggcgttcacgctcacgaaggcgtggtagcggtagtgcgtgccgtcgaggttggcgaccgtgaggtgcgcgaaggtgtcgtccacgatgagcagcttagtgttgttcgcggcgtcgtcccggccgggtaccacgaacttgcgcacggacatgtccacgctgccgacgccaaagtcgtcgaggctgcgcgcggccgagaccgaaagcgggtccgcgttcttccactcggtaatgatcacgcgcacgcgcacgccgcggttgatggccgcgcgcagcagcgcgtcaatgatctgcggccagtactccacggcgctggcgtgcttgatcaccggcaccatcgagagcagcgagaggtcgatgctgttcttggcgttctcgatgcggtgcagcacgaggtcctcgtcgagcgtgcggtagaagcctaggaagcgctccggcgagtccgagaagaatacgccgcccccggagtggtcgaggtggaagttcgtggccgtgggcgtgacgatggcgcagcagagccgcgtgaacggcaccttcggctccacgatcatggagtagaaggtgttgtagcggttcatgaggtcccaggccaggtgcttgttggtggagtagagcccgaggttcttgatggtggacacggacccgcccgtgagcgaggcgcttcccacgtaccagtgcccggcgtccgagagccagaagctgccgagaaggttgccgacgccctccttggtggacaccttgaccttgtagtagttgacgcccgcctcgcgcagctcgtccgcgtccttgtccttgctctgcacgtccacgagcagcgtgacgtctacgccctccttggcgagcgtgcagagcttgtccttgacgtcgacgccctccttggtggagctcaggttgcagcagaagctgcagatgtacaagaacttcttcgcggactcggcgatagcggtgaagcagtcgagggtgctcatgttgccctgcgccaaagacgccacctctgcgggcagcgtctccacgacgcggcagtcggcgcccagggggatggaggagaacggccacatttatttatctcacaaaaataatagggcttcagggaaagtcttttagcaggcgggcgagttcttcgagttcgcttaggagttcttccatttcttcggaagtcagcaactggagctcggacttgatttgaatatcttcgaggaaaccgtctagcatgttcgccatgtcttccggggagcactgcgccacatcttcggggacaggatcgggtgtgggcattaggtctccgcttacttgaacgtcgtccatcatcctgtcgatgaggtcttcgacttctagacggggtccgtagatcagcatatttggtgatggaggtagtttaaggtgcgagagttagtgttatacgaccgccaacgtgtgtttatcgcgcgtacattttcaataattaacaaactccccttcctgcgcctgctcgagaagcagctcgtccagctcctcctgtcggcgcgcggccacgcgtctttccgcgaagagtaccatcagctccagccccacgccgcacagacccaggacgccgaacaccaccgccgccgagatcgacagacccagcagcaccgacatcctcacgcgggcatccggctatttaatcgttctggaaacgtattaatatgggcgtcgtcatgtgcgggtgtctgtttgtgtgggcgggctggatcgcgcgccgcgtgcgcggcctctgcgcggcgctgcgccagagggtgtcgcgcgacaagggctacgtggccgtcatccagacctgcgacgacgactacttcacagaggaggagttcgacgacggcaagcaggtggtcgcgctcctgcgcgacgtctcgcgcgtggttgccgcgcccgcgggcgtgacggaataagttaggataaggagtcgaggggagaaaaacagcggtcacactataaactcgcgcgaggccgattttgacgtgctcatgtctggaagctccgctttctgcagcgcggagcggcacacgaagcacacttccgtgttggtgggagttatgcagtggacgtggtagccgtgcccgcacaccatgacttggaacggacacgcgccgggacaggccgcgtttatgcatccttccggcgagcgcttgttgcagatgtagcacacgtctgagcacgccagcgtgcaggacacggccagcttccactgcttaaccttaacgggcatggctagttgaacacgaccatgggcgagtcgcgagcctcgagtcgggggttcagggcaaaccgtttcacgccgtcaacggttcttctctttgcaattttctctctgcacaggctcgtcagcgtcatctcggccaggcgcgcgtcgttgcctaggtgccgcgcggcgtcctcgaccgtcacgcccgtcttgccggcctcgtccatgagcacaatgcataccaggtgcgcgctagagcatatgacctcctgctcgcgcccgccggcagcggggatggttagctccgcgcgcccgaaggccgccagcggcgccacgtcgtaggcagtgtctgctcgggcgagcgccgactccacggcaccgcggagcgactccggcggcgtcagcgcggccagcggcaccggcgtgggcacggtgtacacgttcacgggcatgagcaccatctccgggtcgtggtggccgctctcttcgccgtcgtgctccatgggctgcggcggcggcagcagcgggagcagcagccgtccggacatgagccggcgcacaaggtcgttgagcgcggacgaggccatcggcgggtacagctccatggccagcttcagcgataggtgcttctcgaggttgacgccggtgtagacgctcttcacgatgcgcgcgaaggccacgcgcgcgaaggccgccagctcctcgcgcggcaggcgctcgatgtaggagagcagcatgtcggtgtcgcacggcggcgccgcgaccaccgcgccgtagagcgccttgcccgagagcttttccagcgcccttgcgtgcaggccgtgggtcttgagcacgtccacgtagttcacgtacaggcagagcgcgcgatcgaggttgctctccgcgacgtgcgtctcgatgcactccacgatgagcgggcccatgcggtccttgatgaggtctatgagcccgccgtaggcgactcgcgcgctcatgaggcacgtgcggcagtacgccatcaggccctcgaggtccgcggctatcacgtcctcgaccacgttcgccacgacgcgctgccagagccgcaccttgctcacgttctggtgccgcaccatgtccacgagctcgtcgtacgagccgccgggctcgtgcgcgcgatcgacgatgcacctcgccatggtgcggctctggcgcatgagctcgttcgtgaagcgcacgcacgcgtcctcggagaagagcgcgctcaggcaggagtagcagcggtccgcgacgaggtgcgggaagcggcactccacgacgccgcggccgatccgcagcacgcactcgccgtacatctcgtccatggcctcgcgcagacagtcgtccagcacgtccgcgttgtgcgcccactggatcacgcagaggtagggctcgatgttctcgcgcgcgttttccacctcctgcaccatgtactcgagcacggtcatgtcctcgtggatgtcggtgcccagcatgcgcccgggcggcagccagctcttgcgcgcgatcgcctctcgcaggcacgccaccgccgtgaaggtgttgacgcggagcttggtcagcagccgccgcagtcgggagatgtgtgccacggagaggtccatctccatggcctgggcgatgaggcgcgtgagttcctcctccatggcggcggctccgcgggcagatatacgcgaacaacggtaagccgtgctatttcatttttggacaaaaagctagtcgtcgacgcgcatgttgtcgaggttccggcacagcgagagcacgtcgtcgcgcgcgcgcctccggcgcagttgattgttcgcgcgccgcgcgtccgcgagcgcctgtctgtacatcgcggagtccgcgtacccgtgcagcggcgagcgccgagcgccgggcctcgggctcgcgcggcgcgggagcggcgttggcgcgcgcctcgagcgccgcgcgaagtgcgcctgcatggccagcaggcaaccgaacggcaccatgtatcggtccatgaggcactggctggccgcggacggctcgcgcgggtgcatcacgccgccgcccacgtcctccatgacgtcgcgcagcacgcagcgcagcatggtctccatgccgtccacgggcttgaaccgcaccggggaggcgtcgacgtagaagccgtcggccacgaagtagagcgcgtccagcccgccgagtttctcgccgagaccgacgaagagctcgtccacgtgccagtccaccaccgaggccttgaagagcaccacgtgccggatgtcgtgcgagcgcgcgagctcccaggtgtcctcgccgatgttgctggcgtcgatgcggcctgtcatgcgcacgctcacgcacggcgtcatcccgttcttgtagcagaactggcgcgcgagctcctcctggcgtacgatgtcgaccatgctctccatgaaggaggtggagagcagcatcgcgccgcgcgcggcgcgggtcgcgttttcgtccacctccacttccatcccgccgccgatcctaatcatctatcgtatttaaattttcggcggagcagacacgcggctgctcgctgcgcgatcgcttcagccgcggcggcgtcacgcacgcgttgcggcggccggcacgcacggacgaccgccggggctcttcgctgagcgagcgccgcggccgcgtgacgcgacagtcgcaggtgggttgccgggagtcgctcgcgcgccttcttcgcatttcgccggaacgccgcgtttatgtaggggattatattttcaacgtaactaaatggacgggggcgtgcacaaacggcctttcatcgtgaacgtggatggcatgggcaaggtgctcgtgctccggtacttgcggatgtgcgaggtgcccgaggccaagtgcgagggctcgcgcgcgtcctgcgtgctcaagatggaccctccccgctcacccagctgcgagcgcaggccgtctctcccgccgtcccccccatgccccatgcgcacgcctcccgggtcgccgctccaggctcccttgatgcgcacgcagatgctccaggggctgttcgacgctgccaaaaacaacggcgagcagatgtgccgccgccagtaacctaggctgcgcagtacgaaagttagtgcgtgatcacgttttttgcaatgtcgatcacgccgtgcgtgcccgtcttgcgctcgcgctccaccacgctagtcacgggccgcgcgtccgagactagcgaccccagcatcgagcgcacggcgccctccgcggcggggtggcgcgtcagcagcaggaacatcacgatgtgcgcggagacgccgcgccggctcagatcgtgcacggcgtcgccgtccatgagcacggtgttcgagaagtacgtgaacagagtgttgtctcgcaccaggaaggccgagttcgagacgctctcgaagtccacgatctcgtcgtcctgcacgcccatgtccaacagcgtctgcacgagcgcgggctcgtccaggaacaccactgcgcgcgcgaacccgcagtccagcgcgcgcgcgtccgcctccaacacgcgcgaggcgccgccctccggcggcaggaaggcgcagggcagcggcgtgcgtccgtccgccggcgcctccccgagctcctcgagcgcgaaggccagcagcgtctccatgcgcgcgcgcgccttgtcgaagttgtccgcgaggtcgcggatgcggtctgtctgcgagaacatcttcagcatcgccatgagctgcacgaaggggtgcagcacgtatatgttgtccacgagcagcgtgggcagcgcgcgcagcgtggcctgccgcacgttgaagctgtccaggatgtgcccgccctcctcgtcctgcagcaccacgtagttcttcaggtagggcacgcgcagcagcacggtctgccgccccgtgacgaagtatatcaggaaggcgaggttgatcaggaacgggcgcgcgttcgtctgcaccatgtcgatgtcgccgtactctatctcggggttcagcaggtgcagggcgtacgagccgtagcacacgcaccgcttgttgtgtcggcgcaggtgctccttcacgagccgcttgaccacctccaccaggtccgagtgcttgtgccgcgccatcggcgcggcctcctcgggcggcggcagcactgcgtacgagttgagcgcgcggctggcgagcgcgcgcgcgcggggcgcgtccacgcgccgcaccgccgcgttgattgcaggcgtcggcgtcgtgagagagcccagcgtgcgcgtgaactcgctcacgatcacgctctgcagctccagcaccgtcaggatctggcccagcttctccaggcgccgctgcctcgagaagtactcctcgatgcgcgcggcgatctccttctcggagccgcctagcttcttgaagaagcgacgacgactctttacaacaagagagagaaaaagcttcctatcgaagttgaggacgcgggtcatgttgcggcgctgcgcgcgcaggagcacgcagcgctccatggaggggcgcgagccgaggtactcttcgatcacgggtggagccatgacagctattttctgaacccgcgattattgtacagcgcaagccgcgcgcagacctgctggcacagcagcgtcgtgtttcgcatgcacacgcgcgaggactcgatcgtgcgcgcgtccggtgcccaggcgcgcagctccatcagttcctgctcgacgaagtccacgggctccacgaagcgctctgcgcagagtccgtccgtgaacgcgttgacgatctgccgcacgagcactaccacgtccacctgctccacgaggcgcacgcccatggcgacgtgcacgaagaggcagcggaagagcgcgtccatggccatctggtggtccgagcagggcccgaccgcggtcttgcagcccagcgcgaagcgcccgatgccgcggtactgcaccatctccgagggcgagaaggagagccgctccatcttgagcacgggcggcgggcccgccggcagtccgcgcgcgaggtccagcaccggcgtccacccgggcgtgaacatgtccgggatcaggaagagcccgtagctggccatgcgcgcgatgtcgaaggcgtggtccacgaccttgttcacggcgctgtccgcgcggtttacgcgcagcgcctgcaggatcacgtttccggaagcgtgccgcgtgatcgcgaggtccgcggtcgcgtacccgcgcaggcccggcaccgcgtacgcggtcagacacacggccaggcgcgcgctgtgctccgaggagaagatctcctgctcgtagccctcctcgggctcctcgcactcgcgagggcgccgcacgtcctcgaccgagcgcagccgcatctctccctccgacaccagacagccaagcgactccctcaccgccggcgcgagcacctccgtggcgcagagcgcgtcgtgcacgcgcttgagtgtgttcggcttcagcgcgtagccgaagagcagccgcgtcatccgcgagcccgagaacgcgaagcggcgcacgtactcctccgcgagctcggggcggtcgttgatccacgaggtagagaagacgtggtcggaggcgaaggggtctgcaccaaccgcgagcagtgtggacagaggtacggtgtcgaggaagtccacgacgtcggggaagttctggcgcacgcaggcctcggcgacgcgtctggtgtgcacgcacatgtcggtgacgggcacccggtggccggactccacgacggacacgcagacgtcctcggtgacggcgtccacgggcatggtccgcagcagcttgccgagcacgtcgccgaacccgccctcgagcgccttgcgccacacgaaccccgcgtcgaacttcccggggaagtccgcgatcaccgaaagctccgcgtgcgagaggttgtccacgttgaggtaggtggcggcgtccacgaagatgggcccgaaggcgccggtgtcggagacgcggtctctgaggtagtccctggcgtagtggaggtactcccgcacctggccggcgcggatgcgctcgagcgcgaaggccttcatggtctcggagcagagcacggagtgccgcagcccgtccagtgtgcgccgcacgtcgtagacgccgcgcatctgcgcgagcatctcgacggcgtccgccggcgtcgccgccgcgagccccgggttcactggaggtatcctgtgttctgcgagcatgcgcttgaggaaacagaggtccagcggccgcgtggtgtacagcgcggaggccatctcggggcgcgtctcgacgatgtcctcgatcatctcgtccgtgaatgccgcgttgatgttgtgcacgctgcgcgcgttcacgtgcaggaggatgtcgcccacgttgtcggggaatcgctcctcgatgagccggacgtcgtcctccgtgatgttcatgtagggaatacagcggcagagcagcgcgtagtccgcgaactgcgcgatgtagggcgtgtggaactcgatgtgtctggcgaagagcgcgccgcagcgccgccgcgagagctcttcgagcaggtcctcgggcgtgacgtgctgcgggcggaagaggtgcaggtgcgtggggtgctcggcggccacgcgcgcgtacagccgccgcgggaggtgtctggggtgcacgccggcgagcacgagatccatggcctctgaggtagacagtgcggcgaacgcgcgctcggtgccgcccgcggcgacagcggcgccgacaaatctcttgagcagctgcagcatcgcgtgtttgggctttcgcggaaggcgcttattttaatgttattggcggtggccggtgcgagataaaaattagaactgatgccgcagttgttgatgatatgatgattgcgctggccggcgcgagataaaaattagaagctgatgccgcagttgttgatgaggatggtgagtgcgctggagcaagcggtgtggcgcgctagcttcttgctggccccgtcggccacggcaacgacctttccggatatcgtgatggtgcaggtgaagcgcggacagtgatcctctccgccagcacgcgtctcgcagaactccagaggtctgtgtgtcatcatgcagaactcgttgaccgcgctgaccgggttaagacttttgaggcgtatcacggcagactgagtcatgatgtcgatgtcgccgccgaaaagcgtatcgcacccagcctcggtctccatgggctcggtgtcggagttttcgtcctcctcggtgggcgcggagggcgcgcactctacgaaccagcggggcgggtttccgtcctcacagcaaacctcgtccgagtccagcaggcggtacagctggcggtttgcctcgtgtttggatatgccgagctccttcgcgatttgcttggccggcagcttgtcgtcggattttctgagaagctcgaggatcagagacgcgcactcgcaggccattgtggcgatttacggggcgtgcgtttttttaggattttggcttgcctttcttttcgcagaacttgggaggattgaaactcttttggcaatttttgcaggcgtacttgatcaagggcggctcgtccgccgagcgcgtctggatcatcatcggcatggtgttcttgctctggcacgaggggcagggcaggttgaacttctcgtcgagcacgttgaagtacccgctgtagtcgtggtccggcacctcctcgatgtcgtagggcacgcgcgcggccacgcacttgaccgcgaagagcaggtaccgcagcgcgtcgtgctccgcgccgctggtcgcgcggatctgcgcgcacaggtccgcgtagtcctcgttcgcgtccacctccaggctgcgcttgttcttgtacgagagccggttcttggcgtccttcgagtactcgatgccgatgttgtgcgcggggtcaaagttggtctcgtcggtgttcgaggtcttggtgttcacgatgttcttcagcgcgaagcgctgcgcgcagtccagcgcccatcgcgcgatgcgcgcggcctccgccgcgtcggtgtgcttcgccgcgaggtcgcgcagccggtcttcgtccatcgcccgattttaggttgggtatattatctcaattccgctcttccgcgggccgcgggcgcgcgcccgcggcaaattaggcgttacaaatggacttcgtgcggcggaagtacatgatacacgccatcgaccgcaacctcgacttcatgaaggccgaggtccagcagaaggtctccatcttctccctcgggcacgtgctcgcgctccactacctggtcaccgcctttccgcaggcggtcatcaccaaggacgtgctcgcgagcacaaacttcttcgtgttcgtgcacatgtcgcagcggcacgaggtcttcgacgccgtgctcaaggcggccttcgacgcgcctcagctctttgtgcgggcgctctcgcggcacttcgaggccttcgttgccgccatccgggcctaccgcgcgacctgcgcggagctgctggccgacgcgcgcttcatggaggtggctgcgcgcgcggccgagctcgcggaggtcattggcgtgaaccacgacatcgccgcgaacccgctcttcgcggacggcgagcccgtgcgcgacgcggagctcattttcgcaaagaccttccgcaagaccgagttccgcgccgtcaagcgcctcgccgtgctgcggctgctggtctgggccttcctcgtgaagaaggaccttggcggcgagtacgcggacaacgaccgccaggacctgtttacgctgctgcagaaggccgcggggcccgtgcgccacagcgcgctcacagagagcatccgcgagtacctcttccccggagacaggcccagccactgggtctggctgaacgcgcgcgtggccgacgacgcagaggtgtaccgcgaccggcccgcgcgcacgctctacgagcgcgtgctcagctacgcgtactcagaggtcaagcaggggcgcgtgaacgccaacacgctcaagctcgtgtaccggctcgaggacgaccccgacatcaagggtctgctgctgcagctcatctacgacgtgcccgcggacatcgtcggcgtcgtggactccgcgaacgaggagtggcggagctacttcgtgagtctgtaccgcgagaacttcgtcgacggacgcaccttcacctcggacgcgcgcttccgcgacgacctcttccgcgtggtcgccgccgtcgatcccgacttcttcgagcccgagcgcatccgcgaggccttcagcgcagacgcgcggctgcgagagcgcttcacggacatggacctcaacaacgccttcatgtcgcacctcatctacgactccgtggaccccgacgtcgccgccgccgagcgcgggctcgcactgcgcgtgcacaacgaggactccgactacttcatccgggagtacaacacctacctcttcctcagcgagaaggacccgctggtgctggaccgcggggcgctcacgcggctctcggacgtccccgccgagcgcttccgcgacctcttcagcgacagtgtgctgcggtacttcctggacgcgaagctgggcacgctcgggctggtgctcgaggactaccgcgaggacgtggtcgccgccatgcttcggcacctgcgccgcgtcgaggacgtgtcttccttcgtgacgtacgccgcgcgcaagaaccccgcctgcgttcccggcgtcgtgcgcgcggtcgtgagcaacttcaaccccgcggtggtcgcggccatgcgccccttcctgcgcgagcacatgacgcgcgtggacgcgctgctggacggaatgccgcacctctcggaggccgaccgtcggtacatccgccgcgtggtgctgcagggccgcgcctgattcgccgtcaataaatcgcgatggtggacagcggcacgcacgacgtggactccgccgcgcaggagcgcacgcccaaccagcagaccttcttcaccaaggggctcagtccgctgatgcgccacacctacatctacaacaactacgcctacggctggattcccgagaccgcgctctggagcagccgtctgggcgactaccgcgtcacggacttctacccgatctcgctgggcatgctcaagaagttcgagttcatgttctcgctgctggcggaccccggcggcgcctgccccgcgtacgagcccaagctcaacaccgagttcctgaaccgcggctccttctcgggacggtacgtgaaccccttccaccgcttcgcggcgctgcccgagcgcgagtacatatccttcctgctgctgagctcggtgcccatcttcaacatcctcttctggtttaagggcgagaccttcgacactgccaagcacagcctgctcggcgccgtgtacaccacgcccgagcggcacatcgagctcgcgcggtacctgcggcgcacgggcgactacaagccgctgttcagccgcctgggcaacgacgacacctactcgaagcccttctctgggttcacgcgcatcagcaaccccacgcccatcgggcggctgccgccctcggacttcgagacgctggccaacctgagcaccattctctactacacgcgctacgacccggtgctctgtttcctggtcttctacgtgccggggctctccgcgaccacgaagatcacgcccggcgtggagttcctcatggagaagctctcgctcgcgcccgagaacgtggtgctgctgtagcctcaaacataaaatataggcgcctttgatcgcactgcttcagttcagacagagctaagatggcttcctacatcagcggcgctagcgccagcgcgaacaccgcccagggcggcgattctcagtacccacagtactattatcacacacgcacctcccaaggcgacatccgcgacgaaagcgaaggttgcttccacaccacggacgacgagcacttggatctgtccgacgactacctcggcgatggcgcaccacactgcggacacagccacaaccacagtcgcagagatggagatcggcaccgccagcgcgcaccgcggctctatgaggacccggtgcccgcgaacatcatggtgcccacgctcagtctagagcagctgctggaggaaacctcggtcgcggggggccttctcggcggcaggacagagagggacgtggaacagctcctggaggagttctccgcactctgtcccggggaccagatcaccgcgctgcgctgcatggcggcctccttttaccgcgacgcgctgttcgcgccgtacgcctgcatgcacctcatcgccagtcggatgcgcgtgcactacgcgcgcgaggtcgtgcacgtggccgaggacctcgcggacgcgatgtctgcgaacagcggcgtctgcttccggcggtaccgaaagcgcgtgctagaggacatgctcgcggaggagatgggcgtgtacaattacctcgcgcgcgccaacgcggacatctgcgaggacaacctgctatcggccgtggagacgctgctgcggcgcttccgtcggatgggctgctaccgctctctgtgcatgctcaagatcctcgcgctgcagcacgaggacctggccggcttcatccgccgcagcataagaaaaacctgcaacttggcacacgcgcgcacgcacacggtttacgtgtagttaccctgtaaagacgggcttgctcccgaacaagcgctcgaagaagagcgtgcacatagccttattgtccagcaagttgactatctctgtacacagcctcttgaagtacacctcgtacatgatccgctcgtttttatccagtctgaaggtcttgtcgaccacgcgctcgtaggacttcacgttcgcgatccggcgccgccaggggccctcctcgcacacgtacgcgaagaagtagcgctcgccgatctcgatggcctccgcgttcgccgcgttgtaccgcgtcactagcgccacgttggggttgtcgggggacttgaagttcttgtggtgcgttcggctcagcaggaaccagtccagcggcatgctgcgcgcctcgaactcgaaggtgagctcgtcctccagcgagcgcaggatctccacgcccacgttcccggagccctcctccgccagcgcgcggcagagcatgtccttgtacttgcggatcatgagcttgtggaagggcgccacgtcgcggcgcgtctcgctggtgcccttgctcacgcgctcgctgccgccgccgtcgctcaccgcaaacttgatcgtggtgtacttcttcttggactgcatgatcaggttgcagtacaccgcttcgaactccaccttgaagttcgcgaagagcacgtgctcgttgatcacgcgctccagacagcgccccacgcgccgcgagaacgcgatgtcggaggcgcccacctccaggaacacggagtcggtgtcgccgtacacgctgcggaagcccacgcgctccgtgcgctcgccggccaccgccgcgtcgatctctagctccgcggcgcggcccgcgaaggcctcgtcgcgcagcagcgggttgtccggcgccgccgccagcgacagccgcgtgccgcacaccgacgcgccgtctagcgtgcgctccaggtacgcgatcatggtgcgcccgatggccgtgcagctcttggccgaggcgtacgagaagagcgcgctgttgcggaagcccatgagcccgtacacggagttggccgtgatcttgtacgtgtactgcatcgagttgtagatctcgcggtccaccgcggtctccgcggccttcatcagcttcttgtacttggcgcgcgcgtccaggaaggagcgcagcagcatcgggatgatgcccttggcctcgcggtcgaagatggccacctcggcgacgagctccggcgagcgcggctcgcagggcaccgcgatgtaccgcggcgccgggaacatccgccggacgtccacggccgcgacctccgcgtcgagccggttgtccgagacgaccacgccgacaagcgtctccggcgacaggttcgcgtagatgcacacgttcgggtacaggctgttgtagtcgaagatgagcacgtgcttgttgtgcatcttctgcttgggcgccatcacgcggccgccctcgtagaagaacttggacttcgtgtccgcgcgcaccatcaccgtgcggttctccagcagcagcttcatcagcgggcccttgatgcaggtgctcgcgcggtactcgaagaccacgctctgcggcagcaggtacgtggacgcggcggccgcgatcttggtctccacgccgtagtgcgaccagaggtagaggcagaggcaggcgtcgtgcaggcagtaccgcgccatgtccagacacacgtccagcgagtagttcgcgtacatgtccgcgaggctgacgtcgtccttgccgaaggccagcgtgacgcggtcgccgggcgcgcgcgccgcggggtccgcgaggtccacggtgaagccgtcctcgtcgacgcgtttgtgcagcacgcggcacacgcgctcgtccacggtcacgtagttgccggtgctgagcacgcgagcgaacacggccgcgttcccgtcggcgtcggtgctgcggtcgccgcgaaagcggaccgcgtccggccgcgcgtcctccacgaccgcggtgcagtggaaggcgttcttggatatggcgtccagcttgtaggagtccagcttctcggtgcgctggatgaaggcgtacaggtcgaagtagatggtcccgttgttgttgttgatgtggaaggtggtgctcgagacgccgccgacgcccttgtggctggacttcgtgcgctcgtacacgcagaagttgactgtctcggtcccgtccggcagccggaagcggatgtgctcgcccgtgagcagcgacagccgcgagtccaggtaccgcaggtcgaagttgtggccgttgaaggtgaccacgaagtccagcggcatctcgagcaggcgcttggccacgcgcagcagcgtcacctcgggacacagcgtgacctccgcgtcgaacttcacgtccgccgggtccaggcagaccgggatctcgcgccgtgccgcctcctcgaggtccgcgtcggagagcatatcggagttcgtaagcgtgaatcgccgctccgcgccgtccttgtccaccacgcagaagctgatgtgcgagacggcgttcttgaagacggaaggaaactttttctcgaagtggcactctatgtcgaggaagagccccgagcgcgtcacgttgaagcgcgggatcttctccgcgaagcacgcgccggggtcgtcgcagtggaagcagttgctccccaggtcgcgcagcagcgcggggtccacgcggtagcacccgtcagggtcgatgtcgtgcgccacgaagaaccaggacacgttcagaaagtcggacatgaagacctctggcggcgcgagcttgcgctcgctggccaccagacacagctctatctccgagcgctggcgctccggaatcttcgccgagcgcgctacgatctcgtcgatgctgaccacggacatgggcccgagcgcgcgcgtccacgccagcggctgggcgatgtcggccaccgcgtccgcgcgcaccacgtagtaaaagtgctgcacgaagcgcaggtacacgacggcgttgtcggcgcggcgcgccttgaggaagaggaaccggctgtcattgccgcggttctcgaaccagttcaaacatttcagctccatttcaaagagcataataacatttcatttaaatggagcctcgcttctggggccgcgccatgtgggcggtgatcttcatcgtgctgcggcgcttcgaggagcaccgcgacctcgagcgctgcaagcggcagctgtacgtgatctgctccacgctgccctgcatcgcgtgccgacgacacgccaccgccgccatcgagaaaaacaacgtcctctccagcgaggaccccaactacgtgctcttcttcttcatcaagctcttcaacaacctcgccttcgacgacagatacaagatcgaccccgcgaaggtgcgcccgctcgtctagagcatgccctcgtacgcgcgcgagttgtccgagtacacggtcaccgcgatgccctcgcgcggcgtcacgtgcacgtgcatggagtccgtgatcacgaagcccacgcccgtgaccggctcctcgcccgcgtcgtccgtgaagagcagcccgtggttgaagaggtagaactcgttctctgcgagcgaaagccgcccgcggtcgcagaggtagtagaagatgtcgtagtcgcgcacggccagcgtgaacgtggactcgctcaccacgatgtacttgtccagctcctccagcacggacgcggccgcgcggcgcgcggtggcgcggcactgcgtcgggcactcgcacgtgtctgcagagtacaggatgcgcgttcgccccgaggcggtctcgagaaaaacgttaatcgcctccatcgcccagaagcgactcgaggatcgcgagcaccgtgcgcagcacgagcccgatggtgcagaagggaacctctcccgactccgagcactcgcggatctccgtctccacgcggtcgtgcacttttatggagggagccgtcgttccagtggcctccatcgcgacggacaccaccttggccacgaactcgcggatcttgctcatgcgccggagcacggtcacgcggaagaagacggccgcgagcaggtactcggccacgcagctcacggcgatgagcgcctgcgcgtgcctggtgttgagcacgtgcgcgtcgggcacgaagtccgagatcttcaggtgcgagagccgcacgagcgcgttggtgtccttgacgccgtcgcaggaaatgttcgcgaagatgagcttctcgtagtcgagcgcctcgaccacgcgctgcgcgtccatgtgccgctcgcccgagagcgcgcggctcaaagggccccagcacaccgagcccgcgaagcaggggtccaccacgccgtgcatcgccagcagcgtcacgtcggccaccgccgcgaggatggccgcgtcgtcgagctcgttcgcgggcgtcgcgcccgtcagggacgcgctgcgcagcgcggacccgcccggcgccgcgtgccgcgcgcagaactcgcacccgcagcccggcggcagcggcggcttcgagagcagacttatgagcccgcccacgtgcccgtgctcggtgatcagaagcgccaggatgctgtcggtgctgccctctatggcggctgtggccgcgctagaaggctctcccgtgacctgccatccgcagacaaccttgagcatcttgcgcttgagctcgttgggcgcctccgaggccagcatcgtgcgcgggaacgtcgcgttgaggcggaagtcctgcagcagcttttcgagcgtcgcggtcttggcgtgccgccctttgcgccactcctcccccaggtgccacatgagctcctcggccgtgtccagcgtcggcgagacgcgggccttgggcacgcgcgccgcgctgcgcatcagcggctccgaggcgcggaagctgccgcgcgcgtgcaggcgcatggacgcagacgaggaccgcatcgagggtctctggtggaagctcgtcatcgtgaagcgccgcgtgagcggacctacctcgtcgcgcgactcgtcgaagtccgggtcagggtccgtcgtgtcggtctcggcgctgtgcgtgctgggcgcgctgctgcgggcgctgcgcgtgctgcgcgtgctgcgggtgctctgcgtgctgaagctgcgcgagcaggagtccgccgtgtccgcttcctcgtagtggaagtccacgtgctcctccggcagccggcgcgagcgcgacttggagatgccgaccatcttgtccgcgccgaccgggcgcacgcagagcgccatcgcgccctcgcgcaccgcctgcgcgaactcgcggctggcaaccatgctggggttgaggaacttgatgatgttgaagtatggccactcgcagacgagccgcgcgcaggtcgcgacgtcgtcgacgcttctgagggccctgttcagcggggggatgttgctgccgtcggccagtgcgacgaggtcctcgggggagatctcgagcttgggaatcatctcgttcacgagcgcggggtcgatggcgtggcagacggtctctacctcggtgcgcgagagcttgagctgcgcgcaggccgcccacggcgcgcacgtgagcgcgaacaccgcggagactcggcccttgccgaccatcgccacaacctcgggctcgtagaccaggccggccttgagcagagcctcgagcacctctatgtcgtcggcggcgagcagcgcgcgcctgtggaagcacaccgcgggcatcatcttcttgcagatgccgcgggtgctcttgagggccgtgataaggtcggggagcctgacgtgctgcggctggaagaacatgacgttggcggggctcgcggccaccgcctcgtcgtacaccgacatcggcaggtcgccgtggagcccgcacggcagcaggctcagcagctgcgcgcgcgagagcttctcggcgcagaagttcttcttggccagggcggccgccgcgtcgcgggccttcttggggtataacaacatggcgggctttaaacacgaaacaaaaatccgggttgtaacatttcaattttgcatgttctgggcctcctcgcagagtttctccaggccgccggccacgatggcgtcgacgaagaggtcggcctcggtgaagcggtggttgccgcgcaccgccaccagcgcgttctcggtgaccaccaccgacagctgctgcgcagccgtgcgcaggtcgaagtgtcggcacgcgagcccgtggcccagagtctggtccagcgccgcgagcacctcctccaggctctcctcgcggtggttgtagaaccacatcagcacgaagtaggccacgtaggtgtagaggtagtgcgcgaccgcgcgggcgcgcaccagcggctggttgcagcgcgcgaaggccattccgctggcgatgaggtcgtcgtccagcgtggcgtagtcgccccagttgagcgcgctgaactgcacgctgtagaccgcgcgcacgaacccggactcgaggatgtcgatctgcgacggcgcgccccaggtcaccagccggtacacgatgccgcgctgcatcagccgcatgaggtcgccgccgacctcgcgcaggcggtgcgtcagcgaggcgaaggccagcttccgctgcgtgtactgcaggcccacggccacgcacccgtccgactccaccagcacgagcttgtggtccgtgcagccgtcgctgcgcaggccgccctcgcgcgccatcatgtcggtgacgaacatgtacttgcgcgcgcgcggcccgaccaggatgcgcttcttgccttccatgcgcagcaccacgtcctctagcagccgcgtgctgtccacgcgctccacctgcgggtggatggtgggctggtagttgtacagcagccgcggggaccagttgtaggcgaacgcgaagaggtgcgtgtccaggagagagatggggaagacgccgccttcggtggcgcgccggaggatggcgagcttggtctccgcgggcagcaggctcccggtcaccgcgccgaagaacatggggtggttgcggaacttgagcgcgtacgcgctcagcacctcctctcgcgagaatatcgagtccgcggggttggagctcgcgggcaggagcacgtcctccacgctcagcacctcgtcgatgaagggcagcaccatgtccacgttggaggcggtgaaccactccatgtccacggcgttgcgctccacgatcacccggatcgtctcctcggatatgttctcggcgaaggcgctctgcagctcgatcaggttctcggtggcgtcgatgctgagcccgaggcgcatgccctgttgcagcacgtcgttgatggggttcacgtacatggccaccgccatgcacccggccggctgcacgcgccagaggttggagtacgcggacacgtccgtgatgcgcagcgtctccagcacgttgtacatcgccgcgcgcatttccagcgtgtccacgtacacctccgcgtgctcgaagatgatgtcgagctcgaacgcggacagcggcagactcacgtagatctggcaggcgtcaaagagttcctgcgcgtactcagagaagcacgcgtacgcgatcacgcccgcgcggttgacgatgtagtccgccttgaacatcttcgtgaggtaggcgtacagcgaccggcaggccacgtgcggccttagctcgtcgagcacggcgtccagcacctcgcggtgctgcagcacggaggggtgcaggaactgcgtgaagtccaccgcggtctcatccatgaagtcggggatggtctcgaccaccaagcggtggttccgcaccgcgcggaagccggtgttcatcggcgcgatgctgtgctggtcgtggacctccagcaggatctcgtagccgatctctcggcagctcagcgccgcgcgcgcctccgtcacgctcgcgttccggaagaactgccgcaggtgctcgtccgtgcggcagagcgtgacggattgggggatgcgggaaaggtcgcaaagagggctgtggacggagatgcgccgcagcgcgtggtctacgaactcaaaaccgcgcctcgacatcgtgaagtcgcggagggcgtacatgttgtaggaacagaggcggaagaggcagtctatgtctagcatgttggaaacgcagtacgcgtgtctgtggaggtgcgggagcatggcgggcacgacctcggtcgtgttctggagcatggtgcatacgaggtcgggcgctgtgaggtcgggggtgacgatgaggatgcaggagctggagaacttgctgagctcggaggccagccggtgaaggttgtagtcgtggcgctggctgaagttgctgtttatcgtgcccgtgaagcccatgagcgccgccagcgtcaggtcctcgcgctcgatggcccagatgtctaggccggaggctatcatgcagcgcaccagcgcggcggcgcggtcgctcgtggggtagctcatggtgctgtcggtcgtgcgaatcagcatgggataatgcttcatttttacggtcgggggttgcggactgtggggcgcacagggcctgcgggcggctcgtgccggtccgcggcgttcgccgaacgcaggaacgggcccatgcgcgcccaggccatccacagccccgccgtcagcgccagcagccagacgaataccacgatcatcttttatgtagctggaactcgcgctcactccccgccgcacggcgacggggagagcccagagccgagctccatgcgcgtgctctgcacggtgagcgactccacgagcttggacacgttcatgcgcgtgttgtcgggcaccaggtgcgcgaggcgcgcgtacacgtcatcgtgcatgcgcttgctgcagcggtccaggctcgcggagagcgcggagactagcgcggtgtgcttcgcgtacacgaagtcgccgagacacacggtctcgagcccgagcacgtcggcgctctccgcgtccttgagcgccacgaatatcttctgctcctcgcgcgtcatcgagcgcatgaggtagtcgtgcagccgcgagcgcgagatgagcccctgagagatctgcgggctgcgcatgaagcgccggcgcatcgcgcacagcagctcctcgtcgacgacgtacatgctgtccttgatggagctcttctcgtcgatcacgagcagaccgtcgttggcgaccacgttgatgaaatcgtccacgtgccgcgcgtctatgtcgtagcgcgtgccgccgcactcgatgtgcgagggcggcgacttgagccggttcgcgagcgccttcacgtcggagacgtcgatgtacagcgaggactcgcgcgggacgcagccgaggatgcgcgtctcgagcggcgtgaggatgagcacgcgctcggcgccgtcgacgagcttgccgtcgtcgggggaaaagaagttgttctccacgatgctcgagacgaggctggcgagcacgccgtcgcggtaggcgccgagcgcaaactcctgcacaaagggcgcgtgcagcaagtccacgggaatgcgcatctccacgcggcgcgcgaccgacttcttcttctgcaggtgccgccggtccaccatctcgtccacgatgtccgatatgcgggagcagaggtacgccttgaggacgttggcgtttaccttgttgaagatgagccgttcgtcctccatttaagctgctcagacgagctttaaatagtggaaacacagcagcacgccgatcgccgccgctatcaggccgattagaaaaacggtggtccaggggacgcccttgggcctatcgcacgccggcttttcggtcattacggtgcgcacgatgtttaggaactcctcgaagtcctcgtccgagttggagaggaaggagccgaagacgccggtgtacagtttgtccatttactactagatattaaacggcgcttccaactcctcgtcctcgaagcccgcgccaggctcgacgacgcccaggccgcgcacgtcctgctcctcgttgaacgtggtctgagtctcgctcatgcgcacacacgtctgctcgccctcgagaccgagcacggtcagcgagcactcgcgcggcatggtgatcttcttaaccgcgaaggtgactttgccctcgccgcccgagcggtagaacaccaccggcgccaggatgagcgtcgccatctgcgcgtcgcgggttgcgaggttttctatctctcgcgtcagcggacatatctgcggctgggtgtcgtcgtcgctggtgaactccaggagagcgccggtcaggcggttgagatacagacatccggacttaaaggtgttgtcgatggccgtttcggtgttgaagttgcgcagcgacggcggaacgcgagcgccggttttgatgttgtcgtatatgttctccagcagctggtagagcagcggactggcgctcacgggcttgaggcgaccgaagtacccgctgtcgttctgccgctgcatgtcggtcttcttgctcgggtagatcttaaactcgcccttcacgacgatgagcggcgagacgagcttggatgctagactttcgacgagacacacgttgatgttggagcagctcgggtactgcgacggagttagagtcacggcctcgatgaccttggtttggctcgacgagagcgacttagcgaagttgatcgcgtcggtgcacgacatcgcctggttctcgccgaaccgcctggcggacgctgcatcctcctgctgaggagcgcggttagacgcgacggtggttttggatacagcgcgtttcattattgcggcgattttaaagtacgtgtatactttcagttttgtcgccgagcgttcagcgcctgcatgcagaggaagtacaggatgatggtgcacgggatcgtggtcagcagcgatacgaagtccatcactgtgaggacgcgcagcgccccgcgcgagcggatgcccagcgagggcgcgccgcggcgcgcgatggtggccccgttcgtcaccactaccagcagcattaggatggtcgcgcccacggcgacgcccaggtcccgcgactccatttatagtacagtatagagcgaccgcgtcacgaactctcggctggccaacacgcgtccgtcgggcgggtgtccgccggccttcccgcggaactccgggacctcgaagctggacttcgtcacgcggtacgtgtacttgccgcgccagaccaggttttccttctggaagacgccgtccatggtcacgcccgccatgaaggcgtccttgacgatgaccagcaccgcgtctagcttgcgcccgttgatgtgcgtgacgaagtccgtgccgctgcggctcgcgcagcggatgtccacgcccgagggcaggtccaccacgaacacgaagcgcttcggcgcgtagagcaccaggtccgaggacggcgacgccgaaggcgccgaggggaactgccggtggtcaaaagggtgcaccacgcccacgatggacgtgacgcggtcgtccgggaactgcgtcgcggcgccgccgccgcggtgccgcgtgaccgtgcttctgcccacgtcgtcgcagaccacgtgcagctccgacacgatcggcagcagcgtggccagcatgcggtcggtctctgtgcgcgtcgcgcagcggtacgcgatcccgcagtgcgcgtcctgcgtgcgcccgaagaagagcaccagcacgctcgcgtcctggtcgaagggacacacggccatcacgcccaccggcggcggcccgtggcctgcgtacgcggaggagaactcctgcacctcgaccacggcgtcctcgcgcgcctcgccgggcaccatcgccgccgccggccgcagcgcccgcacggtctgcttaaccgcacgcgcggcggaggccgcgctcggcgcgactacgcgcacggccgcgtgcgcgcccggcggcggcgcggttccggccatccagcccaccggcgagaagaacacgtcgcagacgtgcacgcccgcggcctgcagcgcgcgcgcgagcgcgcgcacggcctcccactcctcgcgaaaggcgctcgcgaccgcgagcgccttcagcaccgtgtccacggagttgacgggcttctggaagaggttctcgttgttgtagatgaactcggggagctccacgggcactgtgaacagcctaatctcgtgcgcgccgctgggcgtgagccgcgtcgcgggcttgcgcacgccggcgccgatctgcttgaagaagtggttcatggcgccgccggcttctcgggctccggcgggagcagactatttattcgggaggttatcctttccgaaagcacctgcacggacttccgcgtccagcgctccatcttcatgtactccttcatgccgtcgctgagcacctcgacggcctccagcttgggcgctgtcgggtcgaagaggatgctcttgagcagcgtcatcttcttgtccgcgaggaagcggaagtaagtgtagatgcagcgcagcgcgcggaagttctccgggtgcttgatggtgcacaggatcatgaagatgcaggtgaacatgccgcactcggactccatgagctggttgacctcgaggttgatgcagccgcgccgcgccttgaagttgtccacgaagaagcgcatgagcacgtccacgtcgcagttgcggttgtccaggtccgcggtctcggcgttcacgttgaagccgtccgagaaggagtagaagtagaagtacttgcaggggtggaactccgaggggctgttgccgccggagtcgtagaaggacacgagccgcgagacggtgtcgaagatgcagcacttccagtggaacatgtagcagaagccgaacatcacgtagcgccgcccggcgcgctcgatcttgtccttgagcgtgaggctgaccatgttgcagcggaagcggtccgccttttcgtggatggccgcgccgttgaggaagttcaggttgaactggcccaggtacgcgacctcggtgccgaacgcgaagggcgccaccagactctggatgctcacgttgctcatccaggcgctgcggtcgggctttatggcgatgggcactaccttggtgttcacgcccgtgctgacgcccgcgcgcgcgaggtcgtccacgttcagcggcatctgcgagaagtccacggcctcggacaccttctcgcgcaacgagggcttgaagaagaagcccagcgggaccttccactccagcgcgatcgcctcgcggaagccgtagcgacccttgaggctggccagcaacgcggtcttctgcgcgacctcgtccttctcggtgtccggcggcgcggcgtcgatgagcccgcgcttcgcgaagtccagcagcgccgccagcgggatgcacgagacgcgaccggccgtcgcggattcgtcgaagcgccgcaccacgtacccgttgcagttggtcttgaagttggacacgtccaggtgcgcgctgagccccaccaccgagtagatgtggcacagaaggttggtgaaccccagctccgggattttgctcaccactaaatccgtgtacttgtccatttatcatggagaatcatctgccggacatgctgatgtttcccaactgcgtttctgtgtttccctttgagtactcgctggaggacgtgttccgcctccccgaggagcgacggcgcgcgttcgccatggccgtgttcccgctctccaagcaccgctggaggggcgcgcggctccagcgcgacgagcgaagcgtgtggctcagcgtcgaggaggaccgcgggcgcgcgctggacgagcggaactgctcctggctctcggacgtggccgcgcgcatggtcgacgacgagggccgcgcggtcacgcccgaggcgtacgccttcatgcgcgccgcgcccggcgcgcgcgtcgccgagctcgccgcggacgcgggcgtgctagcgggccttgtcgccggcggcaacgcgctgcgcgtcttctcctcggagtccacgcaggcgcgcgagggctggaaggcgcgcagcgtgggcgtgctcggcaacgcggcgccgctggcgcccgtgccgctggcatcgctgcgtccggaagtgcagcgcgagctcttcgccgcctggatcggccgccgccccgtggtgctcacgggcggcacgggcgtggggaagacctcgcaggttcccaagctgctgatgtggttcaactacctcttcggcggcttcgagcgcctggacgccgtccgcgagttcgcggagcgcccgctcgtgctctcgctgccgcgcgtcacgctggtgcgcgcgcacaccgcgacctacctcgcctcgctgggcttcggctcggccgacggctccccggtctcgccgcggtacggcgccatcccggacgccgagcggaacacggccccgcgcgcctacgggctcgtggtggccactcaccggctcacactgactgccatccgccgctacgacacggtcgtagtggacgagatccacgagcacgaccagatgggcgacatcgtggtcgcggtcgcgcggaaactgggctcgaacatgcgatcgctggtgcttatgacggccacgctcgaggacgaccgcgcgcgcctggaggagttcctggaccggcccgcctttgtgcacatagagggcgacacgctcttccccatccgcgaggtctacgtgaagaacacgcaacagccgccgctctcgcgcaagtacgcggaggcggagctgcagaacgtggcgcaggcgctgggcaccttcgtccccgagcagggaaagtgcggcatcctcttcgtagccacggtggcgcagtgcgcgctcttcgcggagaccatcgaggccaagcaccccgggctgctggtgcgcgtggtgcacggaaaggtgccctccgtggccgcggtgctcgaggaggtatacgccgcggaccggcccgcggtgctggtttccacgccgtacctggagtccagcgtgaccgtgcgcaccgccacgcacgtctacgacactgggcgcgtgtacgtgcccgagcccttcggcggccgcgagaccttcgtctccaagtccatgtacacgcagcgcaagggccgcgtgggccgcgtggcgcccggcacctacgtgcgcttcttcgacacgcggctcgcgctgccgctgaagcgcatcgactccgagttcctgcacccgtacgtgctttacgcgcgcatcttcgggctaacgctgcccgacgacctgctcgtgcagcccagcgacctcgcgctgctgcgccgcaccgaggagtacgtcgacggcttcggcatcagcctctcgcgctggacgcagctgctggaccggcactacatgcacatggtcgagtacgcgaaggtgtacgtgcgcggcgggcgcctcgccgccgcgctggacgccttcgagcgcaccggcgtgatgacgcacgaggccaccgaggccatccgcgccgtggacatgctcgcggccgtcctaaacgtgcgcaagtccaaggaccgctaccgcgcggagtgcaaggtgctcttcgggcccttcgcgggcaaaaagttcgtggtcgccgggcggcgtccgcccggctcgcacgtgctcatggtcacagaccgcgtcttcatcgaggccgagcccccattctgaggaccaccttcttggagacgcccgagaagtcgtcggcgacgccgcggcgcgccaccacaaggcagtacgaggtcacgtgcgggcagcgcgcgatgcagcggaaggcttcctcctgcgacagcgagaaggcgaacacgtagaaggtgtgcggggacttcagcggcgtgtggtccatcgagtagatgacaccgagcttcttcatgcgccacataagcgcgttgatgtggtcggcgcgcagcgcgcggcccttgagcacgccgcagacgaagctcgagcaggccacgacgtcgtagcgcgtgttcctgccgaagaccaggtgcggcgcgccggcggcgcgccgcgcggccgcgcgattctccacgatgtcctctatggagcgctcgctcgcaaagaagtccaggaacatgtactggtaggccacggccgggcgcgacttgctgaacttcatgaaggcgtccgagtccatgatggcgtccatgtcctcagcggcgagccggtgctgcagccggatgccctcgaaggtgtggaagagccgcgcgtccgcgtgcatggacagcgcgagagtgacgaagttgagaaggtccgcgtcgccaaagcgcacgagcacgttaccgggcgtgcgcgtcttgcgcatgagccgcgcgggcgcgccgtcgttgtggctgcggcggcgcattttgtcgccgggggactcgggcggcaggtcgatcatgaccagccggtgccgctgcgcgtcctcggcgttgaagatcgaggacgtgaagcccgggtacagcaccacgcagtcgcgctccgagatggcgtgcagcacgtcgcgcttgagcccggccaccagccgctccgcgttctcgacgaagtagttctcgtagtccaggatgtcgtgcgccatccaggggaagttcaggtacgcgttcatggcgtagtcctcggcgtcgaagcagatgcgcgtgtctggcgtcgccgcgatcggaaggtccttgatgccgcggagcagcccgtcgtagtcggactcgtccacgaaggagagcaccacaaagaggtcctcgcccacggtttcgtagtcgaagaggtggtaaagctctcttagcgccagcacggcgagcgcgttgtccagcgaggcgtgcacgcgcgccaggatgctgtagaagggcgtggccatcatcacggccttgccgccctcgcaggcaacggcgcgcgggaaaatgacctccggcgtgcgcggcagccgcccgaacgtcgcgttcagcagcgcgaccgtggccgcgtcgctctggcgcaggaacactaccaccgaggggcccgagatgctgagcatgcgctcgcgcatgcgcgctggcaggtccggcgtggtcacgaggtccgcgaagcggccgccgttgtagaggtcgccgccgccgaggaaggtgagcacgtcgaagcagtgcagcacctcgttgcggaagtagtactcgttctcgagctccttggcgtacgcgcgtatgtccacgttctcgaagtttgttcgcagaccgccgccgtcgaagaaccaggacgcgagctcgcggacggcgtccgcgggcctgttgcggcggctcttgcaccagaagctcatgtagttgcgcgaggtggaggcgttcgccaggaagaagcggtggtcgaaggagatgagcacatgctcgagcaggtgcgcgagccccaggaccgcgcccacgtcgcgcccaaaaccgaagtttgatatccccaggtagacgtcccgtttcatagacggcctcaggaacaccctgacgccgttttccaacactatcattctccggtatttacttacccaaaagtagtatggggagaagtgtttgaacgtcccctcgcctttttaaatcaaaagtagacttctcgcgcccgtgcgccaccgtcacgcgcgcgcggcgcgagtccataccggcgatcaccgcgctgctctgcggtgcgtccggccgcgggaagagcacggtctcggagatcccgtccagctgcgcgtcggtgcgctgccgccacgcgtgcgcgtccgcgagctcgcgcacggccagctgcatcttgttcgtcggcaggaacgtgaacacgtacgccgccgccaggaaggctgcgaagagcacgaactcaaccgcccatgacatttagggagctgattttgttccacgcggcgacgcacgtcgtgacgggcgaccccgaggcgccgcggcgcgcggcctcgctgtgccgcggcttcggcgtggacttccgcgcgattcacgcggagttcgcgcggcggtacccgcgcaccgcggccgccgtggagcgcgcgcagccgctgcccgaagtcgatgccgcctttccgccggacgcgcgccggcaagtcgtgcggctgcgcctcgaggctgcggcgctggtcgtcaaggagtcgcgtgcgctatcggcctccatgcgcggcgtggcggtggtcgacggctgctgcgtgcgcgtgtgccgcgccaacgacgagctgctagagttcctcgcgcggcgctacgaccccgcggtctaccgctacgcggaggtgccctcgccgagcgtgcgcccgggctcgaaagtcttcgcgtgcgcgggccgcagcgtcacctttgcggccgcgcaccggagccgcatcacggccaaccgcccgctgcgcgtggtcgtgaccgaggcctgtgtggacggcgtgctcgcgcgcggcgccgcggaggtcttcgaccgcggctccggcgtgctgccccgcgcgctgcgcgagatcttctaccgcctcgacgaggacggctgtcccacgggccagacgccaggcttcgcggacagtatggcgtcgcgcagctgatctatgtccacctttttctcgtcgatctgcgccacgaccacgaaactgcgaatgtccacagcggccatggtcttggccaccgggtcgtacttgaggagcagcacgtactcgttgccgaagtgctcggtgacctcggtgatgagccggtacacgcccatgccgagcacgttcaccgcaccgtccttggcgaagagcgagaggatgttcacgcacttcagctccatctcgccctcgaggcgcgcgagcatgcgccgggtgacctcgcatactgaacaaagaggcttacctagtaagataagcgttagcttagccgcggtcggtgacgcgtcggaggccatttatggggatcaaaaacttaaaggcgttgctgctcagccacggcgcgctgaccccgcacgagccgggcggcgacgagcgcttccctgccgtgttcgtggacggcttcagcgtcatgatgaccatggcgtactcgtgcgcggacgaagacgagttccgcgcggccgtcgaggagcgcgtgcagcactggatgagcgtgtccgagagcgggcggatcgtggtcttcctcgaccgcggcgagattccgatcaagcagccgctgcgcgaccagcgccgcaaagccacgcgcgaccgcgccgcgcgccaccgcgagttcatcgccgccgcggaggcagacgcggcggcagaggccgttggcgcccgcgaggacaaacaggaggacgagcacgcggagttcgccgaggagatccgcgctgagaagcagctaaagctgcagcgcatccgcttccagctcagcatcgccaaccacgaggtcgttaagtcgctgatagagtccacgctcgcgcgcgctggcgatgccgtggagatcgtcttctgcgacggcgtcgacgcggagatggtcatgtgcgcgcgcggacgcgccgaggccgagcgtcgcgggcgctggccgctgctcgtgaccacggaccaggacgcgcttttgttcacgtccaccgatcgcgacgagaagatagtgagcaccgtctccgcctgctacgcgttcaggcccaccgagacgaccgagtacctgtgcaaacttgcggcgctggccaacggctgcgacttcttccccgggctcggcggcatatgcgtgagtgtggagtcgctgcgccgcgccacgcttttcccggaattctccgtgcgcaacgccgccgtgagtctgtgcacgcggcccatgcggctgtccacgcaggacgcgctggagccagaggccgccgccgaggtcgtggaattcatcaggcggtacgccgccggcgacgagcgcatctaccgcgaggtgccgcccggcgcgtgctgcggacgcgcgtttgtgcgcggagcgctcgcggccgagtgggccgacgcgctgccggcggccacgggtctgagcgtggtcgcggacatgatcgcgtgtctgcccgcgcggcgggaccccgcgcccgaggaggtagagcggctgctggcgctggaggcgcgcgcgcgaggcgcgcgcgtcacggatgcgatgctcgcgcagactgcgcagctgctgggttacggcgcgagtgcgggcgccgacggcgcctccgccttcgcggtctcgggcgccaagggcctgatgtgtcgcctgcgcggcacggccatgttcttcaacgcggagtacgtggaaattgaaagcgaacccagactgttaaagctgcggtagcatggtgttcccgatcgtgtgctcaacgtgcggccgcgacctgtcgcacgagcggtttctgctcatcgtgcgacagcggccgctaaaggttgttttgcggacggtgcgcaacgtctgctgccgtataaagttgtctacacaaatagagccgcaccggaacctgacggtgctgcccatgctcgacataagctgatttttcttttccgctcgtatgcgcgagttcggactcgcggcgcgcatggcccgcgccatcgaggacgtgtgtccgcgcggcgcggtgatattcgtatccagcgccgcgtccatgaccgactgcctgaacccgtcggtgttcaagcacgcggcgatatacgcggggcgcgtggaccgcgcgccgctgccgccgccctcgccggtcccggcggaggccgtgacggagccctgtgcgatagacgccatagcgccttacggcgcgcgcgtggtcctgctctcggagctgctgcggagctgcgtggccgttcaggcctaccgcctggcagtccccggcgccctcgcgctcatgaacctcgcggccgacgcggccttcgagctcgtgggcacgccctacggctttaacagcgaccgaacgtactgcttcaagctcgttgccgactgctttgctagcgtgggcgtgacaacgaagaccaggcgcatcatgggtcgcgacgtcgtgctcagccaggacttcctggagagcggcatgtggaccaaggtgctggactccgccgcggagccgccgtggctggtctagaacagcggcggcgcgcgggtcccgagcacgggccgcgccacctgcagccgctgctgcagcgcgcggcactgcgcctcggcgtcggccgtctcggcggggtcgacgggcgtcggagttgcggaggtggtcctgaacggctgcgtgttcaccgagacgcggatgcgctccttgcaggagcgctgctcgatgcagttggccagcatcttcatcacgtgcaggtactccagcaacacgaacttttcgagggtgatgccgtcgaagggcgacgaccccaccacgcccagcgggctggacaccgcgccgtcgagcacctcgccgcgggactccttgcgcgcgcgctcgagcaggtcctctgtccgagccaccacactgccgaagtcggcggccgcgggggcgggaacaggcgcagcagcgctgtccgcgtccgccggcatctcctcgatcttgagaccggccgcgaactccgaggccgcgtgcacgggcgaggcgccgcgccgcaccatgaagtcgcacagacgcgatagcgcggaggagcgcaccggcatgtcgagcaggcgctcggcctccatctcggcgaccgagtcggcgcacgcgtccggcgcgcccgcccgcacgagctcgtcgcagcaccccgcctccttcatgagcgcgggcatgagcttgtactgcgccatgttcaccagcccgtacttgagctcgagcaggtccgcgagctcggaggccatgggtcggtttttggtgtagatgacgcgctccacggcctccgccatgtccacggcctgcatgagctcgccgacgagcacgctggccacgagcgtggccagcgtgacgcgcacggtgggcacgcagaccgcgaagaaggaggtggagtgggtgaagcgcatgagcgcgccgtgcagacgcgcgaggtccgcgctgttgcccgcgtgcacgaagcgccggcgcagccgcgccagcgcctccacaaggtcctctcgcgtggtcacgcgcacgttcgcgatgcacaggtcgtggatcgcgttggcgatctgcgcgcggcgctgcggcgagctgccgggcagcagccgcgccttggcctcgacgtcgacggtgctcgagagacagccgcaggcggcgccgcggacgacgaacttcaacaacgactcgaacacgcgcgcgcccgcgcggggcgcttgcttggacgactccatttactttaaataatttacgagatcaaaataaaatgactctgcgcatcaaactcgagaagctcaagcagatcgtaacttacttctcggagttcagcgaggaggtctcggtgaacgtggacgtcggcgatggcctcatgtacatattcgcggcgctgggcgggtccgtgaacatctggaccatcgtgccgctcagcgcgagcgtggtatacgacggcgatgtcagccgcgtgttcaacctgcccgtgctcaaggtgaaggcctgtctgtgcagcttccaccccgactcggtggtgagcctggagcccgacctcgaggacaacgtggtgcggctctcgagccaccacgtggtcagcgtggactgcgacaacgagcccgtggcgcaccgcacgaacaccgccatctgcctgggcattaaccagcgcaagtcctacgtgttcaacttccggcgctacgaggagaagtgctgcggccgcaccatcgtcaacctggacctgctgctggggttcatcaagtgcatccaccagtaccagtacatcacggtctgcttccgcgacaagaagatggtgctgcacacgcccgggaaggtggacaacttcttccgcgagtactccatgaccgagtgggcgcccgacctcgagcgcttctcgttcaagatccccatctcctccgtgaacaaactccgcggcttcaagaagcgcgtggtcatgttcgagtcgcgcgtggtcatggacgccgacgacaacatcatcggcatgctcttcaccgaccgcgtgggcatgtaccgcgtgaacgtgttcatgtcctttcaggaccggtctctttcatgcgactaaatactcatgggcgggtcggtgagcctgccctcgcgggacctgccgccgccggtgcgcacgccggagatgaacatcgtgcccgagcgcgacctcgcggacacgatggcgcgcctctccaccgcagacccgccgcagccgctgggcgtcggcgacgacgcgcgcatggccgtgctgaagacgaccttccccgagttcgcgatatcgcggcccgcgacgggcatgctcgccgcgcagcgaatcaggtacgacggcgacccgcgcgtctgctgcggcgggttcgggatctcgcattactgggagaagggggcgcgccgatcgaacgtcgcgttcgagggcgcggcgctgcgcacctgcgaccccacgcgcttcgacgcgggcgcgtgcgacgcgctgctcttccgcgagtgcgccgccggcggcgtcgacgcggacttctgcgcgcactggatcaacgcggccgtgacgcggcgcacggaccgacagtcgcgcgcgcggctgaacgacatgttcgtgcgcgattgccaaaacgacgccgcccggcctcactgcgtggcctggatccgcgcgatgcgaagcgcgcgcgcgacggcggacgacggtctaatagacgccgtgctctcggtgcagagtcccgagttcaagggcaagcacatgcgctgcagctacccctcgccggccactctcgccatggccgcgaacgtggacgagccgcgcgagtgctgggaccccgagtgcgtggccgggaacgtggacttcatgctaagcgataactacacgaacctgggcttgtgtcggctctcgcgctgctccatcggcgtcacacacctgcggattgacgcgcgttcgcggctgcgcatgcggtgcgccggcgcgcttgccgggctcacgaaggcgcccgtgaaccagactgtcgtcgtcggcgacaacctcgcgcgcgccttcgagccgcgcgtggaaacgctcagcgtgttggcgctgtgcgtggtgtatctgctaattgtctggctctaaatgggggccgccgccagcattcagaccaccgtgaccaccgtcagcgagcgcatccgcaacgagctcgagcagagcgcgagcgctagcgcgaccgccgactgcgacgtcaccatcgggagtctgattatccgcaagaacctaggatgcagcgtttccgtccggaacatgtgctcggccaacgccggcgcgcagctggacgccgtcatgaaggccgtgagcagcaccttcaacgacctctcgtcggaccagaaggcctacgtgcccgggctgctcacggccgcgctcaacatccagaccacggtgaacaccgccgtcaaggacttcgagacgtacatgaagcagacctgcacggcggacgcggtcgttcacaacaaaatcaagatccaaaacatcgtcatggaagagtgcgcctctctgccagggagtccggccacgcacctggatttcgtgaacaccggcacggccgtgggcaactgcggcgtgaaggccgtgatggacgtgctcgcgaaggccagcaccaccgtgcgcaacgaccaggaggccggcaagggctaccagaccatcatcatcgcgatcgtggtcgccatcctggcggccatcttcgcctggtacgcgcggcacatgctattcatgtccacctccgacaaaatcaagctcgagctcgccaagaagcccgtggtgcactggaccacctacctggacaccttctttacggaatttccgccgtccgtctagatacgcgcaacattgaaacattatatccacctctcaaacggcggtatggtccgacgcgtcctcctcgagcgcgtggacggcatcgtcgagcactcgcgcgcagaccgacgctacttggaggccattcagcgacacctcgaggggtctacgcccgggctgcggcagatgtggcgcttcctctacgacctgctgctgacggtgttcgtcgtcatgtacatcgtcttccgcctaatcgtgcgcaaccccggcatctgcgccatcctcgcgctcgcggccgcggtgtactacctgtttttgtgtctctttagcatggactgatggcgatcacagacagaccatcgcccgcgcgcgcgtgaccagctccggcgccgcgaagacgtcctgcaccgggaagtcgtcgatctcgaacacggagccgtccgcggaccagatcacgcgcacgttgtcgctcaccgagacctcggtcagcgtcacgcccagcacaaccgcgtcgttggtgctcaccagcaccagcgcgccgggctccgcgcgccggtgcagcggcggccccgagactgagcgccgctgcacgcggaacatgtccgcgaactgcttcgagagcaagtccaggtggttgcggatgatccactcgaagaagtacgcgcaaccgccgccgccgcacaggaagcgcgaacccgcgggcatcagcagccgcacaacgtccatgtagcaggcctgcggcaggctcgcgcggtacagccgcgtcttcggcgagagcaccaccaggctggaggtgctcatctggaagaccagctggctaacggagacggtgagcgtgcacgcgggcacggaaaccacgtccaggcagatgtcgtccagaaagatgctccgctggtagaggtggtacaggatggccacgatctgaaaggccgtggcgtcgctgatggcgcaggggcggtcggcgcagcgcatctgcgcgcaggaccagcccccgaaggactcgaagcagacggtgagcatgcccgtgctcggacagtgtggcgagcgccgacacaccggaaagcccacggccttgcggcagcgcaccatggtcgagagctctatccagcagcctgcctcctcctcgcccatgcccatggctaccggcgtgaaggccgtgacgtcgtcgcagatgcgccgctccagaaaccccacgcccgaggaggggtgcgcggccggcggcgaggtgatgcgcgccgggacgcggctcggagcgggctcgggaggcgagctgcgctcgacccgggcagtcgccgccggccgcgatgccctgcgcgcgggcgcgcgctcgcgcaacttgtttgacttgctggcctcgtcgctagcgtcatcgaagcggtcgttcctgtcgccgcggacgtccgcctcgtcgcccgtcggctgcgcggcgggcgacgtgccgtcccgcgtacggcccgcgttcggcgcgaatgtcacgcgccggtgcacgtacggctccgtagagcccgtgggggcgccgcgcccgcgcccgcggcggaaggcctgccgggacgcgccgaagcgggcgaactcccccttcgcccggccccttttttcttccatgatatttatcacaaaaaaaacttctctaaatgaccaatctgctttcgttggtcgacccggaggacctggccttctgcgccgggttcccgtccttcgacgagaccatgctcgtgatcgcgggggcgcgagtgcgcttcccacgctcgctgctctcgctcttcaacgtggtgccgcgcaccatgacgcgctacgaaaccgagctcgtgggcaccgagatggtggtgggcgccgtgttcaccaccgcgtacaacgtccgccgcaacctaggcctcggcgaggagcccgtgaccatgcgcgacatcgagaagtacttcctggactccgagaacgaggtgctcacgctcatcgtgcacaacaccgacttttccgccatgagcggcgtgcgccggcgcggcggccggcgcatcgccaaccccgtcatcttccgcagcgggtccacgccgctgctcatcgtgatggagtcgcgcaagaagaccaacatctaccgcgagcgcaccgcggagcaggccaacgcctcctacagggaggtcggctcctcgctcgcgctggtcactcggtacgcgggtctgcagctggttgacgtgcacacgcccagctccgtgctaacggtctccgccgtctacggcttcaccgaggacaaggggctcaagaagctgggctccgacaaggagctcgcggactaccagtccacgccgctcaccgaccccatccggctcagcgacttctccaatatattcgacggcgtcaagaagagcatccagctcacgaacgtgcccgtgccctccaccggcgccgaggccgcgccgtaggctttcatgcgcgataaatcggatggcggcgccgacgacgcccgcggtgcacctcacgccggtgttcgtggagcctacgatcgcgcactcgctgctgcgcgcagagtcctacctcgcgatcgcggtccttgagctcgtgctcgcgctcgcgctcgcgctcgtcttcttccgcgacgagctaggctcgctattccgctgcgcgccgcgagcgccttcgccgctggacgcgtacctgcaggcgagcctcgtctgcgacggcgacgcgctgctgatcgagctgcccgagggccgggtgccggcgctcgcgctggacgggcggcccgtcgcgttcccggggtgcgagagccttttgtaccgcataaatggaccacgaaaagtacgtcttgtcgatgttcttggaggaagataactccttcttctcgttcgtcgccgcgctgtccgatgacgaggcgctcggcgccgtgcagtccgctgccgccctcctggacttcctgctctccgtggtggtccgcggcaaggagaagctcgccgccgcggggcaccactacgactccatcgcggacggacgcgcgcgcgccgcgttcgagttccgagacctgcgcgagctggcgcagctcttcgaccggcggccctgcggcgtccaggaccgcgtgcgtgtgcgcgacgggcccgcgcgcgccttcgtggacgcggcactggggctcatgcgcgagcgaggcttcgacggcacgcaggccgcggagcgcgcgcgctacatcgcgccgaacgatctgcccgcgctgggggcaatatcggccacgctctcgccgggtctataacgtaaaaaatattagtaaaattctgaaggtccgtgtgtttcgcgggcggccaacaaaccagtcgcttaaatggagggggtggaaatggacaagccgctcctctacttcgacgagatcgcgggcgcgcgcgactacgacgcggccttcgcggagaagcacgagccgcccaagatccccggccgcggacagatgaagctgctggtctgcgagctcgtgtttctcaaccggctgcacctgcacggcatgctcgacggcagcgtcatcgtgtacgtgggctccgcgcccggacggcacatctgctgcctgcactcgcacttccaggagctcggcgtctcgcttaagtgggtgctcattgacgggcgcaagcacgacccctgtctctcggggctgcggaacgtgaccacggtgacgcgattcgcggacgaggcctacctccgcgagctgcgcggcgagctgcggcgcgccaagatcgtgctcatttcggacatccgctccaaccgcgtggacacagagcccaccaccgcggacctgctgcgcgactacgcgctccagaacaccatggtgagcgtgctcaagcccgtggcctccagcctgaagtggcgctgccccttcccggactcctgggagaaggacttctacgtgccctgcggcaaggagatgctgcagccgttcgcgccgccgttctccgcggagatgcggctgctcaccgtgtactcggagacgcgcccgaagctgcgtctgatcacgctcagcgacgcggtcaactatgaaaagaggatgttctacctcaatagcgtggtccgccagcgcgtaattctgaactttgactatcccaaccaggagtacgacttctttcacatgttctgtctgctctcgtcggtggtgtgctcgtgcgaatttaaatcgcccaaagagaaggtgctgagcctgcagaaccgcttcttccgcttcctgcgcatcccgccctccatcacgctcgggctgcgccggcacgatgaaccgccacaacacgcggtacctggccaagatcctctgcctaaaggccgcggtaagaagcgaccccttcgcggtggtaagtagggacaccgtgcgcatgtacgacatcgaggtcgagtacggcgacctcgtgacggtggtcaccgtcacgcacaaactcgagaccagccgcaccgtcttccaggtcttcaacgagacctctgtcgcgtactcgccgctgccggacgactacggcgagcccatcgtgctcaccacgtacatgcagcgcgagcacaccaagttcccgctctccatgctctacatcgacgtggtcgcctcggacatgttccccacgtttaagcgccccaccgaggaggaggccgcggtggtcgcggccatgcagcgcgtgggcgggcgccgcgagcccgtgctcaagctcccgcgcatgctggacaccgagctcgtgtgcaagatactgcacctgcccgagcacccgctgcgcgtggtgcgcttcctgcgccgaaacatgttcacgggcgtggaggtcgccgaccgctcggtgtccgtggtcctcgactgacgaagggcagcacggtcagcgaggccgccgccaccaagcacagcggcagccacgcgcgcgggtccgccacgggcacgaagacgtgctggttcaggtacttcgcctggaagcgctccgcggtggagtccaccttggacccgcaggcgttggtgaggcgcacgaccgcgtccgcgacgcgcacgtccccgagcgatatcacgcagtcagagacgttgcacccggcgatgtttttcttcagcgcgcgcggcagcagcgcgtccgcgcgcttgcagggcgcgtaccagcagtagtagggcaggcgcgtgtcgcggccggtgtcgaccacggcctggctgggcttgaggcacgcgcagcgctcgtcgtccgggtgcgcgtcgcagaaggcgtaaatctcctcgtcgggcgcgtccggcccgggcgcggtcggcggcgccgcgcgacggcggaagaacatctctgaaaaaatacttcgaccagaaaacgaccaccgatcttatttcaaagataaaaatactattaatacgcactcggagaatcatgtcggtggtggcgcgcgtgtcgtacagcctgtactcgcagagcgagataagcgccacggacgtggtcatcagccagttgaagaacgacgaggacctgggcacggtgaaggacccgcgcctgggcgcctcggacgggtccatatgccgcacctgcgggctcacggagatggagtgtttcgggcactggggcaaggtgcgcatctacgagtcctacatcgtgcgccccgagtacatccccgaggtggtgcggctgctcaaccacctctgcgtgcgctgcgggctgctgcgctcgcgcgacccgtacacgacggacttggccgcgctcagcgtgcacgagatgcgcaagatgaaggaccggatgatgtccaagaagaaggcctgctggaacagcaagtgtctgcagccgtaccagaagatcgtcttctccaagaagaagatctgcttcgtgaacaaggtggacgagatacccgtccccaacgcgctcatctaccagaagctgacctccatccaccgcaagttctggccgctgctggaggtgttccaggaccccgcgaacctgttctacaaggagtacatgcccgtcccgccgctgctcatccggccggcgatcagcttctggatagacaacatccccaaggagaccaacgagctcacctacctgctgggcatgatcgtgaagtactgctccatgaacgccgaggagcaggtcatccagcgcgccgtgatcgagtacgacaacatcaagatcatctcctcgaactcgagcagcatcaacctctcctacatcatcgcgggcaagagcaacatgctgcgcagcttcgtggtcgcgcggcgcaaggaccagaccgcgcgctcggtcatcgggcccgactccgcgctctcggtgtgcgaggtcggcatccccgactacatccggaacacgctcacgcagaaggtgttcgtgaactacctcaccagcaagcgcgtgcgcgcgctgttcgaggaccgcgcggtcaagttctacttcaacaagcggctgcgccagctcacgcgcatcaaggagggcaagttcatcaaggacaagatccacctgctgcccggcgactgggtggagatccccatgtccgagggcacgaacgtgatattcggccgccagccctcgctgcaccgacacaacgtcatatcctcgaccgcgcgcgcctcgcccggctacaccatcaagatcccgcccgggatcgcgaactcgcagaacgcggacttcgacggcgacgaggagtgggccgtgctcgagcagaaccccaagtccgtgatcgagcagagcgtgctcatgtacccggtgactatcttcaagcacgacgcgcacggcgcgccggtgtacgggtccatccaggacgagatcgtggccgcgttctcgctgttccggcaccagaacctctcgctggacgaggtgctgaacctgctcgggcgctacgggcgagacttcgcgccggagcctggccagaagaccttctcgggcgccgacgtcttccgattcatgataggcgcggacataaacttcaagggcgtgctcgagaacgggcgcgtggtggcgccgaacgtcgacagcgacctcgtggtggccatgcgcgcaacctcgctagcggggctgatcgcggactacgccacgaacgtggagggcgtgcgcttcgtggacatggcctcctacgtgtacaagcggtacctggccatctacggcttcggcgtgaccttccgcgacctgcgcccggacccgagtttggttcgccggctgcacgcgctgaacaccgagaagatagagcagatcaaggacgcgtactcgcggtacctgcaggacgtcgcggacgggaagctggtgccgatggcgcccgcggacgaggccgacgcgctggactcgctgctggccaacctgaccaacctcaacgtgcgcgagatcaacgagtacatgcgcgagacgctggagcgcaaccccgataacagcctgctaaagatggcgcgcgccgggtacaaggtcaaccccacagagctcatgtacctgctgggcacctacgggcagcagcgcgtgaacggcgccgtcgccgagaccaagatatacgggcgcgtgctcccgtacgcgttccccgactccgcggacccggaggcgcgcggctacatcatcaactcgctcatgaacggtctctccggctcgcagttctacttcgcgatgctggtggcgcgctcgcagtccacggacatcgtctgcgagacctcgcgcacgggcacgctcgcgcgcaaggtcatcaagaagatggaggacacggtcgtggacgggtacggacagatcgtgagcggctcggtactgctcaagtacgcggccaactacgcgaagatcccggggtccaccaccaagcccgtggagctgctcttcccgcacgagagcatgacctggttcctggagataagcgcgctctggacgaagatccggcacgggttcgtgcgcatgcaccggcagcgcctggccaccaagatcctggcgccgttcaacttcctggtcttcgtgaaaccggcgccctcggaggcggaggcgctctccgcgcgggacctgtaccacatgatccagcgcgtgatgaacgacgtgcgcgagaagtacttcttctcgctggcgaacgtggacttcatggagtacgtcttcctcacgcacctgaacccctcgcgcgtgcgcatcacgcgcgcgaccgccgagctcatcttccgcaagctgtaccagaagctgaacgcgctgctcggcggcggcacgcccgtgggcatcatgtccgcgcaggtgctctgcgagaagttcacgcagcaggcgctctcgagcttccacaccaccgagaagagcggcgccgcgaaggtgaagctgggcttcaacgagttcagcaacctcatcagcatgagccgcaaccacaccgagatagtggcgctgaccgcgccgagcgcggacaagctgatgccgctgaaggtaaacttcgagttcgtgtgtctgggcgagctcgtgcccgagatcgagacccggccctcgggacggccctccgtgcaccgcgtggacatcacggtgcaccgcctgcgcatcaagcgcgcgcacctgaccgaggtcctggtggacaccatcatcgagcgcttcgtgtccttcaacgtgctcgtgaaggagtggggcagcgacatgaccgtggagggcgaccgcgtcacgtacacgctgctgctgcgcttcgtggagccggagcagctcaacttccacaagttcatgctggtgctgcccggcgccgcgaacaagggcaaggtgagcaggttcaagatcccgatcaccgagaccacggtctacgacgacttcgacgccgcgcgcaaggcctaccgcatgaacatcgagctcatgagtctgaaggagctggggatattcgacctcgaggacgtgaacgtggtccccggcatgtggaacaccttcgacatattcggcatcgaggccgcgcgcgggcacctctgcgagagcatgctggacacctacggcacgggcttcgactacctgtttccctcctgcgacctgctcgcgagcctgctctgctccgggtacgagcccgagtccgtaaacaagttcaagttctggaacgcgagcgcgctgaagaaggccaccttcggcgacggccgcgcgctgctgaacgcggcgctgcacaaccgcaccgacgcggtcgcggacaacagcagctgccacttcttcagcaagacgccctgcgtgggcacgggctactacaagtacttcgtgaacgtggagatgttcatgcgcatggagcgcgagatccaggcgcgcgtggcggcgcgcaagatggaggagatcgaggaggccgccgaggaggagttctaggcgcgacggcgccttactttgcgaccgtgtcacgacgacacgacacggttaggacggcgagtcgcagacgaacatttttatgagctggtagcggaagttggcgttttccaggaaggcgccgcggaggtcccggatctcgtagtaggttttgaggaagtacacgaagcgcgcgggctgcgtcatagtcgggttctccgcaagccgcttgtgcatcacgtaccccatggcggcggcgccgctgcggttgacgccggccacgcagtgcacgagcgtgggcttctgctcggcctcgaggcgcgccagcagcttcacgagcgcgggcatgatggaagcgatgttcgtcgtgtcgtcgtctctcagcggaatgtggtacgccgttatccccgcgggcgtcgagtacttggacatggtcatgttaaccagacacttgaagtcgacgccggagtccccccgcagcacggcgcgcgcgtcctcggcgctgcccaagtacacgtggtccgtgagccgcgtcatgcccgagggcagggccagcggcggccccgcgcgcgtgcaccgcagcaggagcctggcgtaccactcgctcttatcgcccatatttatttatatgatacaaatggcagacgtcacaacactgacggccaacggtctgaccctggagttcgcgcgcgagcgcgctctgcgcagtctgcgcgccgcgcgcacctccacgctggtgttcttcacgctcacgctcgcggcctcgctgttcgtgctctggctgcagctaaccgagtttcccgtcttcgaggagctcggcaagtacgcgcgcatcaagagcgcggtgcggtcctggcgcccgctggtggaggctaagacagagatcgagtccgacctcggccggcagaagaccgccgaccggcccgagctcttcgagttcaggtgcgtggacttcggcaagttctacctgccggtgaggtacagccccacgaccttcctgccgcaagccgtgcgccgcggcgcgggcgatggctggatggtgcacaaggcggcggccgtggacctcgccgcgcagcagttctgcgagtccgtgctgcggcaccgcgccaacaacgtcatcacatgcgggtcagagatgatgcggctggtgggctacagcggctacttcgaggacgaccactggtgcgccgcgacgtccggcgtgctgacgtgaacgatcacacgatggccgtgaccagcagcccggcgatgaaccacagcagccgcgagttcggcagcagcagcacgagcaccagcaggtacgccaggatgaagatgtcgaccacgtccacgtcgaagagccccatgaaggagaagagcggcgtggtgaggaagtagatggcgccgggccagaagcgcgccagccacgtggcgagcagcgaccacagggagggcgcgccgctgagccgcgtcttcacctgtatgtagtactcggggtagaccacctgctcggcgccggagagcaccacgcgcgccagagagagccgcttctccagcgtgaacacctcggtgagcaggccgctgcgcagccctccctccttgatgatcgcgtcgtagagcttcttcatgccgccgacgctgatgatgtaggcgtctagcgagacgtcgtacccgccggggtagaccatgagctcggggtcgccggtgccggggacgttggtggccagcgcgccggtcatgtaggtctccttgagctgcgtcatgtaccagccgttcgccttcatcgcctcgatgagcggcttcaccatctcgggcttgcggaaggtcatgtcgttgtcgaccaccaggatgaagtcatcgtcggagtacttggtggggacagtgccggccgatatgctctcccagaggttgaggtggtgcgctgcgcggcgctgcatctccttcggacacgtggacttgcacatgtccgtgaagaagtgcgggtagtctttggagtccacgtctttccattccaccgccttgagcacgtggtcgcccttggggtgcggcgcgggaggagatggcttgggtgccggcgcgggggccggtgcgggggctggggcaggagagggagcaggcgcgggttgaggcttgggcgggtcgtcggcgaggcccaccaggtacggcagcgtggggaacacctccttggtcccgcggccttcggcaaccccgattatgtaggccgtgatttcgggtggatccatttagttattaaaattaatcatatacaactcttttatggcggctatggattcggctatccagtccttgaccgagcccacgatgcccgccaggaacaggaagaaggcgaactccaggtccacgcggttcagagagtcgctgaagtacacgaagacgtcgctgtccgggaagaagctgcgccggaacatgttgtacccgttgaccttgtgcgcgacgtgctccgcgctcagcagcgtctcgtcgaaggggtacgggtcgctgaagcggaacacgtacatggccgggtttgcgtagtagtacttcatggtgtttgtgacgaagaggctcgccagcgagatgatgatttttttcttctcgatctcgatcttgatgtggtcctcgaagcgcttcatgttgtaggcgttggtgtcgtgcacgcggatgagcacgcgcgagtccgacatgatgtcctggaactccgcgcgcgcgtcggggctctcggcgggcgtctccgcgggccgcgccacctccgcgcacaccgtcggcctagcgcgcggcggcgtgcgcatgggccgcgcccccacgcgctgcgaagcgaaaaactccacggcgcgagcctcgcccgcgtccgcgtacgactccaccaggtagttgcggctgcgcgtggtgcggccgatggtgttcagccggtgcagctccgcgaccagccggcggtagtgcgcctccagctcctcgggcatgatggaggtgtacacctcggtgagcagcatcacggtgtcgaagtcctccttgccgcagacgcgcgtcttcacgaggaagtggtgcacagccgtcgcgatagagagccgcagcgtggactcggtgacctcgacgctggcgtccttggtcttcttcgcgctccgcgaggccatgaacgagacgaggaagtccgcgctgctgttgagcacgatgaccagcgcgacgatgaagttgaggttcagcgtcttcgcggactggaacagctcggtggccgacgcgtgcacgtcgagcaggttcgcggagagccgcaggaagaacacgccgcgcttgatctcggccgcgaagcgacgttcgtactcctgccggcgcgcgttgatcgcgatgaggaagttcaggatgagccggttgatgttgtacttcacggcccaggtctgcgtcttcatgatggtgtcgaaggacatcacgatgttgaagatgaagcgctggctgtgcgagaagtagctgtagggctcgctgaggaagatggacttgttggtcgcgggcaccaccacgcccgcgcgcgcgccggacgcgtcggtgttcaggtccgggatgttcatgccgcagatgcggcagtaggccatgccgtcctcaaagtacacgaactcctccacgaactcgttgatcttggcgaagtagtccacgtccacgcgcatcgcgaccgcgagccggatctggtgctcgcagggcggcgactcgaagcgcaccccctcgccccagcccggcggctcgcgcacgaccagcgcggtgcgcgaggccgggcggaacttggcgtcgcgcgcgttgagcagcgccgggaagaggtcgcagaggtgccggctcgagaggaacacgtacttgtacagcagccggcgcgcgtccgcggccatggcgtccacgaaggcgcggccccactccgcgaccgcgggctgctcctccgcaaagttgttcgggtagaccttgtccgtggccgcgaggaacaccttcttcacgtcgaggaagtcgcggatcacgatggggacgcgcgcgccgtcgagctcgtacatgaacacgtagcgcaggttgagcttgcgccgcgagaccgggatgccgatgtgccgacacaggtacgcgaactcgaggtacttcttcgagaagcggatgcggtccaggttcttggagacgtactgcagcatgttgcgcatgttgaaggggatctcgcgcacggcgggctccgcggcgtcgtcgaaggcggtgcgcagatcgctggtgcgctgtacgaccacggcttcgccggtggcgtcgtcgtgcaccagcacgttaacgcgccgctgccggatgaccatgtcgaaggtgttgaagaacatctcgtacatgctgtgccgagtgtcgtccgcgatgcgctcgcccaccgagaggctcgcggtggcgtcgtcacgcacctgcttctcgaacttgtacccgatgtaggagaatatcgagatcagcgtggcgtcgtcggcgtcggggttctgctccatggtcgcgaagagcaggcggatgtcgtcctccgtgatcgcgtccacgttgtacaggttgaccacgaagatggacttgttctcggcgatgaagtccgtgtaggacttggtggccgtgttcgggtcgcgcatgtacgcgcggatcttcggcacgatgctcgcgaggatggactccctggaatccatttaaggacggcaagggcgcgcgagaccgtctcaaaactgaaatcgtataaactcttaaaaaatcggtattgaaagtacgcaccaccaaataaagcgtcgaggtcgggcatgtcttcgtggcgactcaaaatgagcaagtgttcaggttccagcagcgtccagactctcgaggatctgcgtaatcgtcttcgctccgaggccttgggcaacgattgccaagagccccgcgacgacctcttccccagcggcgaggagtgtctggacatcgacgggccctgcccttgcgatgaggcggagcaggagatcgaccaggagcagttgcccgtgcccgaaaccgtgcccgaaccgccggccaagactcctaagcgccgaccagtgaagaaggataaggcagataaggcagataaggacaagtcgaccagaggcgcaaagaaaccgtgcccttcggacgacaaggatgacgagctcaagagcaacgacgtcgacaacaacgaagagtccggcgacacagacggcggcgcgagcgcccgaagccccagcgacatcgacaacgtggacgaaatggacgactccgacctcatggtggcgttctccaccatcctcgcagacttcaaggaccttacccaacgagtgaaagctctttcgtccgtgctcacggacgtgcaggcggccggcatacgcaggagcttctcgacgctcggcaaggctctgacggaggcggcccacatcgccaacaccggatctaagccagtcactgcgcctcgcaagaagaaggccgccgcctgcaagaagtaggcgcactaaatagcgaggctcggtatgcgggcgctgcacctgtcagacggcaaacttttttttgacaaggagctgacgcagccggtccccgacgacaaccccgcgtacgctgtccttgcgaagatccggatcccaccgcacctctcggatgtggtcgtgtacgagcaggacctcgagtctgcgcagcagggcctcatcttcgtcgggcgcgacgccaagggccgaaagcagtacttctacgggcgcggacacgtggagcggcgcacggccgtccgcaacgccgtgttcgtgcgcgtgcaccgcgtcatgaacaagataaacgccttcatcgacgaccacctcgcctccggcagcgaggccgaggcgcagatggccgccttcctgctcatggagacgagcttcttcatccgcgtcggcaagacgcgctacgagcgcgagagcggcaccgtgggcatgctcacgctgcgcaacaagcacctcgccgaggccgagggcggtgaggagatccgcgtcgccttcgtgggcaaggaccgagtcgcgcacgagtttgccgtgcgcgaggggcagcggctcttcgcggcgctgcgtcggctctgggacccgggcgcgcccgacaggctgctgttcgaccggctgagcgagcgccgcgtgtacaccttcatgcgacgcttcggcatccgcgtcaaggacctgcgcacctacggcgtgaactacaccttcctgtacaacttctggtccaacgtgcgctcgctggagccgcgtccctccgtgaagtcgctcatctgcacctccgtgcggcagaccgccgagacggtggggcacacgccctcgatctcgcgcagcgcctacatggccaccgcggtgctcgagctcgtcagggacggcgcgttcctggacagagtcgccgccaccgacacgctcgacgacttcgtggacatcgtcgtggactatgtaaataactctgagcaggtaaatggatgaggcgctgcgcgtggcggcgcgcgtcgtggacgggctccggccgctggacgtggccgtgtgtctcgcgcagctgcgcggagccgcgcccgagcgccgcttcccggcgctcgacgagtgctccggcgaggccttcctggactttgagttcgccggcggggacgtggcgtcgcggtacctctccgcgcacacgcgcgagctccgtgcggcggagcggcgcgagcacatggccgcgatcgcgcgctgcgtcaccgaggccgacctggcgctcgcagaccgcccccggggcaaggcgcgcgcggcgctgcgcgtgtgccgcaaccgcgagaaagtcgcgcgcttggcgaggctgctgcgcgacgccgagagcagcggcgcggacttcgccttcatacgcgcggccgtggcgtagcaaaacgtaaaaacaacacattccctaaatcgccatggacgcgccaagtctcgactgcatgctcgccgcactcgcggcgaaggcggcctcggtggaccgaggcgctcccgaggacgaggtgcaccacgaagtggagctcgtgctcgtggacccgccgctgtccaccctggccgccacgctgcgcctggcctcggagacggagtccttcatcctcttcacggtgaccgcgctcgccaaggaggagggcaagctgcgcgcgcgcgtgcccatgtcgcgcgtcgtcggcctggacgtgaagaacgtgcagctggtcaacgccatcgacagcatcgtctgggagcgcaaggcgctcgtggaggagaccgcgctgcaggaaggctgtctgctgcgccactccaccgagcggcggcacctcttcgtggactacaagaagtacctctcggccatccgcgtggagctggtaaaccgcgtgcgcgtgcgctccaaagaagtcgtcgcggacttcaagttcaagtactttctggggtccggcgcgcaggccaagagctcgctgctgcacgcactcaaccaccccaaggtgcggccctcgcccacgctggagttcgaggtcgtccccgcgggcgaggccgtggacgaggccgccgtgctcgcggagctgcgcgccgtggcgaaggcgctcttcatggcgcccaccgacgccgtcttcctggcgccgccggccgagatgccggtgcgcacgctcatgctgcagaagcaggagatccccgcgctagacctcgacggccttttcgcggtctccaagacggacggcgtctccgcgagcgtgtgcgtggacgaggacggcgtcttctgcgcgttctcgcacctcgcgtacaccatccggtacccgctcgcgcgcgaagtgcagggccggtaccggctctggtgcgaggccgtgcggcccgtgggcgagcgcgtgtggtccatgttcgtgctggtcgtggaggagcctgcgggcgatgaccgcgtcgcggccgtggccggcgccgtggaggcgctgcgcggcgtgtgtgcacgcgtcgagttcaaacctaagcgcgtggacgggcccttctcggcgacctccgagctggtggagcacatcaagagcgcgctgcagacggagccagagggcgtggtgctcttctacgcgcgcggagagaagtccaagcgcgacctcaaggtcaagcgcgacaacacggtggaccagaccacgaacgtgatgttccggtacatgtccagcgagcccatcgtcttcggcgagggctccaccttcctggagttcaagcggtacagcaacgaccgcgggttccccaaggagtacggcgcggggcgcatcttcctgcgcgaggacgtggtctaccacaacaacatctactgcatcgagttcacgaagacgcacctggaggtgggcctccgcagcgtggtcgtgcccgtgaagttcatcggcgagttctcgcaggaggggtacctgctgcggccgcgcctggccaaaacggagtgctacttccgcaacccctcattctacgggaaccagcactcggtggtgctcgagcacactcgcgaccagctgctctcggtgggggacgtgttcgacgagagccgcatggccgccgtcgggcagacgctggccaacgacgccttccgcctgaacccggacacgccctacttcaccaaccgacgcacgcgcgggccgctgggcgtgctctccaactacgtgaagacgctcatgatatcgctgtactgctcgaagaccttcctgaacaacgccgagcgacgcaaggtgctggccgtggacttcggcaacggcgcggacctggagaagtacttcttcggcgagatcgcgtccatggtggccacggacccggacgcgcgcgcgatcgagcgcgccatggagcgctacaaccgcctcaacgcggggctgaagtcgcgctactacaagtttaactacatccaggagaccatccgatccgagacctacgtggagagcatccgccaggtcatgtacttcgggcgcttcaacatcgtggactggcagatggccatccactactccttccacccgcggcacttcgccacggtgatgcgcaacctgcgcgagctcaccgcgcccggctgcaaggtgctcatcaccaccatggacggggacttcctgtcgacgctctccgagaagaccagcttcgtgatcaaccgcaacctgcaggagagcgaaaacttcatgtcgatcgagcgcgtggccgatgaccaggtcatggtctacgcgccctcgaccatggcgcagcccatgacggagtacatcgtgcgccgcgcggacatcgtcaagctcttcgcggacaacggcttcgacctcgtggaccacgcgaacttcgagaccgtgatccggcgcagccgccgcttcgtcgagggcgtctcgcggctggagacgcggccctccaccaagaacttcttcgagctcaaccgcaacgcgctcacggagatggacagcaccgacgtggccgcgctgctaaagatctacgtgctgtacgtcttcagcaagcggtaggcagaaccagggcgtcgattccgcgcccgcgccggcgcggaaggcgttgaacagctccgccagccaggctgcggtctcgcgcgcgtcgatcgggccgccgtcgtccggcggcggctcgcgcgccgcgcgcaacaccagcgtctccgcgggcggcagaggctccagagcctcgaagaccgcgcggctcgggaacagcgcgcgcatcatgcgcgcgcggtggccgaacaccgccttgaccgcgcgcagtgccgagcggttgtccagccgcagcgctcggtcaaaacgatgcacgcgcgcgggcgcgccgcggtggtcgcgctccacgagcacgtgccgccacgccagcgccgcgccgacgcggtccaggctgggcgcgagcgccaccaggcttttcagcgcatgtaaatctccgcgcatggccgacggctccatttactactgcggaggaacgcacgtggtcgcggccgcgccgggcgccgcgcttgtggtgctggacgcgcccggtgcggcggcggcggccgcgcccgcggggcagcgcgtcttcttcgccgagtacggcctcgagaagcgggccggcggcccgatcacggcgcggctgcgccgctccgggttccgcggcgccgcgaacgcctgggcctccgtggcggacttcgaggccggcggccgtccctccgcgtggacgctgcgcgcggaggaggcttcgcgcgtgccgctgccgacggacgcggcgctggtcctggcctggggcgcgcgcgaggagccgctgcgggcgtgcgtgctggcgcgcgcggcagacgcggaggcgccggtgggcgccgcgctcaaagaagccgccttcgacgcgcgggcgccggcggccgcgctgttcgcggcgctgggcgcgcccgcgctcgcgcccccgctgcgggcgcggctagtggcgccgccgggcgcgccgccgcggacgcggctctgcgagaacccggccatgctgcgcgcgttcgcggtgggctggttcggcgcgcagctgggcgaggcctccgaaaatgaaaaggtatttgccgcctttgataaggcgaggtcgtgtttggacgaccgctgatggcgacgcccgcgaacgcgcccgcgctgctcgtcgcggcgctgcgacaccgcccgtaccgcgtggagtaccacccggactgggagccggtcatcgagacgctggtggacgagtacgacgcggtcgcgccctggctgctgcgcgacgcgacgagccccgagcccgagcgcttcttcgcgcagctggcgaagccgctggcggacaagcgagtgtgcgtgtgcggcatcgacccgtacccgcgcggcggcaccggcgtgcccttccagtccccggacttcagcaagaagaccatccgcgcgatcgcgagctcggtcgcgcgcacgaccggcacgcagggctacgcgaactacgacctggacgcggttccgggcgtgctgccctggaactactacctctcctgccgcgagggcgagaccaagagccacgcgatgtactgggagcgcatctcgcggctgctgctgcagcacgtggccaagcacgtgagcgtgctctactgcatggggcgcacggacttccagaacgtgcgcgcgcgcctggacgtgccggtgacgctggtggtgggcttccaccccgcggcgcgcgacgggcagttcgcgcgcgagcgggccttcgaggtcatcaacgccttattggagctcaacgggaagtctcaagtggactgggcgcgaggattttctttttatagtgaaaattaatccgtggtcctaaatggcggcgcccatatgcgataactctcacgtgttcctcctcaagcgcctgggcgtgccgtcttcctgccggcgctcggaggacccgcgcttcgtggagatcctgactcccttcgagctctcaaactacatcgagcggcacccgggatgctgcctcttcgagacgctgcgcgacgaggaggactgctccgtcgtgcgcgtcttcgcggacgtggacatggacagcgtgctcgaggaggaggacttcgtcgcggcgctggaggacctcatcgtggagctcgcggccttcttcgaccgcttcgcgagcggctcctgcggcaccgtgcccggcgaggtcaagcgcgccatgctcgcgaacttctcggtcacgcgatccacggccgagcacaagaccagcttccacctgatcttcacggagacgtacaccacgctggacacgctggtggcggcgaagcgcccgctgctggacctgtgccggcgctcggacaacgtgctgctgcgcgcgctggacacggccgtgtaccgccgcggcgcgacgctgcgcgtggtgggcacgcgcaagacgccggagtcgagcgcggtccaccgcatgcagtcgcccgacgacgacatcaaggactacctgttcacgttcgtggagctctcggacgcgagcgtgtacttcgagctcgcggagcgcgagcagcacacgctgagcaccgtctgctgggagacctcctacatccccttcggcgacgcgatgcggcgcgtgtgccaggcggtggtcaacgacatcgtgaacctccgcgacatcaccgaggacaacttcctcgacacgccgctggtcatcgactacgcgacgcgctgcgcgctgtgcaagaagcccaagcacaagcacgcgcaccacatcaccatgggcaacggctgcctgcgcctggtcaagggcgggaacgcgcacagctgcaaggtcaagatcatccagctcgagggcaaccggctcttcacggccgcgcagatcatcatcgcgtccgaggtcgtgaagctcaccgagcgcaacgactacatcgtgtggctgaacaactcctggcgcttcagcgcggaggagtcgctcatcaccaagctcatcctggacgtgcggcactcgctgcccgcggactacgccaacgacatgctgtgtccgcgcaagcgcaaggtcgtggagaccaacatccgcgacatgctcgtggacatctccgagacggacacgcagtacgacaagctgcccttcacgaacggcgtgctggacctggccacgggcgagttcctcaccggcgaccgcgcgaaggcctgcgtgtgcacggtctccaccgggtacgccttctcgcgcgaggagttcgcggccgcggcggactcggaggccatgcgccggctggtcggcgtcatcgacgacatccagccggacacgcccgagaacgccgataaccgcgcgctgtacgagcgcgccatgtccagcgcgctctgcggcgccacgaagacggtcatcgtcttcttctacggcgacaccatgaccggcaagtccacgagcaagcgtctgctcatgtccgcgctcggcggactcttcatcgagaccgggcagaccgtgctcacggacgtgctcgacaagggcccgaaccccttcgtggccaacatgcacctgcggcgcgcggtcttctgcagcgagctcccggacttcgcctgcaacaacgcgcgcaagctgcgctccgacaacttcaagaagctgaccgagccctgcatcgtgggccggccctgcttctccaacaagatccacaaccgcaaccacgccaccttcatcatcgacaccaactaccgcccggtcttcgaccgcgtggacaacgcgctcatgcgccgcgtggcgctggtgcgcttccgcacgcacttctcctcgtcggccactcgcgcggccgccgcgcacaacgtcgagtacagcgcggtcaaggagatggacgagagcctggacaccaagatccagcgcaactacttccgctacgccttcctgcgcctgctcgtgcagtggttcggcaagtaccacgtcccgcaggtctcgctggcgcccacgcccgacgcggtccccgacttcgccttccaccgccgcgtggccgagctggtggtggccagcaacgacgcgcaccgccgcgcgatggagtcgctgtccaagctggggtacgtgctcgtgggcggcaacgtggccatgcccgcggacgccttccggcagcggctggccgcgcacttcaacgcgcgcgtgcacggcggcgacatagacgccttcatgttcaagcacaagaaggtcgtcaacgtaacggaggagtacgtggagtacgtattcatcgaagatgtcgagaataaatagacgggtatgaactcggacgtgataaagctgttcgtcgggcacgacgagtccgtgcccggcatcctgccgcaccagctcgcgaccgtggacttcctgatacgccgcgttctagacgacaacgtcagcgtgcttctcttccacatcatgggctctgggaagaccgtcatcgcgctgctgttcgcgatggtggcctcgcgcaccaagaaggtgtacatcctggtgcccaacgtgaacgtcatgaacatattcaactacagcatggtcatggtcgctaacctgttcaacgcgcccttcgtggccgagaacatattcgtgtactcgacgactagtttttattcgctaaactgcaacgacggcgtcataaactacaacggcctcggcaagtacgagaactcggtcttcgtggtcgacgaggcgcacaacatcttcgggaacaacaccggcgagctcatgatggtgatcaagaacaagacgcgcgtgcccttcctgctgctctcggcctcgccgatcacgaacacgccgctcacgctcagcagcatcatcagcctcatgtccgagaaggacgtggacgtcggcgacatcgtggtgcagggcaagaaggtgttccagatcctgctgaacgagcacggcgtgcgcgtgatccgcgaggtgctcaaggggcgcatctcctactacgagatgccggacacggacatgcccgaggtgctctaccacgggcgccgcttcctggacacgcgcgtggtctactgccgcatgtcgcgccggcaggaggacgactacctcaccgtgcgccggctctgcaacaacgagatgttcgagaagaacatgaacaacgtgtccatggcggtgctgggcccgctgaacctggtgaacaacctggacgtgctcttccaggcgcaggacaaggacctgtacccgaacctgcgcatcagcaacggcgtgctctacgggaacgagctcaccaagctggacatcagctgcaagttcaagttcttcatctcgaaggtgggcgccatgcgcgggaagcacttcatctacttctccaactcgacctacggcagcctggtcatccgcaacgtgatgctcagcaacgggtactcggagttcggcggctcgcagagcaacaatccgcacaccacgcccgacgggcgcgccaagaccttcgcgatcgtgaccagcaagatgaaggcctcgctggaggagctgctcgaggtgtacaactccgcggagaacaacgacggtggcaagctcatgttcctcttctcctcgaacatcatgtccgagtcctacacgctcaaggaggtgcggcacatctggttcatgaccatccccgacaccttctcgcagttcaaccagatcctgggccgcgccgtgcgcaagttctcctacgcggacgtggccgcgcccgtgaacgtgtacctcatggcggcggtgtactcggacttcgacgaggacatcgtctcgctggaggactacagcgtggaggacatcaacgcgctgcccttcgacgtgaagaagctcttctacctcaagttcaaggccaaggaaaccaaccgcgtgtacgccatcctgcaggagctctcggacgcgtactccgcgcgcccgcacccgcagctcgtggacgtggtgctgggggagatcgtgcgccagttcttcgcacggcactgccgcgtgcccgccgaggacgccgcgctcgtggccgccgtcgaggccgttctcggcacgcgcgaggcagcggccgagtacatccgcgcgatagtggacggacacttcttcgtgaccaacaagaccttcgggaagtgcctgctcttccggcacgagcgcgacatcgtgaccgtgcccttcgagctcgagcacgaccccttcgcgtgggcgatcaacttccgcaaggaggtcagtgtggtgaatatataacggcaaacataaatagaaagactgtccttttgcgcgatgtcgaccttccggcagacggtgtacctggcggtgacgctgcagccgcacgagctcacgctcgacttccgcggcaacgtcgcggaggcggtcatgcgcgagtacctctacaaggagaagggcgggctcatggccaccgacatcgaggtctgcctcggaaacgagatgccgctggggcgcatcgtgaacaacgcggttgtggtctcggtgccctgcaacgtgaccttcaagtactaccgcgtcggcgacaccgtgagcggcacgctcaacgtcgaggacgagaccaacgtcttcgtggactgcggagacctcatctgccagctcggcaagagctcgggcggcgtgaccttcaacgagtccaagtactgcctcgtgcgcaacggagtcgtctacgagcacggcagccgggtctcggctgtgctgcgcgaggcgcgctccggacgcgagtccgcgttcgtgttctccgcagtgctgctggacggcgtccccgccgaggagaaggacgagaagaaggacgagggcgagaaggccgcggaggaggagacgcccgcgagccccgccgccaaaaactagcattattgggccgcgcgaaccttcgataaatgcgcacgtacacgtcgctgctctcgaagctgctcaagagcaaccggcggctcgggagcacgcgcgtcttccgcgacccgctgcagcacatcagcgcgaccgcctttgtgcaccggcgcatcgaccggcaccggcgcgtctccatctgcgccgtgctcaccaccaccgacgggctcgtggtcgcgtgccggcgccggtactcctttttgtcctccgagctcgcggagacgcgctcgcccgcgcggcgcgtgctgctcgcaaccaagcacgcggacgctctcgcgcgcctcggcgccgcgcgcccgcgcgacgacgtcatgtttccgggcggcgccccgctgtccggggagtcgccgctggcgtgcgtgctgcgcgaggtcgaggaggagaccgggctgcgcggcgaccaggtcagcgtggacgagcggctgttcgtgcacgccttcatcgacgacctggtctcgggccgcgacttcgacgcgatcatcttcacgggcgcagtcgcgctttcgagcgcggaggtggcgaagcagttccggcccaacgacgaggtcaaggggctggtcttcctgcaccccgaggacgcggagggcgtgggcgtgatggcgcggctggcggcgttcgcgcgctgcgcggcgcgcctgcgctgctggggcgcggccgtcacgcgatagaggcggggtccaccacgtacacgaggcgcccgccgctcacgcgcacggtgggcgggtcgcccagcgcggtcaggaagttcccgtcgtcgtcgaagaggcgcccgccgcgctcgaggaagcccttgcgcaccgtgaccagcgccgtggaggtggagtaccacacgctctgcccgtccgcgagccgcgccgcgcgcgcgggcccgcgcgcgtccgccgggcgcgccaccagcgcggaccagccggagtcgtcctccagcggcgcgaagtccgtgaaggcctcgcgcacccactccagcgagcagcgcttgagcacgcggaagagctgcgtgaactgccgggacttgtcgcggatgagggccagcaggtcctcgtccacggtggcggcgccggagtcctggcgcgcgaccacgaagtgcacgttcacgtagcggcggtcgggcggcgtcatctcgtggctgttcaggcgcaccgcgcggcccacgatctggcgcagcgaggcctcgttccaggtcatgtccaggatgaagatgtcgttgatggagaggaagctgaggccctcggagccgctcagcgagaacacacagaccttgatcttctcgccgtcggtgttgtcgcaggcgttgaaggcgtccacgagcttggcgcgcgtgtcgcgcgtgcgcgaggagaactccacgctggagacgccgaaggcgcggaagtagagcagcagcatctcgatgccggtcacgttgacgaagggctcgaagaccagacacttgcccggcgaggccaggatgcgcaggcagacctcggtgtacttgcagctgcgctcgcgcagctccgcgagcagcgagacgtccgcggaggtcatgcggtcgccgctgacgggcgcgccgctgcggaagagccgcatggccgcctccgagaagacgcggtccttgacggcgcgcgcgaagtccaggaagagcgcggccacggcctcgtcgtactcctgcttggagagcacggacttgtcgggcgcgtcctcgaaggcgaaggtggccgcgatgcgccggtacacgcggaagaccgcggcgccggacttgcgctccatggcggccgcgcggcggtaggcctcggtctgcttcgcggtcatgtccacgtacatcatgcgcacgcgcttgcgcgcgaaggcggcggagccgtcgacgtcgtcgaagatggaggcctcgttggtgactaagtacgagcacaggccgccgagcttgtccacgaggtcctcggggttcgcgagcgcgccgccgttgaagagcggcgtctgcccgaccacgccggggcgcagcaggttcacggccatggagaactccttgacgctgttcaccaccggcgtggccgtgaggcagagcagcttcccgcggcccatggggatgttcttcgcgaggtagttgtacaccgtgcgcgcgggccgctggcgcccgtcctccttggtcagcgacatcgagatgaagttgtggaactcgtcgatgaccacgcagacgcggctgctcgacgaggcggtcttcatcagcgtgaagaagcggtggtggaagcgcgggtcgtcgtagttgatgaaggtgcacccgggcacggcctcgggcgcgaagcgcatcatcgtcgaggtccagggctgctccacgagcgccttcttcacgagcacgaccaccgtccagtccgtgaagacgtcgcgcaggtgcttgagcacgtacaccgcggtcacggtcttgcccacgcccgtctcgtggaagagcagcagcgagtgcatgctgtccaggcccaggaacacgcgcgccacgaagagctggtagtccttgaggcgcacggactcctccacgccctgcatctcggagggcatgtgcgcggtgcgccgcagcgcgtagtcgatgtaggccgcgtgcgcgctggtcatggcgacggtcggcgctccttttacggggtctgtcgtctatctattgtcggcgcgggtctgatttaggggcagtagttacaaaaacgtttccgctgctcggcgcggcgtttggaggagcggttgcggccgcggcggcgcaggcgcgcgcggcgcgtcttcgtggtgcggtggccgaaccagcgccggtgcatgaccgggtgcgagaccgcggccgcgcgatccgcgctcatgcaggttgcgtaggtgcggcacatgctgcgcagcacgcgccgcgtgcgccgctccacggcgtcgagccgcctcgcgacgatgggaaagagccggcgccagccgcgcacggcgaagagcgggcgctcgcagaccgggcgcgcgagcgcgtggtaggcgcccagcagccgcgggtccagcgagcgcacgtaggtctccacgaagccgttgccgaagacgatggcctgtgcgcatagcgggttcgtcatctccctcttggaggcgatggcgtcgcccacgaaggcgcgcacgccgcagtgccgcagcaccaggcgccgccgcgggaagtgcaggtgcgggccgagcgccgcgcgggcggcggggatgtgcagccgcggagaaaaacgcgcgcgtccctccatggcatctaagcgctccgtctgttttcagttatatcgccgcgggcggctactgcagcagcagcttgagcttgcgctggctctcgttctcgatgctcttggactcggaggtcatgctctcgtagagcagcgagtgcgtgacgtagagcgcctcgtacacgcggctggcgaaggccacgaagcggtccacgaactcgctctctacggggtccttgagcacgcggaagggcacggccagcgcgtcgcgccaggcggcggccttggtgcgctcgcgcacgtgcgtcacgaaggcggcgatggccgcgcgccgcggctcgctggcgaccatgacggcgctgtccttgagccagctgtcgctcacgcacttgaagaggcgcacggtgccgaagaggctgcagtacacgcgcagcgcgtgcaccacgtcggtgccgaagagcgtgggcagcttgagcaccacgaagcgctcctgcgtgatctcgagcagcgggcgcatcaccgcgaaggtgatcgcgtggtagtcagccacgtagaggttgttctcggtgaggtggttgttggagcggatggcgccgcgctccttgcggtagagccggttgcgcgcgctgaggtcgagcacgaccgcgtcggccctgccgcgcgctctggagcgcacgctggtgatgccgtgcgcctcgagcaccttctcgacgtcgcgctcgtcgatcatgaggtcgtgcgtgtacagactcagcatctccgtgggcatgcggttgatgtcgttcacgcgcgagcactgcaggaagtagttggtcccgtaggccaggctgggcaggtgcccgacgccgagctgcaggtccagcgcgggcgtcgagtcgaaggtgggcagcgtcacgctgagcccctcgcggatgctgcggcgcacggcctccaccgcgtccatggccgatttattggacgcacagtctgttttcatttcgcggctactgcgcagtcaccttctcggccacgatccccgcgtcgtagctgagccggtacacctcgttgcacaccacgaccatctgccgcggcacgtacatgagcgggttgtgcgcctccatgtgcgcggtggtcacgcgcaccgccagcttgtccttgcccctggagacgttggagttcagcgcggtgggcgagaagaaggtgctgggcgtaaagttgaactgcagcgtgcgcacgccgggcgtcttgccgaggatctcgccgaagacgcgcgagactgcgctgttctccgagtacagcacctcgttgccgaagcgcacgtccatgcgcgcgatgacgtcgatcttgttcttgaagtccacgcccttgaggaaggggtcggccacgaagaggtccttggcgcgcgcctccggcgagcggttgtcgccgttgtacacgttgcgctggcaggtccacacgcccacgggcacggaggcgtcgccgatgttcacggagtggatggcggtcgtgaagcggatgcgggaggtcgcgcggctgtaggcccccgtgatggcggagaacttcttggacatgttgtacacgacggagttcttcctggtggcgaacactaggatgttcgtgtgcaggaacacgcgcatgcccacgggcacgtcgtcgatgcgcacgaagacgtcggtgtcctggatggacacgacgcccgacgggggcacctcgactatctccgcggtctcggggaagccctcggggtagcagttcgagacgatcaccatgtcctccagcaggcgctccacgaaggccatcacgaagtcgccctcggactgctggaagccggggtacgatatgaagcggttgttggcgtcgctgagcacgggcttcatgtacacggacagggaggtgcacgcgtgcacgtccgtgatcaccgcggtggtgtggttgatctgctccacgcgccgccgcggcatctcgatgaaggccgggcgcgggcacaggttcttgaccatgtagccgatgaagctcagctccatggagtaggggaactccttggcgagcttggcggcgtcgaaggtctcgtcgtagaccatgacgcaggcgacggggttcagcgtgaccgtgaccgtgaccttgctgtcgctgagcttgagcgtgctgaaggtcttgtccgcgtcaaagggcgtcttgatgtaggcgtgcacgcaggcggcctccttgatgacgtcgttgggcgagctcccggtggagaggtcgttgagctcgcgcgagaagcccgagagctccatcacgcgctcgttgtccaggcaggagtcgaacagctcctcgccggaggtctcccagatggtgtccgcggcggagttcacggccacgtggcggatgagcttgtacgcgatgtagggcacgtagcacatcttgcccacgccctttatctcgggcaggtccacgctcagcacgaagttgttcatggccgagatgtacttgtcgcggatctcgaaggtcacggtgaccgcgtcgctggtggtgtccaccacgccctgcgtggtgatgtactgcggcatgtacaccgtgggcgcgcggtggtccgtggcgaacacgctggcgcgccgcacggcgtcgtcgccgcccaccaggctcaccacggagttattcatttattccctgggaaaaccagttaaataaggctcttcagagccatgcgcaccgtccggccgtcgggctccaggtagcagcgcccgtagacgccctccgtggcgcgcgtctcgttgatgagcgcgcgcacgcggtcggggtccgcgtacatctccagcggcagcagctcgatcttgggctcctcgcgcagcgcgacgaggtgccggatggagcccgcgaaggagtcgcggcacaaccgcgagcagaactcgcccacggcgccgccgtcgagcgtctccacggccagcgcggccgtgcccacgcgctgacggcagaaccagcacgtgccgtccgcggcgcgcagcgccagccgctccgcggacaccgtgttgaagtacttcggcagcacgtactcgatgcggcacgccgccggcggcgcgcaggccgacgcccgcggcgcggatatgtccacccgcgagagcgcgatgcgcttcatgggcggcggtggctgctatttatgtcgcccgcggcttttcaaaggtcgagcgagcacgccgcgaagcgcgcgggcgagaacacgtactcgtggccgaactccgggatctgcgcggcgcgcttgcgcgcgcgcatgtgcgcgaggaagttctcccaggtgagctggttgctgttgttcttcgcgtagttcttcacggtctgcggacgcaggttgcgcgtcacgcccgtgacctcgaagatcttgtccaggaagaaggagtagttgatggttttggtgggcgtgatctcctggcagaagaagaccagctgcttgaatatctcgatgacctcgttgatcttctcggtgctgaggtccagcttctcgtttttgacctggttgatgatctcgaagaccagcttgtagtccttcttgttgatcatttcgctgtccttgaggaagctggagacgtagttggcgtccacgtcctcgggccggatctggtgccggtccatcatcgcgcgcaggtcgcggatgacctcctccgagcactgcttggagagcagccgccggagcacgttccgcaggtggatgagcttgttcgacacgtggaagttggacctcttctgcacgcggatgcccatgggaaacacggtctcgcagaacaggcagaactcgtagtccgcgtcggacacgagcccgttgcggcggcagccgccgcacatgcgcaggttcatgcttgctccagccccagcacgcgcaggatctcgcggtccagcactttagtgtccagcgtgcgggttctacagaactggaggaagcccgcgagcgcgcgcgcgcgccctggctgcgagagcagcagcatgcgcgcgttctcggggtcctcgttgatgacgcgcgtgaggttcagcgagcaccgcgtgcagcgccgcggcgggtcgagctcgaccgagtacgccgcgaaccagacgttgtcgcccatgtattatttattaacacagaacgtcgcacatgttgcgcgaggacatgtacgggtcgtactcctgcccgtagatgaggatggtgcagtaccgcgagatcatgagcatggcctcctccatggtgagcaggtcgtcctcgaacatggccttgtgctgcatgtgctggctctgcttggcggccatcgcggccgcgccgtcgccgcgcagccactcgttcagacactggccgtcgccgccggcgccgctctcctgcgcgaaggtgttgcgcatggcgcgcatgagcctggcctccctggccgagcggctgagcacggtcatggggtcgtagagccaggggcctgcgtccgtgaacaggatggtgcagtacccgttggcgaaggcgtccgcgccgctgcagttgtcgatgccgtcgccgaccctgtagcacaccgcggagaccagccggtacatgatgccgttgagcatcatgtcctgcgacacctcgatgggaatgtcgctgatcacgggccgcatgttcgtgaagcagtcgcccgtgctggccatgcccccgcgccggttcaccaggaacaccagcacgccgttcgtgatcacgggcgcgcggtcgcgctcgtagaggtagccgctcgccgcgcacacggcggacgccacgtccgtgcgcgagaccgcgcccatgttctgggcgggcatgtacagcactcgcccggcctccgcggtgcacgagaagggctgctccccgcccacgtggatgggcgccgtcgatgtcgtgatcatcttgctggagtccaccaccaggtagggcaccgtgtgcatggccatgtcgcccatgcccccgatccccgttccgaacgacggccgcgacacgctcacgagcgtcggcttgaacgagactatggagaagatggaggccaagatctgctcctcgtcggtcatgatggaggcgcacgaggggtggatgatcttcattagggcgttgtcgatggactcgtcgctctcgcagtagaagacgcccatgcggaggttcaggatgcaccgccgcaggttggtgtgcagcaccgcgcgctggatctccatggacacggagtcgccgacgccgggcatcacgatgggcgtttcctcggtgagcttgttcaccagcagctggtagttgttgggtcgcacgcggctgttgtggtggagctgcgccaggagcgagaggctgtccccgttgacgaacgcggactcgatggccggcagctttacgccgaacagcgccatggcgatggggtgcacgaagcccacggagtcggacgacttgaacgagaagagcaggtcgcccgaggagctcatgtctttgaagtgcacggactggaactgcgtggcggagagcaagttctggtagctggacatgctctgcaggtcgtcgatctccttcatctgcttgtttacgcgggtcgagttcctcccgtagatgatcaccagcgggtgcgtctggctcacggatagccccgagtccgtcatggcggcgcgcacgctgttcagcagatcgaacagctccgaccggtctctgttctggatcccgacctttgccatcacagacatgagctcctggatggtcatgttcttgtggtctcggcgcgtggaccgcaggtagtcggcgatcatctcgccctccttacggatcttcatctgccagtcgtgcatggaggtcatgcggtccacaggcatgagcacgctgtcggaggacgactgcgcggcgctgccggactggcgcgagccagggcgcgcggacgacgggcgcgcggagctgccgcggctggaggaggacctggacctccgcgaggatcttcgctgcgaagagctgcgcacgggccgctgggcgcgcgccccggcggaaaccatgtcctcgcggtttatgctgaggagcgagctgcagaccgcgcacgacagcgactgctttggaatgtggatgtggtcgcactccagggacatgcccgcattgtcgtagcccgggaccaagtcgaactttgcattaaaaaaatctgatgcgcacgcgggcgattccatttataccggaagtttttatgaggtgccggtattatccacgcgatctcgcagtgtgctgggagactctagcgtagccacggaccccgtgagcagacgacgcaagtcgttgatggccgactgcgtgacggacttcgccgtctcgatgtcgcgcgtaaggctgagcgactcggcgttgaggtcgcgcacgctgtccgcgatgtcggccagctcctttttgatgaaatccttatcattatcggcgttgatgactttgtccggcactctagactctagaaccggtgacgcggcgggcgccttgatcgtcgggcagctggacgccgggtactggggcggcggcaatgattgctgtaggaagatgggcttagcggcaggcgccggttttgtcggcaagggcggcgccggcgggcactgtcgtgtagacggcgggcacgccggcgcggggcacgccgccggtggcggacatgtcggcgcagttgcgggtggacacgcgggcgcgggcgccggcgcgggacacgtcgcggcaggagccgggcacgcgggagccggagctggagccgggcacggcgcggcaggagccgggcacgtcgcggcaggcgcagggcacgcgggagccggagccgggcacgccggcgcaggaggacatgccggcgcagcggcaggacagaccaccgctgtcgcggagcacgcggcaaccggcgtggagcacgcggcgggcattacgttcagaggcgtcgactggaccttggcaccgcggggcagtagcgatggcgctaggggcgactgtccggtggttggacgctgcatgcaggcagtcgcagatttcagacgggactggtaatacctgcccgcttccttcaccgtgtacttgtcgacggagtctacctcctcatctggaggacatggctgctccggtgcgggaacgactgtttgaggacacttggtgaacagactggagctggtctccgatagcaccagtttagacttggccaagtcggaggcaaaccttcttctgagatccatttaagccttcaaaattgaacgtgtacgccgaccgctaaatggaagaatcggtggccgtcgagtacgcggacgaggacgaagatgagattgaggagtacgaggaggaggacgatgacgaggaggaagagtctgccgagggcgccgccgcctcctcggtcagcgacgtagcgctctctgccgccgagaagctggtggcctcggaggtcccggacgacgcggctgccgcggacaccaacgtgcgtcaacgcgtcaccgcgcgcgtggaggagcttaaggcgcgctacacacggcggatgagtctatttgagctcaccggaattgtagcagagagtttcaatcttctgtgtcgcgggcggctgccgctcgtggcggacgccgcagacccggcgctcgacaacgagctaaaagtggtggttcgggagctcgaggagggcgtctgccccatcgtcatcgagaaaaacggcgagttcctctcgccgggcgacttcgaccccgagtgcctgcgctaccacctgacgtacatgaccgacctctggaagtcccaggggcgcatgtagccgcggctacgccgactcggcggcctccgcgattttttcttttatcatgtccagcagctcgcgcaccacgatggggcggccgcagtacgtgatgccgttttcggatatcacgctctgtgcgatgtccaccagcgagccctcgcgctcccagtactcgcgcgcgagcacctccttgtacaacgcgcggtggttggccacgtaccgcaccagcgtctggatgttcttcacgcccacgctcttgaggtcctgcggcgagaacttctcgcgcagcgacacgaagacgtcccggacgagcttgccgatctccacgttggtcttgaactcgttgtacagcaccacgtagagcttgcacacgaccgtagcgaacttcgcgggcttgagatccttgttctggaagaccagcatgctgctcatcaccttcttcatgaaggccaggtacttcgcgcggtcgccctcgacgctcacgctcccgacctcgaggtcggacacgcagcggattccgtgctccgcgctctccgcggagacgcgcagcagctcctggtactccttgagcttctgcttgtccgtcatcagcgagttgtcgaataccgccaccagcttgagcacgtagttctcgtccgagaagaccttgttcagacacttcaccaggaagctgtagtggctctgcaggatcttcatgaccgcgttggcgccgctggctccgcggacgtgcgatatcatctccatgatcttcttagagtcgtcgatgatctcctcggtgtcgttgcgcatgttgcggtacattgcgttcagcgagaccagcgtctgcgcggccaggagcacgtcgcggaacacgcgcgcgaactcgcgcttctcctccgcgtcggtgatgctgttgtacacggacttcgccaccgcgttcgacttcaggaaccagaaggagagcgcctggtagttgaagtgcttcatcagcgccagcacgtccgcctcgctcatttccggcgcaatggggcacaccgagctctcgagcacgggcaccatgctgacgagcgtgtccacgtccgtgtcgaagtccaggcagtccacgcagagcccggtgccgcggctcaggtgatcgcggctgatgtcgtagaagcgctcgtagcaggtgcggaggcggtccatgtcggctgcgttttagagagacacacactcttgaattatggctgcgggtagaactcctgcagcagcgccggcgcacgcgcggagtccggctccactcccagcttcagcgcgcagttcacggaccaggtcttcatgaagcggtcgggcgcgtccgtgaccacgtgccggaagagcttcgcgaagtggcggctcacggcgttgggcacggtcgcgttgcgcacgaaggccgtgaagcgcgaggtcagcttcggcgcgaagcgcttgccgtccacgaagaagcccgaggtggtgagcgagagcccgttctcctcgcgcaccacgcgtcgcgcggccttgtgcggaaacatgctcgcgaggcgcccgctcgcgtcctggtctaggtggatggcgtccgtggccgcgtccttgcggatgcgcaccacgtcgtgcacgatctcctggatgaggatgcgcgtggccgcggtctccgccagccgcatggggaagtagaccatgtccccggagatgagcacgttcccgctagcgtttacgtagctcactatctcggacacggtgcgcagacgcacgatcgcgccttcgcagcagtgcaccacgtagtacccagcggtggcgcgcaggcgcttgttgtccgcctcgaagtccgcctccaacccctcgttgaagtacttgtcgaatatgatgggcaggaaggatagttttgactcggtgaccacctttccgaagttgaggatgtacgggttcagcgcgctgcggtcgacctcttcgtcgtacacgcaggacttgaaggtgtcggtgtgcgcctggctgcgcaggaagcagcacggaatgcagatgcgctgcaggcggtggaagatggagaggaagcccacgctgttgtagcgcccgtcggggtccatgcacgaaaacatgacgccgtttccgtttacgaagacctcgcgcgtctcggacttgaagaagttgttgctgaccttggccatgttcgcgtccagcgactgcacgatcacgggcttgcggttcttggtcttggtgttctggcagatgcgcgaccagtacacggtctccaccttggtgaagtccgaggactgcttcacgttgttgaacatcacgctgatggccacgatcaagaacgtgaagtacttctcgatgttcgggatgtagttctttaccttcacggacacgtgcgacttcgcgaggatgatcgagatgcgcttgtccgtggagagcaggatgttgttggtcgccgtctccacgaagatgaagctcgtttccatgtccagcttcatcttagacgtgatggtcgtgttcagcgacaccttgtaggtgatgtcgcccttgacgcggtccatctttacgtccatgctctcgatgagcttcgtgaacagactcacgtcgttcaccgtgagcgtcttcccgtcgctcgagatgaccaggtcgccttccgggccccacaccgagaggttcagcggctcgtccaccagcaggaagcgagtccccgtcatcgacacgaagaagtcgtccgtcttcgagagcaggatgtcgaagtcgcccacctccgctcgcttgtccggcgactgctgcgcgatcgcgcggagcccggactcgcgcaggttcgtgcggaagatgttgttgaacttggtctccacgttcatgtttaggtcgaggttcgcgaactcgcggatgagccgctcctcgaacttgaggatggagtcgttgggctcctcgaaggagccgaactccggcgcggaggtgtccgccgcgcgcgccacccagaccaccaggaagttgcacgcgtccgcgtacgcgttgtagaggatgccgtccgtgcggatgagcgttttcttttgcgtgggcgagaacgggttgaagatggtgttgtccacgtagctgtactccaggttgttcttgtgcgagtacacgatgatctcgtcctgcaggcccagcaggctccccaagtaccccttgagctgccgcacgcgcatggtcagcaagatgtgtctgcgcacgtgctcggggtccttctggatgtactgcttcgcgaagaagtagatcggcgaggcctcgtccacggagtcgtacagcgataggtacagcacgcgctcgatctcctggtggcgccccaccagcaccaccagctgcggcgcgacggtgtagagcatgttcgcgcgggcgtatttatagccggcgttaaactgaaataaaatacgcgggtcgcgaggcagcgccatgttccagccggtgcccgacatggccgccgaggccgacatcgacctcggcgacgtcagcgtggacgcgacgcgcgcgggcgcgcgcgagaagaccgtcttcttcgcgcgcaacaagcgcatgtacccgcaccgcagcaaggacgaggagcgcaagctgtcgctgggcttcttcttgcagcggctggacttcctcacgtcgcgcgaggtcaacctgcagttccggtcgctggacgcgctgcgcaccgagaacgtcatgaagaagaacaacgtgctcgtggcgccgtacatcctcatcgcgacgctcgcggggcgcggtttccgcatgacggagaccatggtcgagctctacttccccgagctgtaccgcgagaccagcaagcgcttccgcttctgcgcgcagataaaggtcatccaggacttcctggggttcgcccacgacagctaccacacttacgacttcgagacgtacttcgcgttcgtggcgctggtgctgcgcggcgcggactctgcggccgaggccttcgacgtccgcgccgagagcgggcttgtgcgcagcctcaccgagatcacgtaccggctctacgtgatgcagctgcgctccgacgccgcgcagtggagcgtgagcaccggcgccgtagtctcgcaggcggtgaacaccgtgctgtcggtcgtcggcgacctcgctgcgcgcgcggaggccgagcggctcacgcccgtgtgcgacctcgcgcgcgagaacccgctctcgctcgaggacctgcgcaagtacggcccgcggctgcgctcgctgctcacgaccatggcgcgcgcgcgatccttcaagacgaaccggcgggacaaggacgcgctgtcccggttctgccgactgacggcgggccctagcccgtctgcgtgccgcgcgtcgccataggcgtcggcgcgcgctcgccgccggaacactcggggtcgctgaacatgtagatgagcgcgacgcctagcagcaggtacatgatcatgctgatcacggttttgaacacgacggcggcgaacgtgttggaccgcagtcggtgctcgcagaagtgcatgaacaggtgccgcatgaggtcgatggccccgttggccacctggaaaagggcgaggccgccgatggacttgatcaccgtcacgtagcacggccgcattccgacgacgctatttactcactgtcaaaagaaacggcgccatccgaccggaggttgaggttgcgcttcatgttgttccagtacatctcaccgatgctcgagtagtacgccgtcagccgcgatattttttctcgcaccagctcgtaggccttctgcatctccgcaacgccgatctccgcgtcgcccacgtaccggccgctgcggcgcacgatcagcagcagcgccttcaggttctccagcgcgatcatgtccatgtacagcgacttcgagagctgcacgaagaggttgtaccgctccaggatgctgttcttcacctcgtccgcgatcgggactccgaagatgcgctccgtggtgtacacggactgcgtgagctgcttgaagagcgcggagatgcagcaggtcgcgcgcttgacggcgtcgagctgcttctcggagcgcgcgctcgcgatgctcagcgcgctgttcacgacgttgctcgtgtcgcgcacgtagcgcgtcttcagcgcggcgttgatggcatccgcgatctcgttgctgctcacgctcgagtcgtccgagctgcccgagacctcgtccagcagccccgagatcgtgatgtcgggcgagccgccgacggtcaccagacggtcgagcaggttgcagggcatggacatgaggatgccctcgctcgagaggcagccctcgtcgatcatgctctgcaggttgcgcttgaaggccgtgttttcgggcatgaacccgtccacgctcatgagctcgtcgaccgtgctcgcggaaaagatgcccctcacgttgatgcggtccagcatgcccatgtcctgcgagcacagcaccaccgactggtcggccgtctcggcggcgtccttggcgccgccgtagatgatgcgcggaaaccgccagctcgccggaaaagagaaggagggaaaccggcactgcgcgctcgggcctcggtagccctgcgcgtcgcgcacgttggtggccgtgaccatgaactgcagcaggtcgtgcgcggacgccatgatcttctccacctcctccttgctgcagcagaccttgcccaggctgcgcgcgatgttcgttttgctcactgagggcgagaccgtgacggcggtgtgtcggcggctgccgagcgtgtacgcgctcacgctaacgcggtaccccatggcgccgaagagcagcttcacgaagtccaggtagctctccttattgatgtagtgcggcgcgcccttgtcttccatcctcagcccggcgtaggccatgagcacttccttcatcgccgtctcggggtccgagttgcacaccagccgcagcatctggaagaactgcatgaaggcgcgctgcgagagcccgatgtggtggttgggctgcgtcgaccggcgcgggaactccctgggcgtcatggcgttgatgcccgagagcgtctccatcacgagcgcgcccacggtcttctggcccatgacgcgcgggtaaaagcacacgcggaggggctccttgccggccgcgagcgcgtccgagagcagcgagcagtacgtgacgttgtcgtggtcgaagagcgcgaaggtgtagcagacggagctcatgaagagcgagtcggcggtgctcatggacttgaactccgtgtacgcgattccgtcccagaacaggctctttccgggcgcgatcagcggggacgcgcggtcggcgcgcatcagcatggagagcagcgtcacgtagtaacggatgttggcggaaatgtctacgaactgcatgccgggcgaggccacgcgcagggtcgcgcccgaggtagtgagcacctccaggctgtccatgagcgtcacgctggggtgcagctgcgcaaggcgcgccagctggctctggtagaagatggacacggcgaggctggccacgctgccgcgtgccatgcgcaggttttgcccgttgaaggtgagctggcgcagcgagaacacggagtcgaagtactggaagaaggtgagcaggtacttgagcggcatggtcgtcagctcggtatccacctgcggcgtctgtgcgagcacgattccgttcttggccgcggcgtcggggatgtcgtacatggcgtccattctggcgcgggaggcgtcggtgagcagcgcgcgcacgttgagcagcatgagcaggtcccgcgctagcatggtcccgtcgaccagccgggcgcgaaagccgatctcggcggggccggcgatgttggggtagatcaggttcagcaggtacgtgttgtcgaagctcagcgaggggaaggagatgggagacttcgccgggaggccggtggggtagcgcacgtagccgccgcagatgcgcgcgtgcgcctcaaagctggtcacgcgagtcttcagcaggttgcgggtgaagggcggcacgtccttgaaggactgcgtgcagatcacggggttggcggtgtcggtcagcttgaggttggtgggcttaagctccgcgaagttggggcccagcagcacggggacgaggtgcgagttggcggcgctgtcgagcaggaagttgatgccgaactgcttcacggcgacctcggtttcctcgtcgctggcgagcttctccgcgtcctcgaggaagagcgcgtccagcgggtgcacgtacgtgcggttgacgtcgtagctgggcttgaagtccgaacacagcgtggggagcacggtggagacgagctggaacatgtattccgcgccctccacatggtgcaaggccatgtgcacgtttggggccgtcatttatttagtattaaatgacggccgtaccggtaaccgatattcctggagactacgggccgacgtccttttcggaggacaactacccgctgaacaagcactacgagctcaccaaaggccagctctcgatcctgcgcacggtcaacgacaagctgctcgcgcgcaccgtgcagcactcggacggggagagcgatgagagcgaaagcgaggaggacgacatctccagtccgctgccgccggacgaggaggagccggactcgtgcgtggcccgggtcatgccgcgggacgcggacctggcggcgccaaaaaaggccgacggctacatcattgccgccgagcagcagcgccagcagcgcataaacattctggtatccgatcgagaggccgtcgtggagcgggagccggttcagacgtcgttcgcgcgcgtctcggctatcccgatccacggggacggcgcgcgccgcaccaccgcctccttctccgcgaccacgccgtcgctgggcgccgtgttcgacgacgccaagcgcgtgcggctgctggaggaggaggtcaaggagctccgcagaaagtgcgcgacctctcaggataacggaaacctggagaacttcaccaaggtgctgttcggcaaggcgccgcgcgcgagcgagctgaacaagcgcgtggtcatcgtgaactacgccacgctgaacaacgtgacgctgtccatggaggacctcgagaagtgctccgacgaggaagtggaccgcatgtactcggtcatccggcgctacaacgagacgcggaagaagaagatcctggtcacgaacgtggtcatcatcgggatcaccgtgctcgagcacgtgctggtgaagcttggcttctcggaggtgcgcgggctcagcgccgacctctcgtcggagctcatcgacgtggagatcggcgaggactgcgagcacatcgcggagcgcctggggttcgggaacagcccggtgctaaacgtggcgctcttcgtggtaaagctgttcgtgcggaagctgaacctgatctgatcaacacatgccgccgtcgaggtccatggcgttcatgaggttggaggcgcggcggcgcgcgccggtggaagcggtggaggcgctcgaggtcgtggagcagggagtgttgctggaggaggcgcggcggcgggagctagaagcggaactcgaggttccgctggtggtgctgcggcgactcgtgccgctcgtgccgctcctgctagtgccagtgccagtgccgctgcggcgtgaagtaccggtgccggacctgccgctggagcttttcttgcggccgccgttaacgctgtcgatgccgagcaggtcctcgcacacctcgccgacggttccctgcacgtccaacttgccgttcttgacaaccccgtacacgatcttgccgcagttggacacagcctggatggtggtctcgtcgctgtcaaaggcgttcattccgccgcacccgccgtcgttgtttcttcgagaaggcgcgccgctgcggcgactcctggtgctgccgctggaccgagttccggaggacctggagcccgtggaccggctgccggtcgacctggtgccggtagtgcgctttctggacgaagaggaggaggcgcttccgcggcgggtggacgaactagcctccagcgcaccggcgccgcccacacaatccacgtcggcggcggcgcctccgcgaatgacctgctcgttgttgagctgcgtcaggagagatcgcagctgcggcgcgatcttctgcaaggtgctcacgtagtcgtcgtagctgctctgcgggcgctgcgccattttttcggacgccatttattacgcggaatatctacgacgacgcagcactgaatcggtttctcgcgacgggagattccgcggtcggcgccggtgcggggttgtcaccgggcgacgaggtaaccagcgcgtggaaggcgcgcacctggtcgtccgtcatcttgtcctcgaacgaggacgcgcccggggggagcaggtccttgttgcgcggaacggcgggcgccgagacgcacgaccggcggtacatcatgatgacgatgtagcacacgatcgagatgacgatcacggtcagcagcgcgtcgaggagccccatttattacctgtatatgcccgcgtttaccgggcggtgagctcaatgtcggtgttgtttagccgggcgtacgggacgctgccggagcacttcctgtacatgctgaacacgaacagcccgagcagcagcacggcgcccactatgaagcaggttacgcacagcgcgcgccacacgtagtcggtgacgttgttggtgttcttgctgaaatccacgaaggcgaagacgcaggcggccgtcagcagcagcacgccgcatatcagcactccggagtagtaagagctcaaggtctcgaatatgtccatttatctgaggagaaatttaaattactgaatggacgaagtggaatagaaaccacgagaacacgacggactgcagcacgaagatggtgctcagcttcgtcttcatgggcatgcagaagttcgcggccagcgccatacagaagatgaacacgagcaccgccgggtcgtagtcggacaccatttacactacgctaaaaggcatatctcggcgcgcgacgtccacgagcaccagcacgcggacgcccgcgggcgcgccggcggcgaccgcggcgagctgcccggccgtggggttcaccagcagcagtgcgcgcgcggttcgcgggacggggtcttcgtaggccatggtcggcgtggacccgggacgcagcggccgcccctgtctgtcgaagaggccctcgggaaacgaggtgcccggaacggccacgacgacggtgtcgctatctagaaacatttatggtcttggtttccacggatcgcctcgagtagaccgccacgaagtagaagatgacgcccgccgcgagcgccgccaccaggaagggcggcacggcgggcaggttcgcggacgcgttgtcgcgcacgccggggtccgggtctgcgtagcccgcgcccacggccttgccgcagtcggcgatcatgtgcgcgcgcgagttctgcatgaccaggctgtccacgtcgatgcggcagcccacgtagcggcaccgcgagcgctgctcgtcctggctgaagaagagccacttgcggtcgcgcgactggtccgtgcactcgtgcgcgcgacagacgcgcgggcccaggtacttcccgagcgtggtgcccgcgacacacgcgcactccggcgcggcgcggtgcgcgtcgcagtagcgccgcagcgcggagtcgccgaaggcgaaggaggcgggccgcgccacgcgcacgaactccgagcagaagcgcgcgtccatgtgcttggcgcagagcgccgcgtaggtgtccagcgccgcgtagcggcccgtgcgcagccaggccatgcactcgggcgcgtcaggctccaccgcgcagcggctggccataacgccgtcgcagtgcgcggtcttgtacccgttcgcgaacacggacgggcacccgggctccggatttgtgcagcagcgcgccatggcggcgtccgtgggcggcgccgaggcgccgatctcgaacgcgcacatggtgccctggcgcaggtacggcttcgcgatctcgggaacgtagtctgcgcgcagcagcgagcccgggcggaagaagagcgagtcgcagggcgggcctcgcacgagccgcgcgcggctcgccagctctggcgagaggaagcgcccgcactgcccggggtccatggtcggcagcagacagaaccgcggccgtacggtcttcagcttcgggtcggagaaggtttctgattcttccgcgaaggcgaaggtgtccgtggcgctcgtgtgtgtgacgcgcagcgcgtactcgcctggcgtcggcgtgtcgagcacctccaccttggatacggtgtcccccatttgaagacgctatttacgccgctgcctactcggcgaagaataggtcctccgacttggcgcccgcgtacaccgggcaggcgggcgcggcggagcgagtgcgcacgataccgcggccagtgaggcggaaggcgtagatggcgaacagcaggccgagcacgatgtacatgaaagtggtggcgcccacagacccggtcacgtgcgtcacgatgatggtgacgatggacatgatcgtgcacacgatggccatgccggtgttgttggccgcgtaggggtgcatgatctgcatggccgcacagtatccgatgaccaggcacggcagcgggaggataagtgaggcaatacctatcattactagagcgagcacgggggtggacgtcaaggccaatacaaaaatcacaatacctgttagtatgcggatatcctcgtactggaggacgctgtaaggcgcgatattccctccgggcactggcctggggttagccgggactaggggggagtcggcagtgccggggtccttggggagaaaggcattctgctcctccgggctgaagagctcggcgtcctgaacgccgccggcggtgaactcgtcgttatagtaactaaagtagctttccatttatatgttgaaaaatgtttggaggcgtacaggtggacgacaaactctacgcgtacctaaaaaaactcgccggacgcgggcggccgctgtgtctgttccgcgacaacggcgagttcgtcgaagtcttcgcggggtccgcgttccgctttgtgctgcccgtgggcctcttcgcggacctgcgcgtgcgcacgcgcggcgtggccttcccgaaactgcgcgactccgcgcgcatgcgcggcgtgcgggtggacgcgcacacgctgccctcgctgtaccccaaccagcgcatcgtggtggacgaggtgctcgcggcccgcgaccagttgctggccgcgggccgcgccgtgtacgtgacgctgcatctggcttgcggcttcgggaagacgctgaccgcgtgccacctcatcgccacgcacggccgccgcgcggtggtgtgcgtgcccaaccgcatgctggtgccgcagtggcgcgcggccgtggcggagctgcgggtgcccttcgcggtctcctgtgacggcgcggcctcgctgctgcgctcgggcgagctcgaccgcgccatggtggccatcgtggtcagccggcacttcgccaacgacgacttctgccgcgcggtgagccggcagtttgacgtgctcgtgctcgacgagtcgcacacatacaacctcatgaacaacaccgcggtctcgcgcttcttgaccaagtacccgccgcccatgtgcttcttcctgaccgcgacgccgcgcacggccaaccgcatctactgcaaccgcgtggtgaacgtgtccgtggtcagccgcctcaccaaggtagtgcgcgtggtggacgccttcttcgagccgtacaccacgcccaagatccgcacgctcgagcgcagcctcgagggaccccagaacaagtaccacgtcttcaccgagaagatcctcggcgaggacgtgcaccgcaacaagctcatcgtggacaccgtggtcgcggccatggccgcgggcgaggcgcggcgcgtgctcgtgctcaccaagctgcgcgaacacatggtcgggctgcacgccgcgctctgcgagcgcctcggtgcggagacggtctttctcggcgacgccaagaacaggaagacgcccgaggtcacgcgcgcactgcgcgacaaggaccgcttcgtgctcgtgtccacggtcttcttctcgggcacgggcctggacctgcccaacctggacgcgctcgcggtggccgcggccgtgctcaaccgcatggtcatggagcagatgatcggacgcgtgtgtcgcgagtcgcacgccaacacgcgcacgctgttcgtgttcccggactcctccgtgcgcgcgatccgcgacaccgtgtctgcgtttgcgcagcggctcgtggcgctggcggtggacgggctgggcttcgtccgcgagcgcgccgccgccggcgcgaagaacgagccggcgctgtacagcgccatcagcgggcgagatctcgcagcggtgtaagcgcggacccgcacgccgcgcacgagagcgtgctggagcaggcgagtcccagcgacagtgtggacagcctgtccacgtccttgatgctcaccagccgcgagttgcacgacgagcacacggggtcgctactatcatcgaccaccgtggtgacgcggcggcgtctgcgctttttgtttccagcggcgacatcgaccacgcctcccttagagccccccttcgcccccgccttagctttcaccgcgctcatcttttatttatcataaaaacacgtctgcgtacgcgttcgcgcacacgtcccgcaaatccgcgcgcgcgccgcagcgcgtgaagcgcgcggcgtccgcctccgcgatccgcgcgcacggcagcggcgcgcccttctcgtccgccatcacgcgcgcagagatcccggtggcccccagcgcgtacgacaccaccacgtcgccgacgcagcggtacacgttgccggagccggcgaggcggtcgaacgcggcgccctcctggcgcagcttgtcgaatatgcgaggaacgaggatgttaaaaatgagaacgaaatagcagatcagcaaaaacagcgagatcatgacctccgagagcgatttatataccttgaaagagctaatacgacttcgggactcgctgcacctcgccaccggcgccgccgtcgagcgctacaacgcgctcgtggagtgggccgcgcgcacgtactggacggtcgcggtgctgccctccgcgccgtgcgcctccatcgagaagtactactgcgtgtgcaaacccgactgcgcgctcgagcccggcgagtactccgtgagccggctgcacttcggactcacgcacgcctgggtgcgcggcgccgccttcaactcggccagcggcgccgaggtcgagccgccagaggaggtgcgtagggcctgcgaggcgctcgacgccgccttcgcggacctcaccttcgtgcgcttctcggtcttcggccgcgagtggacggtcgacgacgccgtcacagaccactcctcgcgcgacgaggtgctcgccgcgtgcgccgcctccggcgtgcgcgtcgcgcgcacgctgcgtgtgcgcgtgcgggcgggagagtccttcgcgcgcgcagacttcgacgcggtgcacgccgcgctgcgcgcggagggcgacgtcgctcgcggcaccgcggtctgtctcgcgctgcgcgggtcatcgcgccgctggatagcggaccgcgcgcctcgatgcttcatgcgcgtgcgccgcgtggagctcgagcccgtggacgcgcggcaccactgcccggtgctgatctccgcgcgcggcgaccgggtgctctgccgcggcgtggggcacctcgcggacgcgcgcgcgcgcgagggcgtcttcgtggccgtgcgcaggtacccggagtgtctggtgctctgcgacgaggcggccgccggcgcggcggagtgctcgcgcgaggaggcgctgcggctgctggtgcgccgcttcgggcgcgacttcgccgtcagcgaggagggctacgtcttccgcgtgcaggacatggacttgcgcggcgtgtccgcgcgactggggctcgcgccctgcgcgagcctggaggagctgcgccgagcggtggagcgcgaccgcgcgctgatgaggcggctgcgcgcggagggcgccgtgcgcctcgcgtgcgagtgcgtgggatacccgcgccagaacgcggtggagctcataaataatatgcgctttcaaataacggaagaaggcgcggtggcgaactttgagctggcgaacgcgagctgtctcggcaacccgaccgcggagtccatcttcgcgagcttcgcgcagttcgtgccggtcttcaacgtgctctcggcgatcgcgcgcgcgcagccatgatcgtggcggccttcgaccttggcacgcgcaaccccgcgcgcaccgtgctggaggtgctcgacggcacggtgcgcgtggtggacgtggccaagctggactggagccgcgactgggagaagcgcgtgcaccgcgacgtgaccgccttccccgcgaacgtggtgctcgtggagcgccagtgcaagatgtcgcctttttctaagttcatatacttcatacgcgggctgctctacgacgggcggcgccgcacgcgcgtgctcgcggtgccgccggccatgaccggcagcacctaccggcagcgcaagcgccgctcggtgcgcaccttcctcgcgctcgcggagagcttcggcatcctggacgccgtgcccgcgcggaagaagctcgacgacgtcgcggacagcttcaacatggccatcaattacgtgctccgaacaaactgaaatacgactggaacgaataagtcatgctggcgctgttcgagttcctgcggtccgtggaggactgctaccggcgcaccatcttcaacttccacatcgcgcacagcgccgaggcgggcgatgtctacggcgtgctgcgcgaccgcattttggcggccacgcgcttcgaggaggtagcgccgccagggctcgcggacgcgctggccaaggtggtctactgcgacataagcaccaccaagcacctggtcaaccacgcggccttcgcggcgcgcgcgcggccggcgcggcgcggaggcagcctcgcgcagttcttcgacgtgcacgtgggcgaggacgcggagagccgccgcaccgcggagatcttcgaccgcgagcgctcctcgctggtctcgtacgtgaagaccacggccaagcgctgcaagatcgactacggcgagatcaagcgcaccatccacggcgggcggcagacctacttctcggggcggcgctcggacgacttcctgagcaccaccgtgcgcgcggacccgagcaagccctggatcaagtccatctccaagcagctgcgcgtggacatcctgcaccacgcgatctgcacgcgcggcaagagctccatcctgcagaccatcgagatcgtgctcacgaaccgcacctgcgtgaagatattcaaggactcgaccatgcacataatcctctccaaggacgaccgcgagcgcgggctcgcggacctcgcggacaagctcttcgggacctacgcgaccaccttccgcgtcatcgcggccatcaccggcaacgcctgcttcgcggcggtggcggacgcggccgcgcgcgtggtcgcgctcccggacgcggacgcgaagctggcggcggtgcgcgggctcgcggagtgctacggcgtgcgcaacttcaaaatcggcatgttcaacctcaccttcacgggcgccatcgagcacacggtcttcccctcgctgatccccgcggagagcaagatcaagttcttcaagggcaagaagcttaacatcgtcgcggtgcgctccaccgaggagggccgcgagtgcgtggagcaggcgcaggcgctgctcgcggccatgcgcgagcgctccgcgcggctcgcggccgcggacgtggccaccgcgagcgtggacttcctcaaggagctgctggggccatagtgaaataatactgatttcttaaatatggagcaggcgctcggatacaagtttttgttgcccgaccccaaggacgacgtctactaccgcccgctccacttccagtatgagtcatacgccaacttcatcaagcaccggcttaaggacatcctcacggtgcggcgcacgctgctcaccttcaagaacggcaccgagtccatcgtgctcgagatcgacgacgtgaagatctcggcgccggagttctcgcccatcgtggccagcatcaagggccacagctacgaggcgctggtcaccttcacggtgaacatctaccggcacgtgatgaccaaggacggcctcaccgttaccaagatcaacagctacgagggcaccgactcgcacctcgtcaagctcccgctgctcatcggctacgggaacaagaacgcgctggacccctccaagttcgtggtcccgaacgccatcggcggcgtcttcatcaacaagcagtccatcgagaagctcggcatcaacatgatcgagaagatcaccacctggcccaagttccgcgccgtgaaggccaactccttcacgctctccttctcctcgatctcgcccgtgcacgtgatgcccgcgcggtaccgacactacaagatcctgctcgacgtgaaccagcccgacaacttcgtgatctcctccgcgaagaccttcatcaccgtgaacgtgatcgtgatggtgcagttcctcgcggacgtcacgctcgagttcgtggcgcgcaacctctgcttcgacatgccgcccgaggccgcgcacctggccaccgcgctcgtggagagcgcgaagaccgtgcccgcgggcgcggacgtggccgagtacgtgaacgcgctcatcgcggccgagcacgcgaagcagaagtcgacgctgtccaaggaggagttccgctacgagatgctcagcaacttcctcccgcacatgcaggacagcgccaaccagctcaagggcctgtacctgctctcgctggtgcgcaagatggtcttctgcgtgttcttcccgaaccggtacccggaccgcgactcgctggtctgccaccgcgtgtacacctacgggcgctacttcgaggcgctggccatggacgagctcgagacctacatcgggaacatccgcaacgacatcctcgcgaaccacaagaaccgcggcacctgcaccgtgaacatccacgtgctgaccacgcccggcttcaaccacgccttcgcggcgctgctcagcggcaagttccgcaagtccgacggcagcttccgcacgcacccgcactactcctggatgcagagcatctccatcccgcgcagcgtgggcttctaccccgagcaggtcaagatctcgaagatgttcaaggtgcgcatgtaccaccccagccagtacggcttcttctgcgcctcggacgtgcccgagcgcgggccgcaggtcgggctcatctcgcagctctccgtgctcgcctccatctcgaacatccgcaccgcggacttcgtcgagctcaccaagcgcgtctgcgactacgtgcgctcctaccccgcgcgcgacatcagctacttcgagaccgggttcgcggtcaccgtcgagaacgcgctcgtggcctcgctgaaccccgcgatcgtggacgcgttcgtgctcgacctgcgccggcgcaagcggctcggcttcttcgggaaccgcgagatcggcgtcgcgctcgtgcgcgaccgcatgaacgaggtgcgcatcaacttcggcgcgggccggctcatccgcccgctgctcgtggtcgagaacggcgtgctcgtcatggacgcggaggcggagcggctcgagcgcgacctcgccgcgatgaccttctcggacgtgctgcgcgagttcccgcacgtgatcgagatcgtggacgtggagcagttcagcttcagcaacgtctgcgactccgtgcagcgcttccgcacgctgccgcccgaggagcgcgcgctcttcgacttctgcgacttcccggccgagttccgcgacgggtacgtggcctcctcgctcgtgggcatcaaccacaactccgcgccgcgcgccatcctcggctgcgcgcaggccaagcaggccatctcctgcctgagcgcggacctgcgcaacaaggtcgacaacggcatccacctcatgttcgcggagcggcccatcgtggtcagcaaggcgctggagacctccaagatcgcggacaactgcttcgggcaccacgtcaccatcgcgctcatgtccttccgcggcatgaaccaggaggacggcatcatcctgaagcggcagttcgcggagcgcggcgggctcgacatcctcacctgcaagaagtaccaggtcgagatcccgctcgagaacttcaacaaccgcgagcgcgtgcgctccgcggcgtactccaagatcgacgtcaacggcgtggtgcgcctgaacgccttcctcgagcagggcgacgccatcgcgcggaacgtgtcctcgcgcacgctcgacgacgacttcgtcgccgacaaccagatcagcttcgacatcgcggagcggtactcggacatctacgccgcgcgcgtggagcgcgtgcaggccgacctcaccgacaaggtcaaggtgcgcgcgctgaccgtgcgcgagcgccgcgccatcctcggggacaagttcaccacgcgcaccagccagaagggcacggtcgcgtacgtggccgacgagactgagctgccctacgacgagaacgggatcgcgccggacgtgatcatcaactcgacctccatcttctcgcggaagacgctctccatgctcatggaggtcatcctcaccacggcctacgggcacaagcccttcgccgaggacggctccaaccgcccgatctgcttccccagcaccaacgagaccgacttcgagacctacatcgagttcgcgcggcgctgctacgcgctctcgcaccccgaggccgccgcggacgaccccgagttcgagcaccgcgtcttctgcgagcgcgtgctcttcgaccccgagaccgacgagcccttcgcggcgcgcgtcttcttcgggccgctgtactacctgcgtctgcggcacctcacgctggacaaggccacggtgcgctgccgcgggcgcaagaccaagctcatccggcaggccaacgagggccgccgccgcggcggcggcatcaagatcggcgagatggagcgcgactgcatgatctcgcacggcgcggccttcaccgtcgccgagatcctgcgcgactccgaggaggacgcgcaggaggtgctcgtctgcgagaactgcggcgacatcgcggcgcggctcaacggcacgcacgtctgcatccgctgctccaagatgagcctctcgccggtgctcacgcgcatggactccacgcacgtgagcaaggtcttcaccacgcagatgaacgcgcgcggcataaagatccgcgtggagttcgagaagcaggacccctgcttctacgggactccgaaacggttcagcctcgcgcccgacgagtcgctgttctcgccggaggactgaacccgccgtcgcgaccgcgtcgcgacgactagcttatcgttcgactgatgcgaaacgcgcggcggcgccgcgacttagcttatctcgactgatgcgaacgcgcgacctctcgcgactttctagcttctcagactgatgctaccatatcgcggcgtgctggccccaccaccagggcttctcgccgtggctgacgcggggctggctgcgacgcgcgctgcagtagctgcgcgcgccccagtcgccgcgcacgtgcgccgggggcaggctcccgtccagcgcgtgccgcgtcacctcggcgccgggccggcggcacgtgtgcacgtccgtcttgttggagacgagcaccgcgtactgccgcatggtctctatgtgatgctccaagtgcttgcccgccatccggttggactcgcagcacgtttttgcttcggctaaggttttttctagaggggatagtagcttatccacgcgctcgggcaggacgcacgcggagccgtcgaaccctactttgaacggggtcaccttgatgttcccgtcgtagcggtcccacagcatcctgaggtaggttgtaccgtcggggtctgggtctgtccacactctaagcttttcgctacagcggccgtcgtacgtaagacggtctctacgctcgtagtagtttctgcttatgttgttggggtctccatgctcgtagtagtataaatcgtacgcgcctggcttttttaagtcgttttcgtcgttgctgacgtgtatcacgtcgggataataggatatcctaactgcactacaatctatagtatttggtctagtaagctgttcgagatcaccttgttcatcatgatctactgatttgtacacggcaccgtcgtgttccgacggacgtatgaatatgtccatggtaaacgatgtacccactttggaaaacgtatcccatgcagtaaagcatagtccgtccattataaactcaggaacactcataacaaatcgaaatctgtgaagtttttcgaacaccacttttacatggtctttgtcacgaacatcattgccgtttacttcagacatgaattgaaggaacgctaaagagtttcttgtttcttcatgaatctttccattatacgtccatccagtttctagaattctatatatgctttttgcatcgaccccgtaccaccagtacatgggaactccgaaatatatagctgggtttgagtaccaatgggcaagagtgcccattgcgtttaaaaagtcttgacaaaaaaatgcagtttttctgtcgataacttgacttggactacgttcgtggacatcgtacatgtccataattggttcattggtaacggttacatgacccgtcattatctttttaacaatcataagatacagtttgcctaaagtcgaaatatgtataacgttaatttttacatgttctcctaacgtaattgcgtttttacttagccattcgtcgtctacaaaaatcttacgatacataggatttctctctacgtatcttctaaagtatagatttaccggtctaccggcgacattagcgccatctatagcaggagcaagctgtatgtatcgtcgtataatgtctcgtataagctttctgtcttctctgggaatacacgacacggaacttagagactggtgccagtgtctttcaaccaaagacttgaacctagcaaccaacgcgttgtcactctccatttataattaaataattatcccaacttcgtatgttaatccttattaccagatagcaccgctccttcctctccaccacgtactatctaaaggatacctgtaagggtaatgtctggataacgggcgtgtgagccaagacgtgttatggtgtcttccccaccaacggtccacttctctaactaccggagtgctagacgttgtcgatcccactactgttgtttctccattacctgtaatctttgaagcgcaacaagtgttagtctttgccaaatcttctaacggcttaattaggtcgttaagtctgtcaatatccatgcactgaggtgtatcgttaccagtttccccaactttgggaggaacttcctgtttagtgtccaaataacccataaacctgtctctaagcattcttctgtacgtgcttttgtcattgtctatgtctcccaaaaacctgtgatttacgcatccgttgtacgtaagtctgtcccttcgctctcgctcgttatttcctacttcttctattgtactgtaatgttgatagtccaagtaatagccactgttttcatgatttcttgtaaatataatcggtgttttattattgacatcgtgttgcctactgtacgtatcttccatggatctaggaacttgtctagaaaattgaggactagaaatacgccttccaaatcctggatgataaaccaaacgcaatgcactacagtcgacatcgtcactgtctctagttatcccatcaacaactccttctttgtgatgctccactgttttaaagttaactcctctgtatatgttgagtgttaaaaaaatatccacattgaacgcagttccatattttgttattttatcccacgacgtataacacaaaccatccataatgaattccggaaccgatactacaaaattagtatcgaacttatcaaacgaaatgtttactcgcggttgtgcttcgtattttttgtcatacacatctgtcatgtattgtacaaaatctatagctcctctagcatcgggcatgctaacgtctggaaaatcattttttaactgttctagtacgaactgttgattttggtcatctctccaccagtacattggcaatcctatcacaagagacttatttttataataatttgctacagaagctatatgccacatgaaattatagcaaaaataatccatctgtatgtttaaaaccggtttactagtctgctgagagtagctatccatgatagtgtttccctcgccaatacttcctgacattattctgtaaacaaccattaacaaaaatcttcctaccgttgttatgttgtcaaaagttcttatgttttgagcactttcgagtaaccatatgttattttcaaatatacttttgtaaactggatttctgtccacataactctttaaaaatagatttactggaaggccgcttgggttatctccctgtataggcggcgcttgctttatgtattcgcgtaacaaatctcttacaacttttctagttcttctaggtatacatgacctattattcaaaggctttgtccagttagcgtttataaacgttgtaaagtcactgactagcttctccatttataattaaataattacagacggcaacacagcggttatctaatatctgctgtatcctgtctgtacatctatttttctgttgagatcaagaagagctttacgtagactctccaagtgtctttctagtctgtctaaccggttacctgtttctctgcagcaatcagttatagttttgtaactgtctaacaagcttacgaggcgctcttccacactttctttagttggagctccagccgcgtacactccgttggttgaattgcctgtatcatcaggctgagtcaataggttttctccgtcattttcatccatattgagtccaacgaacacaaacgagtaagtgttcctctatttaaagtattgattttagaaaaaggcaagcctcgctgccctgattcggcggcaaacacgggttgaacacgcggaagtcgctcgcggccgtgaagatctcgtccgcgcacgcctccacgctcgcgaagcgcccaggcgagacgccgtcgtgcgagcggaacccgaactccgaggccgccaccgccgcgcccttgaagagcacgcagcgccacttcttgcgcacgtcgaaggcctcgtcgttggggtcgaacacgcgccggtccacgcgcgggccgcccgccgtgcgcgcgaactccagcgccgcgttcgccgcgttgaactcgcggatgttgtcgtagttctcgtagacggcccagagctgcagcgccacgaacatggcggccgccgccgcgagggccacgcagagcgcggacaccgcgtccatcttttatgtgcagaattattcgttggcgcggagctcgcgcagctccgcggcgcgcagccgcgcgaaggctgctttgagcgcgcgcagcagctcctcggtgtccgcgcgcagcatgtcgaagcggtggtagctgtccaggcgcgcgcggcagccgaagaagcgtgcgacgcacgcggtgacgatgtcgttcacgtagagcacgcccgaggccgtgcagtacacggagcgcggctcgcgcgggtccggcggcacgtccacggcgaccgcgtgcgcggccacgtcctcgagcaccttgcgctcgagcacggcgaggaagtcgcgcagctggcggcggttgtccagccaggcgtaggtggtcgcgaagagcgtgagccgcccgcgcggcgcgatcgcggtgtagggcgcgtacccgcggaactcccgggggtgcacgaccttgacgttctcgtgctcgcgacggaaggcctcggtgtcgagcagcgccgcgagcgcgtccacgagcttgtcggagacctccacgcccgcgccgaaggcgatgagctcgatcttctgctcgctcttggggcggaagtcgtggaaggtgtgcagcagcatctcgcggagctgcggcggcttctcgacggcctcgagcgcgtcgccgcggacgaggaagtagtcgaggtcgtgcagcgagacgtgctgcccggcggcgctctgcacaaacttgaggaagacgcagaggcccgcgcggcgctcgagcacgtcctcgacgtgcgcgtggaatacgtgccgcgagggcatggcctcgatcgcggagagccactcctcgttgacgcaggtggtggtgttctccagcaccacgccctgcgtgagcgcgggccactgcaggtggaaggcgaactcgtgcttgatgagcgaggccacggccgggtccaggtccacggccagcgcggcctcgccgacgaggggagcgtccgccatcacgcggaggacgcctggcccatctcctttttcgcctttttattcaggatcattattctttcgttgacgaggtccatgagcatcttgatggcggcggccgcggccgccgcgtcgccgccgcacatctgcgcgatgcgcgtgagcatgtgcagcagcgcggcctcgtttaggtcctcctccatttagaggccgtgagggcgcgcgtcgtcgcgacgaggggacgcctcccgcggcagcgtggtgcgcacggcgaaggcgagcagcgcgccggcgcactgcgtgagcacacactccgcgagcgcgacgaggagctcggagggcgcgagcaccatttagaggcgcgcgcgggtttaattgccgccgtcagagtcggcatcatcgcccttgtcgccgccgtccttgcagtcgcccttggtctcggcgtcgacgatgtcggcgagccgcgtcttcatgtgcgagaactgcgcgagcaggatgccggggtcgagacagcgcttgacgacgctctcgtcggcgaagtcgtagcagatgcgctcctggttctggcagaacaccgagtcttcgatgatcaacaccctcctggtcccggccgaccgcatgatggccatggcccggatgagcctcttcttcgatccgcgtatggacatggaccggagcacgttctccacgtcggagtcggagacgttgcagcagcagaggtgcgtgatgctggcgcgcccgttgacggggatgtgcttgtaggtctggcagagcagcaccagcgacacgttgatgtgccgcccgtagttcatgaggcccaagagtgtgggcgaccgcgtctgcgtgtcgcccatatcgtcgagaatgatgaggaacttctgcttcttcgtctgcgcgtgccgctcgatcttgcgcttggcgaccgagaggttgtactcgagctcctcgtgcgtggtgaccttgtggatgtggtccggccacacgaagccgtcgtaggcggcgttgtagacgggcgtgaagagcaggatgtgcttgaagcggcgcacgagcgtgcggaagagcgagagcaggtaggcggtcttgccggagccggagccgccgacgagcgccatcctgaagggcgcctcgatgagactctcccgcttgaagcgcacctcctgcacgacatccatcgtatatttactgtcactaaattaccggctccgagaaatatagaaattagagcctcctagagcacaccgaggcgcatcggcaagatggcacataacacgttcgaaaacgatagcgagtcggcagctaacaaccagtacgtggcgtcagtcaagcgccagaaaatgattcggcgatacattaagatgttcttccggttcgttacggcgatagctatcattgtcctggctattctagttgtgattctgtcgctatctctagacgaatgtctgcacagagaacaccctcatgactattcacatgtacaaaattcaacatgcgacggcattactttaggtggtgaaaagtgtctcagacttaatttgccagcaacgtgggaagatgctaatagacaatgtggtaatcttgggttttacctaccatctactggccttgaaaagaaatttccttggcttgtgacctatctcgacggaacttggggaaacactcagaactccgtatttggaccaaccggtgacttgcagaatgtcataggaccgaaagaatacaaatatttttgtgtgtccgattagatgattataatctaataaatgggttgctgtaaggtccctaaccgccagtctataaggactttgaaaaaggcgtcctgtccggtcgccagtctcgtcaccattctctccctagtcaccagcctcggtgcgatagtcagatacaccaatttttttctaaaagaagcatgtgacgaaggatggatgcccataaaaaacatatgcattttaaacacgcactttgaagccaccaatgacgatgcccacaggatatgtgaaaacctagacggaaagccgccggccatccctaaccctactctgttaaagggtgtcacagttctcaccggcgaaaagaaattttggatgacccatcacgaagactatactactgtgtttgagcatatagacgataggacgactcctaaaaacacagactatgacagtaaaaaacacacttgtttgatgagcgaggacggattgatacaccataactgcatgatgaacgtgactgtggtatgcatgaaggagatgcacggataactgaaaatatactgtttgaacgcaaagacgccatgtcgcgacttcaaatactgacctcatttggacaaatcttcgcacccgacgaagctcggctgcgcgagatcgcgcgtgatttgggaatatgcaccataaaacgcgcattcggcgacatgctgtacggctttatagacttcgacccggtgcccctgacccaagtaaacatgctcatgtccaactgctacttcgcggtcaacggcaacctgcttccgtgcacggaggacttccggctcagactcccggcaacggagatctctgcggcctacctgacgagaacgggacggacgatcctgtgcggcaaagacttcaacatagtagcgccgtcggggttcaagccgtccatgcggctgcgcgacctcagtcacgtgtctgcgcttgtagagatcctggaagtctacgacgagtccggggagtaccaattcgtgctcggccccagcgcgcagttcatgctgcggctgatggagaaggagaacgtctgtctgttcggcagcgggtggtgcatagtggacctgcgcaagctggacgtacccatataatcagcatccttgtttttatcctgtctttttatcagttttttagctagttaaaacataaatagtaaagctaaaaagaggagttctggagtcttgcaacaaccaggatgaaggcggtgttgttgctggcgttactgggagcgttcaccaacgcagcgcctttgttagaaagccagcgttctaacagtgaggaaaaagcaaatttctgctcgacgcataatgatgaagtgtacgccaggttcaggcttcagatgcgcgtgggtgtacgacacagtccgctctacactcccagcaacatgtgcatgctggacatagaagactccgttgaggacatagaagagtccacagaaaaagaatacgcgtctacggccacgggtgaggcggccggagtgaacgtgtcagtggcactagtgggagaaggcgtgagcataccgtttagttacataggccttggattcaacccgtcgcttgaagatagctacctgtacgtcaacgtctcgtcacgagctccttgggttaaacagacttcggacctatccgcgaacggcggctggggtatcaaacaggttctagaaaaagagttactggccatccaaatagggtgcgacaaccaaaaatttcccgaagaacccacaactacacccccctcacctgtcactacaacgctttcctcaacaactccagatctgaatgaagaaaacacagaaaatacgccgacgaccaccggcgccagtgtagacagaaagcgcaatccagctgacattgacttctcgctgctcgtggacccccgatgcgtgacctctgtagacctgcacgtcgagctcagggacgcgtgcatagactacaaacaagagtcgccgttgtcgctgaaggggaaatatggagacggcgaactagtaaaaaaggagattaaagacgtgggaaagaatcacaatatgtgcagtcttaacctcaaccctggcaattgagctgtttttattcggcaatataataggtgattattgaacattaaacaaaacttatcccacaacgccgcaacaatggaagtgctggtgatcatctctattatcgtcgccgtaatatgcttgaccggagcggtaatgtacctccttattgaactcggcttagccgccgagcgcgctaacaaacgcgcgcgcgtgaagaaaaatatgcgcaaattagccactcaattgggaaatggatctgtcgactccggcataggcataggcccgtgcataatgtcgcgcaccatggactctggacccagtcgctgggacagcgacagtgaggatgacggggacagcctgtccacgacgtccaccagcggaggggggactctcacccgagtgtgggttgggagcgccgggcctatgtacgaaaacttctgcgggaacggcacccgccactcccccaccaacgaccctggctaccactcgcgggagactctctgcagcggacctccccgtcaggcgccggcgctaccgcccaccccgaagcccgacgaggtaacggtggacgtggggcccggtcccgacgaccaacacggtccgtacgaggaacctgatcccattcccccgcaggaacccgagccgccggtgcagattgaggtaaccatcaacgggcccggtggagaaggcgaggcggaaggagaatttttctacgacgagtagccgccaaaactgaataactatcgggcttcgtaaacgcgcagacatgccgctgttccggaagctcatggtttcgcgctccctggtcaaggaatgtctgactctggacttccggcagggcgagcgtctccctacgcgatgcttcctcccggtgcccgcggggacgacattccacagagtctgcgacacctcgccgctgacggacgaagtctcccggcacgtgcaggagcccgtcatgggcaccggacgggtccagtactactacttcgagagcggccagggcatgatcggcgacaacgcgggcatggcgcgcatgctcgtgtgcacgcggtcggcgtacaacggcggcgacgtcgtcgtgcggtccacgcggagcagagcagacaagaccgtggtcgcgccctgccagggcatggcgctgctgctgagccccttctgcgccttcgacatcacgccggtcgagagcggctccgcgatattcgcggaggtcatcgtcaccgcgcccagcatggactacgtcgaggcggtcaccggcacgggcgaggcggccgtgcggatattcaactcgcaccacccgctctggccgcgacacggctcgaacgtctgcttcgcgctgcggttgctgcgagacgtgcgcacgggcgagcgcgtggtcgagcagatgttcatggacgggcgctggcacaccgtgctgaggacgtcctgcggcaacaaggtctgcgtgcccgccgacctcgtgggccagacgaacctcgaggaggtgcccttctgcgacgtgacgcccgagatcatgcgccgcgcgctggcgatcgacccgccgtacgaggccgtggcgcacccgcaccgctgcgtgtacggcgccatggacgtccggtgcgcgaacgagtacctcgtgtactgcaccttcaagacggagccgacacggcgcagcacgtcctcgccgggcccggacagcccgctgtcgcccgcgactccgtcgacctcgcgggccgcggccgcgcgcgtccccacgacgccgcaggaagtggcctcgccgaccacgaggctcctggagacctgtctgcgcgacgccctcgacggactctgacccgaaggacccaccgtccactcacattccactgccagacaactcaagctttttctgcatctacctcgctaataattgaattgttataggacaaacaggcgcactcgagcacaatggcgtgttttatcgaattgttagactccatcttcaaccgacgccaccgtaatttcgggccggaggacatgtacaggccctctgacgccccgccccccaaatcgcacacgcctcgcactccccgcaccccgcggacccagtgtcccggacacccgcggcgacaaagctcctctcccatctacggcgcttatgtggactccctgccgaggaacagaaagcggttccagaatcaacacagttgtcccggagattacgagcggtgtcaactctcggacaccatcagcctggatgcgacgctactcacggttacagtgacgtccatctccagcatatccagctctagtagctcagactctatctctctggggcagtgcagactgtccatggtgtccgcaacatcaacctccacctccacaaccttctcctcctcggaatgagcgccacacttatttttgtataatagtttgtattgaaccttagagacatccacaaatagttaggaagcatgagtagttcaagtagcgagaccacccctaagcccaagcccatccctgctcctcccatgactcaggaggagtttaacaaagaagtgaagaaacgaaaagaacagaaaaaggaaaaatctagaaccgttgaacgtgagtcagaaaccgtaactgtatcttccgacggatcagagataaaaaagacttacgagcgcgagtctgagagaacaaccgaaacagaaaagaacaacacgtcaaccgatgataataagcagaacacccctgtagagaaaccagaggaaaccaagcctgcttctactcctgaaggtgtgaagccagccgagactcctgccccgactactgacccccaacctactacacaaccacccgcagaatcaaaccctggaagtcaacccgcacctgcttcagaaccaacccccgcacctgagcctgcaccggaacccactcagcctgcatcagtaactcaacccgctccaacaccagagccaagtccagcccctaagcctactccggcttctgaaccaaccccagcatctgagcctacttctgctccagaacctacaccatccgcagaaccaactcctcaaccaactgtagaaacaccaccatctgctccagcaccaactcccgaggcccaaccacccgccaacagcaatcccactactgaaactaccactggtaccagcacctcctaagtgagtacgtaagcatttcggagtaacgtcgtagcaagcgctagtccgccgcgagcggttctcgcaagttttttcgggtaaaaagcgtacaccgtcgccttgtcgcggcggtgtacgcttttttcacgccctttttgcaaaatttaaattgtacccgcgccggctctaggaaagatggcgtgcctcagagtgttcctggcggtgctcgcgctgtgcgggagcgtgcactcggcgcaatggatcggcgagcgcgacttctgcacggcccacgcgcaggacgtcttcgcgcggctgcaggtgtggatgcgcattgaccggaacgtgaccgccgcggacaacagctcggcctgcgcgctggcgatagagacgccgccgagcaacttcgacgcggacgtctacgtcgccgcggccggcataaacgtcagcgtgtccgcgatcaactgcggcttcttcaacatgcgccaggtagagacgacgtacaacacggcacgccggcagatgtacgtgtacatggactcctgggacccttgggtgatcgacgacccccagccgctcttcagccaggagtacgaaaacgaaacgcttccgtacctgctggaggttctggagctagcgaggctgtacattcgcgtgggctgcacggtgcccggagagcagcccttcgaggtgatcccggggatcgactacccgcacaccggcatggagtttctccagcacgttctacggccgaaccgccggttcgctccggcgaagctgcacatggacctcgaggtggaccaccggtgcgtgagcgccgtccacgtgaaggcgttcctgcaggacgcctgtagcgcccgcaaggcgcggacgccactctacttcgcggggcatggctgcaaccatccagatcgccggccaaaaaacccagtaccgcgccctcagcacgtatcgtcgccgatctccaggaagtgcagcatgcagacggcgcgctaagggcgctcaccgcgctgacggcggccgtggtgtgcgcgatcgccatcgcgctcgagcgcggggcggaggccgacgccgtggaccttatccttataaaattttcaatgatatgctagtttttatgcgaccttccttagaaaattcggaattcaaaaatgaaataaaacggcgtttagcacgcatattattaataccgaccaccatggcaggcgtccgcagctgccagaagaaagtcccttctactgcgggctccatgtcatttcaacggggcaaccggagcatccagccggcgatgtccgaggcgttgcagaatgatttcagctacaacccgcgaccgcctccgccgagcgcagaagagattgacttcttctgcgtggacatgcgcaaagtactgatggaaattgaggccaagcccagcagctccaagtaccccgatttcatcacccggttgacagcagcccgccgtgcacgccggcgcgcaagcgcaacggcttcggccgcaaggcactgaacaaaacccaggtgccgcagcaggccaagcgtgacggctactcccgctaatgcagtccacacacttcacacactacatcagcactcaagcttataatcactacacaatgaatcagcccaccacgtgcgaagcacacacataaaatcacccacctgtcctgatcgttcccaatactcccaatcaccgtgctttacacgcacgttaatcaccctttccttccttcatgcgttcctgatcgttcctcctccttaatcacacacacaccccgtaattttgtacttttgtactttaatttgtacactttacacactgactttgtactgcctttgtactttatttttgtactgaaattggacgatacttatctttgtattcacatccaagttttgcaaattccacagccggtagcgaaaagtgaaatcgtaccgttttaggcttcgatccccctcccgcgcgaagactcgccagcatggactctcgtaggctcgctcttgccgtcgccttcggaggcgtcctcgccagcatgacacagcgccgccgcctggcttctctcatcgccagcatcggccaacggctgatgggcggcgacggcatgcgtcgcgtcgccgttcggttgatcgaccagctcatggccggacccccggacatcaacgacgaggccttccagcgcgagatccgcgtgggcgagctcttccaggcgctccaccgcgtggtcgagcaggcacgccgagagaagtacttcgaggtctgcggcgccggcaacgacgccgacgcgcccgtcgtcgagatggacaccgcggccgcacccccgcagccccagcccgcgcccctcgtggtcacgccgcagaacgcgttcatgttcgtgccgcaaggcagccacgtgcacgtggacgagagcgtggacccgttcttcggcatgagcccctccatcttcgggcgcgacctcccccctcagccgcccgaggagctgctgagcgactacgacccgctcatgagccaggccggcgagccgccgagcccgcggtcgccctgcgaggccgacctctggtgcttcgagacgctcggcgacagcgacagcgattgagaccgcaccacaccctacctcacccaccccacactccacctcacctcaccctaacacccaacacccaacacctcaaccggacaatgaaggagtcccacatttcactgaaggacgcggatgaagccgcactcccccacatgaaggattggcaacggtcaaacatttcacctgcaatgaaggacgatgcgcggtcgcattggcctgcgaccgacatcgcacacatgaaggacacaattggtttgttaatccggacaatgaaggacaaattgtttttgttaatcaggacaattagaacacaatcaaatttttgtacgatcataaaatcgatatttgatgcacatatattagtaagtatattagactaaattctccggggaggcaagcagttggatacggcggggcggggcacgacgtgcacggagagttcgggcgggtcccccttccccccacccccacgcaccacgatgcgcctaatcttagcgctcgtggcctgcttgttggcggcgccgatgccgttatcgggtcgttcgacaagcactccaaacacgtccccctccgcactcggctcgacgagttcggaaccaagctcggaagacgctgtggcttcgagcacaacgacaagcacactcacaagcactacaagcacactcactatgtccacaagtgtggacaccactactacctcgggcgctacaacgtccgcaaacagcactcctgcagcgagtgtgagctcctccacacccgcaactaccgaggcatcgacggcaccaacgacgccgtcgacgccgacgacagtgaaggtaacgaagggcaaggaagacacgaaggcgtctgcctacctcgttttactaatcacgttcatggtcatgaccacgctcgtgatggtcgtggtcgtggtcgtggtcgtgtacaaacagggactctgtaactgctgctgtaagtttccctgctgcaaagagctcaaggactacctcgacgaggaggagagcgccgggctgtacgacgccttgacgtggagccactcagactccggcctccggctcgtcgtgcgcgcggaccccagatgatgaggatcggataagatcggcgtgtttttcccgcccgtcgcgaacattatgcctctaaatgccgagaattaactgaaattcaaacacgctttgggactcaactccgtgacccacactcaaccatggctggcttcctaggcgcattcagaggcgtgtgctccgacttatggcagtcgctccgtggacacggacaccactcttccagctgcccgcgacgacgcgccaacagcatggacgaccgcgaccggcgccgacaccgccaccgcgagatccccaacagctcggcgtcgctgaacagcgacccgatgccgcaacgcagtgcgggtgcgcgccggcactacgactgccgcccctcggaaaagagcagacactcctccgacaagcaccactcggcggaccgacaccactcggcggaccgacaccaatcggcggacagggacagacaccgtcgcagtcgcaagaactacgactcgcacccgtcgcgcaggaaccgcaactacgagcgggcggactaccagagacacccctcacaaacccacccagacgcccccgcgcagacctcgacgctcaaggtgacctccctaagcaccagctgcagcaccctgtcccaacatcactacgagacccccgaccacatctacgacatcccggaagacagtcgcggggcgtcggctccccctcgcgcggacctcgcgctccccccgctcgccatgcccaaatccaagccgcgtcgcacgcgcccggcgtccatgaacgactgcctgatgaagcactgcggcgccggcagacccaacctccaagacgacatatgcacactatgtactgatatagagacacagctgagcgcactagagaagtctctggagtcagagctcaacttctatcgtcgctacatacaagacactaagacattgctcgccacgcgagcagcaaacatcggcagcaaagctctgatctacaccgacgactacaacggcagtggcgacgtcggcgaaaaggagcactgctcggaggagtgctgcaaagtggaggaagttctgtgagaaagtgcgtttttctgtaatgtgaaataagatagccttatgtgtgcacagacatggcgaacaggctcgtgtttctcgaccccgagaccctagccgaggccgacggcatccccggctatggggtgttcgagcccggcaagaagaaatgcatcttcacaaagatccgcaccagcgtcgcactcgcgtgccggtacgccgtctcggacggcggcctcatcgacgagttcgtcatggctacatacgggaccagacgcgcgtgccggctcgtccggcacctgacgataggcgcggagggcgtgatgacccggcccgccagcaactgcgcgccgcacatggtgctcatctgcctcagaggcgtggccgccgtgtccagcgaggacatgggcttcggtcgctgcatcatggagcgcggcaccatgttcatggtcaagtccgcgcacagcgccgtcgtctgcggcaaccccgcctgcgagctgctcgtcctcttctacgactacttcacccccatcccccggccgctctccggagacgaggtgctgttcacccgcgacctcgcgcacgtggactacgcccccgagtcggcggtcgtcttcaagatggattacaacctcgagaccgacgtggccacgctgtttgtcggggggtacatattccgcgccaagggcctgatgatggagacgcgcgaacaagtgggcgacgagtgcgactgctgccgccacagctcgccggtgctcgtcatggatcgcgagaagatgatgtcgtcgctgcgcatgatccccagcatcgtgcccggccagcgggagatttggcttcgcgagcgcggctgggccgtcctcgagacggacgcccgcggacactgcgagcccggcgtcctgaggctggcgctcgccggcctgcggctgttcgcaggatgcctgcgctccgtcgtggggcggcgcgagctgtcgctgttctgctacggcgtcgctcccaagttcggcggggagttcgaggacgcgccgcgccccatggagatcgacggttagttgtttttatccctgtacatacgccgcaaactgaaactttagggcaccgcgtaatagtgcacgaacgcccagtggaccgcttccgcagccatggaaaacaacgacggcaacgaacgcaacaacgaacacccgcacgttcgagaattcaaggaggcgtccctgtacgggtttctggtggcggccgcggacgtgaccgtcgaagacgtgcaccggtaccttcagttcggcgcggacgtgaactacaggggcgcgtacctgtgcacgccgctgcacgcgtacctgcagtccggctgcgaaaagcgcctagacgtcgtggacgcgctgctggacgccggcgcagacatcaacgccaaggagatctgcggtctcacgcccgtgcacctgtacgcgagctacgcggacgtggacgtagagttcatgcgcgggctcatcgagcgcggcgcgagcgtgtgcggcgagagctcggtcacgggctgcctgtactcgtacctgtacacacacagcgtggacggcggcgcgcgcctggacgtggtcgagctgctcgtgcaggcgggcgcggacgtgaacgtccgcggcgaggcgcgcaagacgccgctgcacgtgcactgcgcgggcttcgaggtggattcggacatcgtggatctgctgctgcgcgcgggcgcggaccccgaggcgctcgacgaacacgggctcacgcccgcggacgtgctcgtgaagtccgtgggcgccaacgtggagacgctgcggctcttcctcgacgcgggcgtgagcgtggccacgtcgcgcgacgcgcgcggacgcacgccgctgcaccaccacgcagactccttccgggcgagtgcgggcatcgtgcgcgaactgctcgccgccggctgcgacgcggcggccgccgacgacctcggaaacacgcccctgcacagcctcgccaccttctgctcgtgccggcgctcggtgctcgaccagctcatcgccggcggcgcggacatcaacgcccgcaaccactacggccacacctgtctgtactacgcgtccatctacaacccctccgtctgctcgcggctcatcgccgcgggtgcggacgtgaccgcgcgcacgccggacggacgcacgccgctctcgggcatgatcatgcgcaagcacacgcgcgccgtgcgcgccgccctggcgacgcggcctcccgcggacgccgtcgccgcgtcgctggacgtcgcagtacagcccgagcccacggacgccactcgcgcgtgcgtgcggtacgtggtgctctgcggcggcacgctctcggcgcgcgtgcggtcgcgacacgcggacttcgtgcgagagtgcgaaagcgaggtggtcgtgctcagaaccaccgtggtggggctgcccggcacctcgctgctggacatcgtgcgtgcggcgcagccgccgccggtactgctctccccgcgcgtgcaccacgtgctgcagaagctgtgcgtgtacgcggagttggtggacgcgcggctgcgcgagatgcggcacaagaccaacctcgtggacgcggtgtcgcggctcgtgtgtccgtgcgcgctgccgccggaggtggtgcgcggcatcctcgtgcacgtgccgatagacagcctgcggcacacgttgaccctcggcgtggcgcaggcctcgcgtttccttccctcgcataaatgaaatattattttttgtggtagaccggatctccccgatggaccccgccggacaacgactgcgcgcgccagggccgtggcgcctgaacccgccgaccgcggccgcgctggaaagcgcgctgctgcggcccgcggcgtcggcgggcgccgaccgctgcgcgaacgcgcacgtggaccgccgcaacatgggcgtcggcgagggccgcgaggtgcccgcggacgtcgaggggctcatgaccgagatccacctgcggtacggaatgacgcgcgtccaccggaacgttcacttcgtgcagttctggcacggcgagcacgtgcgccggcgccccgcgcgacacgtgttcacggtctggatctgcctcagcggcgaggtgcgcatctacgcagagtgctgccaggcggggcacggcttcgtgctctgccgacagatggcggcagggtacatgttcgtgaccgaacccacggactcggtcacggtctcggtgccgcaccgactgcgcaactcgcggtcgccggtgtggctggcggcggtcttcgccacgcggcacttcgagccgctgccgccgcccatgtacgccgtgcccgggcacgtggtgctcgcgcgcagcgcctccatgctctgcgactgctggccgtcggacccgcggcgccgcaacgtgatcttctacatgcggctgtcgggcgcgatggtgcgcgtggtcgtgccgggcgcggagctcgagatcgagtgcacctcggggttccggccggaccacttctccatcaacgacgagtgcgtgtgctgcgagcgtccgcacgtcgcgcgaaccgcggtgtggacgctggcggagatttgccgcggcgccacggtggtgctcgcgccgccactgccccgcgaccgcgccgcggggctgctcgcggagatccgcctggcctcgctgcgatgggtgcgcgtgcgtgcggtccgcagcggcagagaaagcgtgggcccgttcccctcggtggtgtgggcggcggtcttctccgccgttcggctcttcctggacggaaccgtgcctgccttcccggcgtgtgtggagaatggacgcgcggcgtacggcatggtgtacgtgcccccggaggagccgcggatggacgggctctgtgtgttcccgacgcccgccgagccggcggcgctcttcgtccgcggagaccaggtgcttgaggccggcgcggccgccgccataatcgcggccgctgagaagcgcgtccaggccgccaatgggtctcctgctgccgcggaggaggacataggtgcggcggccgatgccgccgcagagagcgtggagcaggaccagcgcgtcgagttcgaccttgggcctgggcctgaccccagccaagaagcgcccgcggacgcgcagcgtgccgattcggacgacgacaccggctccgagaccgagaccggcgacgagagtgtgggcggcgaggatgacagcgactcctcctcctcttactcggtgatgtcggacgacgaaaacgacagcggcgacgagggctggggcgactctagcgactccggcatcgaggacgacgacggcggtgtcggccaggccgccgaggaagaagaggaggaagagcgcgacgtcctcggcgcagcggcccagatgctcggagactgaccggtggtgaaaacataaaaataaactgttcaacacttgtactccgggcaccaacactactatccatacccaccctccctccacacactacaatggcaaacagagaagagattgacgcctccgccgtcatggctgcctacctcgcgagagagtacgcggcggctgtagaagaacagctgacgccgcgcgagcgcgatgcgctcgaagcccttcgcgtttccggcgaggaggtccggtcgccgctgctgcaagaactctcgaacgcgggcgagcaccgcgccaaccccgaaaactcgcacatccccgccgccctcgtctccgcgcttctcgaagcccccacctcccccggccgcatggtcactgcgattgagctctgcgcgcagatgggccgggtatggacgcgcggccgccagctcgtcgaattcatgcggctcgtgtacgtgctcctagaccgtctgccgcccacggccgacgaggacctcagcacctggctgcaggccgtcgcgcgcgtgcacggcacgcggcgccgcctgcaccgcgttctcggcgtcggggccgtcatggcaggcgtcggtatgctgctgctcggcgtgcgcgtgttgcggcgcacataactttttatctcggctcaaactgaaatacgacattggactacgaaacctatgattttgctcacggccgcgcgagataggataataaataacctttgagcaactaacatggccgatgagagagaggccgacggcgcgctgttccggtacctggagagcgaggaccgtccggacgtggagcacatgcgccggctgctggacgagggtgcggacgtgaactacgcgggcccgcgcgggtacgcgccgctgcacatgctcatgcgcggcaacccgctagaccccgacgcggtgcgactgctgctcgccgcgggcgcggacgtgaacgcgacatcgctctgcgggttcacgccgctgcactcctacatgtgcttcgggaccgtgacgccagacacgctgcgtgcgctcatgcgccacggcgcgagcgtcagcgacctcgagcgcaacatcaacgcgctgatcgagtacttcaaccgcgacggctgcatgggcggcgcggaggcgaccgtgatcgcactgctggtggagcacggcgcgcacgtgaacgccaaagacgaccttggacgaacgccgctgcacatctacctgtccggcttcttcgtgtcggcaccggtggcgctcgcgctgatcgcgctcggcgcgaacccgaacgccacggacgcgtacgggcgcacgccactgcacgccttcctgcgctcccgcgacgtggaccccgctgtgctgaagacgctcatcgccgcgggcgcagacccgctcgcgcgcgacatcatccggcgcacggcgctgcactaccactgcgagtccttcaagacgcgcgctagtgttatagagacgctggtggccgccggctgcgaccccgcgagcacagacctgctcgacaacacggcgctgcacagcatggccatgggcagctcctgccgcgcctcgctgatccgcccgctgctggccgcgggcgtgtccgtgaacgcgcgcaacgcgcggctgcagacgccgctgcacctcgcggccgtgttcaacccgccggcctgcgcgcggctgctggccgcgggcgcggaccccgcgctcgcggacctggacgagacaacgccgctgctgagcatggtgcggcacaactgcgcacgcgcgctgcgcacggcgctgcccttggcgccggacgcgctggtggccggcgcggtgaaccgcgtgaacgcgcgcacgccgagcgcggccacgcgagagtgcgtgatggcgctggcgctgcgcggcgcgctggacctgctgagcgcggagagcgttgccacccacgcggccgcgatccgcgcctgcgaggcggaggtcgcgctgctgcggcgcacgcgcctgggcgcgccgccgacgacgctcttcgcgctgctgacaggacgaccgaacacgctggtttccgcgaaggcggcgcgacgcgcgatggcggacgtgtgtgtctaccgcgcggcgctggccgcgcgcgtggagcgcgtgcgccgcaagtcctcgctggtcgagcgcctcaccgccatggtgtgtccgtgcgctctgccgccagagctagtgacgcgcatcctcgcgctcctgaccgtggaggaactcgcttgcgcaatgcgcaaataataatgaactataactaggcttattagaggcactatttgtgcagagtcgttagttatagttagtgtacttacaattggaatgtcgaagaacaaaattctggtgtgttttgtaattattcttacttatacattatacacagatgcgtattgtgttgagtatgaggaaagtgaggaagataaacaacagtgcggtagtagtagtaattttcctgcgagtttaccgcacatgcttagagaactcagggcagcgttcggaaaggtaaaaactttcttccagatgaaagaccaactgaacagtatgctactcacacagtcgctcctcgacgacttcaaaggctacctcgggtgtcaggcactttctgagatgatacagttttacttggaagaggtgatgccgcaggcggaaaatcacgggccggacatcaaagagcacgttaactcgctgggagaaaaactcaaaacgctgcgtcttcgactgcgtcgctgccaccgcttcctgccgtgtgagaacaagagtaaggccgtggagcaagtcaaacgtgtgttcaacatgctgcaggaacgaggtgtttacaaggccatgagcgagttcgacatattcatcaactacatagaatcatacatgactactaaaatgtaaaaatgtatacaacttttagttatcgttcggattctcgtatcgttctgcatactatgtatataaaatgtatattaacatagttacagttacagttacagctatatttttatgctcacaagatgctatataattgaaaggaaattgttcactctctgtcagggcgccatggactttctaggcgccgcgcttcacgactacgttgccgatgcgcccaaggtctgcgccgaggaggtgcggcggctgctggccgcaggcgcctctgtggagtacgcgggcgagttcgggaagaccgcgctgcaccagtacatgggccgttccggcgcggaccccgccgtcgtgcgcgcgctgctggacgccggcgcgcgcgtggacctcccggagacctgctgcggctgcacgcccgtgcacctctgcctcatggccgccaatatcgacgtggaggttctccgcatgctcgtccacgagggccgcgtcgaggactgcggccgcgccgagctcgcctccgtggtgctcaaggagttcgtggtgaaccgcgccttcgacgagaacgtcaccgagcgagtgatgcgcgttcttgtggccgcgggcgcggacgtgaacgccgccagcgtggtcgaccgcacgccgctgcacgtctgcctcacgggcatgtccacgcacccgggcaccatcgccgcgctgctgcgcttcggcgcggacgtgaacgccgtggacctctgcggcatgtcgccgctggcggtgctcgtgcgctcgcgcgcggcgaccgcagagctggtgcgcatgctgctcgacgcgggcgcagacgcacacgctgtcgacagtcgcctggactcgctgctgcaccagcactttcagtccgcgcgcccgcggccggaggtggtgcgcgagctcatccgccacggctgctcgccgcgggcgcggaaccgaatcggcaacacgccgctgcacgaggccgcaaaacactcctcctgcaaacactcgctggtggggccgctgctggctgccggcgcgagcgtggacgcgcgaaataacacgggcaggacgccgctccacttggcggtggcgtccaacccgcgcgcgtgccgccggctgatcgcgcttggggcggacgtggtcgcgcgcagttacgcgggcgtcacgccgctggcgcagctgatcgcggacaataactccgcgctggtgaccgcggcgctgaacacgcagcccgagccgcgggccgtggcagagtcgctgcgagcgaccacgcccgtcggcgagacagcgtgctcgcggctctgtgtggcgtacgtggtggcgcgcgcgccgagcgaggtcctcggcgagcccgagcgcgccctgcacgcggccttcgtggcggagtgcttagcggaggtagcggccatacacgccgtgcgctgcggcacacctccggtctcgctgctggagatcctggtggccgcgcgcccgccgcggagcctgctctcgcgccgcgcgtggcggctggccagccggacgacagtttaccgcgcgccgctccgtgcacgcatcgcggccatgcgccatcgctcgcgactggtggagcgcgcgctgcgcacgctgcgcggctgcgtgctcccgcgcgaggtgctggagcgcgtgctgcggtgtctgtccacacaggacctgcgggcctccggactggccgagtagctttttctgagataagtgaataaacatggtgggattcgatcgcgccgccaacgccacgccatggacgccgccgagatggaggatctcgacatcaacgcggagtcggcgctgtacgactacttcatcctgaacgcggacagagcccgcgtgggcgaggtggttatgcttctcgcacagggcgcggaaataaactacgcggacagcttcgacaagacgccgctgcacctgtacttgcacacgcgacacccgcgctcggacgtgattctggcgctgatggaggcgggcgcggtcgtggacacgccggagcgctgctgcggcgcgaccgcggcgcacctgtacatcctcaacgcggccaaggtcgacctgtcggtgctggaggccatgctgacctggggcgtgcgccagaacgaccagcactcggagcggctgctctcgagcttgttgcgcgagtacgtggtgacccgcgcctactcggatcagaccgagccgatcatggacttgctcatcggcatgggcgccgacgtggacatgccggtcggcgtgagtcgcacggcgctgcacgcctgccttacgggcctgaacgcgaacccgtgcatgattcgcgcgctgcttcggcgcggcgccagcgtgaccgcaaaagacacctacgagatgacgccgctggcggtgctgctgaagtccgcgagcgcgacgccggagctcgtgcgcatcctcgtggaggcaggctccgacgtgagcgccaccgacttccgcctcaacggcatgctgcaccagcacgcgcagtccacgcgcccgcgcgcgagcgtcatgcgcgagctcatccggctggggtgcagcccagcggccaaaaacatgtttgggaacacgccgatgcacatgctggccatggaaagctcctgccgccgctcactgatcctcccgctgctggaggcagggctttccgtgaacgaggagaacccgcactacggcaccgtgcctctgcacgtggcctcggggtacgacaacacgcagggctgcctcaagctcctccggcatggaggagaccccaccgtcgtgtcggccgccggacgcacgccgctctcgaacatgctcgtcaaacgcaaccacgtggcggtcgccggcgcgctgtcgacgcacccgagcgcggcagtagtcgtgcaggctctcgagcaggctctcgagaacgtgctgaacgccgggcccagcgaggcctcgcggctcgccgtggcctttgtggtggcgcgcgccggcgcatccgcgctaccggaggccgtgcgccgtctgcacgagggctttgtcgccgactgcgagcgcgaagtcgagctgctttcccgcaccatgctcggcacaccggccgtgagcgcgctggtcgtgctggtcagcaaggaggtctttggcactgttatctcctcgcgtgcgctgcgcgtcgtgcgggaggtccgcgtgtacgcaaggccgctccgcgaggcgctcataaatctgcgccacaaatgccgcttagtttccagccttaaaaggcaggtgggaccttgctcgctgcccggcgaactggtggagcgcgtgctcgcgaccgtgccactgaccgacttgcgccgctcgtgcggccgccgcgcgcccgagtaactgcccgtcccgttgctacgcgactcgagactgcccgctgtttttctttccccgtttcttcttattaggagttgttgcccgcctccatgatcctcgcacgcgccggcgggcaacctcgcacgcccgcggcggccgcggccgccgccgaggacggcgagcacagtgatcgccggaagcgcaagcgcaagacgcccaactgcgaagacgccgacaactccgacgacgagctagcgcagacgccgtgcgaccgcgagtggccggactgtcgcgcgagctcgatcacgagctccgactcggtctctctcggcgacgagatctacctgcgatacgtggcctcgcaggtggacttcgcgcagacctgggccccgccagtgcggctgctgcgcttcttcgggaacttctcgaaggaaacgctcaaccgcatgtcgcggcgcgggtacgtgaaccgctcctacttccagatggcgcacgcgcgcttctcgcccaccaacgacgacatgtaccacatggccacgggcgggtacggcatcgtgttccgcttcgaccgctacgtggttaagtacgtcttcgagcaccgcaacggcatgtccgagatggacgcctctacggagtacacggtgccgcggttcctgcgcaataacctcaagggcgacgagcgcgagttcgtggtctgcgcgctggccatggggctgaactaccggctgggcttcctgcactcgctgtaccggcgcgtgctgcacacgctgctgctgctcatgcgcgtggaggaaggccagcggccctcggtggagatgtccaagaagccgctgctgcgctggttcgaggcgcgcaaggacagcgagtccttcgtgcgcctgatctcgtacttctacccctcggccgtgcagagcaacgtgaacctgatcaacaacttccaccacctggtgcacttcttcgagcacgagaagcgcgcgcggtacgtgttcgaccgcggggccgtgatcgtgttccctctggcgcgcgggtccgcggactcgatctcgccggaggcggcggcggcgctgggcttcgcgccgcactcggagttcctcaagttcgtgttcctgcagatcgcgctgctgtacctgaagatctacgagctcccgggctgcacgaacttcctgcacgtggacctgaagcccgacaacgtgctcatcttcgacagtgcgcgcgcgctcagcgtgaccgcggccggcgcgactttccgcttcgaggagcccgtgcgcgcggcgctgaacgacttcgacttcgcgcgcgtggccaccatcgagaaccgcaagatcgcgggcagcgtccgcgtgccgcagaactggtactacgacttccacttcttcgcgcacacgctgctgcgcgcgtacccgcacatcgccgcggaggacccgggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgccgcactctcctgagcccacgcccgcggcgccgggctcgctgtacgacgtcttcctcgcgcgcttcctgcgccggctggccgcgcgcgcggcgccggcctcggccgcctgcgccgtgcgcgtgggtgcggtgcgcggccgcctgcggaactgcgagctagtggtgctgaaccgctgccacgcggacgcggccggcgcgctcgcgctagcctccgcggcgctcgccgagacgctggcggagctcccgcgcgcggacaagctcgccgtcgcgctcgagctgggcgtggaccccgagcacccggagctgacgccggaccccgcctgcgcaggcgagagcgcactcgcacagaacatcgacatccagacgctggacctgggcgactgcggagaccccaaaggccgccgactgcgcgtggcgctggtgaacagcggccacgcggccgcgaactgcgcgctcgcgcgcgtggcgaccgcgctgacgcgccgcgtgcccgcgagccggcacggcctcgcggagggcggcacgccgccgtggacgctgctgctggcggtggccgcggtgacggtgctcggcgtggtggcggtttcactgctgcggcgcgcgctgcgggtacgctaccgcttcgcgcggccggccgcgctgcgcgcgtagccgcgcaaaatgtaaattataacgcccaacttttaagggtgaggcgccatgaagttgctcgtcggcatactagtagccgtgtgcttgcaccagtatctgctgaacgcggacagcaacacgaaaggatggtccgaagtgctgaaaggcagcgagtgcaagcctaggccgattgttgttcctgtaagcgagacgcacccagagctgacttctcagcggttcaacccgccgtgtgtcacgttgatgcgatgcggcgggtgctgcaacgacgagagcttggaatgcgtccccacggaagaagtaaacgtgacgatggaactcctgggggcgtcgggctccggtagtaacgggatgcaacgtctgagcttcgtagagcataagaaatgcgattgtagaccacgattcacaaccacgccaccgacgaccacaaggccgcccagaagacgccgctagaactttttatggaccgcagatccaaacgatgatgcgatcaggtcatgcggaagaaggcgccacggagcaaagtgaaaaaggaccgcctagcagtcgagaccctcccgccgcagccgcggacaccccacacccgccttccacccgccagacgccaacaccgcagccaacaagcatgcacccctcgccgcgcaggctgctcggcgcgctcgcgctggtggcgctgggcttcctcctcggcgggctcttccgccccgcggcgccgccgctgccggccgccctcgtggaggcgggccccgtccgcgcgaacggctccgcctcggtgacctgcctgaccgtcggcggcgacgggcggcacatggcggtggtcgcgcacggcggcgggacgctctcgccggtgtacccgctcgccgccggcatgcacgcgaccttcgcctcgctgcgcaagggcgcgctgctgctgaacgtcgcgaccgtgcacatctacgacgtgcgcgcgctcgggccggagttcgagctgacctgcgtcgcggtggcgggcggctacaacgcggcctgggcggccgcgcggcccgcggccgagtggcgccgccagctggcgcggatgcaccgctcggagctgtgaccctctccctcccggtctcccatccgtttttgtaatcggccttagtagattagaccagcatcccgcgcccttgtccgccgcccttcgctcgctcacgagcgaatcagtcaattgattatttttatcgcccgcccgctcactccggtaagggaacgcggttaacccactcacgagaacaagcgaccgccactcacttgaggtaagggaacggttacggtcaactcactcacgagaacaagttgactcactcctcgaggtagagaacgagagaacaagtgaccgtactcactcacgagaacaagttgacgcaccgctcaccgaggaagggaacagagaacaagtaacagtaaccgttacctcactcgccactcctcggaataatagaacgagagaacgagagaacgagttaaccgttgctcactcgctcactcggtgtgagagaacaagtaacgttgctcactcgctcactcggtgtgagagaacaagagaacgagtagctgttgctaactcaatcacccctcggagtaagagaacaagagcagtcaactacccactcagtcttggatgagaggcagaggacgagttgacgagttgaacagttaatcctcactcactcagagcgagagagcgagagagtggaggacgagttaacaagtcaatcctcactcagagtgagagagtgagagagtggaggacgagttaatggttaacagttatcaccactcagagtgagagcggaggacgagtcaaccactcgctcgcccactccgagttagagagggaaccagtgcgagttaacgcgcacacgagcgagagaacgtaaactcgctcgcgcgctcgctcggctaaccgtcggcctctcccaaaactcttcgtaaaactttcccgatgacagttcaccctccaaaactttgtaaaactaaactgttcggaggtcggtctgctgcctctctaactctccgtaaaacgtttgtaaactgtcggaggtcggtgacccgctcaaccgtccgcgaaaacttttcgcaggcagtgtctgcctctctcggactctccgcaaacactttcgcggaacctcggagggtggtcgacctctctccaaactcttgcaaaactttttcgcggaaccgttggaggccagtcctccctccaaactctttgtaaaatcttttcgaggccagtcctcctctccaaaacgttccgcaaaatctttgggaggtcggcctctcctctccagaacgttccgtaaaactcttggaggccgcccgcggcacgcgaggcggaggatccggagggtgtcgacctccctcaaaaactttgtaaaaactttttataaaactttccgcggaacctcggagagtaggtcgacctctctcaaaactcttataaaacttttccgcggaaccgttggaaggtaggtcgacctctctcaaaactcttataaaacttttccgcggaaccgttggaggcaggtcggcctctcaaactctttgcgagaactcttcgcgagaactcttcgataactttaggaggtcaggtcgacctcccaaaacttttgcgagaactctctgtaaaactttaggaggtcggtacctccctcaaaactttttataaaacttttcgcggaacctccggagacgggccgccgcccgcgaccgcgggagcggagaggccgacctcccgagacgttccgcgttaccgtcggggtaggcgtcctctcgagaacgccaaaagacttcgtgcaaaaacttttcggaggggcgcggagggcgggtcagctcccgcgaactcccgcagaaccttttcgcgcgaccgcgaaggccggccgcctctcccacactctcaagagcttttcggaggagaggaagggcaggtcgcccccacctccgacgctttgtaaaaacgtttacgcggaacctcggaggcaggtcgcctccctcgaaaactcctcgcgaaacctttaaaaactttttgcgaaaacttttcggaggatgtcggagggcgggcggctcttccaaacctccgcagaaccttttcgcgcaaccgttggaagacaggtcggcctctctcgaaaacttttaaaactttgtaaacgcgttggcgggaccgtcgcgggagagcggccgcccgcggcacgcgagaggaggaaccgttggaaggcagtcggcctctcccgaaaactttttataaaaacttttccgcggaaccgttggaggcaggtcggcctctctcagagtctaaaaactttttgcgggactcggacggcgcggtcacccgaccacctgactcctgtctacccgactacttgacttctgtttccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactcccgactccctgactccctgactccagagcgaggtctcgcggctgcggggtgccgcctccgcggagtcgcgttcccgcggacgcccgtcctcgaaagcattcagcagttccagcctctgccgtagctcctcccgcaggaactcctggtccgcgttctcgcggcagcgcggctcagccgccgcgggagccggccgccgccccggcgaagccgcggatccgctgttccggcgaaggcaggacagacattttttccagcccataaattaaaaagaacaagaaattacatgtgaaaaaatttgaggttgatttattgacgaaacgttcaactaaatacaatcctaaaagtttttatcatcatgataattttctgttgattctttttaatttatgggctggaaaaaatgtctgtcctgccttcgccggaacagcggatccgcggcttcgccgg

SEQ ID NO: 3 - skipped

SEQ ID NO: 4 - protein, 12 aa, synthetic construct
ESKYGPPCPSCP

SEQ ID NO: 5 - protein, 22 aa, synthetic construct
ESKYGPPCPPCPGGGSSGGGSG

SEQ ID NO: 6 - protein, 39 aa, synthetic construct
IEVMYPPPYLDNEKSNGTIIHVKGKHLCPSPLFPGPSKP

SEQ ID NO: 7 - protein, 48 aa, synthetic construct
AKPTTTPAPRPPTPAPTIASQPLSLRPEACRPAAGGAVHTRGLDFACD

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 228 aa, synthetic construct
ESKYGPPCPSCPAPEFEGGPSVFLFPPKPKDTLMISRTPEVTCVVVDVSQEDPEVQFNWYVDGVEVHQAKTKPREEQFSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, synthetic construct
LCYLLDGILFIYGVILTALFL

SEQ ID NO: 14 - protein, 27 aa, synthetic construct
FWVLVVVGGVLACYSLLVTVAFIIFWV

SEQ ID NO: 15 - protein, 28 aa, synthetic construct
MFWVLVVVGGVLACYSLLVTVAFIIFWV

SEQ ID NO: 16 - protein, 22 aa, synthetic construct
MALIVLGGVAGLLLFIGLGIFF

SEQ ID NO: 17 - protein, 21 aa, synthetic construct
IYIWAPLAGTCGVLLLSLVIT

SEQ ID NO: 18 - protein, 23 aa, synthetic construct
IYIWAPLAGTCGVLLLSLVITLY

SEQ ID NO: 19 - protein, 24 aa, synthetic construct
IYIWAPLAGTCGVLLLSLVITLYC

SEQ ID NO: 20 - protein, 27 aa, synthetic construct
IISFFLALTSTALLFLLFFLTLRFSVV

SEQ ID NO: 21 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, synthetic construct
RSKRSRLLHSDYMNMTPRRPGPTRKHYQPYAPPRDFAAYRS

SEQ ID NO: 23 - protein, 41 aa, synthetic construct
RSKRSRGGHSDYMNMTPRRPGPTRKHYQPYAPPRDFAAYRS

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 42 aa, synthetic construct
ALYLLRRDQRLPPDAHKPPGGGSFRTPIQEEQADAHSTLAKI

SEQ ID NO: 26 - DNA, 10 bp, synthetic construct
  source 1..10: mol_type = other DNA
gggssgggsg

SEQ ID NO: 27 - protein, 12 aa, synthetic construct
ESKYGPPCPPCP

SEQ ID NO: 28 - protein, 22 aa, synthetic construct
ESKYGPPCPPCPGGGSSGGGSG

SEQ ID NO: 29 - protein, 323 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 30 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - DNA, 37 bp, synthetic construct
  source 1..37: mol_type = other DNA
gcgcatatgatctatggattaccatggatgacaactc

SEQ ID NO: 32 - DNA, 28 bp, synthetic construct
  source 1..28: mol_type = other DNA
cgtttaactcgtctaattaattctgtac

SEQ ID NO: 33 - DNA, 98 bp, synthetic construct
  source 1..98: mol_type = other DNA
caggtaaaagtacagaattaattagacgagttaaacgagctcgtcgacggatccgctagcggccgcggaggtaatgatatgtatcaatcggtgtgtag

SEQ ID NO: 34 - DNA, 39 bp, synthetic construct
  source 1..39: mol_type = other DNA
gcggaattcgtaattacttagtaaatccgccgtactagg

SEQ ID NO: 35 - DNA, 41 bp, synthetic construct
  source 1..41: mol_type = other DNA
gcgcatatgtagaagaattgataaatatgaaaccttttaag

SEQ ID NO: 36 - DNA, 29 bp, synthetic construct
  source 1..29: mol_type = other DNA
cctctctagctttcacttaaactgtatcg

SEQ ID NO: 37 - DNA, 100 bp, synthetic construct
  source 1..100: mol_type = other DNA
gaataatcgatacagtttaagtgaaagctagagaggaagcttgagctcgaggatccgctagcggccgctgaagaggatgctagaatcaaggaggagcaag

SEQ ID NO: 38 - DNA, 38 bp, synthetic construct
  source 1..38: mol_type = other DNA
gcggaattctccgggcagtgactttgtagctctcccag

(19) United States
(12) Patent Application Publication
(10) Pub. No.: US 2025/0381237 A1
(43) Pub. Date: Dec. 18, 2025

(54) ONCOLYTIC VIRUS EXPRESSING A CAR T CELL TARGET AND USES THEREOF

(71) Applicant: City of Hope, Duarte, CA (US)

(72) Inventors: Yuman Fong, Pasadena, CA (US); Saul J. Priceman, Valley Village, CA (US); Stephen J. Forman, Duarte, CA (US); Nanhai Chen, Duarte, CA (US); Anthony K. Park, Duarte, CA (US)

(21) Appl. No.: 19/247,030

(22) Filed: Jun. 24, 2025

Related U.S. Application Data
(63) Continuation of application No. 16/637,909, filed on Feb. 10, 2020, now Pat. No. 12,364,724.
(60) Provisional application No. 62/544,707, filed on Aug. 11, 2017.

(51) Int. Cl. A61K 35/768; A61K 38/17; A61K 40/11; A61K 40/31; A61K 40/42; A61P 35/00; C12N 7/00

(52) U.S. Cl. CPC A61K 35/768; A61K 38/177; A61K 40/11; A61K 40/31; A61K 40/4211; A61P 35/00; C12N 7/00; C12N 2710/24021; C12N 2710/24032; C12N 2770/32021; C12N 2770/32022; C12N 2770/32032

(57) ABSTRACT

An oncolytic poxvirus encoding a truncated human CD19 is used in conjunction with a chimeric antigen receptor to treat solid tumors.

### CROSS-REFERENCE TO RELATED APPLICATIONS

This application is a continuation of U.S. application Ser. No. 16/637,909, filed on Feb. 10, 2020, which is a U.S. National Phase Application under 35 U.S.C. § 371 of International Application No. PCT/US2018/046313, filed on Aug. 10, 2018, which claims priority to and the benefit of U.S. Provisional Application No. 62/544,707, filed on Aug. 11, 2017. The entire contents of the foregoing are incorporated herein by reference.

### SEQUENCE LISTING

This application contains a Sequence Listing that has been submitted electronically as an XML file named 40056-0034002_SL_ST26.xml. The XML file, created on Jun. 23, 2025, is 362,924 bytes in size. The material in the XML file is hereby incorporated by reference in its entirety.

### BACKGROUND

Cancer is the second leading cause of death in the United States. In recent years, great progress has been made in cancer immunotherapy, including immune checkpoint inhibitors, T cells with chimeric antigen receptors (CAR T cells), and oncolytic viruses. Oncolytic viruses are naturally occurring or genetically modified viruses that infect, replicate in, and eventually kill cancer cells while leaving healthy cells unharmed.

Oncolytic viruses are naturally occurring or genetically modified viruses that infect, replicate in, and eventually kill cancer cells while leaving healthy cells unharmed (1, 2). A recently completed Phase III clinical trial of the oncolytic herpes simplex virus T-VEC in 436 patients with unresectable stage IIIB, IIIC or IV melanoma was reported to meet its primary end point, with a durable response rate of 16.3% in patients receiving T-VEC compared to 2.1% in patients receiving GM-CSF (3). Based on the results from this trial, FDA approved T-VEC on Oct. 27, 2015.

Oncolytic virus constructs from at least eight different species have been tested in various phases of clinical trials, including adenovirus, herpes simplex virus-1, Newcastle disease virus, reovirus, measles virus, coxsackievirus, Seneca Valley virus, and vaccinia virus. It has become clear that oncolytic viruses are well tolerated in patients with cancer. The clinical benefits of oncolytic viruses as stand-alone treatments, however, remain limited (5). Due to concerns on the safety of oncolytic viruses, only highly attenuated oncolytic viruses (either naturally avirulent or attenuated through genetic engineering) have been used in both preclinical and clinical studies. Since the safety of oncolytic viruses has now been well established it is time to design and test oncolytic viruses with maximal anti-tumor potency. Oncolytic viruses with a robust oncolytic effect will release abundant tumor antigens, resulting in a strong immunotherapeutic effect.

Vaccinia virus, the prototype member of the poxvirus family, was used as smallpox vaccine to eradicate smallpox that is estimated to have killed 500 million people just in the 19^{th} and 20^{th} centuries. It, thus, is arguably the most successful live biotherapeutic agent. The safety of vaccinia virus was well demonstrated in millions of people worldwide. Vaccinia virus is also the first oncolytic virus showing viral oncolysis in the laboratory. Vaccinia virus as an oncolytic virus has been tested in many clinical trials and has been shown to be well tolerated in patients with late-stage cancer (2). Several studies show that in terms of oncolytic activity vaccinia virus is superior to adenovirus (6), one of the best studied oncolytic virus species and the first oncolytic virus approved for cancer treatment in China (7). Besides vaccinia virus, other members in the poxvirus family were also tested as oncolytic viruses, including raccoonpox virus (8), orf virus (9), and myxoma virus (10).

### SUMMARY

Described herein is a recombinant chimeric poxvirus comprising a nucleotide sequence having a sequence identity of at least 70% (80%, 85%, 90%, 95%, 98%) to SEQ ID NO: 1 or SEQ ID NO:2 (or having a having a sequence identity of at least 70% (80%, 85%, 90%, 95%, 98%) to SEQ ID NO: 1 or SEQ ID NO:2 that has been modified by deletion of the TK gene) and further comprising a nucleotide sequence encoding human CD19 or a portion thereof. The recombinant poxvirus is oncolytic and can infect and kill certain cancer cells. It can also cause the infected cells to express cell surface CD19 (or a portion of CD19 that can be expressed on the surface of the cell). The expression of CD19 renders the cells vunerable to killing by CAR T cells targeted to CD19 (“CD19 CAR T cells”). Thus, various cancers can be treated by administering together or sequentially, the recombinant chimeric poxvirus or another oncolytic virus harboring a transgene encoding all or a portion of CD19 (collectively “oncolytic virus expressing CD19”) and CD19 CAR T cells. In some cases, it is preferable to treat virst with oncolytic virus expressing CD19 and then, after time has passed such that cells can become infected and express CD19 (e.g., 1, 2, 3, 4, 5 or more days), treat with CD19 CAR T cells. One or both treatment can be repeated.

In one aspect is provided a recombinant oncolytic virus that includes a transgene, e.g., a transgene in an expression cassette wherein the transgene encodes all or a portion of human CD19 (UniProt ID P15391). The expressed portion of CD19 a portion that can be expressed on the cell surface and can be recognized by an anti-CD19 antibody.

In an another aspect is provided a method of treating cancer in a subject in need thereof, the method including administering to the subject a therapeutically effective amount of a chimeric poxvirus as described herein and, simultaneously or subsequently, T cells expressing a CAR targeted to CD19, thereby treating cancer in the subject. In embodiments, the cancer is, e.g., a B cell cancer, ALL, CLL or B-NHL, diffuse large B-cell lymphoma, follicular lymphoma, or mantle cell lymphoma.

In an aspect the nucleotide sequence having a sequence identity of at least 70% (80%, 85%, 90%, 95%, or 98%) to SEQ ID NO: 1 or SEQ ID NO:2, includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS: (ii) one or more anti-cancer nucleic acid sequences: or (iii) a detectable moiety-encoding nucleic acid sequence.

In another aspect the nucleotide sequence having a sequence identity of at least 70% (80%, 85%, 90%, 95%, or 98%) to SEQ ID NO: 1 includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS: (ii) one or more anti-cancer nucleic acid sequences: or (iii) a detectable moiety-encoding nucleic acid sequence.

In another aspect the nucleotide sequence having a sequence identity of at least 70% to SEQ ID NO:2, includes: (i) nucleic acid fragments from orf virus strain NZ2 and pseudocowpox virus strain TJS: (ii) one or more anti-cancer nucleic acid sequences: or (iii) a detectable moiety-encoding nucleic acid sequence.

In another aspect the nucleotide sequence having a sequence identity of at least 70% (80%, 85%, 90%, 95%, or 98%) to SEQ ID NO:3, includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS: (ii) one or more anti-cancer nucleic acid sequences: or (iii) a detectable moiety-encoding nucleic acid sequence.

In an aspect the nucleotide sequence having a sequence identity of at least 70% (80%, 85%, 90%, 95%, or 98%) to SEQ ID NO: 1 or SEQ ID NO:2, includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS: (ii) one or more anti-cancer nucleic acid sequences: (iii) one or more nucleic acid binding sequences: or (iv) a detectable moiety-encoding nucleic acid sequence.

In another aspect the nucleotide sequence having a sequence identity of at least 70% (80%, 85%, 90%, 95%, or 98%) to SEQ ID NO:1, includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS: (ii) one or more anti-cancer nucleic acid sequences: (iii) one or more nucleic acid binding sequences: or (iv) a detectable moiety-encoding nucleic acid sequence.

In another aspect the nucleotide sequence having a sequence identity of at least 70% (80%, 85%, 90%, 95%, or 98%) (80%, 85%, 90%, 95%, or 98%) to SEQ ID NO:2, includes: (i) nucleic acid fragments from orf virus strain NZ2 and pseudocowpox virus strain TJS: (ii) one or more anti-cancer nucleic acid sequences: (iii) one or more nucleic acid binding sequences: or (iv) a detectable moiety-encoding nucleic acid sequence.

In another aspect the nucleotide sequence having a sequence identity of at least 70% (80%, 85%, 90%, 95%, or 98%) to SEQ ID NO:3, includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS: (ii) one or more anti-cancer nucleic acid sequences; (iii) one or more nucleic acid binding sequences: or (iv) a detectable moiety-encoding nucleic acid sequence.

### BRIEF DESCRIPTION OF THE DRAWINGS

FIG. 1. Chimeric orthopoxvirus isolates #33 (SEQ ID NO:1) and #17 show superior cancer cell killing capability compared to the parental individual wild-type virus strains and the control viruses GLV-1h68 and OncoVEX GFP.

FIG. 2. Chimeric parapoxivirus isolate #189 (SEQ ID NO:2) shows superior cancer cell killing capability compared to the parental individual wild-type virus strains and the control viruses GLV-1h68 and OncoVEX GFP.

FIG. 3. Chimeric orthopoxvirus isolates #17 and #33 (SEQ ID NO:1) show potent cancer cell killing capacity in pancreatic cancer cell lines compared to their parent virus strains and the control viruses GLV-1h68 and OncoVEX GFP.

FIG. 4. Chimeric parapoxvirus isolate #189 (SEQ ID NO:2) shows potent cancer cell killing capability in pancreatic cancer cell lines compared to its parent virus strains and the control viruses GLV-1h68 and OncoVEX GFP.

FIGS. 5A-5C. Chimeric virus isolates #33 (SEQ ID NO:1) and #189 (SEQ ID NO: 2) show superior cell killing activity in gastric cancer cell lines. Cancer cells were infected with each virus at MOIs of 0.01, 0.1, and 1.0. Cell viabilities at 96 hours post infection were plotted against MOIs in MKN-45 (A), OCUM-2M (B), and KATO-3 (C) gastric cell lines.

FIG. 6. CD19 expression of MDA-MB-468 parental and CD19t lines as determined by flow cytometry.

FIG. 7. Schematic depiction of CD19 CAR used in the studies described herein.

FIG. 8. Truncated EGFR expression, which indicates the presence of CD19 CAR, as determined by flow cytometry.

FIG. 9. The co-cultures of MDA-MB-468 tumors, either Mock or CD19 CAR T cell, and 33-CD19t virus were stained and analyzed by flow cytometry to determine CD19t expression on target cells. Top: without CD19 CART cell treatment. Bottom: with CD19 CAR T cell treatment.

FIG. 10. Supernatant taken from a 48-hour long term kill assay with 33-CD19t virus. The T cell production of IL-2 (bottom) and IFN-γ (top) was detected by an enzyme linked immunosorbent assay (ELISA).

FIG. 11. Cell culture images of 24, 48 and 72-hour tumor killing assays (10×) comparing Mock to CAR T cell treatment after treatment with virus.

FIGS. 12A-12C. Long term killing assays of MDA-MB-468 cells. Mock or CD19 CAR T cell and 33-CD19t virus were exposed to the cells and cultures were analyzed to determine CD19t expression and % tumor killing compared to a virus control (left) at (A) 24-hours, (B) 48-hours, and (C) 72-hours.

FIGS. 13A-13E. An oncolytic virus can effectively deliver CD19t to solid tumors and activate CD19-CAR T cells in vitro. (A) Schematic of vaccinia oncolytic virus [CF33-(SE)hCD19t], showing incorporation of human truncated CD19 (CD19t) under the control of the synthetic early promoter (PSE) inserted into the J2R locus replacing the thymidine kinase gene. (B) Immunofluorescence microscopy of MDA-MB-468 cells infected for 24 h with OV19t at MOI 0.025 (left) and MOI 1 (middle), or cells transduced with lentivirus to stably express CD19t (right). Images are at 10× magnification, and insets are at 40× magnification. (C) FACS plots displaying the cell surface expression of CD19t and intracellular expression of vaccinia virus on MDA-MB-468 tumor cells determined by flow cytometry after 24 h of OV19t infection at increasing MOIs. (D) Quantification of CD19t (left), vaccinia (middle), and viability (right) of MDA-MB-468 tumor cells following 24, 48, and 72 h co-culture with indicated MOIs of OV19t. (E) Quantification of CD25 (left) and CD137 (right) expression on Mock (untransduced) or CD19-CAR T cells following a 24 hour co-culture with tumor cells at an effector:tumor (E:T) ratio of 1:2 with or without treatment with indicated MOI of OV19t.

FIGS. 14A-14E. OV19t oncolytic virus can force expression of CD19t in tumor cells, which re-direct activation and cytotoxicity of CD19-CAR T cells in vitro. (A) Representative flow cytometric analysis showing cell surface CD107a (left) and intracellular IFNγ expression (right) in CD8⁺CAR⁺ T cells following a 16 h co-culture with MDA-MB-468 tumor cells at a 1:1 E:T ratio with or without treatment with indicated MOI of OV19t. (B) IFNγ production measured by ELISA in supernatants collected from co-cultures with or without treatment with OV19t at indicated MOIs for 24, 48, and 72 h. (C) Tumor killing assay assessed by flow cytometry comparing Mock or CD19-CAR T cells following a 24, 48, or 72 h co-culture with MDA-MB-468 tumor cells treated with indicated MOIs of OV19t. (D) CD19t expression on tumor cells in killing assay described in (C). (E) Virus titers in supernatant collected from co-cultures of T cells and tumors cells treated with indicated MOIs of OV19t.

FIGS. 15A-15D. Anti-tumor efficacy of combination therapy of OV19t and CD19-CAR T cells in a TNBC xenograft model. (A) Mice were engrafted with subcutaneous MDA-MB-468 tumors (5×10⁶ cells) and intratumorally treated with 0, 10⁵, 10⁶, or 10⁷ plaque-forming units (pfu) per mouse, which were harvested at day 3 (left), 7 (middle), or 10 (right) after treatment for quantification of CD19t expression via flow cytometry. (+) represents MDA-MB-468 tumors previously lentivirally transduced to stably express CD19t. (B) Tumor volume (mm³) in NSG mice bearing subcutaneous MDA-MB-468 (5×10⁶ cells) tumors on day 0, and treated with OV19t (10⁷ pfu) on day 36. On day 46, mice were treated with either Mock or CD19-CAR T cells (5×10⁶ cells). (C) Average tumor volumes on day 73 of in vivo experiment described in (B). (D) Schematic of combination therapy concept utilizing OV to introduce CAR targets to intractable solid tumors.

### DETAILED DESCRIPTION

Described herein are recombinant oncolytic viruses that express all or a portion of human CD19. These viruses can be derived from chimeric poxvirus compositions which are oncolytic or other oncolytic viruses. Suitable recombinant oncolytic virus can be created by inserting an expression cassette that includes a sequence encoding human CD19 or portion thereof into chimeric virus or other oncolytic virus described in PCT/US2017/46163, filed 9 Aug. 2017 and incorporated herein by reference.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus, poxvirus), an envelope including lipids and optionally components of host cell membranes, and/or viral proteins.

The term “poxvirus” is used according to its plain ordinary meaning within Virology and refers to a member of Poxviridae family capable of infecting vertebrates and invertebrates which replicate in the cytoplasm of their host. In embodiments, poxvirus virions have a size of about 200 nm in diameter and about 300 nm in length and possess a genome in a single, linear, double-stranded segment of DNA, typically 130-375 kilobase. The term poxvirus includes, without limitation, all genera of poxviridae (e.g., betaentomopoxvirus, yatapoxvirus, cervidpoxvirus, gammaentomopoxvirus, leporipoxvirus, suipoxvirus, molluscipoxvirus, crocodylidpoxvirus, alphaentomopoxvirus, capripoxvirus, orthopoxvirus, avipoxvirus, and parapoxvirus). In embodiments, the poxvirus is an orthopoxvirus (e.g., smallpox virus, vaccinia virus, cowpox virus, monkeypox virus), parapoxvirus (e.g., orf virus, pseudocowpox virus, bovine popular stomatitis virus), yatapoxvirus (e.g., tanapox virus, yaba monkey tumor virus) or molluscipoxvirus (e.g., molluscum contagiosum virus). In embodiments, the poxvirus is an orthopoxvirus (e.g., cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, or vaccinia virus strain AS). In embodiments, the poxvirus is a parapoxvirus (e.g., orf virus strain NZ2 or pseudocowpox virus strain TJS).

The term “chimeric” used within the context of a chimeric poxvirus, is used according to its plain ordinary meaning within Virology and refers to a hybrid microorganism (e.g., chimeric poxvirus) created by joining nucleic acid fragments from two or more different microorganisms (e.g., two viruses from the same subfamily, two viruses from different subfamilies). In embodiments, each of at least two of the nucleic acid fragments contain the essential genes necessary for replication. The chimeric poxvirus provided herein including embodiments thereof may include one or more transgenes (i.e., nucleic acid sequences not native to the viral genome). For example, the chimeric poxvirus provided herein including embodiments thereof may include an anti-cancer nucleic acid sequence, a nucleic acid binding sequence, a detectable moiety-encoding nucleic acid sequence or any combination thereof. In embodiments, the chimeric poxvirus includes a nucleic acid sequence including an anti-cancer nucleic acid sequence, a nucleic acid binding sequence and a detectable moiety-encoding nucleic acid sequence. In embodiments, the chimeric poxvirus includes a nucleic acid sequence including an anti-cancer nucleic acid sequence and a detectable moiety-encoding nucleic acid sequence. In embodiments, the chimeric poxvirus includes a nucleic acid sequence including a nucleic acid binding sequence and a detectable moiety-encoding nucleic acid sequence. In embodiments, the chimeric poxvirus includes a nucleic acid sequence including an anti-cancer nucleic acid sequence and a nucleic acid binding sequence.

The term “cowpox virus strain Brighton” is used according to its common, ordinary meaning and refers to virus strains of the same or similar names and functional fragments and homologs thereof. The term includes recombinant or naturally occurring forms of cowpox virus strain Brighton or variants thereof that maintain cowpox virus strain Brighton activity (e.g. within at least 30%, 40%, 50%, 60%, 70%, 80%, 90%, 95%, or 100%). The term includes recombinant or naturally occurring forms of cowpox virus strain Brighton or variants thereof whose genome has sequence identity to the cowpox virus strain Brighton genome (e.g. about 65%, 70%, 75%, 80%, 85%, 90%, 95%, 99% or 100% identity to the cowpox virus strain Brighton genome). Cowpox virus strain Brighton may refer to variants having mutated amino acid residues that modulate (e.g. increase or decrease when compared to cowpox virus strain Brighton) cowpox virus strain Brighton activity, expression, cellular targeting, or infectivity. Cowpox virus strain Brighton may be modified as described herein. In embodiments, the cowpox virus strain Brighton refers to the virus strain identified by ATCC (American Type Culture Collection) reference number ATCC VR-302™, variants or homologs thereof.

The term “raccoonpox virus strain Herman” is used according to its common, ordinary meaning and refers to virus strains of the same or similar names and functional fragments and homologs thereof. The term includes recombinant or naturally occurring forms of raccoonpox virus strain Herman or variants thereof that maintain raccoonpox virus strain Herman activity (e.g. within at least 30%, 40%, 50%, 60%, 70%, 80%, 90%, 95%, or 100%). The term includes recombinant or naturally occurring forms of raccoonpox virus strain Herman or variants thereof whose genome has sequence identity to the raccoonpox virus strain Herman genome (e.g. about 65%, 70%, 75%, 80%, 85%, 90%, 95%, 99% or 100% identity to the raccoonpox virus strain Herman genome). Raccoonpox virus strain Herman may refer to variants having mutated amino acid residues that modulate (e.g. increase or decrease when compared to raccoonpox virus strain Herman) raccoonpox virus strain Herman activity, expression, cellular targeting, or infectivity. Raccoonpox virus strain Herman may be modified as described herein. In embodiments, the raccoonpox virus strain Herman refers to the virus strain identified by ATCC reference number ATCC VR-838™ variants or homologs thereof.

The term “rabbitpox virus strain Utrecht” is used according to its common, ordinary meaning and refers to virus strains of the same or similar names and functional fragments and homologs thereof. The term includes recombinant or naturally occurring forms of rabbitpox virus strain Utrecht or variants thereof that maintain rabbitpox virus strain Utrecht activity (e.g. within at least 30%, 40%, 50%, 60%, 70%, 80%, 90%, 95%, or 100%). The term includes recombinant or naturally occurring forms of rabbitpox virus strain Utrecht or variants thereof whose genome has sequence identity to the rabbitpox virus strain Utrecht genome (e.g. about 65%, 70%, 75%, 80%, 85%, 90%, 95%, 99% or 100% identity to the rabbitpox virus strain Utrecht genome). Rabbitpox virus strain Utrecht may refer to variants having mutated amino acid residues that modulate (e.g. increase or decrease when compared to rabbitpox virus strain Utrecht) rabbitpox virus strain Utrecht activity, expression, cellular targeting, or infectivity. Rabbitpox virus strain Utrecht may be modified as described herein. In embodiments, the rabbitpox virus strain Utrecht refers to the virus strain identified by ATCC reference number ATCC VR-1591™, variants or homologs thereof.

The term “vaccinia virus strain WR” is used according to its common, ordinary meaning and refers to virus strains of the same or similar names and functional fragments and homologs thereof. The term includes recombinant or naturally occurring forms of vaccinia virus strain WR or variants thereof that maintain vaccinia virus strain WR activity (e.g. within at least 30%, 40%, 50%, 60%, 70%, 80%, 90%, 95%, or 100%). The term includes recombinant or naturally occurring forms of vaccinia virus strain WR or variants thereof whose genome has sequence identity to the vaccinia virus strain WR genome (e.g. about 65%, 70%, 75%, 80%, 85%, 90%, 95%, 99% or 100% identity to the vaccinia virus strain WR genome). Vaccinia virus strain WR may refer to variants having mutated amino acid residues that modulate (e.g. increase or decrease when compared to vaccinia virus strain WR) vaccinia virus strain WR activity, expression, cellular targeting, or infectivity. Vaccinia virus strain WR may be modified as described herein. In embodiments, the vaccinia virus strain WR refers to the virus strain identified by ATCC reference number ATCC VR-1354™, variants or homologs thereof.

The term “vaccinia virus strain IHD” is used according to its common, ordinary meaning and refers to virus strains of the same or similar names and functional fragments and homologs thereof. The term includes recombinant or naturally occurring forms of vaccinia virus strain IHD or variants thereof that maintain vaccinia virus strain IHD activity (e.g. within at least 30%, 40%, 50%, 60%, 70%, 80%, 90%, 95%, or 100%). The term includes recombinant or naturally occurring forms of vaccinia virus strain IHD or variants thereof whose genome has sequence identity to the vaccinia virus strain IHD genome (e.g. about 65%, 70%, 75%, 80%, 85%, 90%, 95%, 99% or 100% identity to the vaccinia virus strain IHD genome). Vaccinia virus strain IHD may refer to variants having mutated amino acid residues that modulate (e.g. increase or decrease when compared to vaccinia virus strain IHD) vaccinia virus strain IHD activity, expression, cellular targeting, or infectivity. Vaccinia virus strain IHD may be modified as described herein. In embodiments, the vaccinia virus strain IHD refers to the virus strain identified by ATCC reference number ATCC VR-156™, variants or homologs thereof.

The term “vaccinia virus strain Elstre” is used according to its common, ordinary meaning and refers to virus strains of the same or similar names and functional fragments and homologs thereof. The term includes recombinant or naturally occurring forms of vaccinia virus strain Elstre or variants thereof that maintain vaccinia virus strain Elstre activity (e.g. within at least 30%, 40%, 50%, 60%, 70%, 80%, 90%, 95%, or 100%). The term includes recombinant or naturally occurring forms of vaccinia virus strain Elstre or variants thereof whose genome has sequence identity to the vaccinia virus strain Elstre genome (e.g. about 65%, 70%, 75%, 80%, 85%, 90%, 95%, 99% or 100% identity to the vaccinia virus strain Elstre genome). Vaccinia virus strain Elstre may refer to variants having mutated amino acid residues that modulate (e.g. increase or decrease when compared to vaccinia virus strain Elstre) vaccinia virus strain Elstre activity, expression, cellular targeting, or infectivity. Vaccinia virus strain Elstre may be modified as described herein. In embodiments, the vaccinia virus strain Elstre refers to the virus strain identified by ATCC reference number ATCC VR-1549™, variants or homologs thereof.

The term “vaccinia virus strain CL” is used according to its common, ordinary meaning and refers to virus strains of the same or similar names and functional fragments and homologs thereof. The term includes recombinant or naturally occurring forms of vaccinia virus strain CL or variants thereof that maintain vaccinia virus strain CL activity (e.g. within at least 30%, 40%, 50%, 60%, 70%, 80%, 90%, 95%, or 100%). The term includes recombinant or naturally occurring forms of vaccinia virus strain CL or variants thereof whose genome has sequence identity to the vaccinia virus strain CL genome (e.g. about 65%, 70%, 75%, 80%, 85%, 90%, 95%, 99% or 100% identity to the vaccinia virus strain CL genome). Vaccinia virus strain CL may refer to variants having mutated amino acid residues that modulate (e.g. increase or decrease when compared to vaccinia virus strain CL) vaccinia virus strain CL activity, expression, cellular targeting, or infectivity. Vaccinia virus strain CL may be modified as described herein. In embodiments, the vaccinia virus strain CL refers to the virus strain identified by ATCC reference number ATCC VR-1774™, variants or homologs thereof.

The term “vaccinia virus strain Lederle-Chorioallantoic” is used according to its common, ordinary meaning and refers to virus strains of the same or similar names and functional fragments and homologs thereof. The term includes recombinant or naturally occurring forms of vaccinia virus strain Lederle-Chorioallantoic or variants thereof that maintain vaccinia virus strain Lederle-Chorioallantoic activity (e.g. within at least 30%, 40%, 50%, 60%, 70%, 80%, 90%, 95%, or 100%). The term includes recombinant or naturally occurring forms of vaccinia virus strain Lederle-Chorioallantoic or variants thereof whose genome has sequence identity to the vaccinia virus strain Lederle-Chorioallantoic genome (e.g. about 65%, 70%, 75%, 80%, 85%, 90%, 95%, 99% or 100% identity to the vaccinia virus strain Lederle-Chorioallantoic genome). Vaccinia virus strain Lederle-Chorioallantoic may refer to variants having mutated amino acid residues that modulate (e.g. increase or decrease when compared to vaccinia virus strain Lederle-Chorioallantoic) vaccinia virus strain Lederle-Chorioallantoic activity, expression, cellular targeting, or infectivity. Vaccinia virus strain Lederle-Chorioallantoic may be modified as described herein. In embodiments, the vaccinia virus strain Lederle-Chorioallantoic refers to the virus strain identified by ATCC reference number ATCC VR-118™, variants or homologs thereof.

The term “vaccinia virus strain AS” is used according to its common, ordinary meaning and refers to virus strains of the same or similar names and functional fragments and homologs thereof. The term includes recombinant or naturally occurring forms of vaccinia virus strain AS or variants thereof that maintain vaccinia virus strain AS activity (e.g. within at least 30%, 40%, 50%, 60%, 70%, 80%, 90%, 95%, or 100%). The term includes recombinant or naturally occurring forms of vaccinia virus strain AS or variants thereof whose genome has sequence identity to the vaccinia virus strain AS genome (e.g. about 65%, 70%, 75%, 80%, 85%, 90%, 95%, 99% or 100% identity to the vaccinia virus strain AS genome). Vaccinia virus strain AS may refer to variants having mutated amino acid residues that modulate (e.g. increase or decrease when compared to vaccinia virus strain AS) vaccinia virus strain AS activity, expression, cellular targeting, or infectivity. Vaccinia virus strain AS may be modified as described herein. In embodiments, the vaccinia virus strain AS refers to the virus strain identified by ATCC reference number ATCC VR-2010™, variants or homologs thereof.

The term “orf virus strain NZ2” is used according to its common, ordinary meaning and refers to virus strains of the same or similar names and functional fragments and homologs thereof. The term includes recombinant or naturally occurring forms of orf virus strain NZ2 or variants thereof that maintain orf virus strain NZ2 activity (e.g. within at least 30%, 40%, 50%, 60%, 70%, 80%, 90%, 95%, or 100%). The term includes recombinant or naturally occurring forms of orf virus strain NZ2 or variants thereof whose genome has sequence identity to the orf virus strain NZ2 genome (e.g. about 65%, 70%, 75%, 80%, 85%, 90%, 95%, 99% or 100% identity to the orf virus strain NZ2 genome). Orf virus strain NZ2 may refer to variants having mutated amino acid residues that modulate (e.g. increase or decrease when compared to orf virus strain NZ2) orf virus strain NZ2 activity, expression, cellular targeting, or infectivity. Orf virus strain NZ2 may be modified as described herein. In embodiments, the orf virus strain NZ2 refers to the virus strain identified by ATCC reference number ATCC VR-1548™, variants or homologs thereof.

The term “pseudocowpox virus strain TJS” is used according to its common, ordinary meaning and refers to virus strains of the same or similar names and functional fragments and homologs thereof. The term includes recombinant or naturally occurring forms of pseudocowpox virus strain TJS or variants thereof that maintain pseudocowpox virus strain TJS activity (e.g. within at least 30%, 40%, 50%, 60%, 70%, 80%, 90%, 95%, or 100%). The term includes recombinant or naturally occurring forms of pseudocowpox virus strain TJS or variants thereof whose genome has sequence identity to the pseudocowpox virus strain TJS genome (e.g. about 65%, 70%, 75%, 80%, 85%, 90%, 95%, 99% or 100% identity to the pseudocowpox virus strain TJS genome). Pseudocowpox virus strain TJS may refer to variants having mutated amino acid residues that modulate (e.g. increase or decrease when compared to pseudocowpox virus strain TJS) pseudocowpox virus strain TJS activity, expression, cellular targeting, or infectivity. Pseudocowpox virus strain TJS may be modified as described herein. In embodiments, the pseudocowpox virus strain TJS refers to the virus strain identified by ATCC reference number ATCC VR-634™, variants or homologs thereof.

In embodiments, cowpox virus strain Brighton is cowpox virus strain Brighton ATCC VR-302™. In embodiments, raccoonpox virus strain Herman is raccoonpox virus strain Herman ATCC VR-838™. In embodiments, rabbitpox virus strain Utrecht is rabbitpox virus strain Utrecht ATCC VR-1591™. In embodiments, vaccinia virus strain WR is vaccinia virus strain WR ATCC VR-1354™. In embodiments, vaccinia virus strain IHD is vaccinia virus strain IHD ATCC VR-156™. In embodiments, vaccinia virus strain Elstree is vaccinia virus strain Elstree ATCC VR-1549™. In embodiments, vaccinia virus strain CL is vaccinia virus strain CL ATCC VR-1774TM. In embodiments, vaccinia virus strain Lederle-Chorioallantoic is vaccinia virus strain Lederle-Chorioallantoic ATCC VR-118™. In embodiments, vaccinia virus strain AS is vaccinia virus strain AS ATCC VR-2010™. In embodiments, orf virus strain NZ2 is orf virus strain NZ2 ATCC VR-1548™. In embodiments, pseudocowpox virus strain TJS is pseudocowpox virus strain TJS ATCC VR-634™.

### I. Chimeric Poxvirus Compositions

In an aspect, is provided a chimeric poxvirus comprising a nucleotide sequence having a sequence identity of at least 70% (75%, 80%, 85%, 90%, 92%, 94%, 96%, 98%, or 99%) to SEQ ID NO: 1 or SEQ ID NO:2 and a nucleotide sequence encoding human CD19 or a portion thereof that can be expressed on the cell surface. The sequence having at least 70% identify to SEQ IN NO: 1 or 2 in some embodiments includes nucleotide sequences (“nucleic acid fragments”) from at least two poxvirus strains selected from the group including cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS, e.g, nucleotide sequences of at least 100 contiguous nucleotides

The chimeric oncolytic poxviruses as described herein include transgene encoding human a truncated human CD19 (CD19t) that lacks a functional signaling domain, but includes the extracellular domain and transmembrane domain. The truncated human CD19 comprises the amino acid sequence (or a sequence at least 95%, 97%, 98% or 99% identical to)

MPPPRLLFFLLFLTPMEVRPEEPLVVKVEEGDNAVLQCLKGTSDGPTQQLTWSRESP LKPFLKLSLGLPGLGIHMRPLAIWLFIFNVSQQMGGFYLCQPGPPSEKAWQPGWTVN VEGSGELFRWNVSDLGGLGCGLKNRSSEGPSSPSGKLMSPKLYVWAKDRPEIWEGE PPCVPPRDSLNQSLSQDLTMAPGSTLWLSCGVPPDSVSRGPLSWTHVHPKGPKSLLS LELKDDRPARDMWVMETGLLLPRATAQDAGKYYCHRGNLTMSFHLEITARPVLWH WLLRTGGWKVSAVTLAYLIFCLCSLVGILHLQRALVLRRKR (SEQ ID NO: 29). In some cases, the CD19t comprises or consists of amino acids 22-323 of SEQ ID NO: 29. Amino acid 1-21 of SEQ ID NO: 3 are a signaling domain and can be replaced with a different signaling domain. Thus, the oncolytic virus comprises a sequence comprising a nucleotide sequence encoding a truncated human CD19 operably linked to an expression control sequence (e.g., an early promoter).

In embodiments, the nucleic acid fragments are from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

In embodiments, the nucleic acid sequence includes nucleic acid fragments from cowpox virus strain Brighton and raccoonpox virus strain Herman. In embodiments, the nucleic acid sequence includes nucleic acid fragments from cowpox virus strain Brighton and rabbitpox virus strain Utrecht. In embodiments, the nucleic acid sequence includes nucleic acid fragments from cowpox virus strain Brighton and vaccinia virus strain WR. In embodiments, the nucleic acid sequence includes nucleic acid fragments from cowpox virus strain Brighton and vaccinia virus strain IHD. In embodiments, the nucleic acid sequence includes nucleic acid fragments from cowpox virus strain Brighton and vaccinia virus strain Elstree. In embodiments, the nucleic acid sequence includes nucleic acid fragments from cowpox virus strain Brighton and vaccinia virus strain CL. In embodiments, the nucleic acid sequence includes nucleic acid fragments from cowpox virus strain Brighton and vaccinia virus strain Lederle-Chorioallantoic. In embodiments, the nucleic acid sequence includes nucleic acid fragments from cowpox virus strain Brighton and vaccinia virus strain AS. In embodiments, the nucleic acid sequence includes nucleic acid fragments from cowpox virus strain Brighton and orf virus strain NZ2. In embodiments, the nucleic acid sequence includes nucleic acid fragments from cowpox virus strain Brighton and pseudocowpox virus strain TJS.

In embodiments, the nucleic acid sequence includes nucleic acid fragments from rabbitpox virus strain Utrecht and vaccinia virus strain WR. In embodiments, the nucleic acid sequence includes nucleic acid fragments from rabbitpox virus strain Utrecht and vaccinia virus strain IHD. In embodiments, the nucleic acid sequence includes nucleic acid fragments from rabbitpox virus strain Utrecht and vaccinia virus strain Elstree. In embodiments, the nucleic acid sequence includes nucleic acid fragments from rabbitpox virus strain Utrecht and vaccinia virus strain CL. In embodiments, the nucleic acid sequence includes nucleic acid fragments from rabbitpox virus strain Utrecht and vaccinia virus strain Lederle-Chorioallantoic. In embodiments, the nucleic acid sequence includes nucleic acid fragments from rabbitpox virus strain Utrecht and vaccinia virus strain AS. In embodiments, the nucleic acid sequence includes nucleic acid fragments from rabbitpox virus strain Utrecht and orf virus strain NZ2. In embodiments, the nucleic acid sequence includes nucleic acid fragments from rabbitpox virus strain Utrecht and pseudocowpox virus strain TJS.

In embodiments, the nucleic acid sequence includes nucleic acid fragments from vaccinia virus strain WR and vaccinia virus strain IHD. In embodiments, the nucleic acid sequence includes nucleic acid fragments from vaccinia virus strain WR and vaccinia virus strain Elstree. In embodiments, the nucleic acid sequence includes nucleic acid fragments from vaccinia virus strain WR and vaccinia virus strain CL. In embodiments, the nucleic acid sequence includes nucleic acid fragments from vaccinia virus strain WR and vaccinia virus strain Lederle-Chorioallantoic. In embodiments, the nucleic acid sequence includes nucleic acid fragments from vaccinia virus strain WR and vaccinia virus strain AS. In embodiments, the nucleic acid sequence includes nucleic acid fragments from vaccinia virus strain WR and orf virus strain NZ2. In embodiments, the nucleic acid sequence includes nucleic acid fragments from vaccinia virus strain WR and pseudocowpox virus strain TJS.

In embodiments, the nucleic acid sequence includes nucleic acid fragments from vaccinia virus strain IHD and vaccinia virus strain Elstree. In embodiments, the nucleic acid sequence includes nucleic acid fragments from vaccinia virus strain IHD and vaccinia virus strain CL. In embodiments, the nucleic acid sequence includes nucleic acid fragments from vaccinia virus strain IHD and vaccinia virus strain Lederle-Chorioallantoic. In embodiments, the nucleic acid sequence includes nucleic acid fragments from vaccinia virus strain IHD and vaccinia virus strain AS. In embodiments, the nucleic acid sequence includes nucleic acid fragments from vaccinia virus strain IHD and orf virus strain NZ2. In embodiments, the nucleic acid sequence includes nucleic acid fragments from vaccinia virus strain IHD and pseudocowpox virus strain TJS.

In embodiments, the nucleic acid sequence includes nucleic acid fragments from vaccinia virus strain Elstree and vaccinia virus strain CL. In embodiments, the nucleic acid sequence includes nucleic acid fragments from vaccinia virus strain Elstree and vaccinia virus strain Lederle-Chorioallantoic. In embodiments, the nucleic acid sequence includes nucleic acid fragments from vaccinia virus strain Elstree and vaccinia virus strain AS. In embodiments, the nucleic acid sequence includes nucleic acid fragments from vaccinia virus strain Elstree and orf virus strain NZ2. In embodiments, the nucleic acid sequence includes nucleic acid fragments from vaccinia virus strain Elstree and pseudocowpox virus strain TJS.

In embodiments, the nucleic acid sequence includes nucleic acid fragments from vaccinia virus strain CL and vaccinia virus strain Lederle-Chorioallantoic. In embodiments, the nucleic acid sequence includes nucleic acid fragments from vaccinia virus strain CL and vaccinia virus strain AS. In embodiments, the nucleic acid sequence includes nucleic acid fragments from vaccinia virus strain CL and orf virus strain NZ2. In embodiments, the nucleic acid sequence includes nucleic acid fragments from vaccinia virus strain CL and pseudocowpox virus strain TJS.

In embodiments, the nucleic acid sequence includes nucleic acid fragments from vaccinia virus strain Lederle-Chorioallantoic and vaccinia virus strain AS. In embodiments, the nucleic acid sequence includes nucleic acid fragments from vaccinia virus strain Lederle-Chorioallantoic and orf virus strain NZ2. In embodiments, the nucleic acid sequence includes nucleic acid fragments from vaccinia virus strain Lederle-Chorioallantoic and pseudocowpox virus strain TJS.

In embodiments, the nucleic acid sequence includes nucleic acid fragments from vaccinia virus strain AS and orf virus strain NZ2. In embodiments, the nucleic acid sequence includes nucleic acid fragments from vaccinia virus strain AS and pseudocowpox virus strain TJS. In embodiments, the nucleic acid sequence includes nucleic acid fragments from orf virus strain NZ2 and pseudocowpox virus strain TJS.

### II. Chimeric Antigen Receptors (CAR) Targeted to CD19

A variety of CD19 CAR have been described and can be used, including those described in U.S. Pat. No. 7,446,179 and Park et al. 2016 Blood 128:4035. The CD19 CAR can include an scFv that binds CD19, e.g., FMC63 (Zola et al. 1991 Immunol Cell Biol 69:411) or SJ25Cl (Bejcek et al. 1995 Cancer Research 55:2346), both of which are commercially available.

Described herein is a nucleic acid molecule encoding a CAR comprising: an scFv targeted to CD19 (e.g., Asp Ile Gln Met Thr Gln Thr Thr Ser Ser Leu Ser Ala Ser Leu Gly Asp Arg Val Thr Ile Ser Cys Arg Ala Ser Gln Asp Ile Ser Lys Tyr Leu Asn Trp Tyr Gln Gln Lys Pro Asp Gly Thr Val Lys Leu Leu Ile Tyr His Thr Ser Arg Leu His Ser Gly Val Pro Ser Arg Phe Ser Gly Ser Gly Ser Gly Thr Asp Tyr Ser Leu Thr Ile Ser Asn Leu Glu Gln Glu Asp Ile Ala Thr Tyr Phe Cys Gln Gln Gly Asn Thr Leu Pro Tyr Thr Phe Gly Gly Gly Thr Lys Leu Glu Ile Thr Gly Ser Thr Ser Gly Ser Gly Lys Pro Gly Ser Gly Glu Gly Ser Thr Lys Gly Glu Val Lys Leu Gln Glu Ser Gly Pro Gly Leu Val Ala Pro Ser Gln Ser Leu Ser Val Thr Cys Thr Val Ser Gly Val Ser Leu Pro Asp Tyr Gly Val Ser Trp Ile Arg Gln Pro Pro Arg Lys Gly Leu Glu Trp Leu Gly Val Ile Trp Gly Ser Glu Thr Thr Tyr Tyr Asn Ser Ala Leu Lys Ser Arg Leu Thr Ile Ile Lys Asp Asn Ser Lys Ser Gln Val Phe Leu Lys Met Asn Ser Leu Gln Thr Asp Asp Thr Ala Ile Tyr Tyr Cys Ala Lys His Tyr Tyr Tyr Gly Gly Ser Tyr Ala Met Asp Tyr Trp Gly Gln Gly Thr Ser Val Thr Val Ser Ser (SEQ ID NO: 30) or a variant thereof having 1-5 (e.g., 1 or 2) amino acid modifications (e.g., substitutions): a transmembrane domain selected from: a CD4 transmembrane domain or variant thereof having 1-5 (e.g., 1 or 2) amino acid modifications (e.g., substitutions), a CD8 transmembrane domain or variant thereof having 1-5 (e.g., 1 or 2) amino acid modifications (e.g., substitutions), a CD28 transmembrane domain or a variant thereof having 1-5 (e.g., 1 or 2) amino acid modifications (e.g., substitutions), and a CD3ξ transmembrane domain or a variant thereof having 1-5 (e.g., 1 or 2) amino acid modifications (e.g., substitutions): a costimulatory domain (e.g., a CD28 co-stimulatory domain or a variant thereof having 1-5 (e.g., 1 or 2) amino acid modifications (e.g., substitutions): or a 4-1 BB co-stimulatory domain or a variant thereof having 1-5 (e.g., 1 or 2) amino acid modifications (e.g., substitutions): or both a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions): and a CD3ξ signaling domain or a variant thereof having 1-5 (e.g., 1 or 2) amino acid modifications.

In various embodiments: the costimulatory domain is selected from the group consisting of: a CD28 costimulatory domain or a variant thereof having 1-5 (e.g., 1 or acid modifications, a 4-1 BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1 BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions). In various embodiments the 1-5 (e.g., 1 or 2) amino acid modification are substitutions.

In some cases there is a short sequence of 1-6 amino acids (e.g. GGG) between the co-stimulatory domains and the CD3ξ signaling domain and/or between the two co-stimulatory domains.

Additional embodiment the CAR comprises: an scFv targeted to CD19; two different costimulatory domains selected from the group consisting of: a CD28 costimulatory domain or a variant thereof having 1-5 (e.g., 1 or 2) amino acid modifications, a 4-1 BB costimulatory domain or a variant thereof having 1-5 (e.g., 1 or 2) amino acid modifications and an OX40 costimulatory domain or a variant thereof having 1-5 (e.g., 1 or 2) amino acid modifications: two different costimulatory domains selected from the group consisting of: a CD28 costimulatory domain or a variant thereof having 1-2 amino acid modifications, a 4-1 BB costimulatory domain or a variant thereof having 1-2 amino acid modifications and an OX40 costimulatory domain or a variant thereof having 1-2 amino acid modifications: a CD19 scFv or a variant thereof having 1-2 amino acid modifications: a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3ξ transmembrane domain or a variant thereof having 1-2 amino acid modifications: a costimulatory domain (e.g., a CD28 co-stimulatory domain or a variant thereof having 1-5 (e.g., 1 or 2) amino acid modifications (e.g., substitutions); or a 4-1 BB co-stimulatory domain or a variant thereof having 1-5 (e.g., 1 or 2) amino acid modifications (e.g., substitutions); or both a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions); and CD3ξ signaling domain of a variant thereof having 1-2 amino acid modifications: a spacer region located between the CD19 scFv or variant thereof and the transmembrane domain (e.g., the spacer region comprises an amino acid sequence selected from the group consisting of SEQ ID NOs: 2-12 and 42 (Table 3) or a variant thereof having 1-5 (e.g., 1 or 2) amino acid modifications): the spacer comprises an IgG hinge region: the spacer region comprises 1-150 amino acids: there is no spacer: the 4-1 BB signaling domain comprises the amino acid sequence of SEQ ID N0:24 the CD3& signaling domain comprises the amino acid sequence of SEQ ID N0:21 and a linker of 3 to 15 amino acids that is located between the costimulatory domain and the CD3ξ signaling domain or variant thereof. In certain embodiments where there are two costimulatory domains, one is a 4-1 BB costimulatory domain and the other a costimulatory domain selected from: CD28 and CD28gg. In various embodiments the 1-5 (e.g., 1 or 2) amino acid modification are substitutions, e.g., conservative substitutions.

Also disclosed is a population of human T cells transduced by a vector comprising an expression cassette encoding a chimeric antigen receptor, wherein chimeric antigen receptor comprises: an scFv targeted to CD19; a transmembrane domain selected from: a CD4 transmembrane domain or variant thereof having 1-5 amino acid modifications (e.g., 1 or 2) amino acid modifications (e.g., substitutions), a CD8 transmembrane domain or variant thereof having 1-5 amino acid modifications (e.g., 1 or 2) amino acid modifications (e.g., substitutions), a CD28 transmembrane domain or a variant thereof having 1-5 amino acid modifications (e.g., 1 or 2) amino acid modifications (e.g., substitutions), and a CD3ξ transmembrane domain or a variant thereof having 1-5 amino acid modifications (e.g., 1 or 2) amino acid modifications (e.g., substitutions); a costimulatory domain (e.g., a CD28 co-stimulatory domain or a variant thereof having 1-5 (e.g., 1 or 2) amino acid modifications (e.g., substitutions); or a 4-1 BB co-stimulatory domain or a variant thereof having 1-5 (e.g., 1 or 2) amino acid modifications (e.g., substitutions): or both a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions): and CD3ξ signaling domain of a variant thereof having 1-5 amino acid modifications (e.g., 1 or 2) amino acid modifications (e.g., substitutions). In various embodiments: the population of human T cells comprises central memory T cells (TCM cells) e.g., at least 20%, 30%, 40%, 50% 60%, 70%, 80% of the cells are TCM cells, or the population of T cells comprises a combination of central memory T cells, naive T cells and stem central memory cells (T_{CM/SCM/N} cells) e.g., at least 20%, 30%, 40%, 50% 60%, 70%, 80% of the cells are T_{CM/SCM/N} cells. In either case, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells).

Also described is a method of treating cancer in a patient comprising administering a population of autologous or allogeneic human T cells (e.g., autologous or allogenic T cells comprising central memory T cells (TCM cells) or a combination of central memory T cells, naive T cells and stem central memory cells (i.e., the T cells are TCM/SCM/N cells) at least 20%, 30%, 40%, 50% 60%, 70%, 80% of the cells are TCM/SCM/N cells. In either case, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+and at least 4%, 5%, 8%, 10%, 20 of the CD3+ cells are CD8+ cells) transduced by a vector comprising an expression cassette encoding a chimeric antigen receptor.

The CD19 CAR can include a spacer region located between the CD19 binding domain (e.g., a CD19 scFv) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG |
| | | (SEQ ID NO: 26) |
| IgG4 hinge | 12 aa | ESKYGPPCPPCP |
| (S→P)(S228P) | | (SEQ ID NO: 27) |
| IgG4 hinge | 12 aa | ESKYGPPCPSCP |
| | | (SEQ ID NO: 4) |
| IgG4 hinge | 22 aa | ESKYGPPCPPCPGGGSSGGGSG |
| (S228P) + | | (SEQ ID NO: 5) |
| linker | | |
| CD28 hinge | 39 aa | IEVMYPPPYLDNEKSNGTIIHVKGKHL |
| | | CPSPLFPGPSKP |
| | | (SEQ ID NO: 6) |
| CD8 hinge- | 48 aa | AKPTTTPAPRPPTPAPTIASQPLSLRP |
| 48aa | | EACRPAAGGAVHTRGLDFACD |
| | | (SEQ ID NO: 7) |
| CD8 hinge- | 45 aa | TTTPAPRPPTPAPTIASQPLSLRPEAC |
| 45aa | | RPAAGGAVHTRGLDFACD |
| | | (SEQ ID NO: 8 |
| IgG4(HL-CH3) | 129 aa | ESKYGPPCPPCPGGGSSGGGSGGQPR |
| (includes | | EPQVYTLPPSQEEMTKNQVSLTCLVK |
| S228P | | GFYPSDIAVEWESNGQPENNYKTTPP |
| in hinge) | | VLDSDGSFFL YSRLTVDKSRWQEGNV |
| | | FSCSVMHEALHNHYTQKSLSLSLGK |
| | | SEQ ID NO: 9) |
| IgG4(L235E, | 229 aa | ESKYGPPCPSCPAPEFEGGPSVFLFPP |
| N297Q) | | KPKDTLMISRTPEVTCVVVDVSQEDPE |
| | | VQFNWYVDGVEVHQAKTKPREEQF |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK |
| | | (SEQ ID NO: 10) |
| IgG4(S228P, | 229 aa | ESKYGPPCPPCPAPEFEGGPSVFLFPP |
| L235E, N297Q) | | KPKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSL TCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK |
| | | (SEQ ID NO: 11) |
| IgG4(CH3) | 107 aa | GQPREPQVYTLPPSQEEMTKNQVSLT |
| | | CLVKGFYPSDIAVEWESNGQPENNYK |
| | | TTPPVLDSDGSFFLYSRLTVDKSRWQ |
| | | EGNVFSCSVMHEALHNHYTQKSLSLSL |
| | | GK(SEQ ID NO: 12) |
| HL | 22 aa | ESKYGPPCPPCPGGGSSGGGSG |
| | | (SEQ ID NO: 28) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fe hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine: 2) Amino acids with uncharged polar R groups: Glycine, Serine, Threonine, Cysteine, Tyrosine, Asparagine, Glutamine: 3) Amino acids with charged polar R groups (negatively charged at pH 6.0): Aspartic acid, Glutamic acid: 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

For amino acid positions in immunoglobulin discussed herein, numbering is according to the EU index or EU numbering scheme (Kabat et al. 1991 Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al. 1969 Proc Natl Acad Sci USA 63:78-85).

A variety of transmembrane domains can be used in the. Table 2 includes examples of suitable transmembrane domains. Where a spacer domain is present, the transmembrane domain is located carboxy terminal to the spacer domain.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVT |
| | | | VAFIIFWV (SEQ ID |
| | | | NO: 14) |
| CD28(M) | NM_006139 | 28 aa | MFWVLVVVGGVLACYSLLV |
| | | | TVAFIIFWV (SEQ ID NO: |
| | | | 15) |
| CD4 | M35160 | 22 aa | MALIVLGGVAGLLLFIGLG |
| | | | IFF (SEQ ID NO: 16) |
| CD8tm | NM_001768 | 21 aa | IYIWAPLAGTCGVLLLSLV |
| | | | IT (SEQ ID NO: 17) |
| CD8tm2 | NM_001768 | 23 aa | IYIWAPLAGTCGVLLLSLV |
| | | | ITLY (SEQ ID NO: 18) |
| CD8tm3 | NM_001768 | 24 aa | IYIWAPLAGTCGVLLLSLV |
| | | | ITLYC (SEQ ID NO: 19) |
| 41BB | NM_001561 | 27 aa | IISFFLALTSTALLFLLFF |
| | | | LTLRFSVV (SEQ ID NO: |
| | | | 20) |

Many of the CAR described herein include one or more (e.g., two) costimulatory domains. The costimulatory domain(s) are located between the transmembrane domain and the CD3ξ signaling domain. Table 3 includes examples of suitable costimulatory domains together with the sequence of the CD3ξ signaling domain.

TABLE 3
CD3zeta Domain and Examples of
Costimulatory Domains

| Name | Accession | Length | Sequence |
|---|---|---|---|
| CD3ζ | J04132.1 | 113 aa | RVKFSRSADAPAYQQGQN |
| | | | QLYNELNLGRREEYDVLD |
| | | | KRRGRDPEMGGKPRRKNP |
| | | | QEGLYNELQKDKMAEAYS |
| | | | EIGMKGERRRGKGHDGLY |
| | | | QGLSTATKDTYDALHMQA |
| | | | LPPR (SEQ ID NO: |
| | | | 21) |
| CD28 | NM_006139 | 42 aa | RSKRSRLLHSDYMNMTPR |
| | | | RPGPTRKHYQPYAPPRDF |
| | | | AAYRS (SEQ ID NO: |
| | | | 22) |
| CD28gg* | NM_006139 | 42 aa | RSKRSRGGHSDYMNMTPR |
| | | | RPGPTRKHYQPYAPPRDF |
| | | | AAYRS (SEQ ID NO: |
| | | | 23) |
| 41BB | NM_001561 | 42 aa | KRGRKKLLYIFKQPFMRP |
| | | | VQTTQEEDGCSCRFPEEE |
| | | | EGGCEL (SEQ ID NO: |
| | | | 24) |
| OX40 | | 42 aa | ALYLLRRDQRLPPDAHKP |
| | | | PGGGSFRTPIQEEQADAH |
| | | | STLAKI (SEQ ID NO: |
| | | | 25) |

### EXAMPLES

### Example 1. Novel Potent Chimeric Poxviruses for Oncolytic Immunotherapy of Cancer

Described bellow are chimeric poxviruses that are oncolytic and can be used to prepare recombinant chimeric poxviruses that express human CD19 in cells infected by the recombinant virus. Because the recombinant virus will infect cancer cells and cause them to express CD19. The infected cells can be targeted by a CAR directed to CD19.

A Chimeric poxviruses used to create recombinant poxvirus have potential to combine favorable features from different virus species, thus, are superior to individual wild-type viruses. Since orthopoxviruses and parapoxviruses are antigenically distinct the potent chimeric orthopoxvirus and the potent chimeric parapoxvirus generated in this study can be potentially combined into the same treatment regimen to achieve the maximum therapeutic efficacy. As explained in greater detail in PCT/US2017/46163 filed 9 Aug. 2017, chimeric poxvirus, including isolates #33 (SEQ ID NO:1) and #189 (SEQ ID NO:2) were generated from pools of chimeric orthopoxviruses and chimeric parapoxviruses. Several chimeric orthopoxvirus and parapoxvirus isolates, including isolates #33 and #189 showed superior killing capacity in a panel of the NCI 60 cancer cell lines compared to their parental individual wiltype viruses.

### Generation of Chimeric Virus Pools and Isolation of Individual Chimeric Viruses.

A pool of chimeric orthopoxviruses was generated by co-infecting CV-1 cells with cowpox virus strain Brighton, raccoonpox virus strain Herman, rabbitpox virus strain Utrecht, and vaccinia virus strains WR, IHD, Elstree, CL, Lederle-Chorioallantoic and AS at a multiplicity of infection (MOI) of 0.01 per virus. To generate a pool of chimeric parapoxviruses, MDBK cells were co-infected with orf virus strain NZ2 and pseudocowpox virus strain TJS at an MOI of 0.1. Applicants' pilot experiments indicate that CV-1 cells are susceptible to all the orthopoxviruses used in this study and that both orf virus and pseudocowpox virus infect and form plaques in MDBK cells.

100 chimeric orthopoxvirus plaques and 100 chimeric parapoxvirus plaques were picked from CV-1 cells infected with the chimeric orthopoxvirus pool and MDBK cells infected with the chimeric parapoxvirus pool, respectively. These two hundred plaques were further plaque-purified two more times in respective cells to yield 200 clonally purified individual chimeric virus isolates. Viruses #14-113 are chimeric orthopoxvirus isolates whereas viruses #114-213 are chimeric parapoxvirus isolates.

Identification of novel potent chimeric poxvirus isolates by high throughput screening in the NCI-60 cell lines. Tumor cell-killing activity of 200 chimeric orthopoxvirus and chimeric parapoxvirus isolates, together with 11 parental virus strains and 2 control oncolytic viruses (GLV-1h68 and OncoVEX GFP) were evaluated and compared in a panel of the NCI-60 cell lines (Table 4). GLV-1h68 is one of the best studied oncolytic vaccinia viruses, and is currently in clinical development. OncoVEX GFP has the same backbone as T-VEC, an oncolytic herpes simplex virus-1 and the first oncolytic virus approved by the FDA. Each cell line was infected with each virus at an MOI of 0.01. Cell viability was measured at 96 h post infection using MTS assays. The virus amount used (MOI 0.01) in this high throughput screening experiment was intentionally kept low, and optimized to compare cell killing in adherent cell lines (the majority of cell lines in the NCI-60 panel are adherent cells) so potent new virus isolates can stand out. This amount of virus, however, was too low to see any significant and consistent cell killing in suspension cell lines. Therefore, the results from 6 leukemia cell lines were not included in the analysis for the purpose of virus comparison.

TABLE 4
Catalog of the NCI-60 cell lines

| Name | Species | Organ of Origin | Culture |
|---|---|---|---|
| BT549 | Human | Breast | Adherent |
| HS 578T | Human | Breast | Adherent |
| MCF7 | Human | Breast | Adherent |
| MDA-MB-231 | Human | Breast | Adherent |
| MDA-MB-468 | Human | Breast | Adherent |
| T-47D | Human | Breast | Adherent |
| SF268 | Human | CNS | Adherent |
| SF295 | Human | CNS | Adherent |
| SF539 | Human | CNS | Adherent |
| SNB-19 | Human | CNS | Adherent |
| SNB-75 | Human | CNS | Adherent |
| U251 | Human | CNS | Adherent |
| Colo205 | Human | Colon | Adherent & Suspension |
| HCC 2998 | Human | Colon | Adherent |
| HCT-116 | Human | Colon | Adherent |
| HCT-15 | Human | Colon | Adherent |
| HT29 | Human | Colon | Adherent |
| KM12 | Human | Colon | Adherent |
| SW620 | Human | Colon | Adherent |
| 786-O | Human | Kidney | Adherent |
| A498 | Human | Kidney | Adherent |
| ACHN | Human | Kidney | Adherent |
| CAKI | Human | Kidney | Adherent |
| RXF 393 | Human | Kidney | Adherent |
| SN12C | Human | Kidney | Adherent |
| TK-10 | Human | Kidney | Adherent |
| UO-31 | Human | Kidney | Adherent |
| CCRF-CEM | Human | Leukemia | Suspension |
| HL-60 | Human | Leukemia | Suspension |
| K562 | Human | Leukemia | Suspension |
| MOLT-4 | Human | Leukemia | Suspension |
| RPMI-8226 | Human | Leukemia | Suspension |
| SR | Human | Leukemia | Suspension |
| A549 | Human | Lung | Adherent |
| EKVX | Human | Lung | Adherent |
| HOP-62 | Human | Lung | Adherent |
| HOP-92 | Human | Lung | Adherent & Suspension |
| NCI-H226 | Human | Lung | Adherent |
| NCI-H23 | Human | Lung | Adherent |
| NCI-H322M | Human | Lung | Suspension |
| NCI-H460 | Human | Lung | Adherent |
| NCI-H522 | Human | Lung | Adherent |
| LOX IMVI | Human | Melanoma | Semi-Adherent |
| M14 | Human | Melanoma | Adherent |
| MALME-3M | Human | Melanoma | Adherent & Suspension |
| MDA-MB-435 | Human | Melanoma | Adherent |
| SK-MEL-2 | Human | Melanoma | Adherent |
| SK-MEL-28 | Human | Melanoma | Adherent |
| SK-MEL-5 | Human | Melanoma | Adherent |
| UACC-257 | Human | Melanoma | Adherent |
| UACC-62 | Human | Melanoma | Adherent |
| IGROV1 | Human | Ovary | Adherent |
| OVCAR-3 | Human | Ovary | Adherent |
| OVCAR-4 | Human | Ovary | Adherent |
| OVCAR-5 | Human | Ovary | Adherent |
| OVCAR-8 | Human | Ovary | Adherent |
| SK-OV-3 | Human | Ovary | Adherent |
| NCI-ADR-RES | Human | Ovary | Adherent |
| DU145 | Human | Prostate | Adherent |
| PC-3 | Human | Prostate | Adherent |

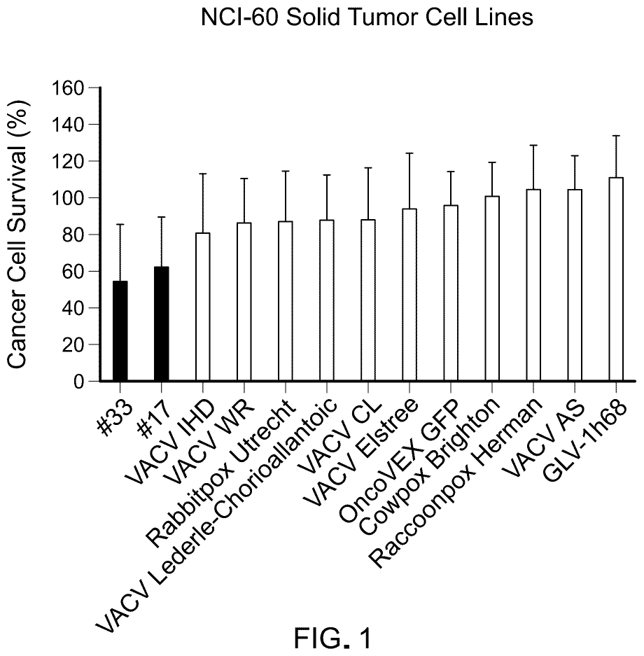

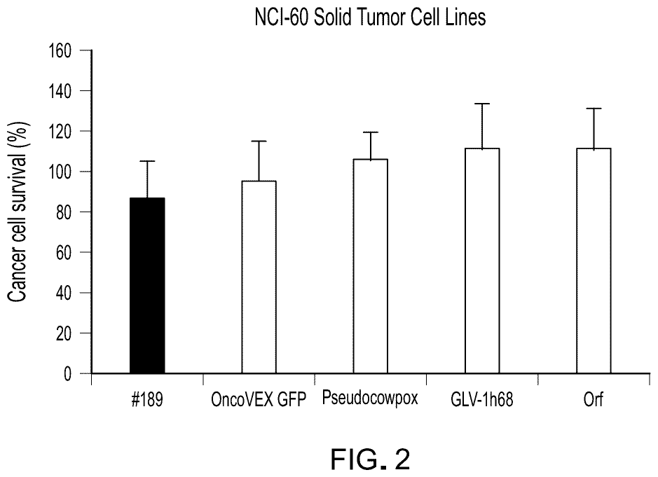

Among 100 new chimeric orthopoxvirus isolates, isolates #17 (SEQ ID NO:3) and #33 (SEQ ID NO:1) demonstrated significantly better cell killing in the NCI-60 solid tumor cell lines than did 9 parental orthopoxvirus strains and two control viruses (FIG. 1). Out of 100 new parapoxvirus isolates, isolate #189 (SEQ ID NO:2) stood out, showing the significantly better cell killing than did two parent parapoxvirus strains and the control viruses (FIG. 2). All three novel chimeric virus isolates (#17, #33, and #189) caused significant cell death in the majority of the NCI-60 solid cancer cell lines even at the low MOI of 0.01. In general, orthopoxvirus strains and chimeric orthopoxvirus isolates were more potent than parapoxvirus strains and chimeric parapoxvirus isolates in killing cancer cells at the low MOI of 0.01.

Genomic DNAs of novel poxvirus isolates #33 and #189 were isolated from purified virions and subject to next-generation sequencing using Illumina Hiseq 2500 with more than 1000× coverage. The gaps were PCR amplified and sequenced by Sanger sequencing. 189,415 base pairs (bps) of the #33 genome were fully sequenced whereas 138,203 bps of the 189 genome were obtained. Initial BLAST against GenBank indicated that the genomic sequences of both #33 and #189 are not identical to any genomic sequences in GenBank. #33 is more close to vaccinia virus strains than to any other orthpoxvirses. #189 is very close to orf virus NZ2 strain, one of the parental parapoxviruses. The nucleotide sequences of all ORFs identified in the orf virus NZ2 strain are identical to that in #189. There is one “G” insertion at position 6755 in the genome of #189 compared to orf virus NZ2. In the inverted terminal repeat regions there are one copy of a repeat element deleted and one copy of another repeat element inserted in the #189 genome. Overall, both #33 and #189 represent novel unique poxvirus isolates.

All cancer cell lines were grown in RPMI-1640 (Mediatech, Manassas, VA). African green monkey kidney fibroblast cells (CV-1) and cow kidney epithelial cells (MDBK) were obtained from American Type Culture Collection (ATCC: Rockville, MD, USA) and grown in DMEM (Mediatech, Manassas, VA). All media were supplemented with 10% FBS (Mediatech, Manassas, VA) and 1% penicillin-streptomycin solution (Mediatech, Manassas, VA). Cells were cultured at 37° C. under 5% CO₂.

Viruses: cowpox virus strain Brighton, raccoonpox virus strain Herman, rabbitpox virus strain Utrecht, vaccinia virus strains WR, IHD, Elstree, CL, Lederle-Chorioallantoic and AS, orf virus strain NZ2 and pseudocowpox virus strain TJS were purchased from ATCC. All orthopoxvirus strains were grown and titrated in CV-1 cells were parapoxvirus strains were grown and titrated in MDBK cells.

Generation of chimeric orthopoxvirus and chimeric parapoxvirus pools and isolation of individual clonal chimeric virus isolates: A pool of chimeric orthopoxviruses was generated by co-infecting CV-1 cells with cowpox virus strain Brighton, raccoonpox virus strain Herman, rabbitpox virus strain Utrecht, and vaccinia virus strains WR, IHD, Elstree, CL, Lederle-Chorioallantoic and AS at a multiplicity of infection (MOI) of 0.01 per virus. To generate a pool of chimeric parapoxviruses, MDBK cells were co-infected with orf virus strain NZ2 and pseudocowpox virus strain TJS at an MOI of 0.1. Infected cells were harvested at 3 days after infection. The initial chimeric orthopoxvirus pool was further passaged for three times in CV-1 cell at an MOI of 0.1 where the initial chimeric parapoxvirus poos was further passaged for three times in MDBK cells at an MOI of 0.1. 100 chimeric orthopoxvirus plaques and 100 chimeric parapoxvirus plaques were picked from CV-1 cells infected with the final chimeric orthopoxvirus pool and MDBK cells infected with the final chimeric parapoxvirus pool, respectively. These two hundred plaques were further plaque-purified two more times in respective cells to yield 200 clonally purified individual chimeric virus isolates.

NCI-60 cancer cell lines and a panel of pancreatic cancer cell line including PANC-1, MIA-PaCa2, BxPC3, FG, Capan-2 and Su.86.86 were dispensed into 96-well plates (3000 cells/well for solid tumor cell lines and 5000/well for leukemia cell lines) using an epMotion 5075 liquid handler (Eppendorf) under a sterile condition, incubated overnight at 37° C. under 5% (v/v) CO₂. Cells were then infected with 200 chimeric orthopoxvirus and chimeric parapoxvirus isolates, together with 11 parental virus strains and 2 control oncolytic viruses GLV-1h68 and OncoVEX GFP at an MOI of 0.01. Cell viability was determined at 96 h post infection using MTS assays (Promega). Absorbance at 490 nm was measured using an automated BMG PHERAstar plate reader (BMG Labtech). Each experiment was performed in duplicate. Cell viability for mock-infected cells was set to 100%.

MKN-45, OCUM-2M and KATO-3 cells were seeded into 96-well plates at a concentration of 3,000 cells per well, and incubated overnight at 37° C. under 5% (v/v) CO₂. Cells were infected with #33, #189, GLV-1h68 and OncoVEX GFP at MOIs of 0.01, 0.1 and 1. Cell viability was monitored daily for 4 days using MTS assays. 37° C. under 5% (v/v) CO₂.

Genomic DNAs of #33 and #189 were extracted from purified virions using Wizard Genomic DNA Purification kit (Promega) and fragmented by sonication. Libraries were prepared using KAPA LTP Library Preparation Kit. Sequencing was done using Illumina Hiseq 2500

### Example 2. High Throughput Screening in Pancreatic Cancer Cell Lines

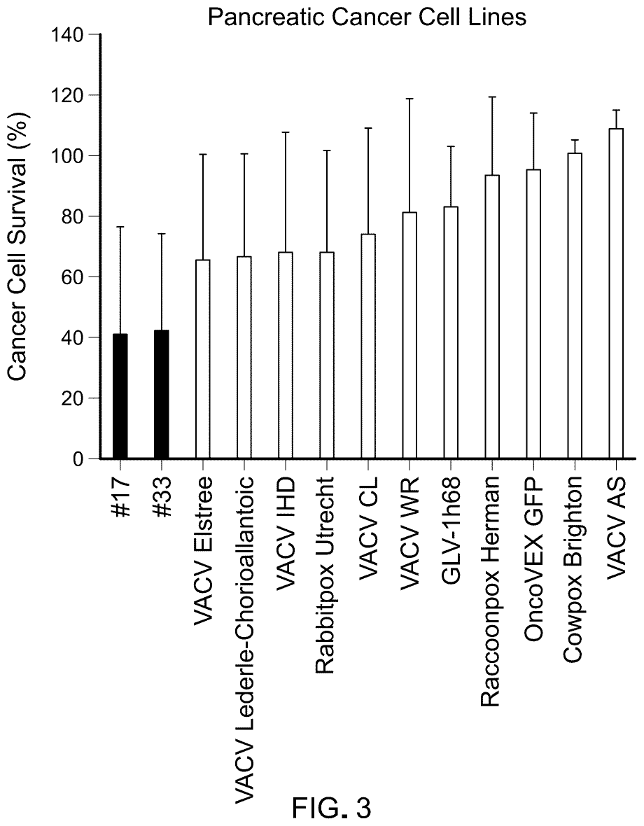

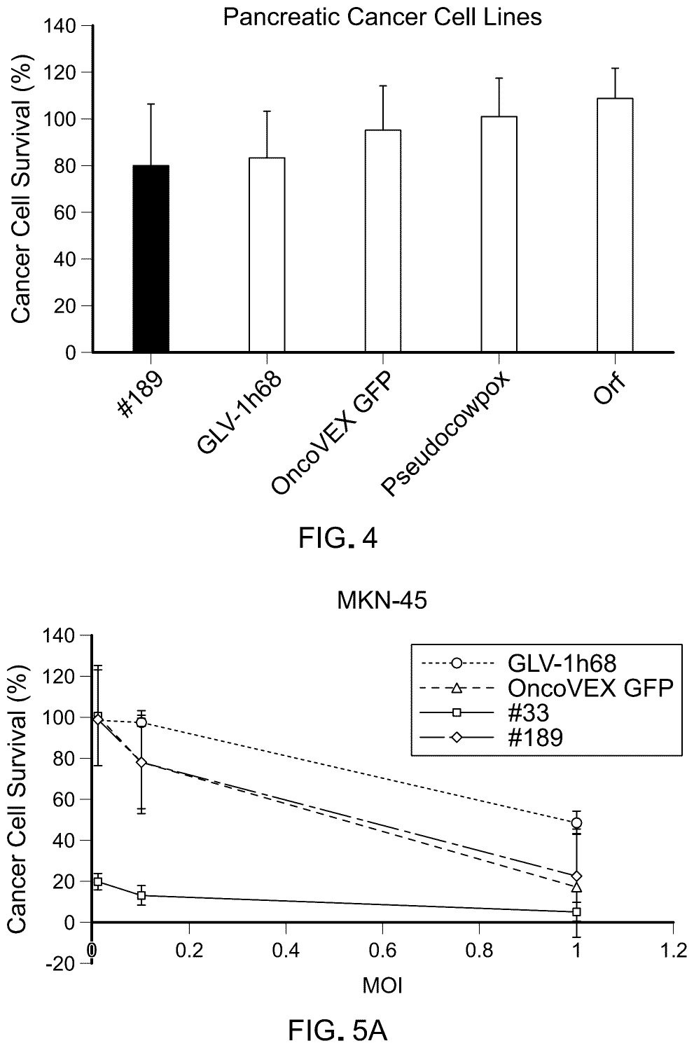

NCI-60 cancer cell lines only contain solid cancers from 8 different organs (see Table 4). To investigate if the results from the NCI-60 cancer cell lines would be reproduced in solid cancers from other organs. Six pancreatic cancer cell lines (BxPC3, FG, MIA PaCa-2, Capan-2, PANC-1, and SU.86.86) were infected at an MOI of 0.1 with the same viruses used in the high throughput screening in the NCI-60 cancer cell lines. Cell viability was again measured at 96 h post infection using MTS assays. Chimeric orthopoxvirus isolates #17 and #33 showed the best cell killing among all the chimeric orthopoxvirus isolates whereas the chimeric parapoxvirus isolate #189 demonstrated the best cell killing among all the chimeric parapoxvirus isolates. They were all better in killing pancreatic cancer cell lines, as shown in Table 5 and FIG. 3 and FIG. 4, than their respective parent virus strains and the control viruses GLV-1h68 and OncoVEX GFP. Thus, the results from the NCI-60 cancer cell lines were very well reproduced in a panel of pancreatic cancer cell lines.

TABLE 5
Pancreatic Cancer Cell Survival.

| | | MIA | | | | | | |
|---|---|---|---|---|---|---|---|---|
| BxPC3 | FG | PaCa2 | Capan 2 | PANC-1 | SU.86.86 | | | |
| Ave. | Ave. | Ave. | Ave. | Ave. | Ave. | Ave. | stdev | Virus Name |
| 20.38 | 18.22 | 24.06 | 109.76 | 20.31 | 52.27 | 40.83 | 36.09 | #17 |
| 4.46 | 26.75 | 52.88 | 85.75 | 13.93 | 69.41 | 42.20 | 32.29 | #33 |
| 31.01 | 60.98 | 102.04 | 113.08 | 57.20 | 30.54 | 65.81 | 34.93 | VACV Elstree |
| 20.78 | 40.62 | 104.90 | 104.86 | 68.57 | 60.23 | 66.66 | 33.91 | VACV Lederle- |
| | | | | | | | | Chorioallantoic |
| 18.21 | 33.81 | 93.44 | 106.36 | 45.64 | 109.87 | 67.89 | 40.05 | VACV IHD |
| 16.40 | 66.23 | 90.91 | 93.81 | 43.05 | 100.69 | 68.51 | 33.31 | Rabbitpox Utrecht |
| 19.57 | 50.96 | 97.52 | 108.60 | 68.27 | 101.81 | 74.46 | 34.78 | VACV CL |
| 17.51 | 75.42 | 102.31 | 123.05 | 68.27 | 103.55 | 81.69 | 37.29 | VACV WR |
| 50.01 | 79.85 | 89.31 | 97.55 | 78.14 | 106.86 | 83.62 | 19.70 | GLV-1h68 |
| 61.29 | 96.76 | 122.32 | 112.34 | 62.99 | 107.42 | 93.85 | 25.91 | Raccoonpox Herman |
| 63.81 | 99.06 | 114.44 | 80.60 | 108.15 | 104.16 | 95.04 | 19.14 | OncoVEX GFP |
| 106.79 | 95.12 | 101.38 | 102.37 | 95.09 | 103.55 | 100.71 | 4.71 | Cowpox Brighton |
| 101.00 | 106.93 | 118.90 | 111.92 | 107.05 | 108.34 | 109.02 | 5.99 | VACV AS |

### Example 3. Novel Chimeric Orthopoxvirus Isolate #33 and Chimeric Parapoxvirus Isolate #189 Show Potent Cell Killing in Gastric Cancer Cell Lines

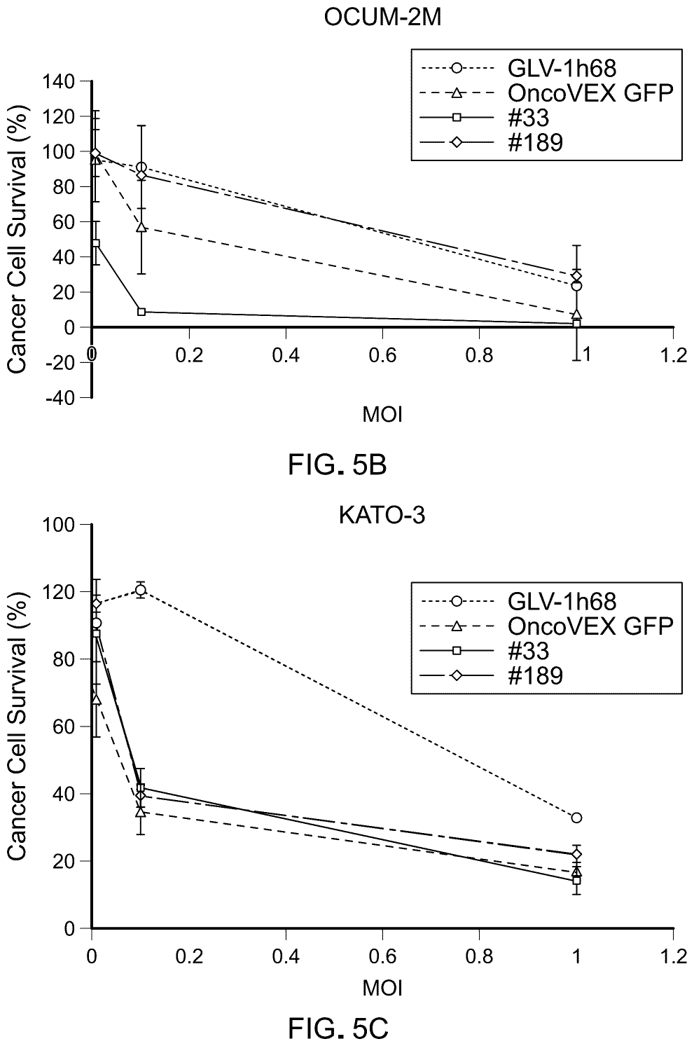

Based on the high throughput screening results from the NCI-60 cancer cell lines and the panel of pancreatic cancer cell lines, novel chimeric orthopoxvirus isolate #33 and chimeric parapoxvirus isolate #189 were chosen for further characterization. Tumor cell killing activity of the isolates #33 and #189 were further investigated in three gastric cancer cell lines. MKN-45, OCUM-2M and KATO-3 cells were infected with #33, #189, GLV-1h68 and OncoVEX GFP at MOIs of 0.01, 0.1 and 1. Cell viability was monitored daily for 4 days using MTS assays. MKN-45 and OCUM-2M cell lines were most sensitive to #33, intermediately sensitive to OncoVEX GFP, and least sensitive to GLV-1h68. While KATO-3 was most sensitive to OncoVEX GFP at the low MOI of 0.01, #33, #189, and OncoVEX GFP killed KATO-3 cells equally well at higher MOIs (0.1 and 1). KATO-3 cells were least sensitive to GLV-1h68. Overall, #33 killed gastric cancer cell lines most efficiently while GLV-1h68 kill gastric cancer cell lines lest efficiently (FIGS. 5A-5C).

The 90 genes that are present in all sequenced ChPVs are listed together with their function when known, as reported in Table 6 (Abbreviations: IMV, intracellular mature virus; IEV, intracellular enveloped virus; EEV, extracellular enveloped virus). Genes are named after their VACV-COP counterpart. The asterisk, *, indicate genes also present in the two EnPVs. Table 3 is adapted from Gubser et al. (Gubser, C., Hue, S., Kellam, P., and Smith, G. L. (2004). Poxvirus genomes: a phylogenetic analysis. J Gen Virol 85, 105-117) and which is incorporated herein by reference in entirety and for all purposes.

TABLE 6
Minimal Gene Complement of Chordopoxviruses.

| ORF | Putative function | ORF | Putative function |
|---|---|---|---|
| F9L* | Unknown | H6R* | DNA topoisomerase I |
| F10L* | IMV serine-threonine protein | H7R | Unknown |
| | kinase | | |
| F12L | IEV protein | D1R* | mRNA capping enzyme, large |
| | | | subunit |
| F13L | EEV protein/phospholipase | D2L | IMV core protein |
| F15L | Unknown | D3R* | IMV core protein |
| F17R | IMV core phosphoprotein, | D4R* | Uracil-DNA glycosylase |
| | VP11/DNA-binding protein | | |
| E1L* | Poly(A) polymerase catalytic | D5R* | Nucleoside triphosphatase |
| | subunit | | |
| E2L | Unknown | D6R* | Early transcription factor small |
| | | | subunit, VETF-1 |
| E4L | Poly(A) polymerase catalytic | D7R* | RNA polymerase subunit rpo18 |
| | subunit, rpo30/VITF-1 | | |
| E6R* | Unknown | D9R | 29 kDa mutT-like protein |
| E8R | Unknown | D10R* | 29 kDa mutT-like protein, |
| | | | negative regulator of gene |
| | | | expression |
| E9L | DNA polymerase | D11L* | Nucleoside triphosphate |
| | | | phosphohydrolase I |
| E10R | IMV membrane-associated protein | D12L* | mRNA capping enzyme small |
| | | | subunit, intermediate transcription |
| | | | factor, VITF |
| I1L | IMV core/DNA-binding protein | D13L* | IMV protein, rifampicin resistance |
| I2L | Unknown | A1L* | Late transcription factor/VLTF-2 |
| I3L | Phosphoprotein, binds ssDNA | A2L* | Late transcription factor/VLTF-3 |
| I5L | IMV structural protein, VP13K | A2-5L | Thioredoxin-like protein |
| I6L | Unknown | A3L* | IMV major core protein, P4b |
| I7L* | IMV core protein | A4L | IMV core protein |
| I8R* | Nucleoside triphosphate | A5R* | RNA polymerase subunit rpo19 |
| | phosphohydrolase II, RNA | | |
| | helicase, NTPase | | |
| G1L* | Metallo-endoproteinase/virion | A6L | Unknown |
| | morphogenesis | | |
| G2R | Late transcription/IBT-dependent | A7L* | Early transcription factor large |
| | protein | | subunit, VETF |
| G3L | Unknown | A8R | Intermediate transcription factor, |
| | | | VITF-3 |
| G4L | Glutaredoxin 2, membrane protein, | A9L* | IMV protein, role in |
| | virion morphogenesis | | morphogenesis |
| G5R* | Unknown | A10L* | IMV major core protein P4a |
| G5-5R | RNA polymerase subunit rpo7 | A11R* | Unknown |
| G6R* | Unknown | A12L | IMV core protein |
| G7L | IMV core protein, VP16K | A13L | IMV membrane-associated |
| | | | protein/p8 |
| G8R | Late transcription factor, VLTF-1 | A14L | IMV protein, p16 |
| G9R* | Myristyl protein | A14-5L | IMV protein |
| L1R* | Myristylated IMV protein | A15L | Unknown |
| L2R | Unknown | A16L* | Myristyl protein |
| L3L* | Unknown | A17L | IMV membrane protein, |
| | | | morphogenesis factor |
| L4R* | IMV core protein VP8, DNA and | A18R* | DNA helicase, DNA-dependent |
| | RNA-binding protein | | ATPase, transcript release factor |
| L5R* | Unknown | A19L | Unknown |
| J1R | Dimeric virion protein | A20R | DNA polymerase processivity |
| | | | factor |
| J3R* | Poly(A) polymerase stimulatory | A21L* | Unknown |
| | submit, VP39 | | |
| J4R | RNA polymerase subunit rpo22 | A22R* | Holiday junction resolvase |
| J5L* | Unknown | A23R* | Intermediate transcription factor, |
| | | | VITF-3 |
| J6R* | RNA polymerase subunit rpo147 | A24R* | RNA polymerase subunit rpo132 |
| H1L | Tyrosine-serine phosphatase, virion | A28L* | Unknown |
| | maturation | | |
| H2R* | Unknown | A29L* | RNA polymerase subunit rpo35 |
| H3L* | Immunodominant IMV envelope | A30L | Unknown |
| | protein p35 | | |
| H4L* | RNA polymerase-associated | A32L* | ATP- and GTP-binding motif A, |
| | transcription specificity factor, | | DNA packaging |
| | RAP 94 | | |
| H5R | Late transcription factor, VLTF-4 | A34R | EEV glycoprotein |

### Example 4. Construction of Recombinant Chimeric Poxviruses

Construction of Shuttle Vectors for Insertion of Foreign Gene Expression Cassettes into the Genome of the #33 Chimeric Poxvirus

To construct thymidine kinase (TK) shuttle vector. the left and right flanking sequences of the TK gene of #33 chimeric poxvirus were PCR-amplified from #33 genomic DNA using Q5 High-Fidelity 2× Master Mix (New England Biolabs Inc., Ipswich. MA) and the primers: 5′-GCGCATATGATCTATGGATTACCATGGATGACAACTC-3 (SEQ ID NO: 31) and 5′-CGTTTAACTCGTCTAATTAATTCTGTAC-3′ (left flank. SEQ ID NO: 32). 5′-CAGGTAAAAGTACAGAATTAATTAGACGAGTTAAACGAGCTCGTCGACGGATCC GCTAGCGGCCGCGGAGGTAATGATATGTATCAATCGGTGTGTAG-3′ (SEQ ID NO: 33) and 5′-GCGGAATTCGTAATTACTTAGTAAATCCGCCGTACTAGG-3′ (right flank: SEQ ID NO: 34). The two fragments were joined together using the method of gene splicing by overlapping extension. The resulting fragment was digested with NdeI and EcoRI and cloned into the same-cut plasmid pGPT to yield p33NC-TK. The flanking sequences of TK in the shuttle vector were confirmed by sequencing. p33NC-TK contains the left and right flanking sequences of TK separated by SacI, SalI, BamHI, NheI and NotI, and Escherichia coli guanine phosphoribosyltransferase (gpt) gene driven by the vaccinia virus (VACV) early promoter p7.5E as a transient dominant selectable marker.

The F14.5L shuttle vector was constructed similarly. The left and right flanking sequences of the F14.5L gene of #33 chimeric poxvirus were PCR-amplified from #33 genomic DNA using Q5 High-Fidelity 2× Master Mix (New England Biolabs Inc., Ipswich, MA) and the primers: 5′-GCGCATATGTAGAAGAATTGATAAATATGAAACCTTTTAAG-3′ (SEQ ID NO: 35) and 5′-CCTCTCTAGCTTTCACTTAAACTGTATCG-3′ (left flank: SEQ ID NO: 36). 5′-GAATAATCGATACAGTTTAAGTGAAAGCTAGAGAGGAAGCTTGAGCTCGAGGAT CCGCTAGCGGCCGCTGAAGAGGATGCTAGAATCAAGGAGGAGCAAG-3′ (SEQ ID NO: 37) and 5′-GCGGAATTCTCCGGGCAGTGACTTTGTAGCTCTCCCAG-3′ (right flank: SEQ ID NO: 38). The two fragments were joined together using the method of gene splicing by overlapping extension. The resulting fragment was digested with NdeI and EcoRI and cloned into the same-cut plasmid pGPT to yield p33NC-F14.5L. The flanking sequences of F14.5L in the shuttle vector were confirmed by sequencing. p33NC-F14.5L contains the left and right flanking sequences of F14.5L, separated by HindIII, SacI, XhoI, BamHI, NheI and NotI, and Escherichia coli gpt driven by the VACV early promoter p7.5E as a transient dominant selectable marker.

### Example 5. Recombinant Chimeric Poxvirus Expressing CD19t Provide a Target for CAR Targeted to CD19

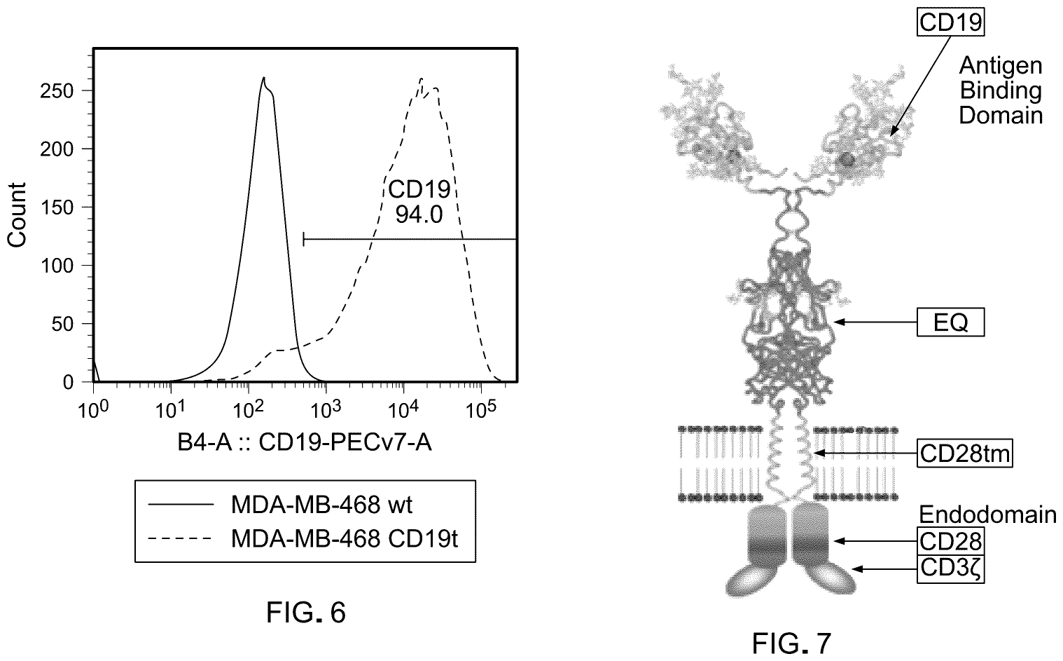

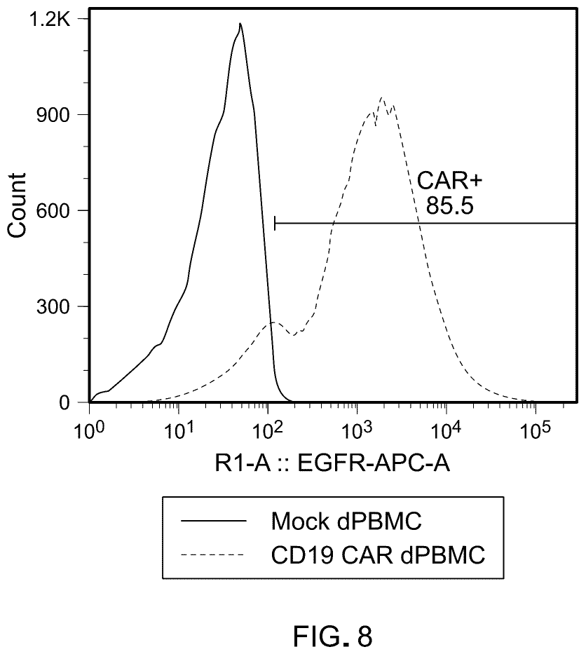

Two breast cancer cell lines, MDA-MB-468 wt (a triple negative breast cancer cell line), the parental line, and a variant of MDA-MB-468 modified to express a truncated human CD19 lacking a signaling domain (MDA-MB-468-CD19t) were used in the studies below. As shown in FIG. 6, MDA-MB-468-CD19t expresses CD19t. T cells modified to express a CAR targeted to CD19, described in greater detail below, were also used. The CAR expressed by these T cells is shown schematically in FIG. 7 and includes: a scFv targeted to CD19, and IgG4 spacer, a CD28 transmembrane domain, a CD28 co-stimulatory domain, and CD3zeta. A sequence encoding this CAR and a truncated EGFR was inserted into a lentiviral vector and this recombinant vector was used to transduce PBMC. The truncated EGFR can be used as marker for expression of the CAR. As shown in FIG. 8, which compares EGFR expression by mock transfected PBMC and PBMC transfected with the recombinant lentivirus expressing the CD19 CAR and truncated EGFR, show that the transfected cells express effectively express the transgene.

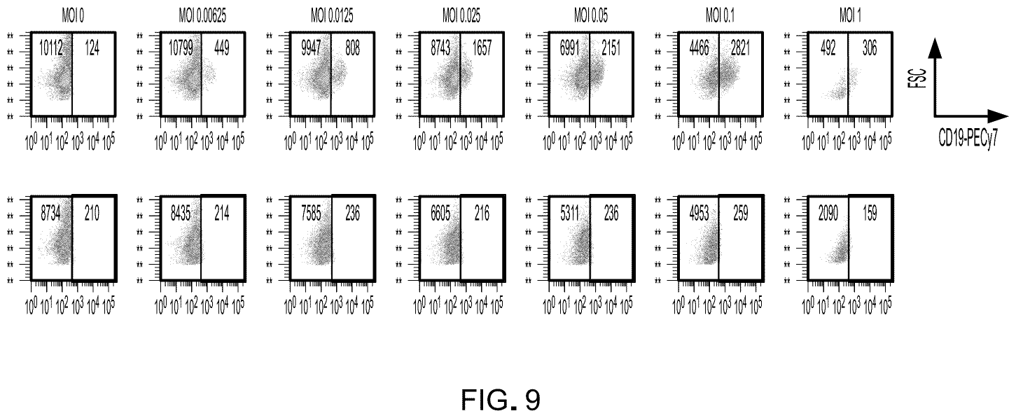

Chimeric poxvirus #33 was engineered to express truncated CD19 recombinant oncolytic virus (33-CD19t). MDA-MB-48 tumor cells, which do not express CD19, were exposed to recombinant oncolytic virus 33-CD19t at various MOI and to mock transfected T cells. As shown in the upper panels of FIG. 9, increased levels of recombinant oncolytic virus 33-CD19t increased CD19 expression and reduced cell number. The lower panel of FIG. 9 depicts the results when the MDA-MB-48 tumor cells were exposed to recombinant oncolytic virus 33-CD19t, at various MOI, and to transfected T cells expressing the CD19 targeted CAR. As can be seen, the number of CD19t-expressing MDA-MB-468 cells was greatly reduced.

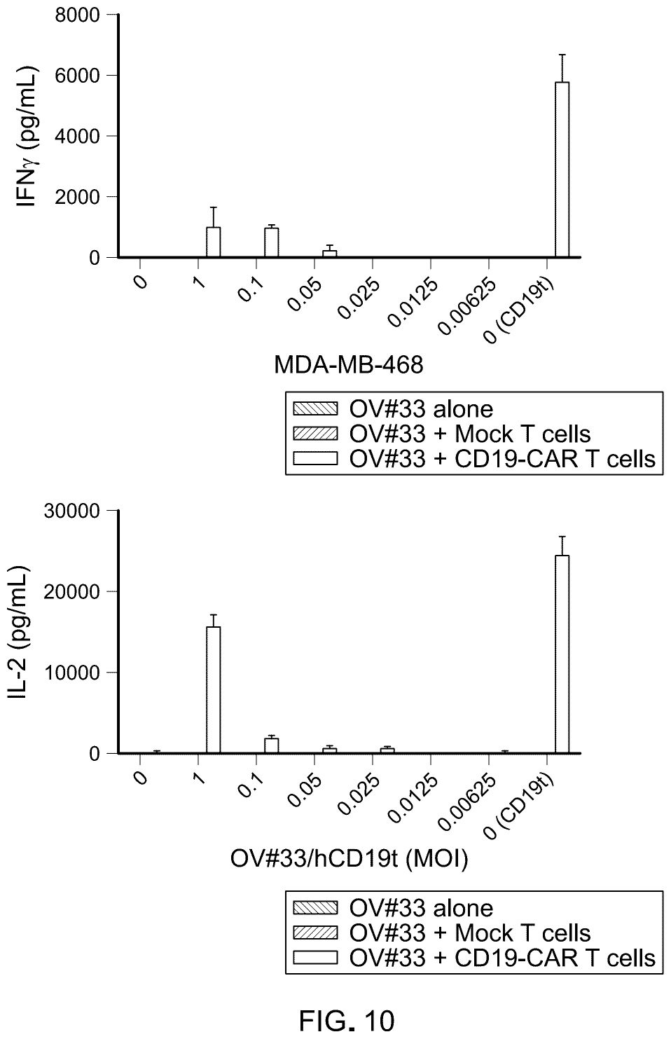

FIG. 10 depicts the results of a study measuring IL-2 (lower panel) and IFN-gamma (upper panel) levels in a long term killing assay. In this study, MDA-MB-468 cells were exposed to recombinant oncolytic virus 33-CD19t only: recombinant oncolytic virus 33-CD19t and mock transfected T cells: or recombinant oncolytic virus 33-CD19t and transfected T cells expressing the CD19 targeted CAR.

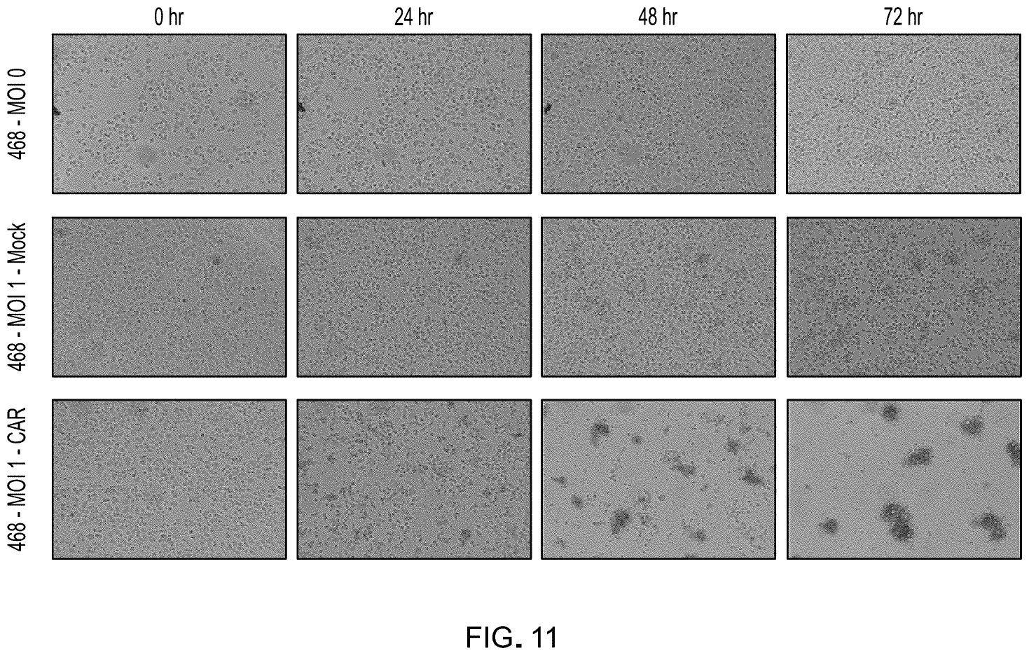

FIG. 11 is a series of images of cell cultures from a cell killing assay. In this study, MDA-MB-468 cells were exposed to recombinant oncolytic virus 33-CD19t at MOI=1 in the presence of mock transfected T cells or transfected T cells expressing the CD19 targeted CAR. The top series of images are of MDA-MB-468 cells cultured in the absence of either recombinant oncolytic virus 33-CD19t or transfected T cells expressing the CD19 targeted CAR.

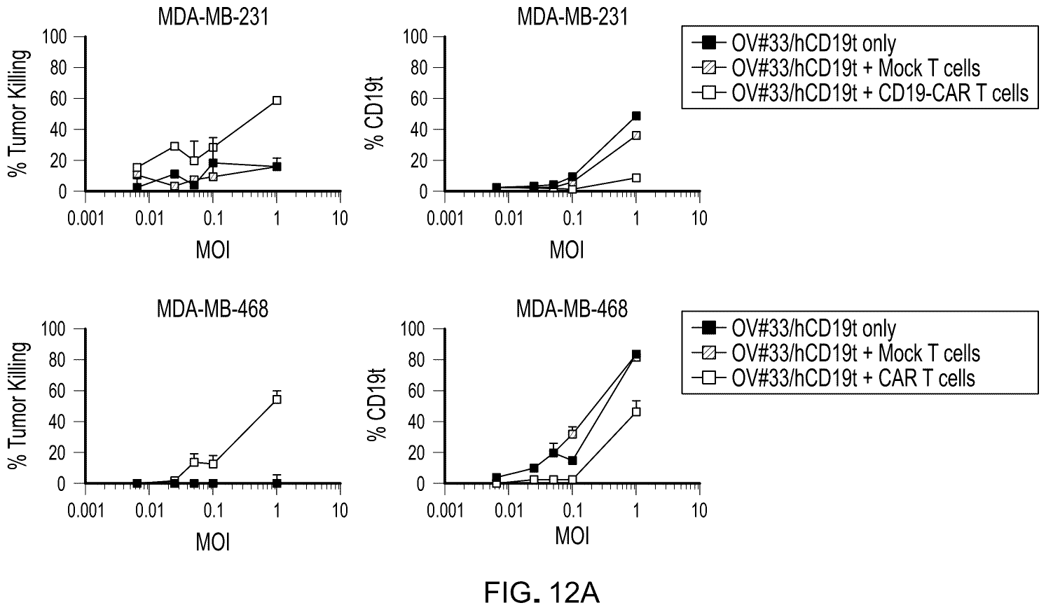

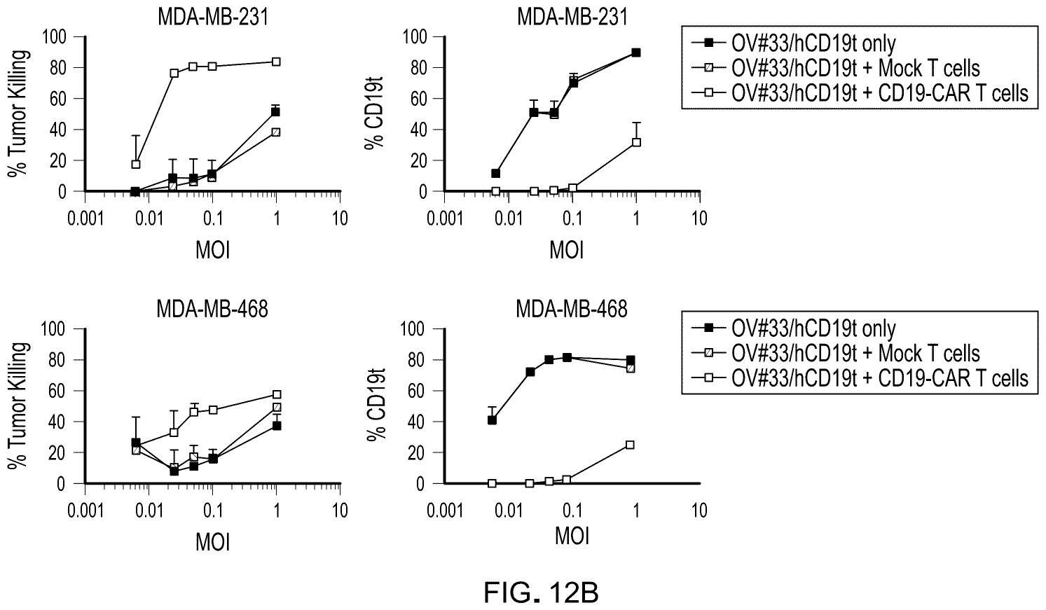

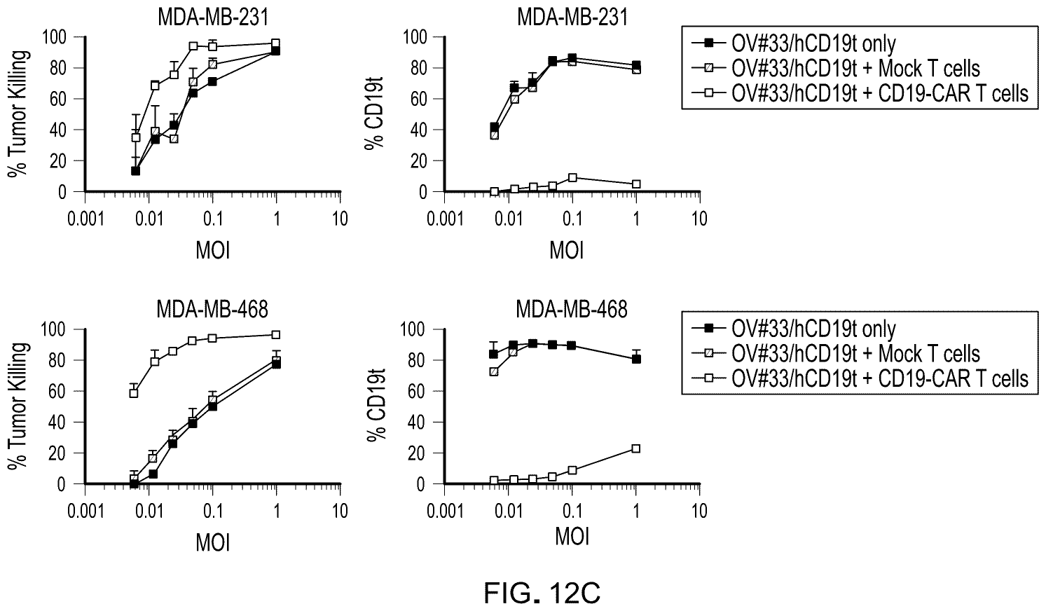

FIGS. 12A-12C depict the results of a long term cell killing assay in which MDA-MB-468 cells or MMDA-MB-231 cells were cultured with recombinant oncolytic virus 33-CD19t only (at various MOI): recombinant oncolytic virus 33-CD19t (at various MOI) and mock transfected T cells: or recombinant oncolytic virus 33-CD19t (at various MOI) and transfected T cells expressing the CD19 targeted CAR. Tumor cell killing and CD19t expression were measured.

Taken together, the studies in this example demonstrate that: 1) in MDA-MB-48 cells exposed to recombinant oncolytic virus 33-CD19t, CD19t expression increases with higher MOI: 2) in MDA-MB-48 cells exposed to recombinant oncolytic virus 33-CD19t in the presence of CD19-CAR T cells, the T cells produce higher levels of IL-2 and IFN-gamma as the MOI is increased; and 3) CD19 CAR T cells show efficacy against in MDA-MB-48 cells exposed to recombinant oncolytic virus 33-CD19t.

### Example 6. Recombinant Chimeric Poxvirus Expressing CD19t Together with a CD19 CAR are Effect in a TNBC Xenograft Model

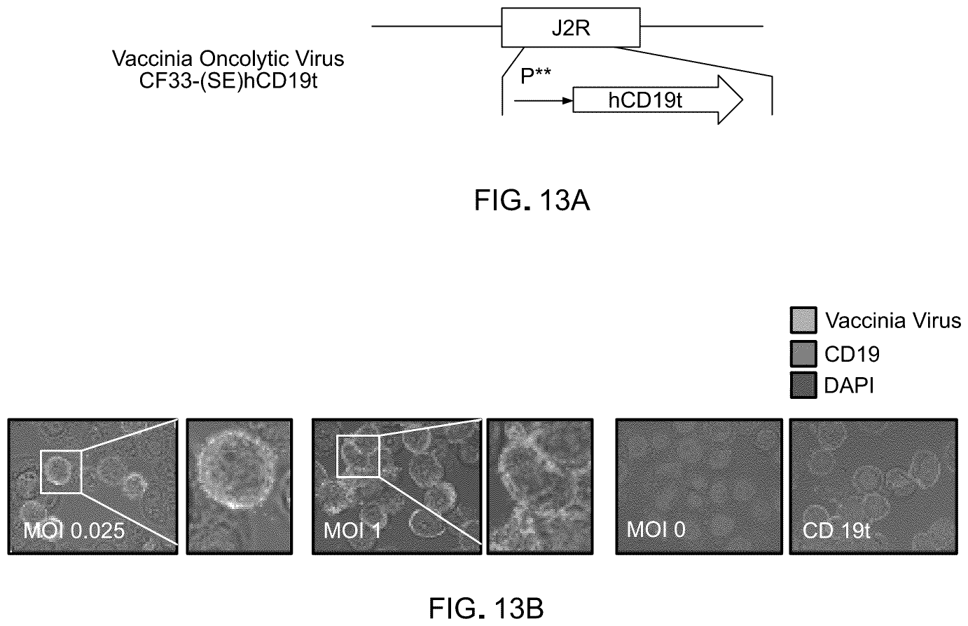

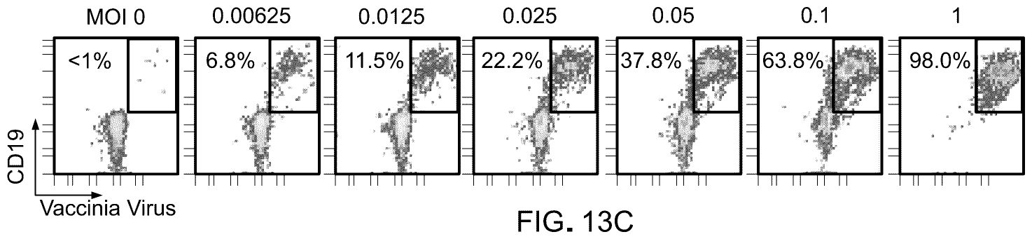

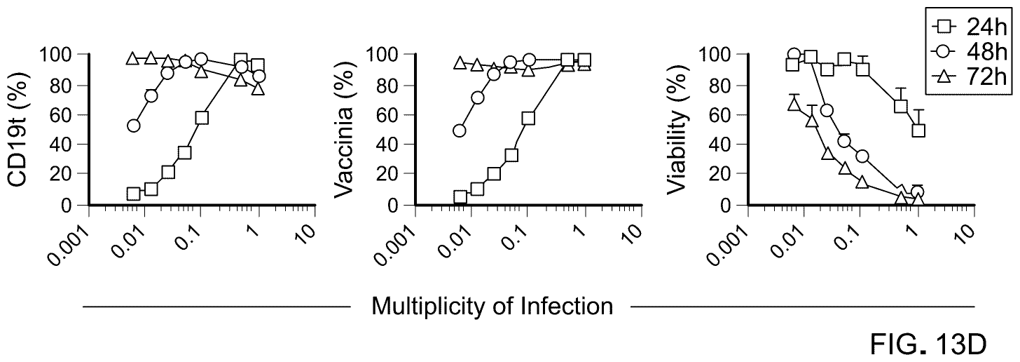

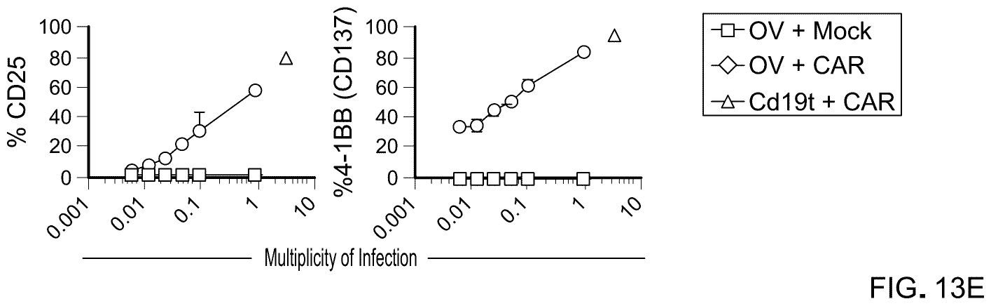

As shown in FIGS. 13A-13E, an oncolytic virus can effectively deliver CD19t to solid tumors and activate CD19-CAR T cells in vitro. FIG. 13A is a schematic of vaccinia oncolytic virus [CF33-(SE)hCD19t], showing incorporation of human truncated CD19 (CD19t) under the control of the synthetic early promoter (P_{SE}) inserted into the J2R locus replacing the thymidine kinase gene. FIG. 13B depicts munofluorescence microscopy of MDA-MB-468 cells infected for 24 h with OV19t at MOI 0.025 (left) and MOI 1 (middle), or cells transduced with lentivirus to stably express CD19t (right). Images are at 10× magnification, and insets are at 40× magnification. FIG. 13C is a set of FACS plots displaying the cell surface expression of CD19t and intracellular expression of vaccinia virus on MDA-MB-468 tumor cells determined by flow cytometry after 24 h of OV19t infection at increasing MOIs. FIG. 13D presents quantification of CD19t (left), vaccinia (middle), and viability (right) of MDA-MB-468 tumor cells following 24, 48, and 72 h co-culture with indicated MOIs of OV19t. FIG. 13E show a quantification of CD25 (left) and CD137 (right) expression on Mock (untransduced) or CD19-CAR T cells following a 24 hour co-culture with tumor cells at an effector:tumor (E:T) ratio of 1:2 with or without treatment with indicated MOI of OV19t.

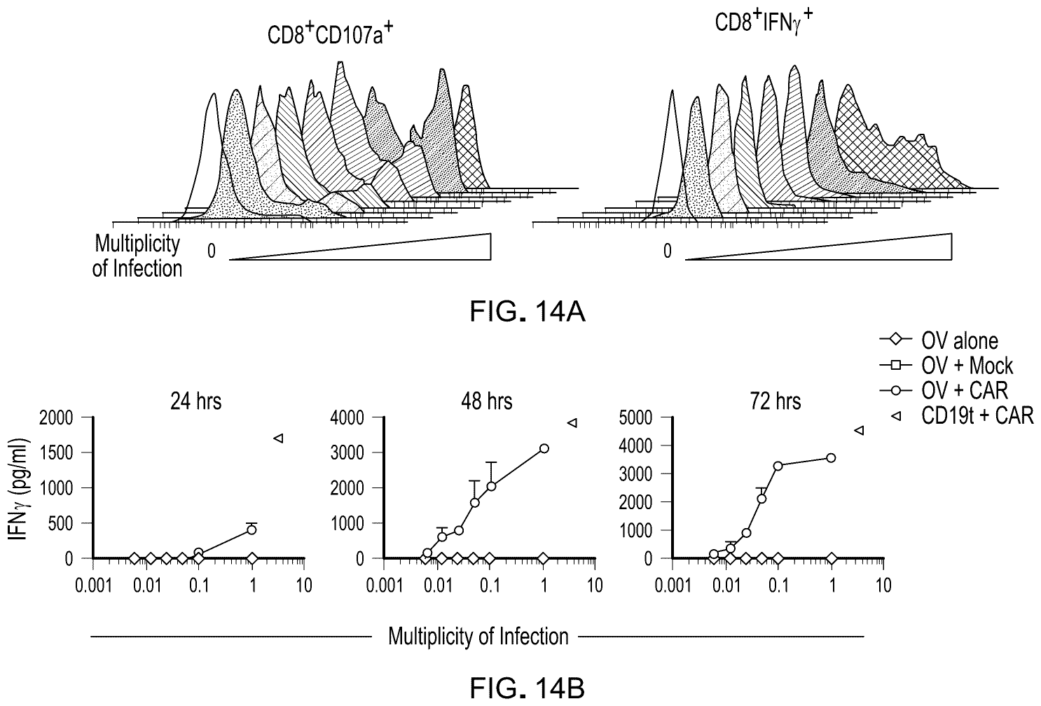

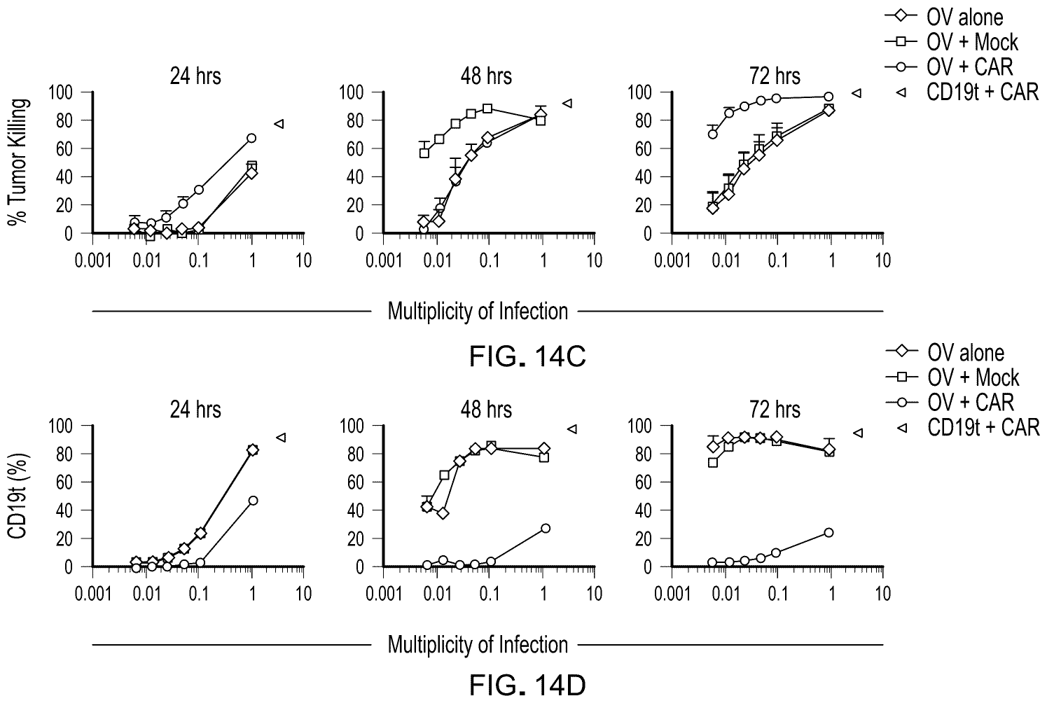

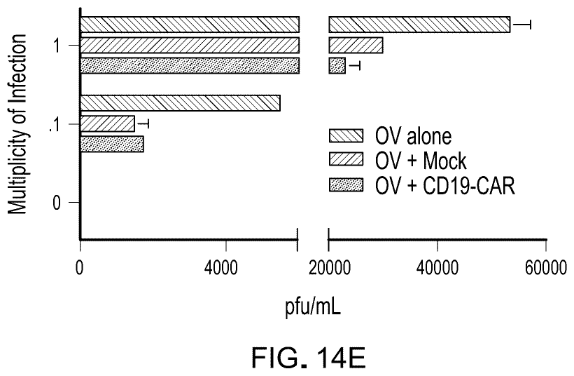

As shown in FIGS. 14A-14E, OV19t oncolytic virus can force expression of CD19t in tumor cells, which re-direct activation and cytotoxicity of CD19-CAR T cells in vitro. FIG. 14A is a representative flow cytometric analysis showing cell surface CD107a (left) and intracellular IFNγ expression (right) in CD8⁺ CAR⁺ T cells following a 16 h co-culture with MDA-MB-468 tumor cells at a 1:1 E:T ratio with or without treatment with indicated MOI of OV19t. FIG. 14B shows IFNγ production measured by ELISA in supernatants collected from co-cultures with or without treatment with OV19t at indicated MOIs for 24, 48, and 72 h. FIG. 14C shows tumor killing assay assessed by flow cytometry comparing Mock or CD19-CAR T cells following a 24, 48, or 72 h co-culture with MDA-MB-468 tumor cells treated with indicated MOIs of OV19t. FIG. 14D show CD19t expression on tumor cells in killing assay described in panel C. FIG. 14E shows virus titers in supernatant collected from co-cultures of T cells and tumors cells treated with indicated MOIs of OV19t.

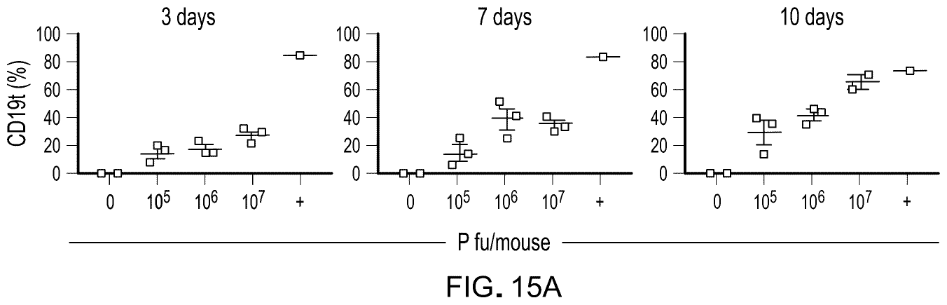

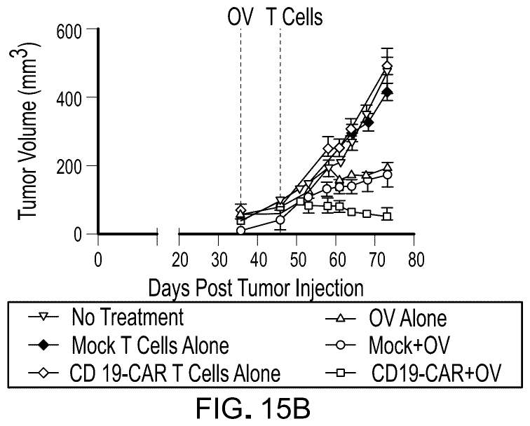

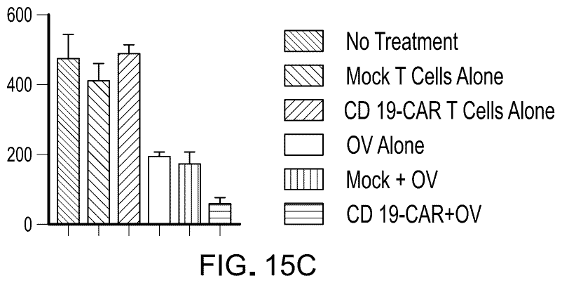

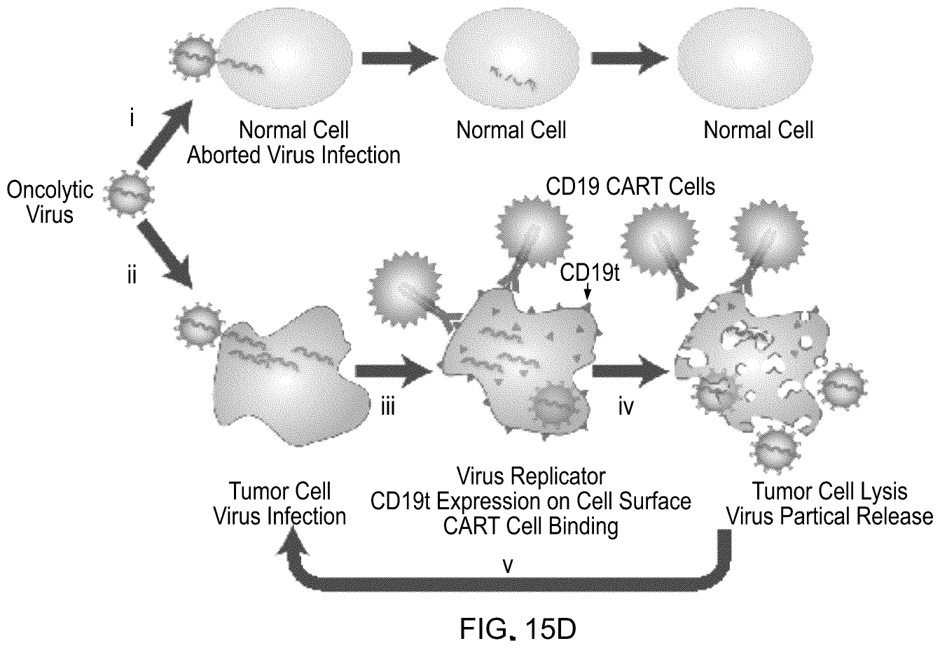

As shown in FIGS. 15A-15D, combination therapy with OV19t and CD19-CAR T cells is effective in a TNBC xenograft model. FIG. 15A shows ice were engrafted with subcutaneous MDA-MB-468 tumors (5×10⁶ cells) and intratumorally treated with 0, 10⁵, 10⁶, or 10⁷ plaque-forming units (pfu) per mouse, which were harvested at day 3 (left), 7 (middle), or 10 (right) after treatment for quantification of CD19t expression via flow cytometry. (+) represents MDA-MB-468 tumors previously lentivirally transduced to stably express CD19t. FIG. 15B shows tumor volume (mm³) in NSG mice bearing subcutaneous MDA-MB-468 (5×10⁶ cells) tumors on day 0, and treated with OV19t (10⁷ pfu) on day 36. On day 46, mice were treated with either Mock or CD19-CAR T cells (5×10⁶ cells). FIG. 15C shows average tumor volumes on day 73 of in vivo experiment described in panel B. FIG. 15D is a schematic of combination therapy concept utilizing OV to introduce CAR targets to intractable solid tumors.

### REFERENCES

1. Chen N G & Szalay A A (2011) Oncolytic virotherapy of cancer. Cancer Management in Man: Chemotherapy, Biological Therapy, Hyperthermia and Supporting Measures, Cancer Growth and Progression, ed Minev BR (Springer, New York), Vol 13, pp 295-316.
2. Chen N G & Szalay A A (2010) Oncolytic vaccinia virus: a theranostic agent for cancer. Future Virol. 5 (6):763-784.
3. Andtbacka R H, et al. (2015) Talimogene Laherparepvec Improves Durable Response Rate in Patients With Advanced Melanoma. J Clin Oncol 33 (25):2780-2788.
4. Anonymous (2015) First Oncolytic Viral Therapy for Melanoma. Cancer discovery.
5. Russell S J, Peng K W, & Bell J C (2012) Oncolytic virotherapy. Nat Biotechnol 30 (7):658-670.
6. Thorne S H, et al. (2007) Rational strain selection and engineering creates a broad-spectrum, systemically effective oncolytic poxvirus, JX-963. J Clin Invest 117 (11):3350-3358.
7. Yu W & Fang H (2007) Clinical trials with oncolytic adenovirus in China. Curr Cancer Drug Targets 7 (2):141-148.
8. Evgin L, et al. (2010) Potent Oncolytic Activity of Raccoonpox Virus in the Absence of Natural Pathogenicity. Mol Ther.
9. Rintoul J L, et al. (2012) ORFV: a novel oncolytic and immune stimulating parapoxvirus therapeutic. Mol Ther 20 (6):1148-1157.
10. Chan W M & McFadden G (2014) Oncolytic Poxviruses. Annu Rev Virol 1 (1):119-141.

1. A recombinant oncolytic virus comprising a nucleotide sequence encoding a truncated human CD19 lacking a functional signaling domain and comprising amino acids 22-323 of SEQ ID NO:29, wherein the nucleotide sequence encoding the truncate truncated CD19 is operably linked to a promoter.

2. The recombinant oncolytic virus of claim 1 wherein the oncolytic virus is a poxvirus.

3. The recombinant oncolytic virus of claim 2 wherein the poxvirus is a chimeric poxvirus.

4. The recombinant oncolytic virus of claim 3 wherein the chimeric poxvirus comprises at least 100 contiguous nucleotides of at least two poxvirus strains selected from the group consisting of: of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS.

5. The recombinant oncolytic virus of claim 1 wherein the portion excluding the nucleotide sequence encoding the truncated human CD19 is at least 98% identical to SEQ ID NO:1 or SEQ ID NO:2, but lacks a sequence encoding thymidine kinase.

6. The recombinant oncolytic virus of claim 1 wherein the promotor is a viral early promoter.

7. The recombinant oncolytic of claim 2 wherein the promoter is a poxvirus early promoter.

8. A method for treating a patient suffering from cancer comprising:
administering to the patient the recombinant oncolytic virus of claim 1; and, simultaneously or subsequently,
administering to the patient a T cell population expressing a chimeric antigen receptor (CAR) that is targeted human CD19.

9. The method of claim 8 wherein the T cell population comprises T cells transduced with a lentiviral vector encoding the CAR.

10. The method of claim 9 wherein the T cell population comprises T cells transfected with an RNA molecule encoding the CAR.

11. The method of claim 9 wherein the T cell population is administered 1-20 days after administration of the recombinant oncolytic virus.

12. The method of claim 9 wherein the T cell population is administered 5-100 days after administration of the recombinant oncolytic virus.

13.-18. (canceled)

19. The method of claim 8 wherein the cancer is a solid tumor.

20. The method of claims 19 wherein the solid tumor is ovarian cancer or pancreatic cancer.

21. The recombinant oncolytic virus of claim 1 wherein the portion excluding the nucleotide sequence encoding the truncated human CD19 is at least 99% identical to SEQ ID NO: 1 or SEQ ID NO:2, but lacks a sequence encoding thymidine kinase.
